(19) United States
(12) Patent Application Publication
(10) Pub. No.: US 2026/0152750 A1
(43) Pub. Date: Jun. 4, 2026

(54) RNA-BASED COMPOSITIONS AND METHODS OF USE THEREOF

(71) Applicants: THE TRUSTEES OF PRINCETON UNIVERSITY, Princeton, NJ (US); CAMBRIDGE ENTERPRISE LIMITED, Cambridge, Cambridgeshire (GB)

(72) Inventors: Arend Jan Wouter TE VELTHUIS, Princeton, NJ (US); Emmanuelle PITRE, Le pallet (FR)

(21) Appl. No.: 19/109,267

(22) PCT Filed: Sep. 6, 2023
(86) PCT No.: PCT/US2023/073557

§ 371 Date: Mar. 6, 2025

Related U.S. Application Data
(60) Provisional application No. 63/403,914, filed on Sep. 6, 2022.

(51) Int. Cl. C12N 15/113; A61K 31/7105; A61K 39/00; A61P 31/16; C12N 9/99; G16B 15/10; G16B 15/30

(52) U.S. Cl. CPC C12N 15/1137; A61K 31/7105; A61K 39/001102; A61P 31/16; C12N 9/99; C12N 15/1131; G16B 15/10; G16B 15/30; C12N 2310/531; C12Y 207/07048

(57) ABSTRACT

The present disclosure relates to RNA sequences, compositions, and methods of use to prevent viral replication, prevent RNA polymerase activity, activate innate immune responses, or combinations thereof.

### CROSS REFERENCE TO RELATED APPLICATIONS

This application is a U.S. National Stage application filed under 35 U.S.C. § 371 of PCT/US2023/073557 filed Sep. 6, 2023, which claims the benefit of priority to U.S. Provisional Patent Application No. 63/403,914, filed Sep. 6, 2022, which are incorporated by reference herein in their entirety.

### REFERENCE TO SEQUENCE LISTING

The sequence listing submitted on Feb. 12, 2026, as an .XML file entitled “11676-003US1_ST26” created on Jan. 7, 2026, and having a file size of 928,518 bytes is hereby incorporated by reference pursuant to 37 C.F.R. § 1.52(e)(5).

### FIELD

The present disclosure relates to RNA sequences, compositions, and methods of use to prevent viral replication, prevent RNA polymerase activity, activate innate immune responses, or combinations thereof.

### BACKGROUND

Influenza A viruses (IAV) are important human pathogens that generally cause a mild to moderately severe respiratory disease. A range of viral, host, and bacterial factors can influence the outcome of infections with IAV. One important factor is the activation of host protein retinoic acid-inducible gene I (RIG-I) by double-stranded 5′ di- or triphosphorylated RNA. Activated RIG-I translocates to mitochondria, and triggers oligomerization of mitochondrial antiviral signaling protein (MAVS) and subsequent phosphorylation of IRF3 and NF-kB, leading to the expression of innate immune genes, including interferon-β (IFN-β) and IFN-λ. Innate immune gene expression typically leads to a protective antiviral state, but results in an overproduction of cytokines and chemokines when dysregulated. This phenomenon underlies the lethal pathology of infections with 1918 H1N1 pandemic or highly pathogenic avian IAV. Various viral and host factors have been implicated in causing immunopathology, including the products of aberrant viral replication.

The emergence of viral and/or host factors that contribute to immunopathological events within the host, presents the need to develop compositions and methods to prevent viral functions and activate the host immune systems against viral pathogens.

The compositions and methods disclosed herein address these needs.

### SUMMARY

The present disclosure provides ribonucleic acid (RNA) compositions and methods of use to prevent, reduce, and/or decrease viral replication, increase and/or activate an innate immune response, or a combination thereof to be used for treatment and/or prevention of an infection.

In one aspect, disclosed herein is an engineered RNA sequence comprising a stem-loop, wherein the stem-loop comprises a stem portion with at least 5 base pairs (bps) in length and a loop portion with about 20 nucleotides in length, wherein the loop portion matches a footprint of an RNA polymerase of a target virus, and wherein the stem loop is flanked by a 5′ promoter and a 3′ promoter RNA sequence of the target virus.

In some embodiments, the stem portion comprises at least 14 bps in length.

In some embodiments, the target virus comprises a negative-sense RNA virus. In some embodiments, the negative-sense RNA virus comprises Influenza A virus (IAV), Ebola virus, Nipah virus, Hanta virus, Hendra virus, Lassa virus, or Rabies virus.

In some embodiments, the RNA sequence comprises between about 40 to about 80 nucleotides. In some embodiments, the RNA sequence comprises at least 60% sequence identity to any one of SEQ ID NO: 4-30. In some embodiments, the RNA sequence comprises at least 70% sequence identity to any one of SEQ ID NO: 4-30. In some embodiments, the RNA sequence comprises at least 80% sequence identity to any one of SEQ ID NO: 4-30. In some embodiments, the RNA sequence comprises at least 90% sequence identity to any one of SEQ ID NO: 4-30. In some embodiments, the RNA sequence comprises any one of sequences selected from SEQ ID NO: 4-30.

In some embodiments, the footprint of the RNA polymerase comprises an area within the RNA polymerase capable of holding a designated number of nucleotides. In some embodiments, the designated number of nucleotides comprises about 20 nucleotides. In some embodiments, the 5′ promoter comprises about 12 nucleotides in length. In some embodiments, the 3′ promoter comprises about 13 nucleotides in length.

In one aspect, disclosed herein is a cell expressing the engineered RNA of any preceding aspect.

In one aspect, disclosed herein is a method of reducing viral replication, inducing activation of an innate immune response to a target virus, or a combination thereof, the method comprising engineering an RNA sequence comprising a stem-loop, wherein the stem-loop comprises a stem portion with at least 5 base pairs (bps) in length and a loop portion with about 20 nucleotides in length, wherein the loop portion matches a footprint of an RNA polymerase of a target virus, and wherein the stem loop is flanked by a 5′ promoter and a 3′ promoter RNA sequence of the target virus, and contacting the RNA sequence to the RNA polymerase of the target virus, wherein the RNA sequence forms a template loop (t-loop) around the RNA polymerase to reduce viral replication, and wherein the RNA sequence comprises an agonist to activate the innate immune response.

In one aspect, disclosed herein is a method of preventing RNA polymerase activity, the method comprising engineering an RNA sequence comprising a stem-loop, wherein the stem-loop comprises a stem portion with at least 14 base pairs (bps) in length and a loop portion with about 20 nucleotides in length, wherein the loop portion matches a footprint of an RNA polymerase of a target virus, and wherein the stem loop is flanked by a 5′ promoter and a 3′ promoter RNA sequence of the target virus, and contacting the RNA sequence to the RNA polymerase of the target virus, wherein the RNA sequence forms a template loop (t-loop) around the RNA polymerase to stop RNA polymerase activity.

In one aspect, disclosed herein is a method of treating and/or preventing a viral infection, the method comprising engineering an RNA sequence comprising a stem-loop, wherein the stem-loop comprises a stem portion with at least 5 base pairs (bps) in length and a loop portion with about 20 nucleotides in length, wherein the loop portion matches a footprint of an RNA polymerase of a target virus, and wherein the stem loop is flanked by a 5′ promoter and a 3′ promoter RNA sequence of the target virus, and administering the RNA sequence to a subject, wherein the RNA sequence contacts the RNA polymerase of the target virus, and wherein the RNA sequence reduces viral replication and/or activates the innate immune response in the subject relative to an untreated control subject.

In one aspect, disclosed herein is a method of performing a template loop (t-loop) analysis, the method comprising identifying a template loop RNA sequence, blocking off a portion of the template loop RNA sequence to represent a footprint of a virus RNA polymerase, determining a t-loop ΔG, an upstream ΔG for a stretch of 10 nucleotides of the footprint, and a downstream ΔG for a stretch of 10 nucleotides of the footprint, and determining a ΔΔG for a likelihood of t-loop formation by subtracting the upstream ΔG and downstream ΔG from the t-loop ΔG, and moving the footprint in a one-nucleotide increment along the template loop RNA sequence, and repeating step c) until the ΔΔG values have been determined for an entire template loop RNA sequence.

In some embodiments, the stem portion comprises at least 14 bps in length.

In some embodiments, the target virus comprises a negative-sense RNA virus. In some embodiments, the negative-sense RNA virus comprises Influenza A virus (IAV), Ebola virus, Nipah virus, Hanta virus, Hendra virus, Lassa virus, or Rabies virus.

In some embodiments, the RNA sequence comprises between about 40 to about 80 nucleotides. In some embodiments, the RNA sequence comprises any one of sequences selected from SEQ ID NO: 4-30.

In some embodiments, the footprint of the RNA polymerase comprises an area within the RNA polymerase capable of holding a designated number of nucleotides. In some embodiments, the designated number of nucleotides comprises about 20 nucleotides. In some embodiments, the 5′ promoter comprises about 12 nucleotides in length. In some embodiments, the 3′ promoter comprises about 13 nucleotides in length.

In some embodiments, the stem-loop inhibits the RNA polymerase from replicating a genome of the target virus. In some embodiments, the RNA sequence forms a template loop (t-loop) around the RNA polymerase to reduce viral replication.

In some embodiments, the innate immune response comprises binding a host pathogen receptor to the RNA sequence. In some embodiments, the host pathogen receptor comprises a retinoic acid-inducible gene I (RIG-I).

In one aspect, disclosed herein is a non-transitory computer-readable storage medium comprising instructions that, when executed, cause at least one processor to perform the method of any preceding aspect.

In one aspect, disclosed herein is a computer-implemented method for generating at least one optimal ribonucleic acid (RNA) sequence for reducing viral replication and/or inducing activation of an innate response to a target virus, the computer-implemented method comprising retrieving, by one or more processors, an RNA polymerase of the target virus, performing, by the one or more processors, a template loop (t-loop) analysis operation on the RNA polymerase, determining, by the one or more processors, at least one optimal RNA sequence corresponding with the RNA polymerase of the target virus based on results of the t-loop analysis operation, and outputting, by the one or more processors, a ΔG, a location of the ΔG, a t-loop structure, or combinations thereof. In some embodiments, the output comprises the at least one optimal RNA sequence.

In some embodiments, the computer-implemented method further comprises receiving, by the one or more processors, one or more user-defined parameters associated with the RNA polymerase of the target virus, and performing, by the one or more processors, the t-loop analysis operation based at least on the one or more user-defined parameters. In some embodiments, the t-loop analysis operation is used interchangeably with a sliding window operation. In some embodiments, the computer-implemented method further comprises determining, by the one or more processors, at least one criterion of a composition for administration to a subject based on the at least one optimal RNA sequence.

### BRIEF DESCRIPTION OF FIGURES

The accompanying figures, which are incorporated in and constitute a part of this specification, illustrate several aspects described below.

FIGS. 1A, 1B, 1C, and 1D show the sequence-dependent reduction of mvRNA replication and induction of IFN-β promoter activation. FIG. 1A shows the schematic of mvRNA formation via intramolecular template switching. This process has the potential to create novel RNA structures. Produced mvRNAs are bound by RIG-I, leading to the expression of innate immune responses. FIG. 1B shows the IFN-β promoter activation following transfection of in vitro transcribed segment 5 mvRNAs of 47 or 76 nt in length. FIG. 1C shows the replication of model mvRNAs in HEK 293T cells by the influenza virus A/WSN/33 (H1N1) RNA polymerase. RNA levels were analyzed by primer extension and the ability of mvRNA replication to induce IFN-β promoter activity was analyzed using a luciferase-based IFN-β reporter assay. Non-specific primer extension signals in FIG. 1C are indicated with *. FIG. 1D shows the RT-qPCR analysis of IFN-β mRNA levels. Data from three biological repeats are shown.

FIGS. 2A, 2B, and 2C show that t-loops induce RNA polymerase stalling. FIG. 2A shows the schematic of RNA structure formation upstream, around (t-loop), and downstream of the RNA polymerase. FIG. 2B shows the ΔG values for RNA structures forming upstream (green), around (t-loop; orange) or downstream (purple) of the RNA polymerase were computed using a sliding window approach. The difference in ΔG (ΔΔG) between the formation of a t-loop and structures forming upstream or downstream the RNA polymerase was computed and shown in the bottom graph. Heatmap shows zoom in on ΔΔG values computed for middle of the mvRNA templates used. FIG. 2C shows the replication of the NP71.1, NP71.2 or the destabilized NP71.6 mvRNA templates in HEK 293T cells by the WSN RNA polymerase. RNA levels were analyzed by primer extension and the ability of mvRNA replication to induce IFN promoter activity was analyzed using a luciferase reporter assay. Non-specific primer extension signals in FIG. 2C are indicated with *. Data from three biological repeats are shown.

FIGS. 3A, 3B, and 3C show that mvRNA t-loops stall RNA polymerase activity and induce IFN-β promoter activation. FIG. 3A shows the heat map showing ΔΔG as estimate for the likelihood of t-loop formation. FIG. 3B shows the replication of mvRNA templates. Top panel shows primer extension analysis. Graphs show quantification of template loop RNA level and IFN-j promoter activation. Data from three biological repeats are shown. FIG. 3C shows the analysis of IAV RNA polymerase activity in vitro.

FIGS. 4A, 4B, 4C, 4D, and 4E show the mutation near template exit channel increases RNA polymerase sensitivity to t-loops. FIG. 4A shows the structure of the pre-termination complex of the bat influenza A virus RNA polymerase (PDB 6SZU). The 5′ and 3′ ends of the template are shown in black and gold, respectively. The body of the template is shown in dark blue and the nascent RNA in red. Location of PB1 K669 and PB2 T81 are indicated. FIG. 4B shows the amino acid alignment of PB1 C-terminus. PB1 K669 is indicated with an arrow. FIG. 4C shows the IFN-j promoter activity and segment 6 viral RNA levels in the presence of wildtype and K669A RNA polymerases. For the segment 6 vRNA template, the mRNA, cRNA and vRNA species are indicated. 5S rRNA and western blot for PB1 subunit expression are shown as loading control.

FIGS. 4D and 4E show the IFN-β promoter activity and mvRNA template levels for five NP71 mvRNA templates in the presence of wildtype and K669A RNA polymerases. RNA levels were analyzed by primer extension. 5S rRNA and western blot for PB1 subunit, NP, and tubulin expression are shown as loading control. Data from three biological repeats are shown.

FIGS. 5A, 5B, 5C, 5D, 5E, 5F, and 5G show the reduced mvRNA replication in viral infections is correlated with IFN-β promoter activation. FIGS. 5A and 5B show the number of mvRNA copies per unique mvRNA sequence detected in ferret lungs infected with BM18 or A549 cells infected with WSN. In each graph two biological repeats are shown. FIG. 5C shows the ΔΔG heat map of negative sense template of WSN segment 2 derived mvRNAs. Half of the mvRNA template is indicated with a dotted line. FIG. 5D shows the replication of segment 2-derived mvRNAs identified by NGS in HEK 293T cells by the WSN RNA polymerase. RNA levels were analyzed by primer extension. The ability of mvRNA replication to induce IFN-β promoter activity was analyzed using a luciferase reporter assay. Asterisk (*) indicates non-specific radioactive signal. Data from four biological repeats are shown. FIG. 5E shows the IFN-β induction is negatively correlated with template replication level. PB1-mvRNA G and I were excluded from the fit to the exponential decay because they are shorter than the IFN-β promoter induction cut-off of 56 nt. FIG. 5F shows the IFN-β induction is negatively correlated with ΔΔG of first half of template mvRNA. FIG. 5G shows the schematic of aberrant RNA synthesis by the IAV RNA polymerase. T-loops present in some mvRNAs lead to reduced RNA polymerase processivity. This may induce template release and/or binding of the mvRNA template to RIG-I. Host factor ANP32A, which plays a key role during cRNA and vRNA synthesis, is not shown for clarity.

FIGS. 6A, 6B, 6C, and 6D show the effect of a single spinach aptamer on mvRNA replication. FIG. 6A shows how to rule out that a single RNA structure upstream of the RNA polymerase reduces activity, the NP56 mvRNA template was engineered and the RNA aptamer Spinach was inserted, creating NP56-S1. FIG. 6B shows a Spinach-containing mvRNAs generated in vitro demonstrated fluorescence after addition of DFHBI relative to the 56-nt control mvRNA, confirming that the aptamer folds properly in the context of the IAV promoter sequence. FIG. 6C shows the TIRF microscopy image of a 56-nt mvRNA or Spinach-containing mvRNA generated in vitro in the presence of DFHBI. FIG. 6D show the after transfection of plasmids encoding the Spinach-containing mvRNA NP56-SI into HEK293T cells and analysis of the RNA produced using primer extension, no difference in replication was observed relative to our NP56 control mvRNA.

FIG. 7 shows the fractionation of HEK 293T cells transfected with IAV RNA polymerase and a 76 nt-long segment 5-derived mvRNA. Top panel shows primer extension analysis of RNA extracted from fractions. Bottom four panels show western blot analysis. For each lane, comparable amounts were loaded.

FIGS. 8A, 8B, and 8C show the aberrant RNA sequences, effect of exogenous mvRNAs on infection, and re-transfection of total RNA. FIG. 8A shows the alignment of aberrant RNA products isolated from in vitro replication assays. FIG. 8B shows the IFN-β promoter activity induced by the re-transfection of total RNA isolated from HEK 293T cells transfected with plasmids expressing segment 5-derived mvRNAs and the viral RNA polymerase subunits. Luciferase signal was normalized to the NP47 mvRNA, which does not trigger innate immune responses. FIG. 8C shows the induction of IRF3 phosphorylation by pre-transfected, exogenous mvRNAs during infection with IAV A/WSN/33 (H1N1). mvRNA amplification could not be detected by primer extension, likely because exogenous templates poorly compete with endogenous viral RNAs for replication.

FIGS. 9A, 9B, and 9C show the characterization of MAVS^{-/-} HEK 293 cells and mvRNA replication in MAVS^{-/-} HEK 293 cells. FIG. 9A shows the western blot analysis of MAVS expression in wildtype or M 4VS HEK 293 cells. FIG. 9B shows the IFN-β promoter activity in wildtype or MAVS^{-/-} HEK 293 cells following transfection of a plasmid expressing MAVS-flag. Bottom panel shows western blot of MAVS-flag expression. FIG. 9C shows the IFN-β promoter activity of HEK 293 MAVS^{-/-} cells expressing mvRNAs NP71.1-NP71.5. and primer extension analysis of mvRNA NP71.1-NP71.5 replication in HEK 293 MAVS^{-/-} cells. P-values were computed using student t-test.

FIG. 10 shows the replication of model mvRNAs in HEK 293T cells by the IAV A/Brevig Mission/1/18 (BM18) or A/duck/Fujian/01/02 (FJ02) RNA polymerases. The ability of these reactions to induce IFN-β promoter activity was analyzed using a luciferase reporter assay.

FIGS. 11A, 11B, 11C, 11D, and 11E show the sliding-window analysis of the free energy (ΔG) of transient secondary RNA structure formation near the IAV RNA polymerase. FIG. 11A shows how to calculate the ΔG for a t-loop, 10 nt on either side of the polymerase were allowed to fold using the duplex-fold algorithm of the ViennaRNA package. To calculate the ΔG for alternative structures, co-fold of the ViennaRNA package was used. The ΔΔG for the likelihood of t-loop formation was calculated by subtracting the upstream and downstream ΔG values from the t-loop ΔG value. FIG. 11B shows an example of Python script output (“&” represents the loop structure). FIG. 11C shows the ΔΔG values for positive sense 71-nt long mvRNA templates. FIG. 11D shows the ΔΔG values for negative sense 71-nt long mvRNA templates. FIG. 11E shows the t-loop ΔG values for positive sense and negative sense NP47, NP56 and NP76 mvRNA templates.

FIGS. 12A, 12B, 12C, and 12D show the replication of engineered mvRNAs and their ability to induce IFN-β promoter activity. FIG. 12A shows the alignment of t-loops (shaded orange) in NP71 mvRNA templates. Location of RNA polymerase entry (En) and exit (Ex) channel is shaded gray. FIG. 12B shows the primer extension analysis of RNA extracted from HEK 293T cells expressing NP71 mvRNA templates. FIG. 12C shows the alignment of t-loops (shaded orange) of additional NP71 mvRNA templates. The location of RNA polymerase entry and exit channel is shaded gray. FIG. 12D shows the primer extension analysis of RNA extracted from HEK 293T cells expressing NP71 mvRNA templates. Second and third panels show western blot analysis. Top graph shows quantification of mvRNA template level, while bottom graph shows IFN-β promoter activity analysis using a luciferase reporter.

FIGS. 13A and 13B show the analysis of IAV RNA polymerase template binding and release on different mvRNA templates in vitro. FIG. 13A show the binding of radiolabeled RNA templates by mOrange-tagged, immobilized IAV RNA polymerase in the absence and presence of ApG and NTPs. FIG. 13B shows the binding of radiolabeled RNA products by mOrange-tagged, immobilized IAV RNA polymerase in the absence and presence of free inactive IAV RNA polymerase (PB1a).

FIGS. 14A and 14B show the selection of segment 2 mvRNAs. FIG. 14A shows the segment 2 mvRNA sizes and abundance in three next generation sequencing experiments. FIG. 14B shows the relation between mvRNA level in transfection assay and mvRNA read counts in next generation sequencing. The mvRNAs F, G and H are indicated.

FIGS. 15A and 15B show the relation between the mvRNA template length and the IFN-β promoter activity induction (FIG. 15A), or the mvRNA template length and mvRNA replication (FIG. 15B).

FIGS. 16A, 16B, and 16C show the segment 2 mvRNA replication in MAVS^{-/-} HEK 293 cells. FIG. 16A shows the IFN-β promoter activity measured after re-transfection of total RNA extracted from HEK 293T cells expressing segment 2 mvRNAs into HEK 293T cells. FIG. 16b shows the IFN-β promoter activity induced by expression of segment 2 mvRNAs in MAVS^{-/-} HEK 293 cells. FIG. 16C shows the primer extension analysis of segment 2 mvRNAs levels in wildtype and MAVS HEK 293 cells. P-values were computed using one-way ANOVA with multiple-corrections relative to template A or the empty plasmid control.

FIG. 17 shows the induction of IFN beta promoter activity by segment 3 and 4 mvRNAs. P-values were computed using one-way ANOVA with multiple-corrections relative to the NP71.1 mvRNA template.

FIG. 18 shows an example computing device.

FIG. 19 shows a flowchart diagram of a computer-implemented method for generating at least one optimal RNA sequence for reducing viral replication and/or inducing activation of an innate response to a target virus in accordance with certain embodiments of the present disclosure.

FIGS. 20A and 20B show that the RIG-I helicase is required for mvRNA extraction.

FIG. 21 shows that mvRNAs are more potent in IFN activation.

FIGS. 22A, 22B, and 22C show that RNA templates containing t-loops do not trigger template release in vitro.

FIGS. 23A and 23B show a radio-labeled product RNA binding assay and demonstrate that full length RNA is not released by the replication complex.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” means lowering of an event or characteristic (e.g., viral replication or viral infection). It is understood that this is typically in relation to some standard or expected value, in other words it is relative, but that it is not always necessary for the standard or relative value to be referred to. For example, “reduces viral infection” or “reduces viral replication” means reducing the rate viral infection or rate of viral replication relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of infection), during early onset (e.g., upon initial signs and symptoms of infection), or after an established development of infection.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “promoter,” as used herein, refers to a sequence in DNA that mediates the initiation of transcription by an RNA polymerase. Transcriptional promoters may comprise one or more of a number of different sequence elements as follows: 1) sequence elements present at the site of transcription initiation; 2) sequence elements present upstream of the transcription initiation site and; 3) sequence elements down-stream of the transcription initiation site. The individual sequence elements function as sites on the DNA, where RNA polymerases and transcription factors facilitate positioning of RNA polymerases on the DNA bind.

“Expression” as used herein refers to the process by which information from a gene is used in the synthesis of a functional gene product that enables it to produce a peptide/protein end product, and ultimately affect a phenotype, as the final effect.

As used herein, the term “polymerase” refers to an enzyme that synthesizes long chains of polymers or nucleic acids. RNA polymerases are used to assemble RNA molecules, respectively, by copying a nucleic acid template strand using base-pairing interactions.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

“Composition” refers to any agent that has a beneficial biological effect. Beneficial biological effects include both therapeutic effects, e.g., treatment of a disease or other undesirable physiological condition, and prophylactic effects, e.g., prevention of a disease or other undesirable physiological condition (including, but not limited to Influenza). The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, the composition disclosed herein comprises an engineered RNA sequence and a pharmaceutically effective carrier.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

A “nucleotide” is a compound consisting of a nucleoside, which consists of a nitrogenous base and a 5-carbon sugar, linked to a phosphate group forming the basic structural unit of nucleic acids, such as DNA or RNA. The four types of DNA nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material. Herein the terms “nucleic acid” and “polynucleotide” are used interchangeably throughout the disclosure.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

A “genome” refers to a complete set of genes or genetic material present within a cell, tissue, or organism, including, but not limited to pathogenic genomes (i.e.: viral genomes).

Variants comprising a fragment of a reference nucleotide sequence are contemplated herein. A “fragment” is a portion of a nucleotide sequence which is identical in sequence to but shorter in length than the reference sequence. A fragment may comprise up to the entire length of the reference sequence, minus at least one nucleotide. For example, a fragment may comprise from 5 to 1000 contiguous nucleotides of a reference polynucleotide. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides of a reference polynucleotide. Fragments may be preferentially selected from certain regions of a molecule, for example the 5′-terminal region and/or the 3′ terminal region of a polynucleotide. The term “at least a fragment” encompasses the full-length polynucleotide.

A “host” refers to any animal (either vertebrate or invertebrate) or plant that harbors a smaller organism; whether their relationship is parasitic, pathogenic, or symbiotic, where the smaller organism generally uses the animal or plant for shelter and/or nourishment. The smaller organism can be a microorganism, such as bacteria, viruses, fungi, a parasite, including, but not limited to worms and insects.

“Serotype” as used herein refers to a distinct variation within a species of bacteria or virus or among immune cells of different individuals. These microorganisms, viruses, or cells are classified together based on their surface antigens, allowing the epidemiologic classification of organisms to the subspecies level.

As used herein, the term infection refers to the invasion of tissues by pathogens, their multiplication, and reaction of host tissues to the infectious agent and any toxins they release. Infections can be caused by a wide range of pathogen, most common are viruses.

As used herein, “downstream” refers to the relative position of a genetic sequence, either DNA or RNA. Downstream relates to the 5′ to 3′ direction relative to the start site of transcription, wherein downstream is usually closer to the 3′ end of a genetic sequence.

As used herein, “upstream” refers to the relative position of a genetic sequence, either DNA or RNA. Upstream relates to the 5′ to 3′ directions relative to the start site of transcription, wherein upstream is usually closer to the 5′ end of a genetic sequence.

A “virus” is a microscopic infectious agent that replicates only inside the living cells of an organism. Viruses can infect all life forms, including mammalian and non-mammalian animals, plants, and other microorganisms. A complete virus, also known as a virion, consists of nucleic acid genetic material surrounded by a protective coat of protein called a capsid. Virus can have a lipid envelope derived from the infected host cell membrane. In general, there are five morphological virus types including helical, icosahedral, prolate, enveloped, and complex virus. A virus can either have a DNA or RNA genome, though a vast majority have RNA genomes. Irrespective of the type of nucleic acid genome, a viral genome can be either a single-stranded genome or a double-stranded genome.

### RNA Sequences and Compositions

RNA viruses are a group of viruses that have ribonucleic acid (RNA), in the form of single stranded RNA or double stranded RNA, as its genetic material. RNA viruses can be classified according to the polarity of the RNA into negative-sense RNA viruses or positive-sense RNA viruses. Viral RNA from a negative-sense RNA virus is complementary to messenger RNA (mRNA), and thus must be converted to positive-sense RNA by an RNA-dependent RNA polymerase before translation into viral proteins. Positive-sense viral RNA, on the other hand, is identical to mRNA, and can thus be translated immediately.

RNA viruses generally comprise very high mutation rates because viral RNA polymerases lack the proofreading functions of DNA polymerases. This contributes to a genetic diversity of RNA molecules (viral RNA or vRNA) produced by RNA viruses. Viral RNA polymerases are also shown to generate aberrant RNA molecules called mini viral RNAs (mvRNAs) during replication of the viral genome. Given the genetic diversity of RNA viruses, there is a need to generate RNA sequences of compositions to prevent RNA virus replication in a host.

In one aspect, disclosed herein is an engineered RNA sequence comprising a stem-loop, wherein the stem-loop comprises a stem portion with at least 5 base pairs (bps) in length and a loop portion with about 20 nucleotides in length, wherein the loop portion matches a footprint of an RNA polymerase of a target virus, and wherein the stem loop is flanked by a 5′ promoter and a 3′ promoter RNA sequence of the target virus.

In one aspect, disclosed herein is an engineered RNA sequence comprising a stem-loop, wherein the stem-loop comprises a stem portion with at least 5 base pairs (bps) in length and a loop portion with about 20 nucleotides in length, wherein the loop portion matches a footprint of an RNA polymerase of influenza A virus, and wherein the stem loop is flanked by a 5′ promoter and a 3′ promoter RNA sequence of the influenza A virus.

In some embodiments, the RNA sequences disclosed herein comprise or match a footprint of an RNA polymerase from a target virus. The footprint refers to an area or region within the RNA polymerase that encloses or surrounds an RNA molecule. The footprint also allows for a designated or optimal number of nucleotides to be held within the RNA polymerase. The footprint includes the entry and exit pathways for the RNA molecule to enter upon conversion to positive-sense RNA and exit after conversion to positive-sense RNA.

In some embodiments, the RNA sequences disclosed herein are suitable for RNA polymerases from a negative-sense RNA virus. The RNA sequences disclosed herein traps the viral replication complex (RNA polymerase) or competes with endogenous vRNAs to inhibit viral replication. The RNA sequences disclosed herein are potent inhibitors of viral infections including, but not limited to infections caused by influenza A virus, influenza B virus, influenza C virus, Ebola virus, Nipah virus, Hanta virus, Hendra virus, Lassa virus, or Rabies virus. Thus, such viruses are sensitive to template loops (t-loops) and are inhibited in similar manners. The RNA sequences disclosed herein can be coupled to a ubiquitin ligase recognition signal (e.g., PROTAC) and trigger the degradation of viral proteins by the proteasome.

In some embodiments, the RNA sequences disclosed herein can activate the immune response upon viral infection and can induce a protective antiviral response. There are various previous methods on using defective interfering RNA viruses/particles, but these rely on using natural sequences. The sequences and the algorithm that are disclosed herein engineer viruses capable of inducing a protective response more robustly, for instance by inserting the sequences into the genome or creating viruses in which one of the viral genome segments is replaced with an RNA capable of trapping the viral replication complex. The RNA sequences disclosed herein can be added to live-attenuated vaccines and act as an adjuvant. In the vaccines, the RNA sequence(s) is bound by the live virus upon infection. After initial steps of infection, the RNA sequences limit viral replication and/or trigger a protective immune response. Said vaccines can be administered during an outbreak of a known or novel virus to provide subjects with a protective immune response before traditional or RNA vaccines are available. Said vaccine can also be administered to healthcare workers before exposure to viruses.

In some embodiments, disclosed herein are RNA sequences used to inhibit influenza virus replication and trigger activation of the innate immune system while the RNA is bound by the viral replication complex (RNA polymerase). The template loop (t-loop) portion of the RNA sequence inhibits other negative viruses, not limited to influenza viruses. Thus, the RNA sequences disclosed herein inhibit viral RNA polymerase activity and/or viral replication in RNA viruses that are similar in size and work mechanistically in similar ways to influenza viruses and other negative-sense RNA viruses.

Under appropriate parameters, the RNA sequence(s) hybridizes, or binds internally to form the t-loop. To reduce viral replication and induce activation of the innate immune response, the stem of the t-loop comprises at least 5 bp long. To prevent the activity of the RNA polymerase entirely, the stem comprises at least 14 bp long. The t-loop is flanked by the natural influenza A virus 5′ and 3′ promoter RNA sequences, which are 12 and 13 nt respectively.

In some embodiments, the RNA sequence can reduce or completely stop the activity of the RNA polymerase.

In some embodiments, the stem portion comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 bps in length. In some embodiments, the loop portion comprises about 10, 11, 12, 13, 14, 15, 16, 17, 18, 19, 20, 21, 22, 23, 24, 25 nucleotides in length.

In some embodiments, the target virus comprises a negative-sense RNA virus. In some embodiments, the negative-sense RNA virus comprises Influenza A virus (IAV), Ebola virus, Nipah virus, Hanta virus, Hendra virus, Lassa virus, or Rabies virus. It should be understood that negative-sense viruses can comprise multiple types of genomes ranging from a single RNA molecule up to eight segments of RNA polymers. For example, IAV and Influenza V virus (IBV) comprise eight segments, while Influenza C virus comprises seven segments. Thus, in some embodiments the engineered RNA sequence targets at least one single RNA molecule or targets up to 8 segments of RNA.

In some embodiments, the RNA sequence comprises between about 40 to about 130 nucleotides. In some embodiments, the RNA sequence comprises between about 52 to about 71 nucleotides. In some embodiments, the RNA sequence comprises about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 nucleotides. It should be noted that the RNA sequences disclosed herein are shorter than defective interfering influenza virus RNAs (including, but not limited to defective interfering (DI) RNAs or DI viruses) described in the art.

In some embodiments, the RNA sequence comprises at least 60% sequence identity to any one of SEQ ID NO: 4-30. In some embodiments, the RNA sequence comprises at least 70% sequence identity to any one of SEQ ID NO: 4-30. In some embodiments, the RNA sequence comprises at least 80% sequence identity to any one of SEQ ID NO: 4-30. In some embodiments, the RNA sequence comprises at least 90% sequence identity to any one of SEQ ID NO: 4-30. In some embodiments, the RNA sequence comprises at least 95% sequence identity to any one of SEQ ID NO: 4-30. In some embodiments, the RNA sequence comprises at least 99% sequence identity to any one of SEQ ID NO: 4-30. In some embodiments, the RNA sequence comprises any one of sequences selected from SEQ ID NO: 4-30.

In some embodiments, the RNA sequence comprises SEQ ID NO: 4, or a variant thereof. In some embodiments, the RNA sequence comprises SEQ ID NO: 5, or a variant thereof. In some embodiments, the RNA sequence comprises SEQ ID NO: 6, or a variant thereof. In some embodiments, the RNA sequence comprises SEQ ID NO: 7, or a variant thereof. In some embodiments, the RNA sequence comprises SEQ ID NO: 8, or a variant thereof. In some embodiments, the RNA sequence comprises SEQ ID NO: 9, or a variant thereof. In some embodiments, the RNA sequence comprises SEQ ID NO: 10, or a variant thereof.

In some embodiments, the RNA sequence comprises SEQ ID NO: 11, or a variant thereof. In some embodiments, the RNA sequence comprises SEQ ID NO: 12, or a variant thereof. In some embodiments, the RNA sequence comprises SEQ ID NO: 13, or a variant thereof. In some embodiments, the RNA sequence comprises SEQ ID NO: 14, or a variant thereof. In some embodiments, the RNA sequence comprises SEQ ID NO: 15, or a variant thereof. In some embodiments, the RNA sequence comprises SEQ ID NO: 16, or a variant thereof. In some embodiments, the RNA sequence comprises SEQ ID NO: 17, or a variant thereof. In some embodiments, the RNA sequence comprises SEQ ID NO: 18, or a variant thereof. In some embodiments, the RNA sequence comprises SEQ ID NO: 19, or a variant thereof. In some embodiments, the RNA sequence comprises SEQ ID NO: 20, or a variant thereof.

In some embodiments, the RNA sequence comprises at least 60% sequence identity to any one of SEQ ID NO: 13. In some embodiments, the RNA sequence comprises at least 70% sequence identity to any one of SEQ ID NO: 13. In some embodiments, the RNA sequence comprises at least 80% sequence identity to any one of SEQ ID NO: 13. In some embodiments, the RNA sequence comprises at least 90% sequence identity to any one of SEQ ID NO: 13. In some embodiments, the RNA sequence comprises at least 95% sequence identity to any one of SEQ ID NO: 13. In some embodiments, the RNA sequence comprises at least 99% sequence identity to any one of SEQ ID NO: 13.

In some embodiments, the RNA sequence comprises at least 60% sequence identity to any one of SEQ ID NO: 14. In some embodiments, the RNA sequence comprises at least 70% sequence identity to any one of SEQ ID NO: 14. In some embodiments, the RNA sequence comprises at least 80% sequence identity to any one of SEQ ID NO: 14. In some embodiments, the RNA sequence comprises at least 90% sequence identity to any one of SEQ ID NO: 14. In some embodiments, the RNA sequence comprises at least 95% sequence identity to any one of SEQ ID NO: 14. In some embodiments, the RNA sequence comprises at least 99% sequence identity to any one of SEQ ID NO: 14.

In some embodiments, the RNA sequence comprises SEQ ID NO: 21, or a variant thereof. In some embodiments, the RNA sequence comprises SEQ ID NO: 22, or a variant thereof. In some embodiments, the RNA sequence comprises SEQ ID NO: 23, or a variant thereof. In some embodiments, the RNA sequence comprises SEQ ID NO: 24, or a variant thereof. In some embodiments, the RNA sequence comprises SEQ ID NO: 25, or a variant thereof. In some embodiments, the RNA sequence comprises SEQ ID NO: 26, or a variant thereof. In some embodiments, the RNA sequence comprises SEQ ID NO: 27, or a variant thereof. In some embodiments, the RNA sequence comprises SEQ ID NO: 28, or a variant thereof. In some embodiments, the RNA sequence comprises SEQ ID NO: 29, or a variant thereof. In some embodiments, the RNA sequence comprises SEQ ID NO: 30, or a variant thereof.

In some embodiments, the RNA sequence comprises at least 60% sequence identity to any one of SEQ ID NO: 25. In some embodiments, the RNA sequence comprises at least 70% sequence identity to any one of SEQ ID NO: 25. In some embodiments, the RNA sequence comprises at least 80% sequence identity to any one of SEQ ID NO: 25. In some embodiments, the RNA sequence comprises at least 90% sequence identity to any one of SEQ ID NO: 25. In some embodiments, the RNA sequence comprises at least 95% sequence identity to any one of SEQ ID NO: 25. In some embodiments, the RNA sequence comprises at least 99% sequence identity to any one of SEQ ID NO: 25.

In some embodiments, SEQ ID NO: 1, SEQ ID NO: 2, and/or SEQ ID NO: 3 can be used as a control sequence (such as, for example a negative control or a positive control).

In some embodiments, the footprint of the RNA polymerase comprises an area within the RNA polymerase capable of holding a designated number of nucleotides. In some embodiments, the designated number of nucleotides comprises about 20 nucleotides. In some embodiments, the designated number of nucleotides comprises about 10, 11, 12, 13, 14, 15, 16, 17, 18, 19, 20, 21, 22, 23, 24, 25 nucleotides.

In some embodiments, the 5′ promoter comprises about 12 nucleotides in length. In some embodiments, the 5′ promoter comprises about 10, 11, 12, 13, 14, 15 nucleotides in length. In some embodiments, the 3′ promoter comprises about 13 nucleotides in length. In some embodiments, the 3′ promoter comprises about 10, 11, 12, 13, 14, 15 nucleotides in length.

In one aspect, disclosed herein is a cell expressing the engineered RNA of any preceding aspect. In some embodiments, the cell expresses or comprises a vector encoding the engineered RNA.

In some embodiments, the cell is a mammalian cell or a bacterial cell.

It should be understood that any vector capable of stably expressing the engineered RNA sequences can be used. The word “vector” refers to any vehicle that carries a polynucleotide into a cell for the expression of the polynucleotide in the cell. The vector may be, for example, a plasmid, a virus, a phage particle, or a nanoparticle. Once transformed or transduced into a suitable host, the vector may replicate and function independently of the host genome, or may in some instances, integrate into the genome itself. In some embodiments, the vector comprises a nucleic construct containing a nucleotide sequence which is operably linked to a suitable control sequence capable of effecting the expression of the nucleic acid in a suitable host cell. Such control sequences can include a promoter to effect transcription, an optional operator sequence to control such transcription, a sequence encoding suitable RNA ribosome binding sites, and sequences which control the termination of transcription and translation.

In some embodiments, the vector comprises a lipid nanoparticle. Lipid nanoparticles can be used to deliver engineered RNA sequences to a cell. In some embodiments, the RNA sequence can be introduced into a host cell through genetic modification. In some embodiments, the RNA sequence is introduced into a host cell by any DNA or RNA delivery technology known in the art, or combinations thereof.

In some embodiments, the vector comprises a plasmid. In some embodiments, the vector comprises an RNA polymerase I (pol I) promoter. In some embodiments, the vector comprises flanking the RNA sequence with a hepatitis delta virus ribozyme sequence. A non-limiting example of expression of an IAV is described in Fodor et al. “Rescue of Influenza A Virus from Recombinant DNA”. Journal of Virology. November 1999 pg. 9679-9682, which is incorporated herein in its entirety as a reference for its teaching of designing a vector (such as, for example a plasmid) comprising IAV genes and expressing said vector in cells.

Methods of Treating, Preventing, Reducing, and/or Decreasing Viral Infections

Viral replication refers to the formation process of biological viruses during an infection in host cells. Because viruses can only multiply within a living host cell, the host cell must supply the energy, replicative machinery, and the low molecular weight precursors for synthesis of viral proteins and nucleic acids. The process of viral replication occurs in seven stages, including: 1) Attachment, wherein the virus attaches to the cell membrane of the host cell and injects genetic material into the host to initiate infection; 2) Entry, wherein the host cell membrane invaginates, or internalizes, the virus particles; 3) Uncoating, wherein the enzymes from the host cell strips away the virus protein coat to expose viral genome; 4) Transcription, wherein viral RNA can be directly translated into viral protein (positive-sense RNA viruses), or must first be transcribed into positive-sense RNA before protein translation (negative-sense or DNA viruses); 5) Synthesis of viral components, wherein the components of the virus are manufactured using existing enzymes and organelles of the host; 6) Viral assembly, wherein the newly synthesized genome and proteins are assembled into a new, active virus; and 7) Release, wherein the newly assembled viruses are released by sudden rupture of the host cell or gradual extrusion of viruses from the host cell. It should be noted that during step 4, negative-sense RNA polymerases can generate aberrant RNA sequences (vRNAs and/or mvRNAs).

It has been contemplated that generation of mvRNAs trigger an innate immune response against viral infections. The innate immune response refers to the first line of defense against invading pathogens that includes immune systems cells and proteins that protect against pathogens that have entered the host body. “The innate and adaptive immune systems” is incorporate herein by reference, in its entirety, for the teachings of the components and functions of the innate immune system (InformedHealth.org. Cologne, Germany: Institute for Quality and Efficiency in Health Care (IQWiG); 2006—. The innate and adaptive immune systems. Available from: www.ncbi.nlm.nih.gov/books/NBI279396). A non-limiting example of an innate immune response includes the retinoic acid-inducible gene I (RIG-I) that is an RNA sensor important for detecting viral infections. The RIG-I sensor is a host pathogen receptor that, upon binding a target RNA molecule, initiates a cascade of signaling pathways that leads to interferon (IFN) expression. This innate response causes release of IFN proteins that activate and allow communication between immune system cells to eradicate the infectious pathogen.

The present disclosure provides methods that manipulate and/or optimize the above mentioned processes to prevent, reduce, and/or decrease viral replication; prevent, reduce, and/or decrease RNA polymerase activity; activate and/or increase an innate immune response; treat and/or prevent a viral infection; or any combinations thereof.

In one aspect, disclosed herein is a method of reducing viral replication, inducing activation of an innate immune response to a target virus, or a combination thereof, the method comprising engineering an RNA sequence comprising a stem-loop, wherein the stem-loop comprises a stem portion with at least 5 base pairs (bps) in length and a loop portion with about 20 nucleotides in length, wherein the loop portion matches a footprint of an RNA polymerase of a target virus, and wherein the stem loop is flanked by a 5′ promoter and a 3′ promoter RNA sequence of the target virus, and contacting the RNA sequence to the RNA polymerase of the target virus, wherein the RNA sequence forms a template loop (t-loop) around the RNA polymerase to reduce viral replication, and wherein the RNA sequence comprises an agonist to activate the innate immune response.

In one aspect, disclosed herein is a method of preventing RNA polymerase activity, the method comprising engineering an RNA sequence comprising a stem-loop, wherein the stem-loop comprises a stem portion with at least 14 base pairs (bps) in length and a loop portion with about 20 nucleotides in length, wherein the loop portion matches a footprint of an RNA polymerase of a target virus, and wherein the stem loop is flanked by a 5′ promoter and a 3′ promoter RNA sequence of the target virus, and contacting the RNA sequence to the RNA polymerase of the target virus, wherein the RNA sequence forms a template loop (t-loop) around the RNA polymerase to stop RNA polymerase activity.

In one aspect, disclosed herein is a method of treating and/or preventing a viral infection, the method comprising engineering an RNA sequence comprising a stem-loop, wherein the stem-loop comprises a stem portion with at least 5 base pairs (bps) in length and a loop portion with about 20 nucleotides in length, wherein the loop portion matches a footprint of an RNA polymerase of a target virus, and wherein the stem loop is flanked by a 5′ promoter and a 3′ promoter RNA sequence of the target virus, and administering the RNA sequence to a subject, wherein the RNA sequence contacts the RNA polymerase of the target virus, and wherein the RNA sequence reduces viral replication and activates the innate immune response in the subject relative to an untreated control subject.

Disclosed herein are methods to activate the innate immune response using RNA-based inhibition of influenza virus replication.

In some embodiments, the method comprises a stem portion with 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 bps in length. In some embodiments, the method comprises a loop portion with about 10, 11, 12, 13, 14, 15, 16, 17, 18, 19, 20, 21, 22, 23, 24, 25 nucleotides in length.

In some embodiments, the method comprises an RNA sequence with between about 40 to about 80 nucleotides. In some embodiments, the method comprises an RNA sequence with between about 52 to about 71 nucleotides. In some embodiments, the method comprises an RNA sequence with about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 nucleotides.

In some embodiments, the target virus comprises a negative-sense RNA virus. In some embodiments, the negative-sense RNA virus comprises Influenza A virus (IAV), Ebola virus, Nipah virus, Hanta virus, Hendra virus, Lassa virus, or Rabies virus.

In some embodiments, the method comprises any one of sequences selected from SEQ ID NO: 4-30.

In some embodiments, the footprint of the RNA polymerase comprises an area within the RNA polymerase capable of holding a designated number of nucleotides. In some embodiments, the designated number of nucleotides comprises about 20 nucleotides. In some embodiments, the designated number of nucleotides comprises about 10, 11, 12, 13, 14, 15, 16, 17, 18, 19, 20, 21, 22, 23, 24, 25 nucleotides.

In some embodiments, the method comprises a 5′ promoter with about 12 nucleotides in length. In some embodiments, the method comprises a 5′ promoter with about 10, 11, 12, 13, 14, 15 nucleotides in length. In some embodiments, the method comprises a 3′ promoter with about 13 nucleotides in length. In some embodiments, the method comprises a 3′ promoter with about 10, 11, 12, 13, 14, 15 nucleotides in length.

In some embodiments, the stem-loop inhibits the RNA polymerase from replicating a genome of the target virus. In some embodiments, the stem-loop inhibits the RNA polymerase from replicating negative-sense RNA. In some embodiments, the RNA sequence forms a template loop (t-loop) around the RNA polymerase to reduce viral replication. In some embodiments, the method inhibits transcription, synthesis, assembly, and/or release of viral components.

In some embodiments, the innate immune response comprises binding a host pathogen receptor to the RNA sequence. In some embodiments, the host pathogen receptor comprises a retinoic acid-inducible gene I (RIG-I). In some embodiments, the method comprises expression and/or release of IFN proteins against the viral infection.

### Methods of Performing Analyses

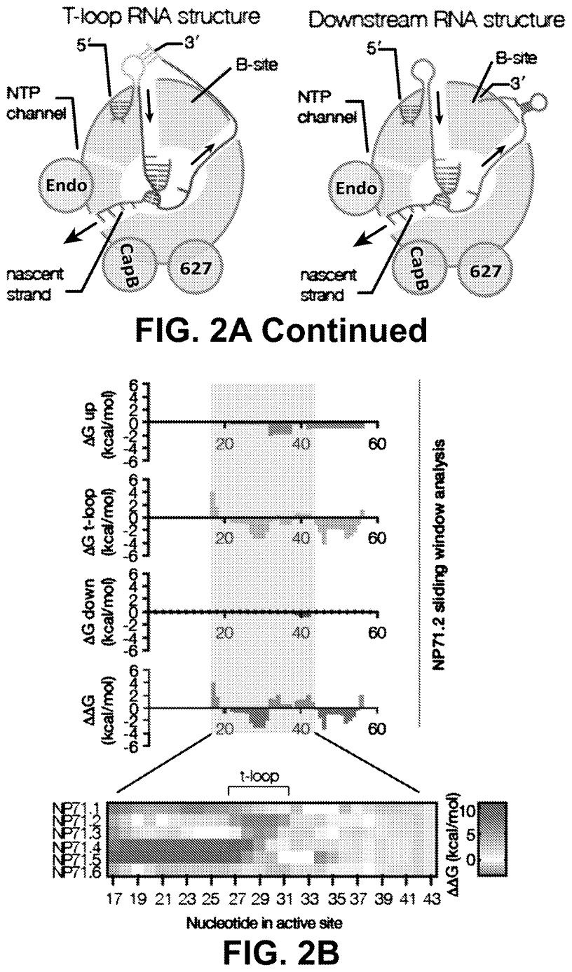

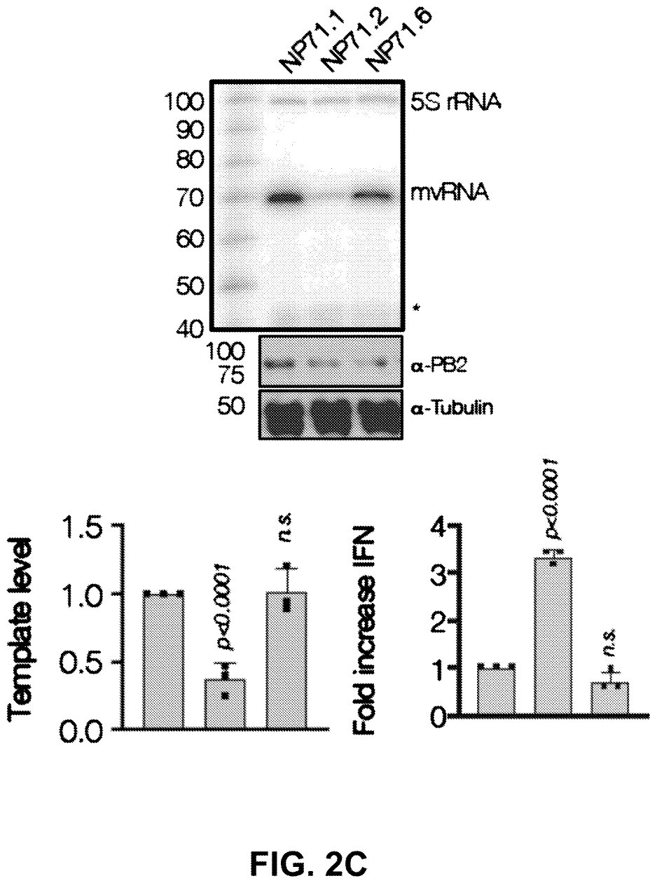

Template loop (t-loop) structures are RNA sequences that form a nucleic acid complex around an RNA polymerase to stall or prevent replication. Under optimized parameters, the t-loop can also initiate an innate immune response (such as, for example triggering a RIG-I RNA sensor). To optimally prevent RNA polymerase functions, such as, for example replication, the t-loop matches or comprises a footprint of the RNA polymerase from a target virus, such as, for example, influenza A virus. A matching footprint allows the RNA sequence to reside within the entry, exit, and interior channels of the RNA polymerase (See FIG. 2A (2^{nd} image; T-loop RNA structure)). The t-loop structure forms around the RNA polymerase when the 3′ terminus of the RNA sequence (generally located at the exit channel of the RNA polymerase) can hybridize, or bind, to the upstream 5′ terminus of the RNA sequence (generally located at the entry channel of the RNA sequence). The hybridizing, or binding, of the 3′ terminus to the 5′ terminus causes the internal nucleotides of the RNA sequence to loop around and through the RNA polymerase. The formation of at least one t-loop around an RNA polymerase can stall the RNA polymerase from replicating the viral genome. It should be understood that RNA sequence can form 1, 2, 3, 4, 5, 6, 7, 8, 9, 10 or more t-loops around and through the RNA polymerase.

Depending on the purpose of the RNA sequence (inhibition, innate immune activation, or both), the stem of a template loop (t-loop) can be modified, and the overall length of the RNA sequence optimized. Since the mechanism of action of t-loops is well-defined, an algorithm was designed to evaluate and optimize the sequences in silico. Additional t-loop sequences, aside from those sequences disclosed herein, can be quickly identified using the in silico analyses disclosed herein. The specific parameters can be defined to broadly claim the whole RNA sequence space that yields t-loop sequences with similar properties. Thus, the present disclosure provides methods of analyzing t-loop sequences for use in inhibiting RNA polymerase activity, inhibiting viral replication, activating an innate immune response, or combinations thereof.

In some embodiments, the analysis includes detecting or identifying the free energy of the t-loop, the upstream sequence, and downstream sequence of an RNA sequence template. In some embodiments, the analysis includes determining the differences in free energies between the t-loop, the upstream sequence, and the downstream sequence of the RNA sequence template.

Free energy, also referred to as ΔG, refers to a thermodynamic property that defines an energy property or function of a system in thermodynamic equilibrium. Free energy has the dimensions of energy, and its value is determined by the state of the system. Free energy is used to determine how systems change and how much work (in the form of energy) they can produce. When referring to nucleic acid systems, nucleic acid free energy refers to how external and internal factors (such as, for example temperature, the number of nucleotides, and/or the type of nucleotides) affect the formation and/or binding of secondary or tertiary nucleic acid structures (including, but not limited to hairpins, stem loops, and/or t-loops). Detection of a ΔG for a given RNA sequence template informs on the parameters needed to optimally form a t-loop structure. Such parameters include, but are not limited to the appropriate temperature, the optimal number of nucleotides, and/or the optimal positioning of adenine, thymine, cytosine, and guanine nucleotides for hybridization of the RNA sequence template.

In some embodiments, the temperature parameter for forming t-loops ranges from about 35° C. to about 40° C. In some embodiments, the temperature parameter for forming t-loops ranges from about 36° C. to about 38° C. In some embodiments, the temperature parameter for forming t-loops comprises about 37° C. In some embodiments, the temperature parameter for forming t-loops comprises about 36.0° C., 36.1° C., 36.2° C., 36.3° C., 36.4° C., 36.5° C., 36.6° C., 36.7° C., 36.8° C., 36.9° C., 37.0° C., 37.1° C., 37.2° C., 37.3° C., 37.4° C., 37.5° C., 37.6° C., 37.7° C., 37.8° C., 37.9° C., 38.0° C., 38.1° C., 38.2° C., 38.3° C., 38.4° C., 38.5° C., 38.6° C., 38.7° C., 38.8° C., 38.9° C., or 39.0° C.

Additional optimization is required by determining the differences in free energies (ΔΔG) between the ΔG of the t-loop, ΔG of the upstream (5′) terminus sequence, and ΔG of the downstream (3′) terminus sequence (or promoters) of the RNA sequence template. Determining the ΔΔG can be repeat as many times as necessary until the ΔΔG values have been determined for an entire template loop RNA sequence.

In one aspect, disclosed herein is a method of performing a t-loop analysis, the method comprising identifying a template loop RNA sequence, blocking off a portion of the template loop RNA sequence to represent a footprint of a virus RNA polymerase, determining a t-loop ΔG, an upstream ΔG for a stretch of at 10 nucleotides of the footprint, and a downstream ΔG for a stretch of at 10 nucleotides of the footprint, and determining a ΔΔG for a likelihood of t-loop formation by subtracting the upstream ΔG and downstream ΔG from the t-loop ΔG, and moving the footprint in a one-nucleotide increment along the template loop RNA sequence, and repeating the previous step until the ΔΔG values have been determined for an entire template loop RNA sequence.

In one aspect, disclosed herein is a method of designing single stranded RNA molecules that regulate viral replication and stimulate activation of the innate immune response leading to the production of interferons. In some embodiments, the method regulates influenza A viral replication.

In some embodiments, the stretch of nucleotides comprises 5, 6, 7, 8, 9, 10, 11, 12, 13, 14, 15, 16, 17, 18, 19, or 20 nucleotides of the footprint.

### Computing Devices and Methods of Use

Disclosed herein is an algorithm (Python code) to test the design of new RNA sequences in silico.

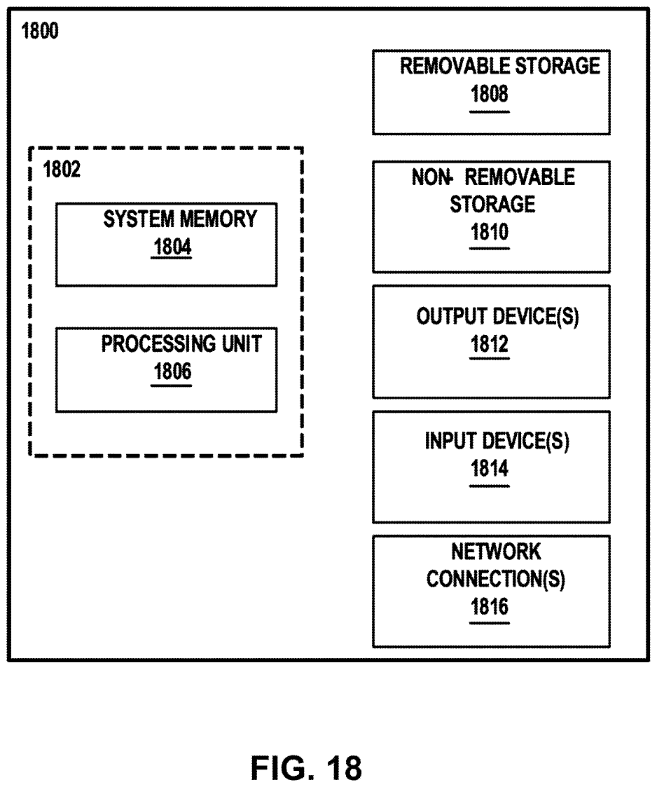

It should be appreciated that the logical operations described herein with respect to the various figures may be implemented (1) as a sequence of computer-implemented acts or program modules (i.e., software) running on a computing device (e.g., the computing device described in FIG. 18), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special-purpose digital logic, and any combination thereof. It should also be appreciated that more or fewer operations may be performed than shown in the figures and described herein. These operations may also be performed in a different order than those described herein.

Referring to FIG. 18, an example computing device 1800 upon which embodiments of the invention may be implemented is illustrated. It should be understood that the example computing device 1800 is only one example of a suitable computing environment upon which embodiments of the invention may be implemented. Optionally, the computing device 1800 can be a well-known computing system including, but not limited to, personal computers, servers, handheld or laptop devices, multiprocessor systems, microprocessor-based systems, personal network computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, the computing device 1800 typically includes at least one processing unit 1806 and system memory 1804. Depending on the exact configuration and type of computing device, system memory 1804 may be volatile (such as random-access memory (RAM)), non-volatile (such as read-only memory (ROM), flash memory, etc.), or some combination of the two. This most basic configuration is illustrated in FIG. 18 by the dashed line 1802. The processing unit 1806 may be a standard programmable processor that performs arithmetic and logic operations necessary for the operation of the computing device 1800. The computing device 1800 may also include a bus or other communication mechanism for communicating information among various components of the computing device 1800.

Computing device 1800 may have additional features/functionality. For example, the computing device 1800 may include additional storage such as removable storage 1808 and non-removable storage 1810 including, but not limited to magnetic or optical disks or tapes. Computing device 1800 may also contain network connection(s) 1816 that allow the device to communicate with other devices. Computing device 1800 may also have input device(s) 1814 such as a keyboard, mouse, touch screen, etc. Output device(s) 1812, such as a display, speakers, printer, etc., may also be included. The additional devices may be connected to the bus in order to facilitate communication of data among the components of the computing device 1800. All these devices are well-known in the art and need not be discussed at length here.

The processing unit 1806 may be configured to execute program code encoded in tangible, computer-readable media. Tangible, computer-readable media refers to any media that is capable of providing data that causes the computing device 1800 (i.e., a machine) to operate in a particular fashion. Various computer-readable media may be utilized to provide instructions to the processing unit 1806 for execution. Example of tangible, computer-readable media may include but is not limited to, volatile media, non-volatile media, removable media and non-removable media implemented in any method or technology for storage of information such as computer-readable instructions, data structures, program modules or other data. System Memory 1804, removable storage 1808, and non-removable Storage 1810 are all examples of tangible computer storage media. Examples of tangible, computer-readable recording media include but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1806 may execute program code stored in the system memory 1804. For example, the bus may carry data to the system memory 1804, from which the processing unit 1806 receives and executes instructions. The data received by the system memory 1804 may optionally be stored on the removable storage 1808 or the non-removable storage 1810 before or after execution by the processing unit 1806.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, for example, through the use of an application programming interface (API), reusable controls, or the like. Such programs may be implemented in a high-level procedural or object-oriented programming language to communicate with a computer system. However, the program(s) can be implemented in assembly or machine language if desired. In any case, the language may be a compiled or interpreted language, and it may be combined with hardware implementations.

In one aspect, disclosed herein is a non-transitory computer-readable storage medium comprising instructions that, when executed, cause at least one processor to perform the method of any preceding aspect.

In some implementations, the techniques described herein relate to a computer-implemented method for generating at least one optimal RNA sequence for reducing viral replication and/or inducing activation of an innate response to a target virus, the computer-implemented method including: retrieving, by one or more processors, an RNA polymerase of the target virus; performing, by the one or more processors, a template loop (t-loop) analysis operation on the RNA polymerase; determining, by the one or more processors and based on results from the t-loop analysis operation and/or sliding window operation, at least one optimal RNA sequence corresponding with the RNA polymerase of the target virus; and outputting, by the one or more processors, the at least one optimal RNA sequence (e.g., via a display device or other output device).

In some implementations, the techniques described herein relate to a computer-implemented method, further including receiving, by the one or more processors, one or more user-defined parameters associated with the RNA polymerase of the target virus; and performing, by the one or more processors, the t-loop analysis operation based at least on the one or more user-defined parameters.

In some implementations, the techniques described herein relate to a computer-implemented method, further including determining, by the one or more processors, at least one criterion of a composition for administration to a subject based on the at least one optimal RNA sequence.

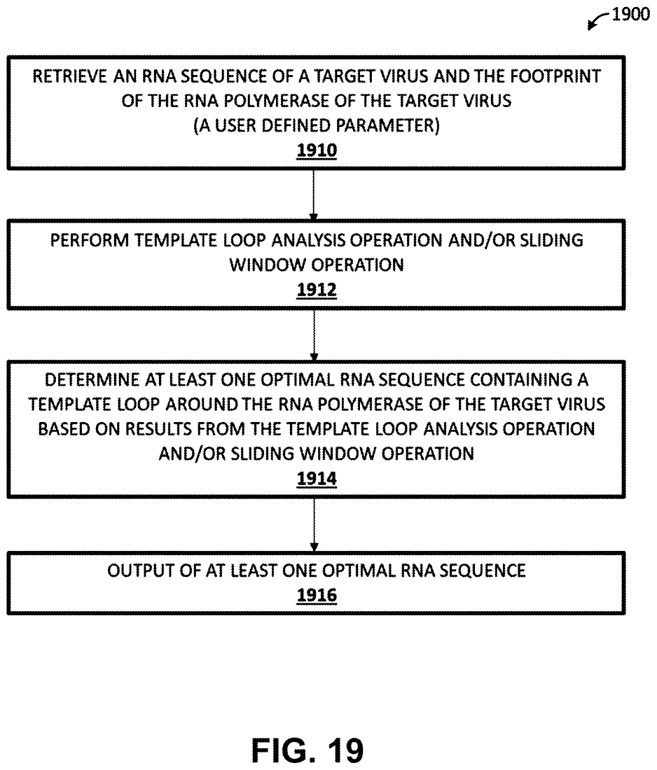

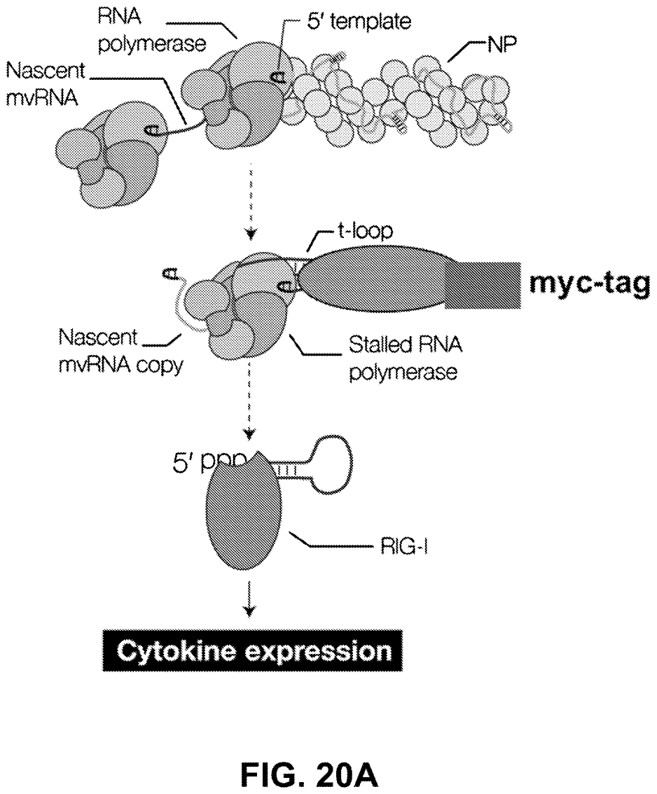

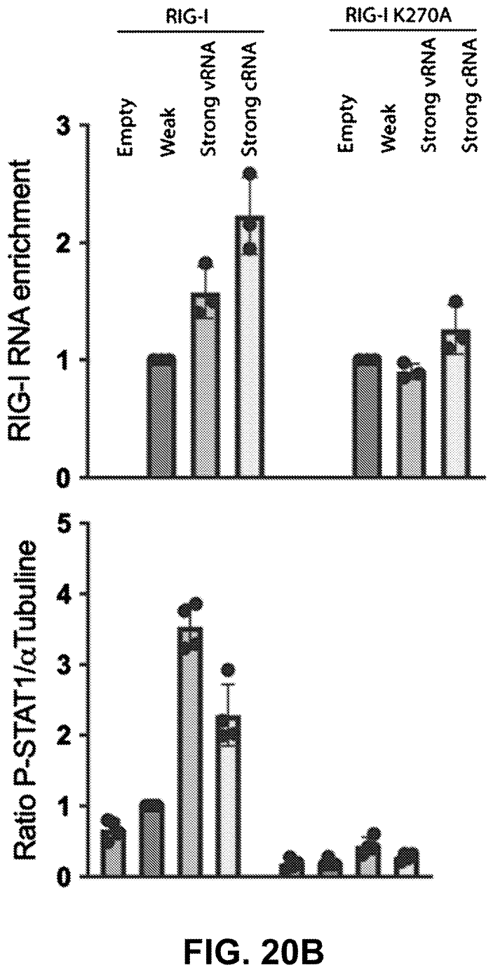

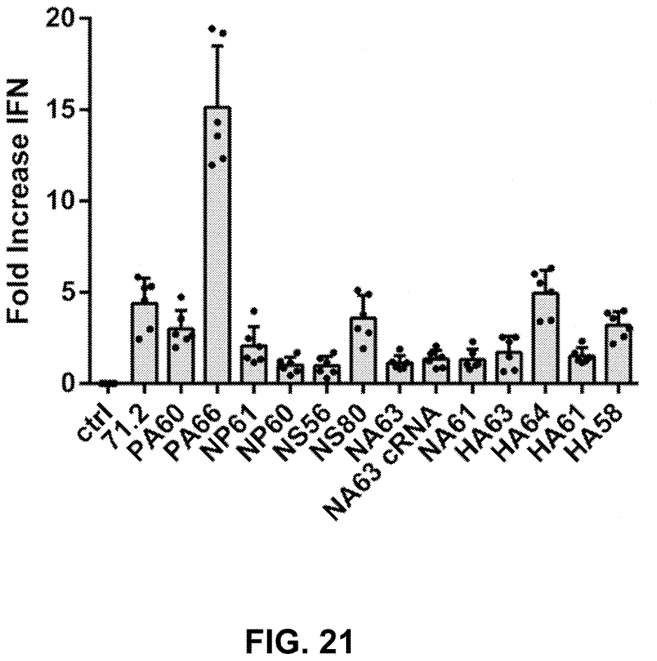

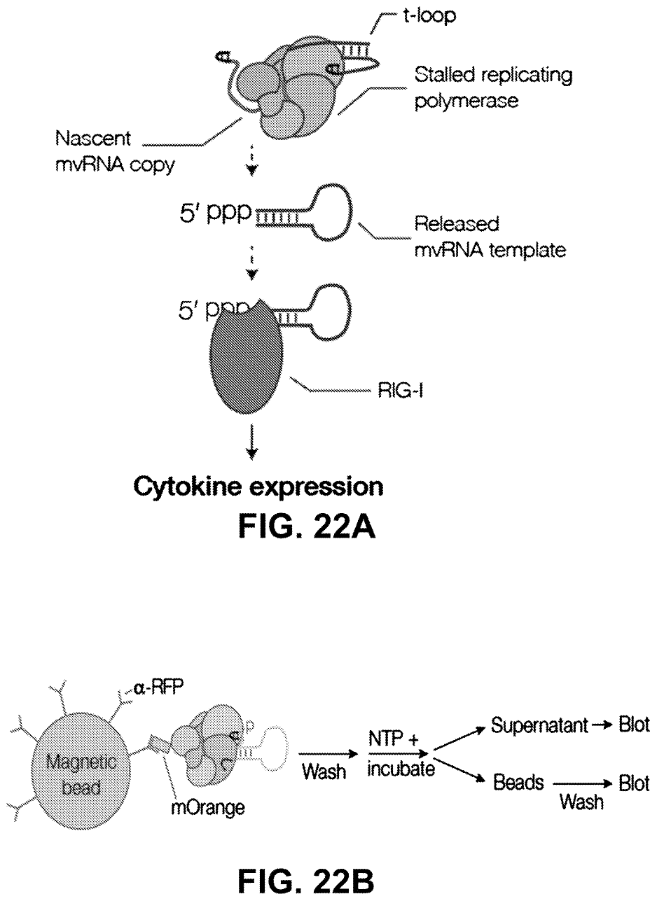

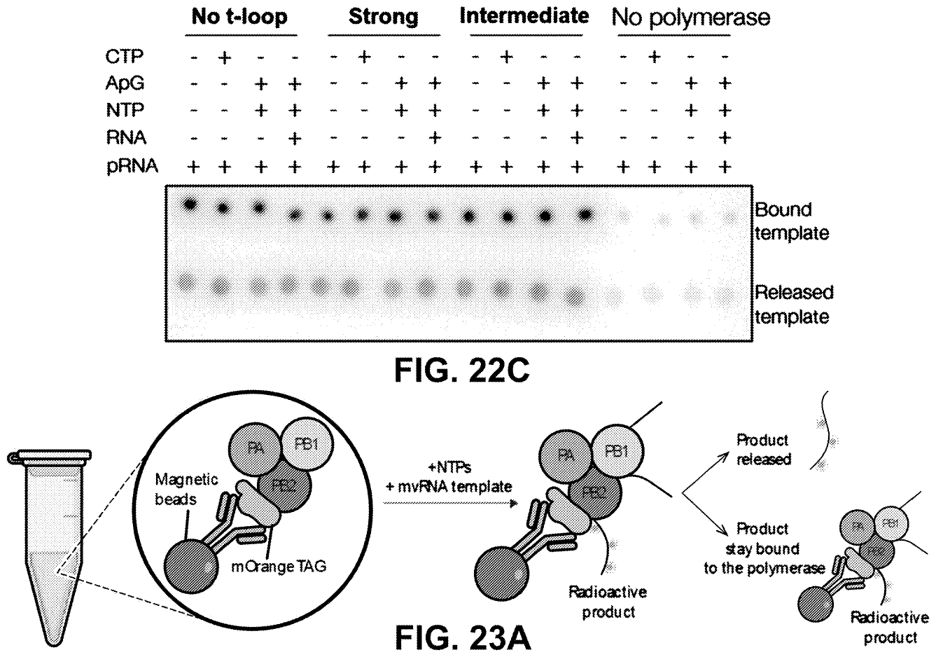

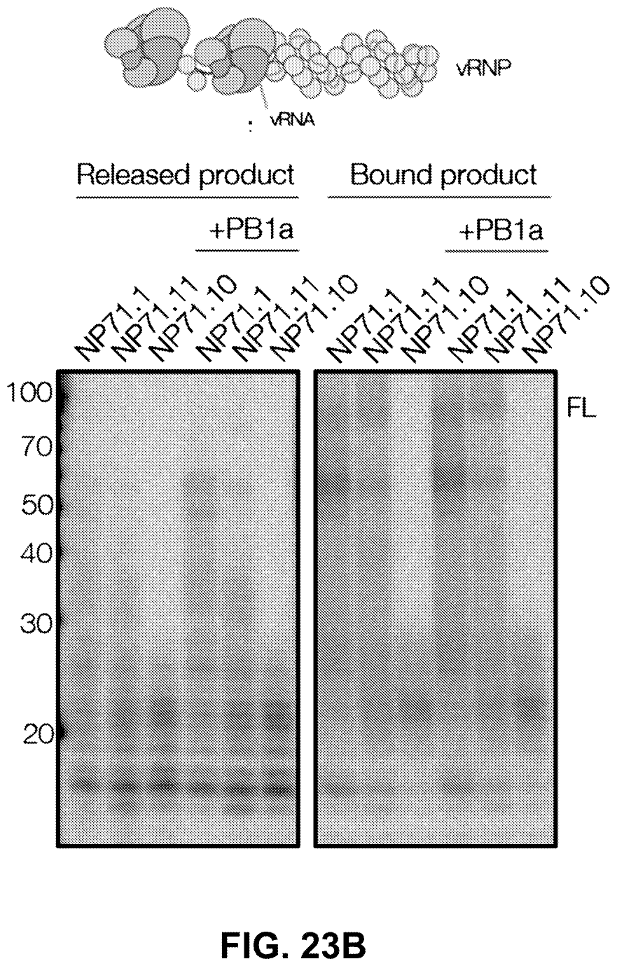

FIG. 19 is a flowchart of an example computer-implemented method 1900 for generating at least one optimal RNA sequence for reducing viral replication and/or inducing activation of an innate response to a target virus. In some implementations, the method 1900 can be performed by a processing circuitry (for example, but not limited to, an application-specific integrated circuit (ASIC), or a central processing unit (CPU)). In some examples, the processing circuitry may be electrically coupled to and/or in electronic communication with other circuitries of an example computing device, such as, but not limited to, the example computing device 1800 described above in connection with FIG. 18. In some examples, embodiments may take the form of a computer program product on a non-transitory computer-readable storage medium storing computer-readable program instruction (e.g., computer software). Any suitable computer-readable storage medium may be utilized, including non-transitory hard disks, CD-ROMs, flash memory, optical storage devices, or magnetic storage devices.

At step/operation 1910, at least one processor (such as, but not limited to, at least one processor or processing circuitry of the computing device 1800) retrieves an RNA polymerase of the target virus. In some implementations, at step/operation 1910, the at least one processor retrieves/receives one or more user-defined parameters associated with the RNA polymerase of the target virus.

At step/operation 1912, the at least one processor performs a template loop (t-loop) analysis operation on the RNA polymerase. In some implementations, the at least one processor performs the t-loop analysis operation based at least in part on the retrieved/received one or more user-defined parameters. In some embodiments, the t-loop analysis operation comprises a sliding window operation as described in more detail herein. Example 6 below provides an example algorithm (Python script) for performing a t-loop analysis operation/sliding window operation in accordance with embodiments of the present disclosure.

At step/operation 1914, the at least one processor determines at least one optimal RNA sequence corresponding with the RNA polymerase of the target virus based on results (e.g., one or more determined values) from the t-loop analysis operation. In some embodiments, the at least one optimal RNA sequence comprises a stem-loop. In some examples, the stem-loop further comprises a stem portion with at least 5 base pairs (bps) in length and a loop portion with about 20 nucleotides in length, and wherein the loop portion matches a footprint of the RNA polymerase of the target virus. In some examples, the stem-loop is flanked by a 5′ promoter and a 3′ promoter RNA sequence of the target virus.

At step/operation 1916, the at least one processor outputs (e.g., via a display device) the at least one optimal RNA sequence. For example, the processor may output user-interface data to an end user via a graphical user interface or generate and provide (e.g., send, transmit) a text file. Additionally, and/or alternatively, the at least one processor can output at least one criterion of a composition (or instructions for engineering the composition) for administration to a subject based on the at least one optimal RNA sequence.

In one aspect, disclosed herein is a computer-implemented method for generating at least one optimal ribonucleic acid (RNA) sequence for reducing viral replication and/or inducing activation of an innate response to a target virus, the computer-implemented method comprising retrieving, by one or more processors, an RNA polymerase of the target virus, performing, by the one or more processors, a template loop (t-loop) analysis operation on the RNA polymerase, determining, by the one or more processors, at least one optimal RNA sequence corresponding with the RNA polymerase of the target virus based on results of the t-loop analysis operation, and outputting, by the one or more processors, a ΔG, a location of the ΔG, a t-loop structure, or combinations thereof. In some embodiments, the output comprises the at least one optimal RNA sequence.

In some embodiments, the computer-implemented method further comprises receiving, by the one or more processors, one or more user-defined parameters associated with the RNA polymerase of the target virus, and performing, by the one or more processors, the t-loop analysis operation based at least on the one or more user-defined parameters. In some embodiments, the t-loop analysis operation is used interchangeably with a sliding window operation. In some embodiments, the computer-implemented method further comprises determining, by the one or more processors, at least one criterion of a composition for administration to a subject based on the at least one optimal RNA sequence.

In some embodiments, the method can be used to optimize virus genome sequences and alter their growth kinetics. In some embodiments, the method can be used to optimize the growth of viral strains used for vaccine production.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Transient RNA Structures Cause Aberrant Influenza Virus Replication and Innate Immune Activation

The influenza A virus (IAV) RNA polymerase produces both full-length and aberrant RNA molecules, such as defective viral genomes (DVG) and mini viral RNAs (mvRNA), during infection. Subsequent innate immune activation involves the binding of host pathogen receptor retinoic acid-inducible gene I (RIG-I) to viral RNAs. However, it is not clear what factors determine which influenza A virus RNAs are RIG-I agonists. Herein, evidence is provided that RNA structures, called template loops (t-loop), stall the viral RNA polymerase and contribute to innate immune activation by mvRNAs during influenza A virus infection. Impairment of replication by t-loops depends on the formation of an RNA duplex near the template entry and exit channels of the RNA polymerase, and this effect is enhanced by mutation of the template exit path from the RNA polymerase active site. Overall, these findings are supportive of a mechanism involving polymerase stalling that links aberrant viral replication to the activation of the innate immune response.

During an IAV infection, the virus introduces eight ribonucleoproteins (RNP) into the host cell nucleus. These RNPs consist of oligomeric viral nucleoprotein (NP), a copy of the viral RNA polymerase, and one of the eight segments of single stranded negative sense viral RNA (vRNA) that make up the viral genome. The vRNA segments range from 890 to 2341 nt in length, but all contain conserved 5′ triphosphorylated, partially complementary 5′ and 3′ termini. These termini serve as promoter for the RNA polymerase, but also as agonist of RIG-I. In the context of an RNP, the termini are bound by the RNA polymerase subunits PB1, PB2, and PA, and during viral replication, a second RNA polymerase is recruited to the RNP to encapsulate nascent RNA. It has been contemplated that binding of the viral RNA polymerase to the vRNA termini reduces RIG-I binding to the vRNA segments and it is not clear when or where RIG-I gains access to viral RNAs.

In addition to full-length vRNA and cRNA molecules, the viral RNA polymerase can produce aberrant RNAs that are shorter than the vRNA or cRNA template from which they derive. Such aberrant RNAs include defective viral genomes (DVGs) and mini viral RNAs (mvRNA), which contain internal deletions between the conserved 5′ and 3′ termini. Both DVGs and mvRNAs can bind RIG-I and activate innate immune responses, but only DVGs require viral NP during viral replication while mvRNAs do not. It is presently not fully understood what determines the ability of DVGs and mvRNAs to activate RIG-I or how they are made. Interestingly, the RNA polymerases of highly pathogenic avian H5N1 and pandemic 1918 H1N1 IAV produce higher mvRNA levels than the RNA polymerase of lab adapted H1N1 IAV, showing that there is a correlation between adaptive mutations in the RNA polymerase, mvRNA production, and innate immune activation in infections with highly pathogenic IAV.

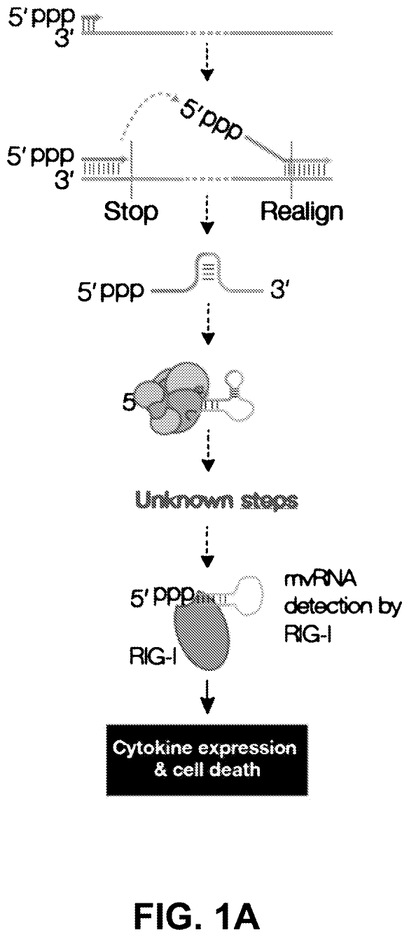

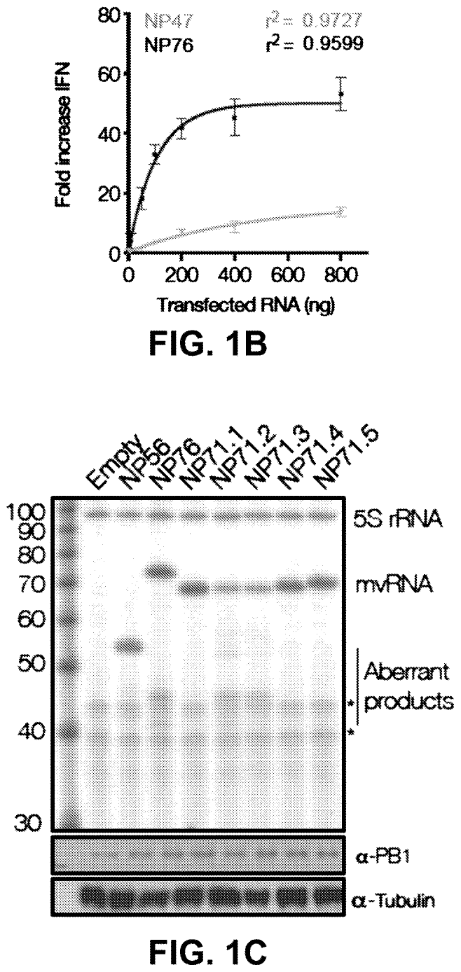

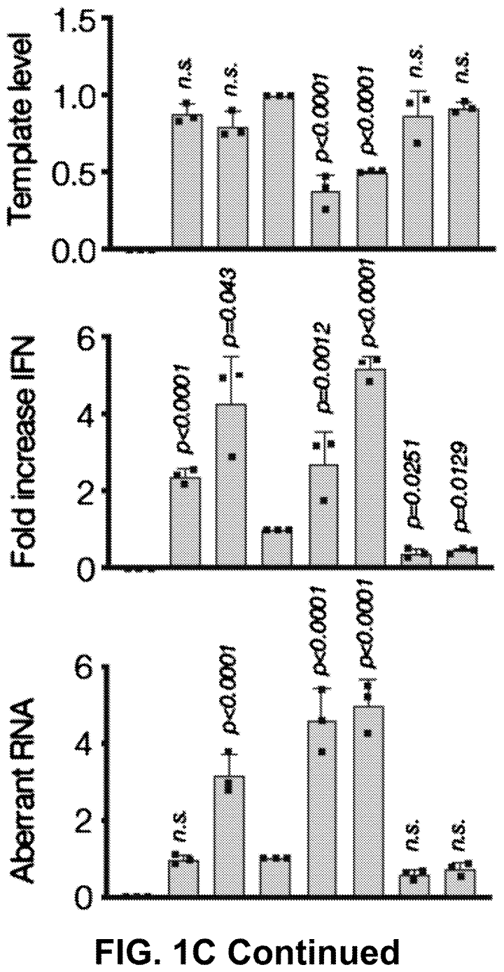

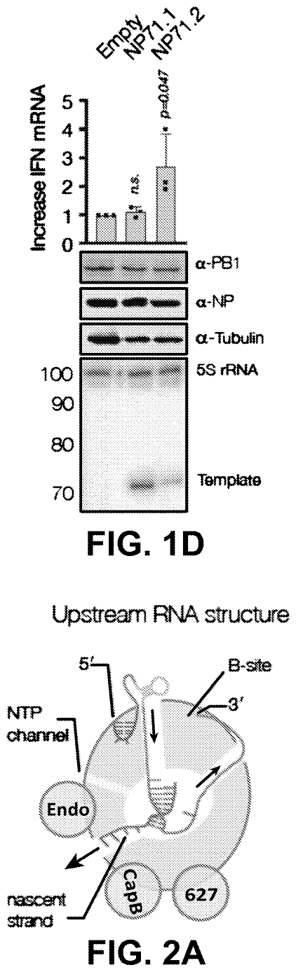

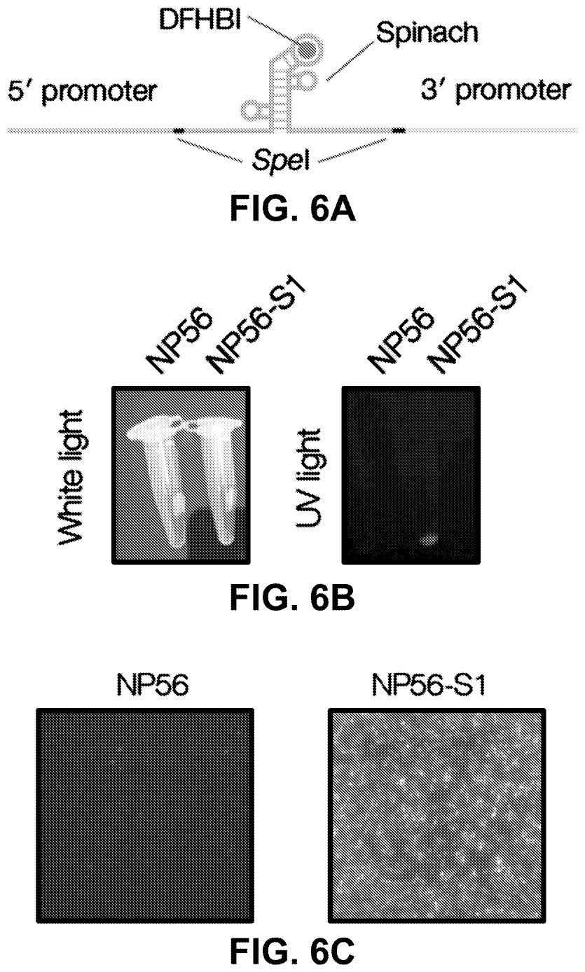

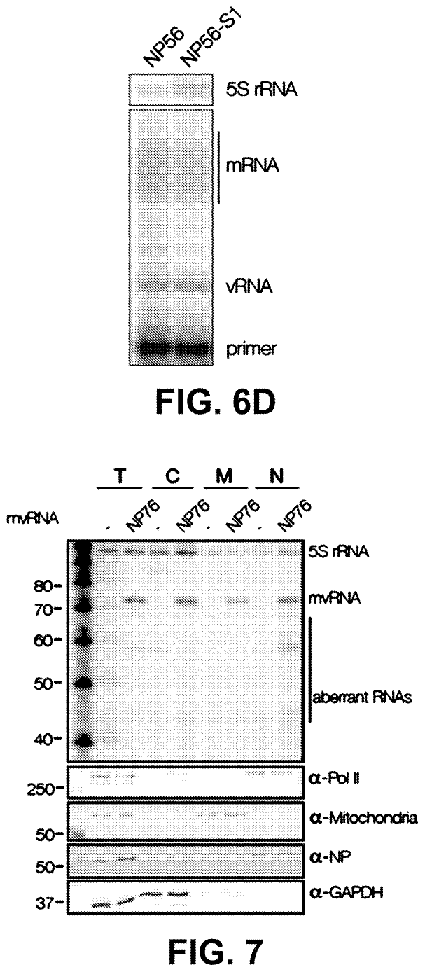

Herein, this example examines the role of mvRNAs in innate immune activation in more detail. mvRNAs are generated, in part, via a copy-choice mechanism that results in the loss of an internal genome segment sequence, similar to what has been observed for DVGs (FIG. 1B). As a result, RNA sequences or structures that do not normally reside side-by-side in the full-length genome segments are brought closer to each other in the nascent RNA, resulting in the formation of novel RNA structures (FIG. 1B). Once generated, mvRNAs can be replicated by the viral RNA polymerase in the absence of NP. Inherently, the RNA polymerase is not impaired by RNA structures in an mvRNA template and it can replicate and transcribe an mvRNA containing a copy of the aptamer Spinach, a highly-structured RNA capable of stabilizing the fluorophore DFHBI (FIG. 6). However, it is not known if other RNA secondary structures or certain sequence combinations could impair mvRNA replication or play a role in the activation of the innate immune response during IAV infection, as for instance has been observed for paramyxovirus infections. Herein, a previous model is advanced on the effect of mvRNAs on the innate immune response and provide evidence that mvRNAs capable of inducing innate immune responses contain RNA structures that can reduce the activity of the IAV RNA polymerase.

### Results

Induction of IFN-β Promoter Activation by mvRNAs is Sequence Dependent

mvRNAs bind RIG-I and activate the MAVS signaling cascade, but it is unclear what determines whether an IAV mvRNA is an inducer of the innate immune response. To systematically investigate if the sequence or secondary structure of an mvRNA can affect IAV RNA polymerase activity and innate immune activation, five segment 5-derived mvRNA templates were engineered. Each engineered mvRNA had a length of 71 nt (NP71.1-NP71.5), but a different internal sequence (Table 1). The positive control mvRNAs were 56- and 76-nt long mvRNAs, which were previously constructed from segment 5 (NP56 and NP76, respectively), while the negative control mvRNA was a 47-nt long mvRNA derived from segment 5 (NP47) that is unable to bind RIG-I and induce a strong IFN signal. To validate the test setup, increasing amounts of in vitro transcribed NP76 was transfected into HEK 293T cells and a strong increase in IFN-β promoter activity was found that saturated at a ˜50-fold induction, while the NP47 induced a lower activity (FIG. 1B). These observations show how these RNAs differentially induce IFN-β promoter activity when they are transfected into the cytoplasm.

Subsequently the IFN-β promoter activation was validated by the NP47, NP56 and NP76 mvRNA templates during replication by the IAV RNA polymerase. To this end, plasmids expressing the viral RNA polymerase subunits PB1, PB2 and PA, a plasmid expressing NP, and a plasmid expressing the NP76 template mvRNA were transfected into HEK 293T cells. Primer extension analysis showed efficient amplification of NP47, NP56 and NP76, as well as the production of several smaller aberrant RNA products that were shorter than template the mvRNA in the case of NP56 and NP76 (FIGS. 1C and 7). IFN-β promoter activation by NP56 and NP76, but not NP47 was observed. These results thus indicate that like the full-length vRNA segments, mvRNAs themselves can also serve as template for aberrant RNA synthesis. Fractionation of cells in which NP76 was replicated showed that the NP76 mvRNA template was present in the nuclear, cytoplasmic, and mitochondrial fractions, whereas the aberrant RNAs produced from the NP76 template were present in the nucleus only (FIG. 7). Since IAV RNA is predominantly detected in the cytoplasm of the host cell, these results show that the mvRNA template, and not aberrant products shorter than the template mvRNA, play a role in innate immune activation.

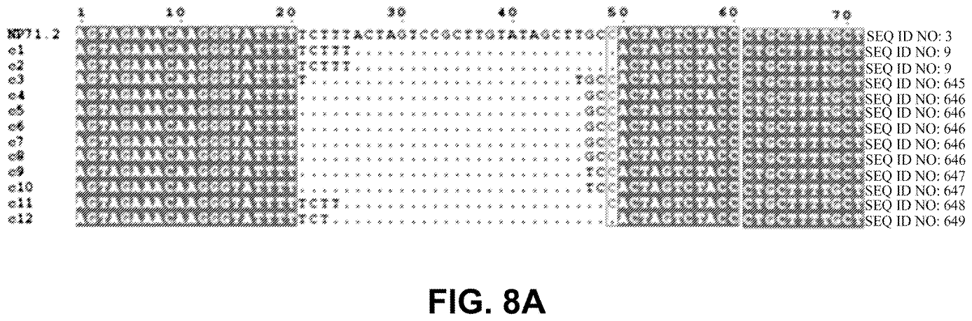

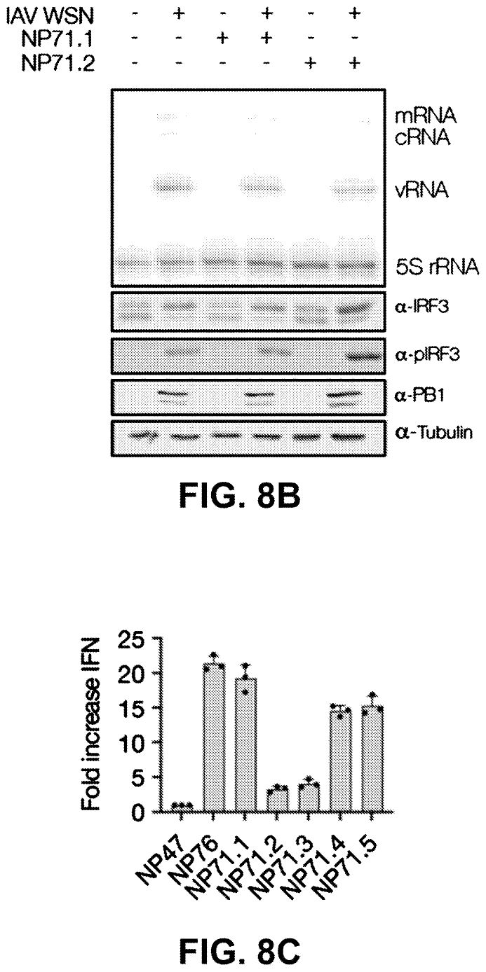

Following the characterization of these assays, the replication and IFN-β promoter activation was analyzed by the engineered mvRNA templates and found that three of these templates were efficiently replicated (NP71.1, NP71.4 and NP71.5), while the other two (NP71.2 and NP71.3) were not (FIG. 1C). Interestingly, among the engineered mvRNAs, templates that were poorly replicated showed higher IFN-β promoter activity and aberrant RNA synthesis (i.e., the production of RNA products containing deletions relative to the template; FIG. 8A) than the three mvRNA templates that were efficiently replicated (FIG. 1C). RT-qPCR analysis of cells replicating NP71.1 and NP71.2 confirmed that endogenous IFN-β mRNA levels were increased when NP71.2 was replicated (FIG. 1D). To confirm that the NP71.2 had the ability to induce innate immune activation during viral infection, NP71.1 and NP71.2 was pre-expressed in absence of viral RNA polymerase and NP in HEK 293T cells. After 24 hours, the cells were infected with 3 MOI influenza virus A/WSN/1933 (H1N1) for 8 hours. As shown in FIG. 8B, pre-expression of NP71.1 and NP71.2 had no effect on segment 6 replication or PB1 protein expression. In addition, phosphorylation of IRF3 after pre-expression of NP71.1 but not NP71.2 was observed. While this is shows MAVS signaling pathway activation through replication of mvRNA by the viral RNA polymerase, amplification of the exogenous mvRNAs could not be detected because they need to compete with the eight endogenous vRNA templates for binding to the viral RNA polymerase expressed by the virus. Thus, it was not determined whether the engineered mvRNAs affect the MAVS signaling pathway in the same way during viral infection as in the RNP reconstitution experiments.

To exclude that a differential recognition of the engineered mvRNAs by host pathogen receptors of the host cell was responsible for the observed increased IFN-β promoter activity on the NP71.2 and NP71.3 mvRNAs, total RNA was isolated from HEK 293T transfections and re-transfected equal amounts of these RNA extracts together with IFN-β and Renilla reporter plasmids into HEK 293T cells. Re-transfection of NP71.1-NP71.5 showed an inverse pattern of IFN-β promoter activation in comparison to FIG. 1C, whereby abundant mvRNAs induced more IFN-β promoter activity than the least abundant mvRNAs (FIG. 8C), showing that there is no inherent difference between the mvRNA in their ability to activate IFN-β promoter activity. Instead, these results indicated that impaired active viral replication determines whether an mvRNA will activate innate immune signaling in the context of an RNP.

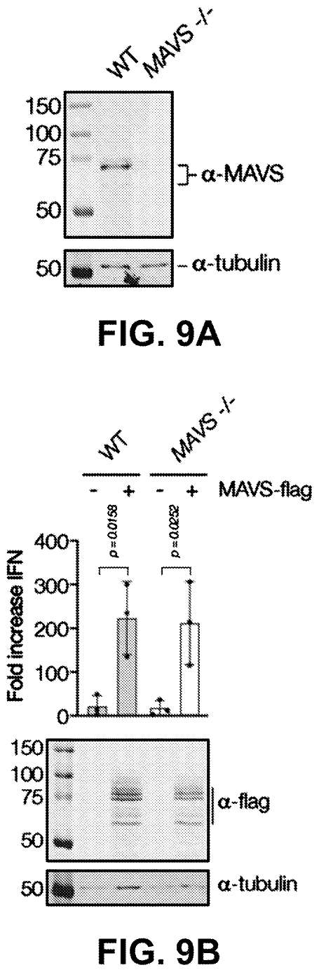

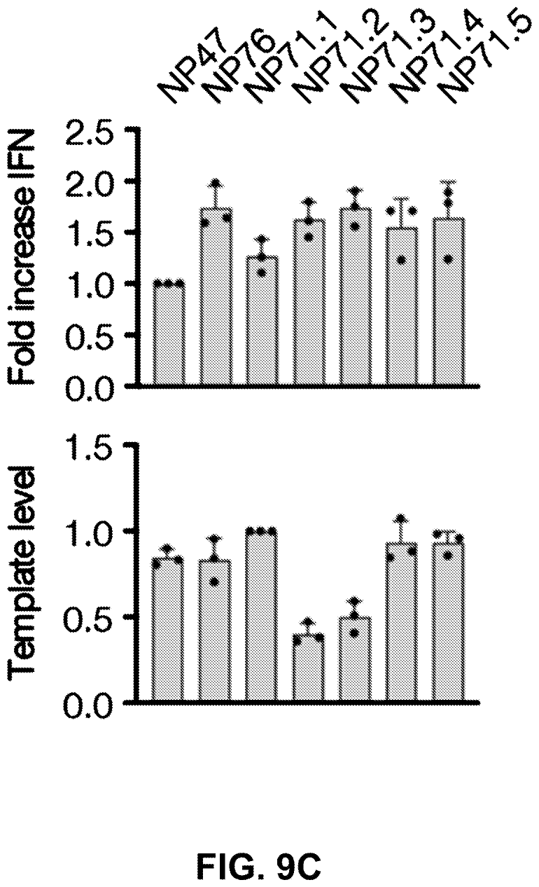

To verify that the different replication efficiencies had not been the result of the effect of NP71.2 and NP71.3 on the innate immune response, these mvRNAs and the WSN RNA polymerase were also expressed in MAVS^{-/-} IFN::luc HEK 293 cells. These MAVS^{-/-} cells do not express endogenous MAVS (FIG. 9A), blocking any RIG-I mediated innate immune signaling, but overexpression of a MAVS-FLAG plasmid still triggers IFN-β promoter activity, indicating that the IFN-β reporter is still functional (FIG. 9B). Expression of NP71.1-NP71.5 in the MAVS^{-/-} cells did not induce IFN-β promoter activity (FIG. 9C). Subsequent primer extension analysis showed that the differences in replication between NP71.1-NP71.5 had been maintained in the MAVS^{-/-} cells (FIG. 9C), demonstrating that the differential replication efficiency is not dependent on the innate immune response.

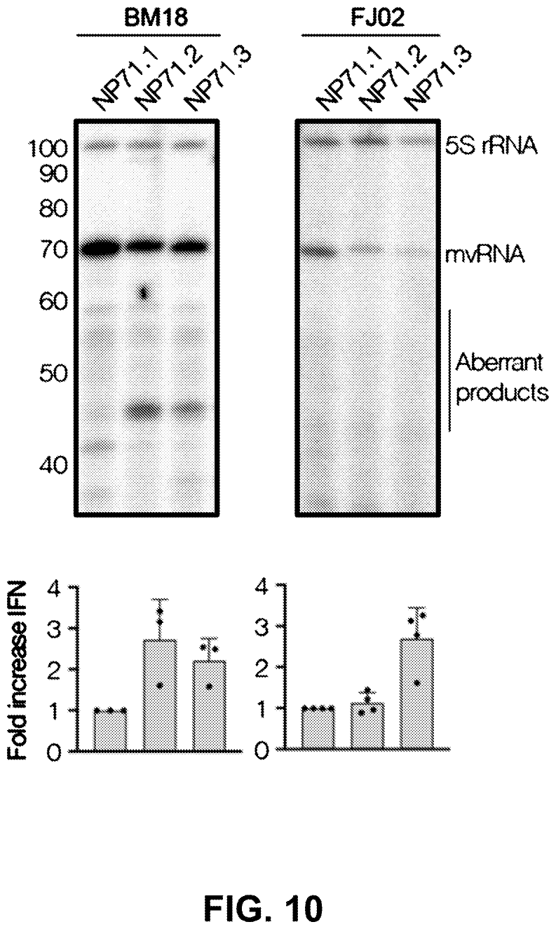

To investigate whether the effect of the NP71.3 and NP71.4 mvRNAs was specific to the WSN polymerase, these mvRNAs were expressed alongside the pandemic H1N1 A/Brevig Mission/1/18 (abbreviated as BM18) or the highly pathogenic avian H5N1 A/duck/Fujian/01/02 (abbreviated as FJ02) RNA polymerases. The BM18 and FJ02 RNA polymerases were impaired on the NP71.2 and NP71.3 mvRNA templates and triggered a stronger IFN-β promoter activity on the NP71.3 template relative to the NP71.1 template (FIG. 10). The BM18 RNA polymerase also produced short aberrant RNA products, while the FJ02 RNA polymerase did not, despite inducing IFN-β promoter activity (FIG. 10). Together, these results show that the mvRNA template is the innate immune agonist, rather than the aberrant RNA products derived from the mvRNA template, and that innate immune activation is dependent on a sequence-specific interaction between the active IAV RNA polymerase and the mvRNA template.

### T-Loops Affect Viral Polymerase Activity and IFN-β Promoter Activation

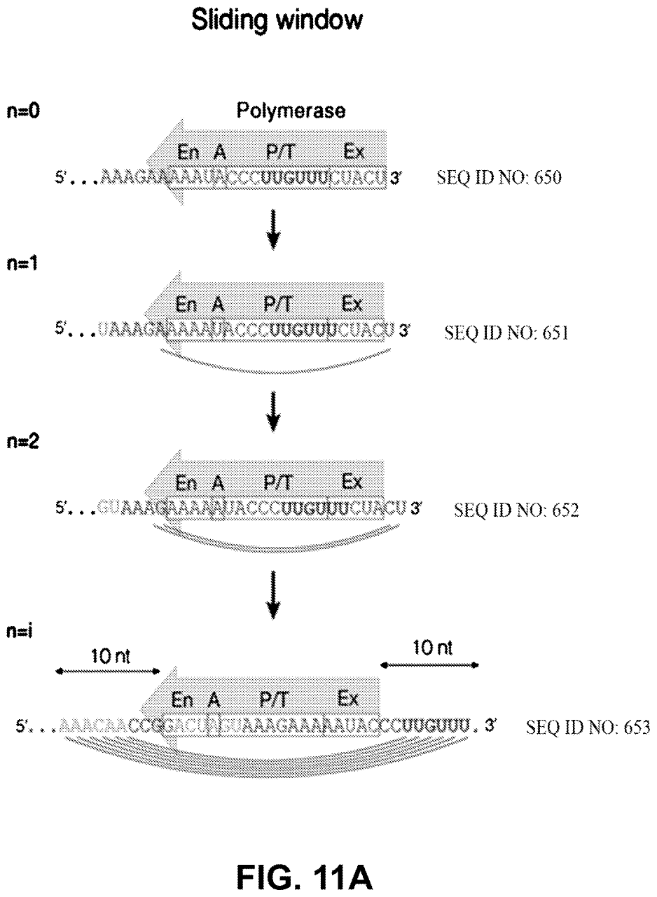

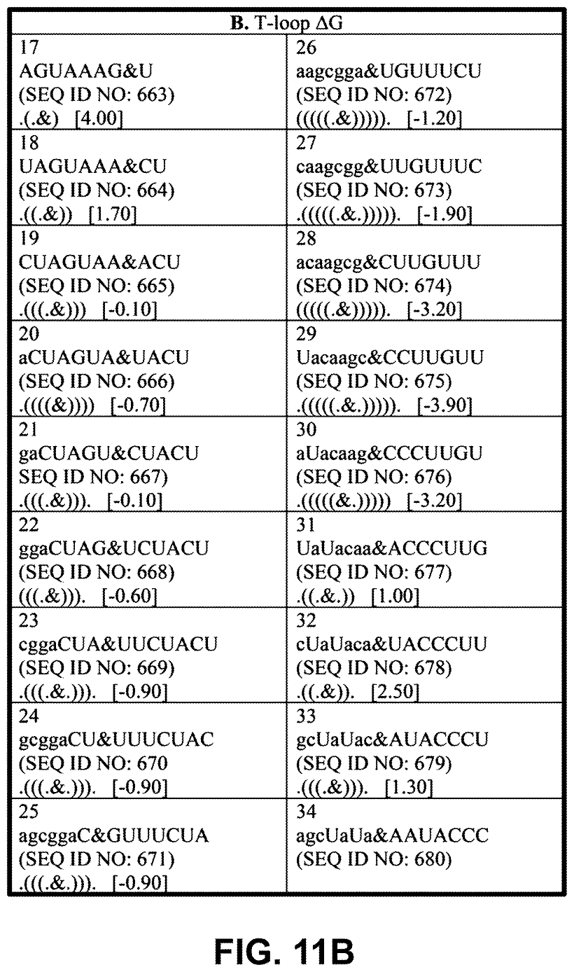

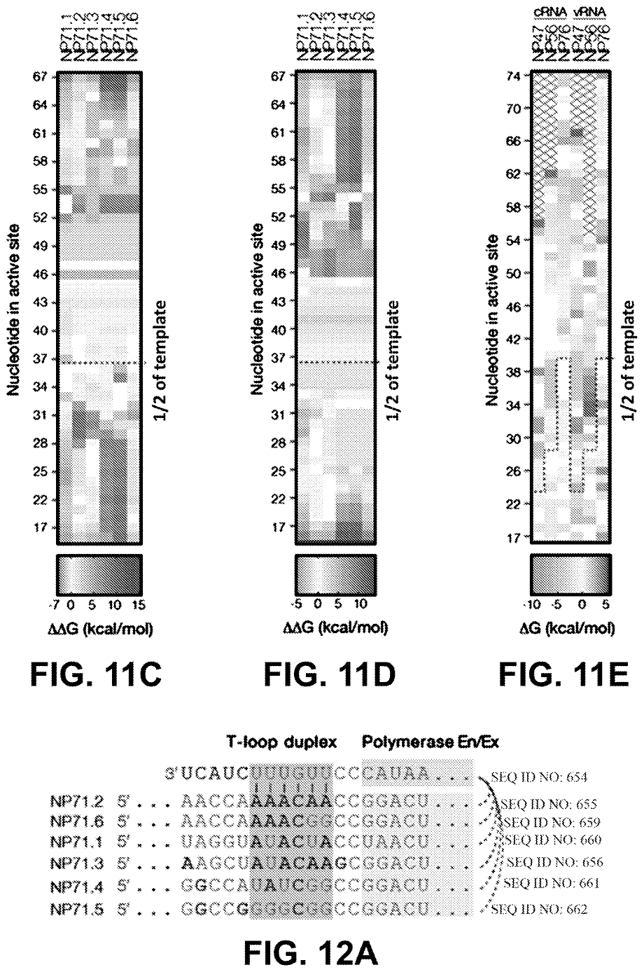

The viral RNA template enters and leaves the active site of the IAV RNA polymerase as a single strand through the entry and exit channels, respectively (FIG. 2A). However, the IAV genome contains various RNA structures that need to be unwound. Moreover, unwinding of these structures may lead to the formation of transient RNA structures upstream or downstream of the RNA polymerase that may modulate RNA polymerase activity (FIG. 2A), while base pairing between a part of the template that is entering the RNA polymerase and a part of the template that has just been duplicated may trap the RNA polymerase in a template loop (t-loop) (FIG. 2A). To systematically analyze what (transient) RNA structures are present during replication, a sliding-window algorithm was used to calculate the minimum free energy (ΔG) for every putative t-loop as well as every putative secondary RNA structure upstream and downstream of the RNA polymerase (FIGS. 11A and 11B). For each position analyzed, 20 nt were excluded from the folding analysis for the footprint of the IAV RNA polymerase and 12 nt from the 5′ terminus, which is stably bound by the RNA polymerase prior to replication termination. As shown in FIG. 2B and FIG. 11C, the analysis revealed that NP71.2 and NP71.3 are unique among the engineered mvRNA templates in forming t-loop structures around nucleotide 29 of the positive sense replicative intermediate (cRNA), but not the negative sense (FIG. 11D), showing that t-loops in the positive sense mvRNA template modulate RNA polymerase activity. The likelihood that the t-loops form in the context of other secondary structures was calculated as the difference (ΔΔG) between the computed ΔG values for the individual structures (FIG. 2B).

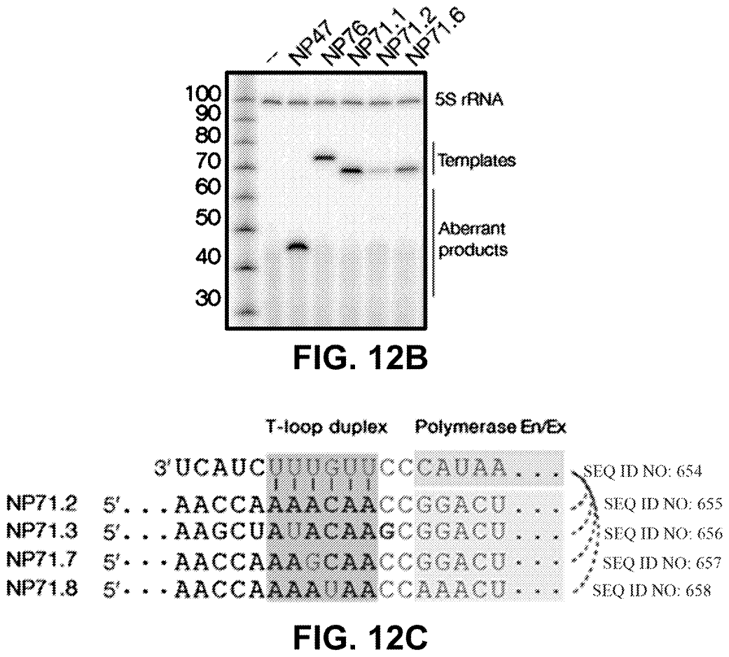

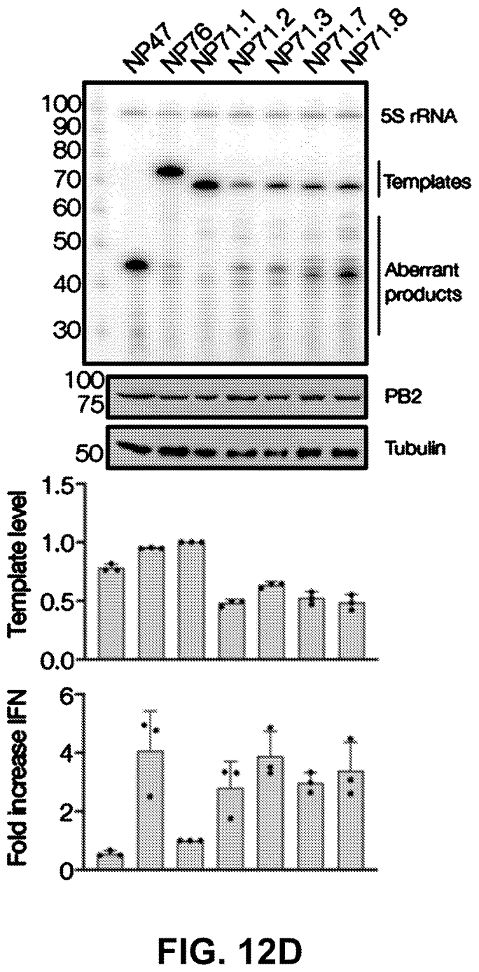

To confirm that t-loops affect RNA polymerase processivity and IFN-β promoter activity, two A-U base pairs of the NP71.2 t-loop duplex were replaced with two G-U base pairs, creating NP71.6 (FIGS. 2B and 12A). Using the sliding window analysis, this mutation was confirmed to make t-loop formation near nucleotide 29 less favorable (FIG. 2B). Following expression of NP71.1, NP71.2 and NP71.6, replication of the NP71.6 mvRNA was increased relative to the NP71.2 mvRNA, our control mvRNAs, and the NP71.1 mvRNA (FIGS. 2C and 12B). In addition, destabilization of the t-loop reduced the induction of the IFN-β promoter activity (FIG. 2C). By contrast, mutating the stem of the t-loop of the NP71.2 mvRNA template such that the t-loop around nucleotide 29 was maintained (FIG. 12C; NP71.7-8), replication remained reduced and IFN-β reporter activity increased relative to the NP71.1 mvRNA (FIGS. 12C and 12D). Replication of mvRNAs with a t-loop led to the production of short aberrant RNA products that likely contained internal deletions. However, increases in aberrant RNA levels were not correlated with increases in IFN-β reporter activity, in line with the results in FIG. 1C and FIG. 6, and indicated that the mvRNA template is the agonist of IFN-β reporter activity. Analysis of control mvRNA templates showed that NP56 and NP76 contain weak t-loops in the first half of both the positive and negative sense template, while stronger t-loops exist in the second half for the NP56 template (FIG. 11E). Together these results indicate that t-loops can negatively affect IAV RNA synthesis and stimulate innate immune signaling during IAV replication.

### T-Loops Reduce RNA Polymerase Processivity In Vitro

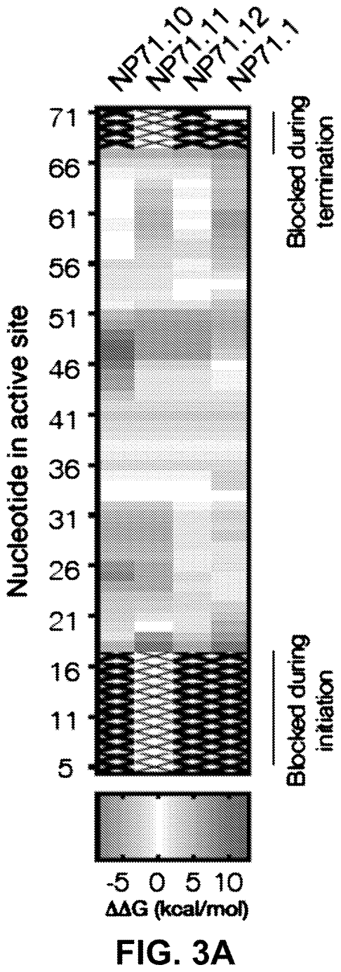

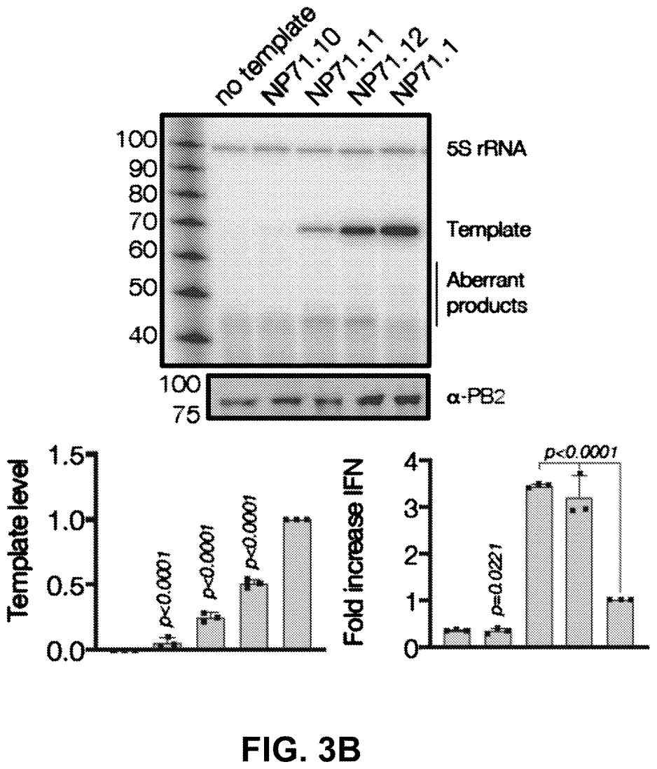

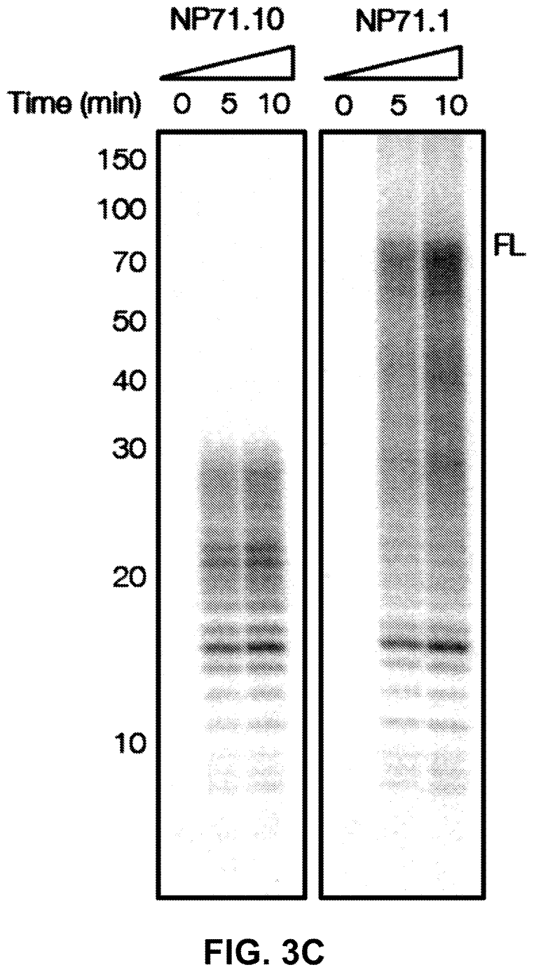

The mvRNAs NP71.2 and NP71.3 contain a t-loop in the first half of the positive sense mvRNA template. To confirm that t-loops also affect RNA polymerase activity in the negative sense, three additional 71-nt long mvRNA templates were engineered with t-loops in different locations of the template (NP71.10-12) (Table 1; FIG. 3A). Expression of these mvRNA templates together with the subunits of the viral RNA polymerase in HEK 293T cells led to strongly reduced NP71.10 and NP71.11 mvRNA levels and slightly reduced NP71.12 mvRNA levels (FIG. 3B). In line with other results (FIGS. 1C and 2C), IFN-β promoter activity was increased for the NP71.11 and NP71.12 templates relative to the NP71.1 mvRNA, while the NP71.10 mvRNA did not induce IFN-β promoter activity because it was too poorly or not fully replicated (FIG. 3B).

To investigate if t-loops affect RNA polymerase processivity in vitro, the WSN RNA polymerase was purified from HEK 293T cells using tandem-affinity purification (TAP) and incubated the enzyme with the NP71.1 and NP71.10 mvRNA templates in the presence of NTPs. Following denaturing PAGE and autoradiography, a main product of approximately 71 nt in reactions was observed containing the NP71.1 control mvRNA (FIG. 3C). By contrast, incubations with the NP71.10 mvRNA template resulted in products up to approximately 27 nt in length, in agreement with the location of the t-loop in the first half of the mvRNA template (FIG. 3A). Moreover, the observed partial extension of the product offered a possible explanation for the reduced RNA levels in cell culture and the lack of IFN-β promoter activity induction by the NP71.10 template (FIG. 3B).

### T-Loops do not Induce Template Release In Vitro

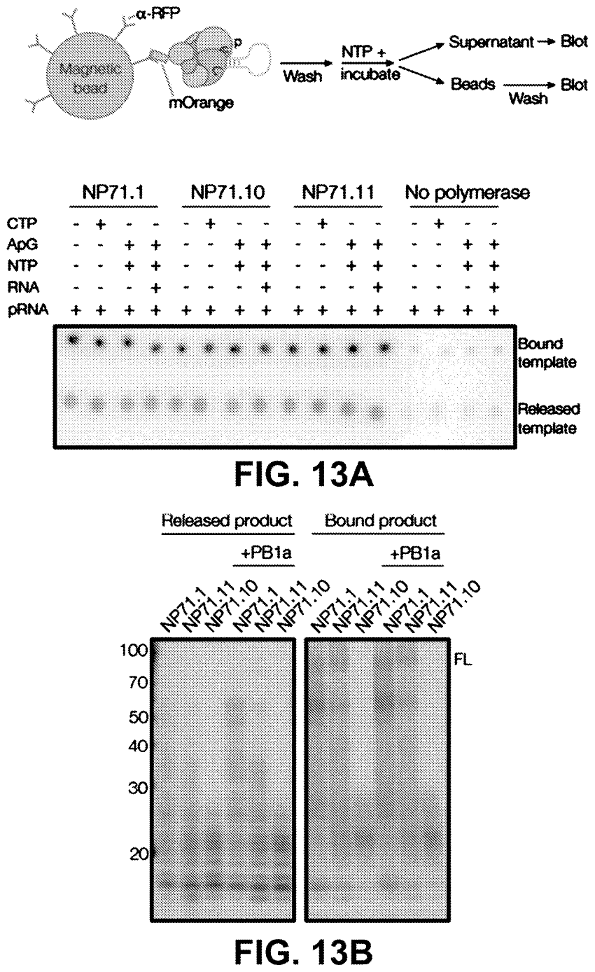

To investigate if t-loop containing templates remained stably bound to the RNA polymerase or triggered template dissociation, mOrange-tagged RNA polymerase was immobilized on magnetic RFP-trap beads, and incubated these immobilized complexes with radiolabeled template. After removal of unbound template by three washes with binding buffer, ApG and nucleotides were added to initiate RNA synthesis and complexes incubated at 30° C. for 15 min. Next, the immobilized complexes were washed three times to remove dissociated RNA, and the reactions stopped with formaldehyde/EDTA loading dye. Analysis of the bound and unbound radiolabeled RNA levels by dot blot and autoradiography showed no difference between the NP71.1, NP71.10 and NP71.11 templates (FIG. 13A). To rule out that released template were rebound upon dissociation from the RNA polymerase, excess unlabeled NP71.1 template was added as RNA polymerase trap at the start of the reaction. Again, no difference between the templates was observed (FIG. 13A).

To confirm that the immobilized RNA polymerases were active, RNA polymerase bound to unlabeled template mvRNA was immobilized on magnetic beads as described above. Next, ApG, NTPs, and radiolabeled GTP was added and incubated the immobilized complexes at 30° C. for 15 min. Following removal of unincorporated NTPs by three washes with binding buffer, the nascent RNA in solution as well as associated with the immobilized complexes was analyzed by denaturing PAGE and autoradiography. As shown in FIG. 13B, partially extended and full-length nascent RNAs remained associated with the immobilized RNA polymerases. Partially extended nascent RNAs were also found in the unbound fraction. Addition of inactive RNA polymerase (PB1a) to serve as encapsulating polymerase in RNA polymerase dimers increased the release of partially extended nascent RNAs, but not the release of full-length RNAs. Together, these results show that t-loops do not induce template release upon RNA polymerase stalling and that partially extended nascent strands can be separated by RNA polymerase from the template strand and released.

PB1 K669A Increases t-Loop Sensitivity and IFN-β Promoter Activation

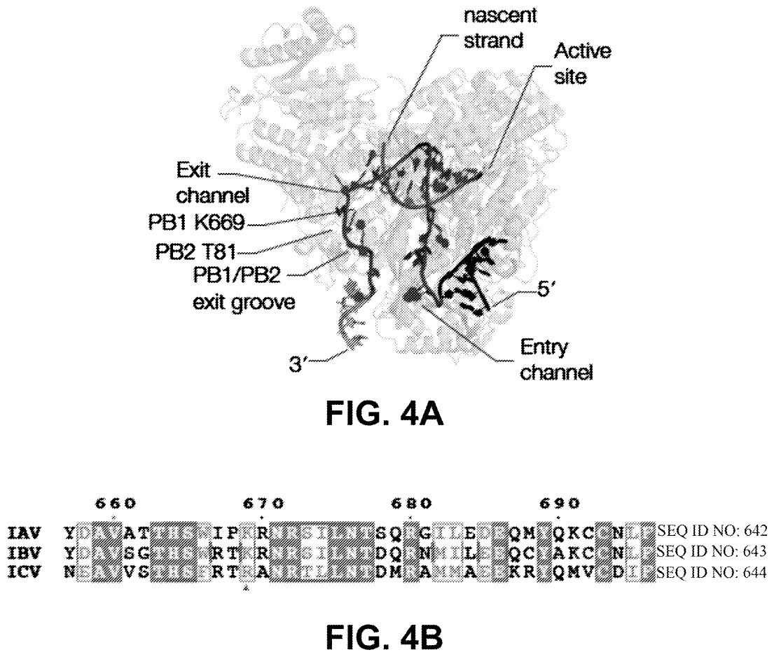

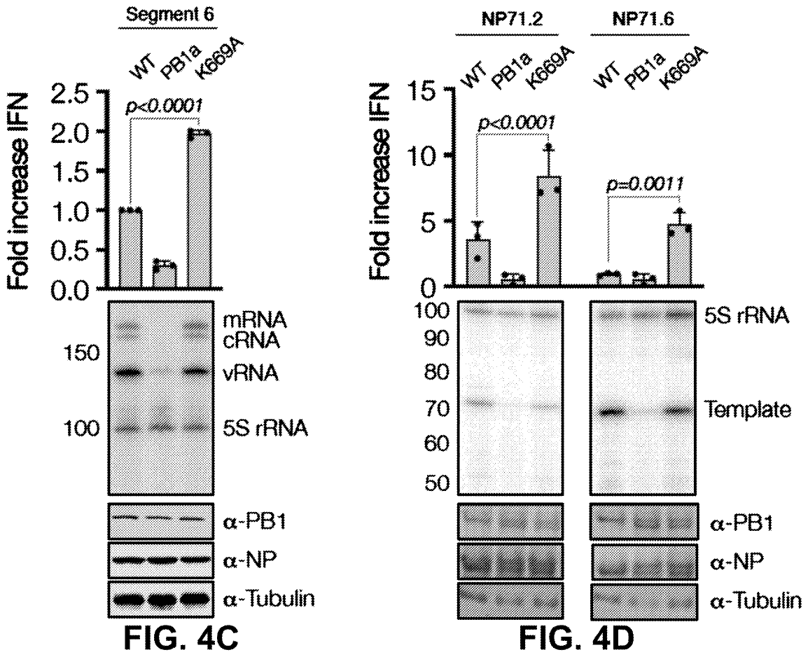

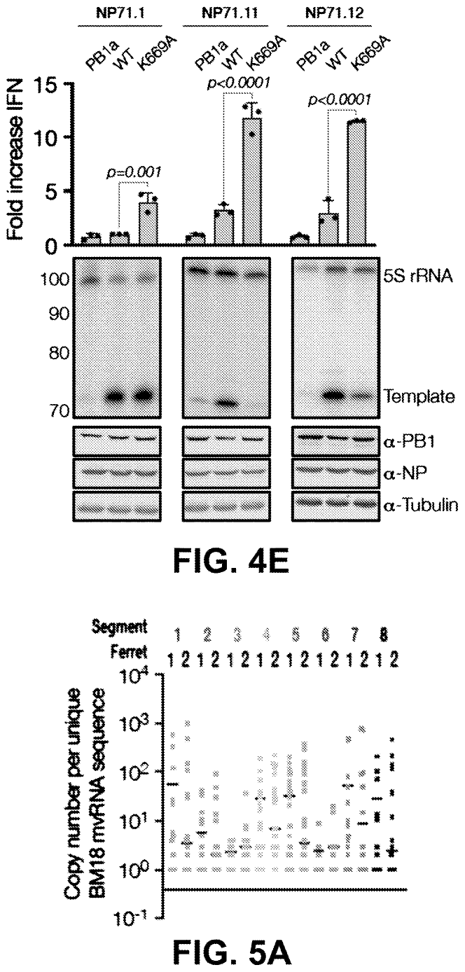

In the IAV RNA polymerase elongation complex, the 3′ terminus of the template is guided out of the template exit channel via an exit groove on the outside of the thumb subdomain. This groove consists of PB1 and PB2 residues, and leads to promoter binding site B (FIGS. 2A and 4A). Since this exit groove and the template entry channel reside next to each other at the top of the RNA polymerase (FIGS. 2A and 4A), perturbation of the path of the 3′ terminus out of the exit channel may stabilize t-loop formation, reduce RNA polymerase activity, and increase IFN-β promoter activation (FIGS. 2A and 4A). It was observed that avian adaptive mutations of highly pathogenic IAV RNA polymerases that increase IFN promoter activation in vitro, such as PB2 M81T (FIG. 4A), reside next to the template exit groove. It was therefore contemplated that other mutations near the template exit channel may make the IAV RNA polymerase more sensitive to t-loops.

To test if dysregulation of the exit groove leads to more IFN-β promoter activation, PB1 lysine 669, which resides at the start of the exit groove (FIG. 3B), were mutated to alanine (K669A). Mutation of this residue had no effect on RNA polymerase activity in the presence of a full-length segment 6 template (FIG. 3C) or the NP71.1 and NP71.6 mvRNA templates that do not contain a stable t-loop (FIGS. 3D and 3E). However, when the K669A mutant was expressed together with the NP71.2, NP71.11 or NP71.12 mvRNAs, which do contain a t-loop in either the positive or negative sense, the K669A mutant displayed greatly reduced RNA polymerase activity (FIGS. 3D and 3E), showing that the K669A mutation increases the processivity defect induced by t-loops. In contrast, the effect of K669A on IFN-β promoter activity was more difficult to interpret, because while the IFN-β promoter activity was considerably increased on the t-loop containing templates (FIGS. 3B and 3E), the K669A mutant also induced significantly higher IFN-β promoter activity relative to the wild-type RNA polymerase on the control templates. These results show that the K669A mutation has two effects: increase the base-level of the RNA polymerase to trigger FN-β promoter activity on templates without a known or destabilized t-loop through an unknown mechanism, and make the RNA polymerase more sensitive to disruption by a t-loop and trigger additional IFN-β promoter activity through this mechanism.

Differential IFN-β Promoter Activation by Natural mvRNAs

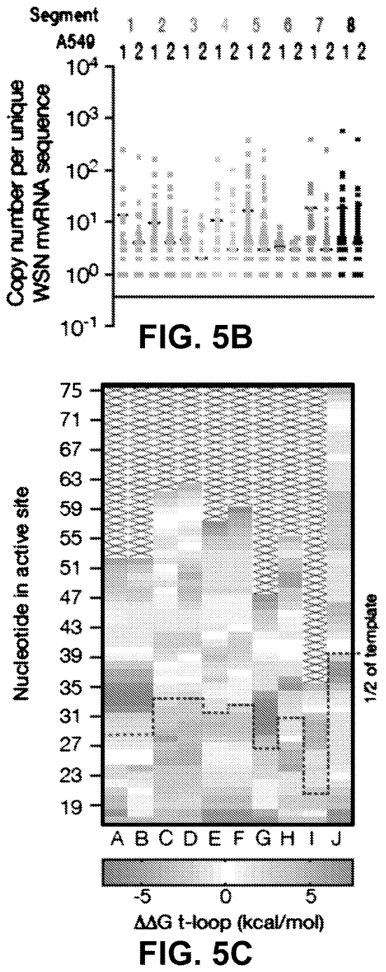

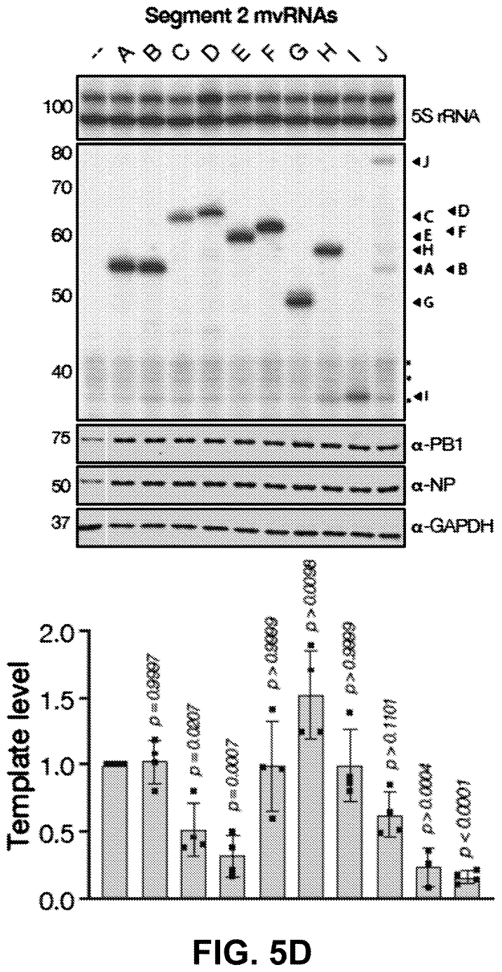

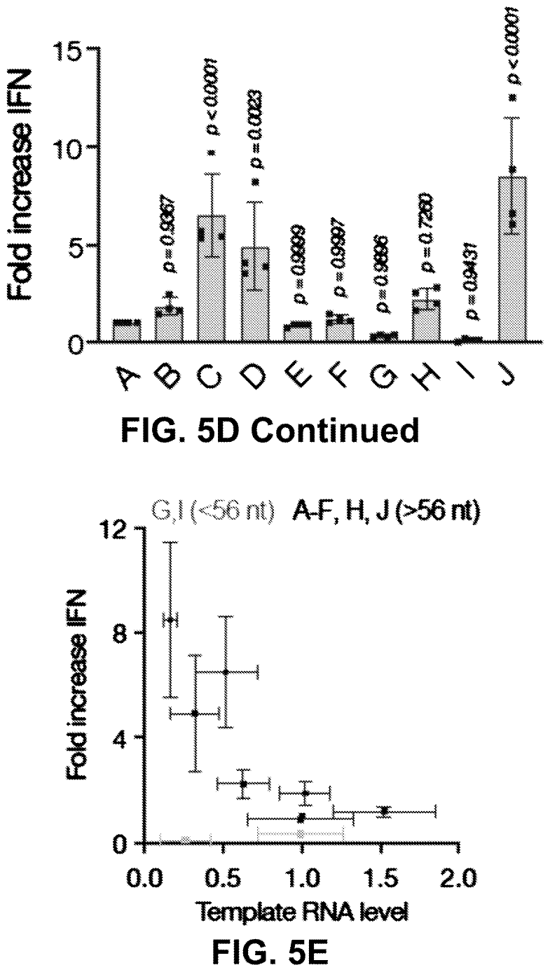

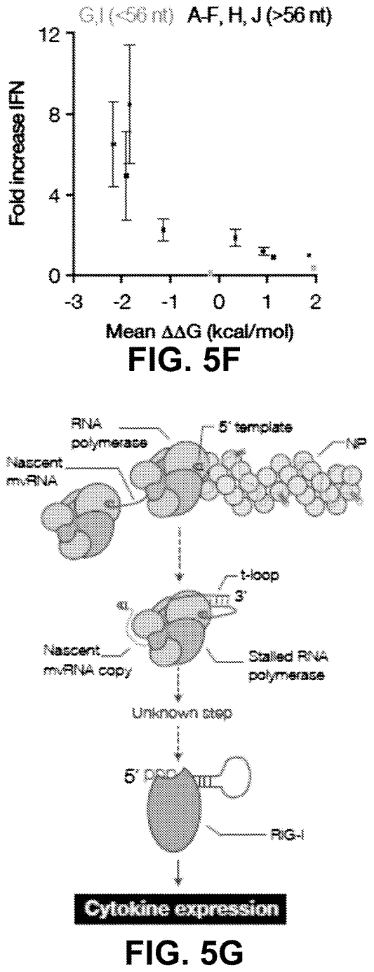

mvRNAs are produced during IAV infection in vitro and in vivo. To study how their sequence and abundance varies, RNA extracted from ferret lungs was examined 1 day post infection with BM18 for 1 day and A549 cells infected with WSN for 8 hours (see Examples 2-5 for mvRNA sequences). Although no quantitative comparisons can be made due to the different infection conditions, a strikingly similar variation in mvRNA sequence and abundance was found (FIGS. 5A and 5B).

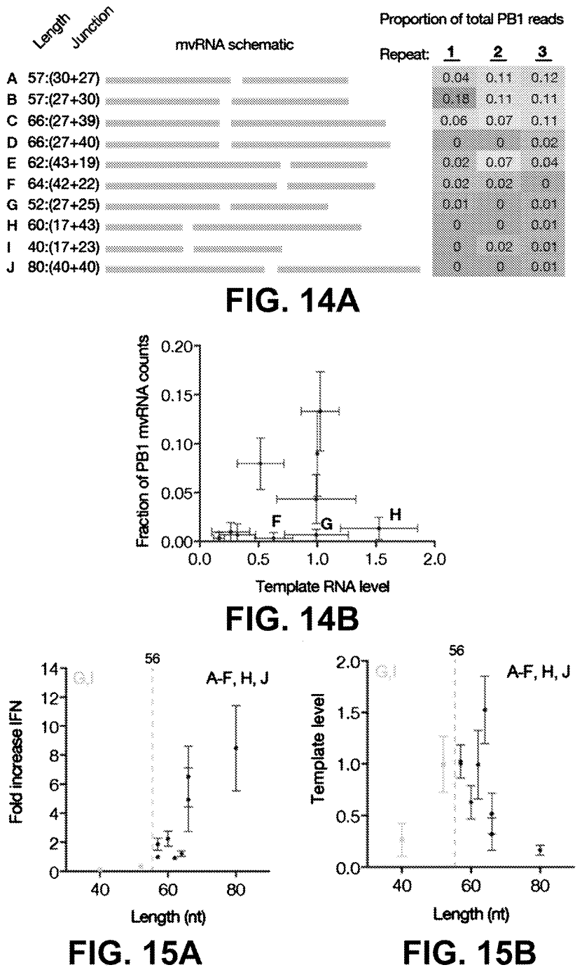

To investigate the implications of these mvRNA differences on the activation of the IFN-β promoter, ten WSN segment 2 mvRNAs (randomly selected over a range of copy numbers and lengths; FIG. 14A) were cloned into pPolI plasmids (mvRNAs A-J; Table 2). Analysis of the ΔΔG values for these mvRNAs revealed potential t-loops in the first half of the sequence for mvRNAs C, D, H and J, and t-loops in the second half of the sequence for mvRNAs E, F and G (FIG. 5C). Subsequent expression of the authentic WSN mvRNAs alongside the WSN RNA polymerase in HEK 293T cells showed significant differences in mvRNA amplification (FIG. 5D). These differences were correlated with the abundance detected by next generation sequencing (NGS) for seven of the cloned mvRNAs (FIG. 14B). In addition, replication of mvRNAs C, D and J leads to the appearance of products shorter than the template mvRNA (FIG. 5D), and the appearance of these products is correlated with a reduced replication of the template mvRNA, in line with the findings in FIG. 1.

To investigate if the different segment 2 mvRNA levels influenced the innate immune response, the IFN-β promoter activity was measured. IFN-β promoter activity varied greatly, with mvRNAs C, D and J inducing the strongest response (FIG. 5D). Templates I and G, the two shortest mvRNAs at 52 and 40 nt long, induced the lowest IFN-β promoter activity, in line with previous observations that short mvRNAs<56 nt do not stimulate RIG-I and FIG. 1C. With mvRNAs I and G excluded due to their short size, these observations indicate that the IFN-β promoter activity is negatively correlated with mvRNA template level for mvRNAs>56 nt (FIG. 5E). Moreover in FIG. 1, t-loops in the first half of the template affect RNA polymerase processivity, the IFN-β promoter activity was negatively correlated with the mean ΔΔG of the first half of the template (FIG. 5F). Weaker correlations were observed between the mvRNA length and IFN-β induction, or the mvRNA length and mvRNA replication (FIGS. 15A and 15B).

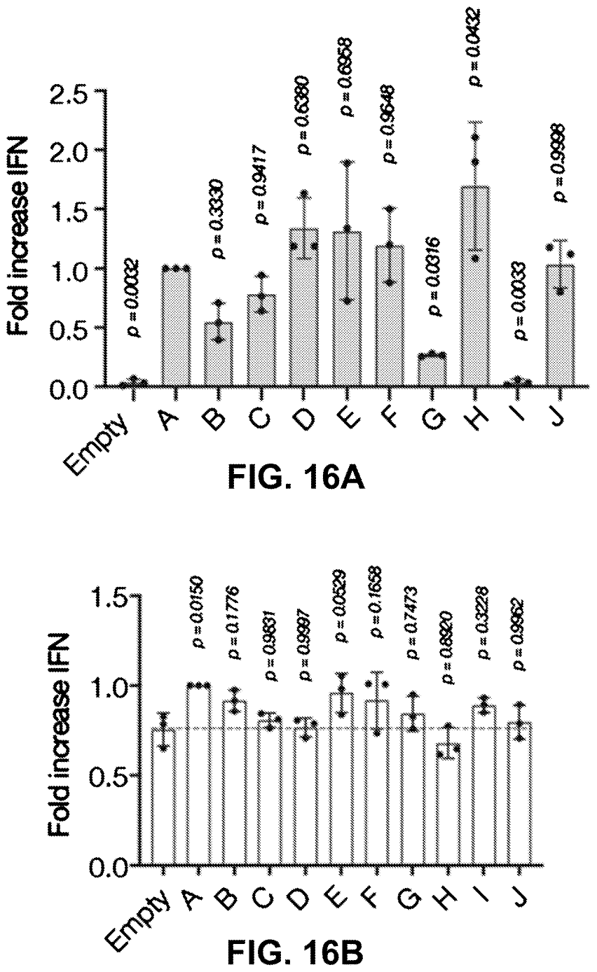

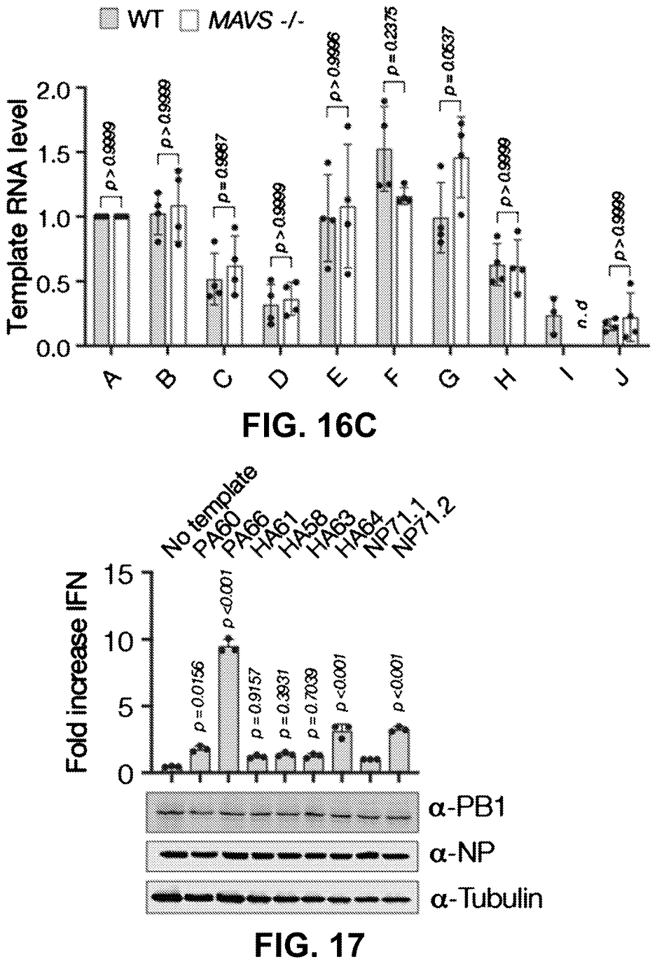

To exclude that a differential recognition of the mvRNAs was responsible for the observed anti-correlation, total RNA was isolated from HEK 293T transfections and re-transfected equal amounts of these RNA extracts into HEK 293T cells. As shown in FIG. 16A, no significant difference among the segment 2 mvRNAs longer than 56 nt was observed. The mvRNAs G and I failed to induce a strong response due to their short length. To exclude that the different mvRNA levels had been the result of their different effects on the innate immune response, the segment 2 mvRNAs were also expressed in MAVS^{-/-} IFN::luc HEK 293 cells. Following expression of the segment 2 mvRNAs, no IFN-β promoter activity was observed (FIG. 16B). Primer extension analysis showed no significant reduction in mvRNA steady-state levels compared to wildtype cells (FIG. 16C), indicating that the replication of authentic mvRNAs is not impacted by innate immune activation.

To confirm that mvRNAs from other viral segments have differential effects on the innate immune response, two segment 3 mvRNAs and four segment 4 mvRNAs (Table 3) were cloned from the mvRNA sequences identified during infection into pPol expression plasmids and transfected these plasmids into HEK 293T cells. As shown in FIG. 17, PA and HA mvRNAs induced both high and low levels of IFN-β promoter activity compared our NP71.1 control. Together, these results indicate that viral infections produce mvRNAs with different mechanisms to induce IFN-β promoter activity, and that t-loops play a key role in the mvRNAs inducing IFN-β promoter activity by affecting the ability of the RNA polymerase to efficiently replicate them.

### Discussion

Two factors important for inducing an innate immune response in IAV infections are active viral replication and the binding of viral RNA molecules to RIG-I. Herein, the effect of IAV mvRNAs, which do not need viral NP to be replicated by the viral RNA polymerase, was examined. Evidence is provided that impeded viral RNA polymerase processivity by t-loops is a mechanism that contributes to the activation of innate immune signaling by mvRNAs. While there is no direct assay to measure or visualize t-loop formation in mvRNAs yet and can thus not rule out other or additional mechanisms, it is postulated that t-loops form when the 3′ terminus or a sequence near the 3′ terminus of the template can hybridize with an upstream part of the template (FIG. 2A). RNA polymerase is thought to unwind a single t-loop, but the formation of several successive t-loops in the first half of the mvRNA stalls the RNA polymerase (FIG. 3C). It is presently still unclear why a strong correlation between reduced processivity and t-loops is observed with t-loops in the first half of the template and not with downstream t-loops.

It is unclear how RIG-I gains access to the t-loop containing mvRNA once the polymerase has stalled (FIG. 4G). RNA polymerase stalling does not result in release of the RNA template from the active site (FIG. 3D) because the RNA polymerase remains associated with the 5′ terminus of the template prior to replication termination. This means that it is still unclear how mvRNA templates accumulate in the cytoplasm and mitochondria (FIG. 7).

One mechanism is that t-loops affect influenza RNA polymerase activity on full-length viral RNAs or DVGs. However, it is more likely that t-loops form only on partially formed RNPs or NP-less templates, since NP may modulate the presence and location of secondary RNA structures. During viral RNA synthesis, NP dissociates and binds viral RNA in a manner that is coordinated by the viral RNA polymerase. When NP levels are reduced, aberrant RNPs or NP-less RNA products form in which secondary RNA structures that are absent in the presence of NP contribute to t-loop formation and RNA polymerase stalling. Indeed, this model explains how reduced viral NP levels stimulate aberrant RNA synthesis and innate immune activation. A mutation near the template exit channel increases the RNA polymerase sensitivity to t-loops (FIG. 4). It was previously observed that avian adaptive mutations, such as PB2 N9D or M81T, reside near the template exit channel of highly pathogenic IAV RNA polymerases and that they stimulate IFN-β promoter activity. Thus, mutations make the RNA polymerase more sensitive to mvRNAs, which are produced at high levels by highly pathogenic IAV RNA polymerases, and this sensitivity leads to increased RNA polymerase stalling by t-loops and IFN-β promoter activation.

During viral infection, mvRNA molecules of various lengths and abundances are produced. It was found that mvRNA abundance is not the best estimate for innate immune activation. An updated model in which mvRNAs that are poorly replicated contribute most to the activation of the innate immune system is contemplated, and thus that activation of the innate immune response is dependent on a template sequence context.

### Material and Methods

### Viral Protein and RNA Expression Plasmids

pcDNA3-based plasmids expressing influenza A/WSN/33 (H1N1) proteins PB1, PB2 PA, NP, PB2-TAP and the active site mutant PB1a (D445/D446A). Mutation K669A was introduced into the pcDNA3-PB1 plasmid by site-directed mutagenesis. mvRNA templates were expressed under the control of the cellular RNA polymerase I promoter from pPolI plasmids. PB1 mvRNA templates were generated by site-directed mutagenesis PCR deletion of pPolI-PB1. Short vRNA templates were created based on the pPolI-NP47 plasmid using the SpeI restriction site.

### Luciferase Assay

Firefly luciferase reporter plasmid under the control of the IFNB promoter (pIFΔ(-116)lucter) and constitutively expressing Renilla luciferase plasmid (pcDNA3-Renilla). The MAVS-FLAG expression vector and corresponding empty vector were cloned based on the pFS420, using the MAVS WT plasmid (pEF-HA-MAVS).

### Cells, Transfections and Infections

Human embryonic kidney (HEK) 293T, Madin-Darby Canine Kidney (MDCK), and A549 cells were originally sourced from the American Type Culture Collection. All cells were routinely screened for Mycoplasma. HEK293 wild-type and MAVS^{-/-} cells expressing luciferase under the control of the IFNB promoter were a kind gift from Dr J. Rehwinkel. All cell cultures were grown in Dulbecco's Modified Eagle Medium (DMEM) (Sigma) with 10% fetal bovine serum (FBS) (Sigma) and 1% L-Glutamine (Sigma). Transfections of HEK293T or HEK293 cell suspensions were performed using Lipofectamine 2000 (Invitrogen) and Opti-Mem (Invitrogen) following the manufacturer's instructions, and transfection of confluent, adherent HEK 293T cells were performed using PEI (Sigma) and Opti-Mem. Infections were performed at MOI 3.

### Antibodies and Western Blotting

IAV proteins were detected using rabbit polyclonal antibodies anti-PB1 (GTX125923; GeneTex), anti-PB2 (GTX125926; GeneTex), and anti-NP (GTX125989; GeneTex) diluted 1:1000 in TBS™ (TBS/0.1% Tween-20 (Sigma)/5% milk). Cellular proteins were detected using the rabbit polyclonal antibodies anti-GAPDH (GTX100118; GeneTex) diluted 1:4000 in TBS™, and anti-RNA Pol II (ab5131; Abcam) diluted 1:100 in TBS™; the mouse monoclonal antibodies anti-MAVS E-3 (sc-166583; Santa Cruz) diluted 1:200 in TMS™, and Mito tracker [113-1](ab92824; Abcam) diluted 1:1000 TBS™; and the rat monoclonal antibody anti-tubulin (MCA77G; Bio-Rad) diluted 1:1000 in TBS™. Mouse monoclonal antibody anti-FLAG M2 (F3165; Sigma) diluted at 1:2000 was used to detect MAVS-FLAG. Secondary antibodies IRDye 800 Donkey anti-rabbit (926-32213; Li-cor), IRDye 800 Goat anti-mouse (926-32210; Li-cor), IRDye 680 Goat anti-mouse (926-68020; Li-cor), and IRDye 680 Goat anti-rat (926-68076; Li-cor), were used to detect western signals with a Li-cor Odyssey scanner.

### RNP Reconstitution Assays and RNA Sequence Analysis

Infections and RNA analyses using primer extensions were performed as described previously. mvRNA identification from next generation sequencing data was essentially performed, using data deposited in the Sequence Read Archive under accession number SUB3758924. Aberrant RNA products observed in various experiments were gel extracted, Topo cloned and sequenced using Sanger sequencing. Alignments were analyzed using Clustal Omega and visualized using Espript 3. T-loop analysis was performed using a custom Python script. Briefly, 20 nt of the template sequence were blocked off to represent the footprint of the viral RNA polymerase. This footprint was then moved in 1 nt increments along the template (FIGS. 11A and 11B). T-loop formation was assessed by computing the ΔG of duplex formation between a stretch of 10 nt upstream of the footprint and 10 nt downstream of the footprint. The formation of upstream and downstream structures was computed for 24 nt windows (the footprint of NP) upstream and downstream of the moving footprint. The ΔΔG was computed by subtracting The ViennaRNA package commands duplex-fold and cofold were used to compute the ΔG values.

### Luciferase-Based IFN Expression Assays

To measure IFN expression in RNP reconstituted HEK293T or HEK293 cells, luciferase assays were carried out 24 h post-transfection. RNP reconstitutions were carried out in a 24-well format by transfecting 0.25 μg of the plasmids pcDNA3-PB1, pcDNA3-PB2, pcDNA3-PA, pcDNA3-NP and a pPolI plasmid expressing a mvRNA template. HEK293T and HEK293 cells were additionally co-transfected with 100 ng of the plasmids pIFΔ(-116)lucter and pcDNA3-Renilla. Cells were harvested in PBS and resuspended in an equal volume of Dual-Glo Reagent (Promega) followed by Dual-Glo Stop & Glo reagent (Promega). Firefly and Renilla luminescence were measured after 10 minutes incubation with each reagent respectively as per manufacturer's instructions for the Dual-Glo Luciferase Assay System (E2920, Promega) using the Glomax luminometer (Promega).

### IAV Polymerase Purification and In Vitro Activity Assay

Influenza virus A/WSN/33 (H1N1) recombinant polymerases were purified from HEK293T cells. Ten cm plates of adherent cells were transfected with 3 μg of pcDNA3-PB1, pcDNA3-PB2-TAP and pcDNA3-PA with PEI (Sigma). Forty-eight hours post-transfection, cells were harvested in PBS and lysed on ice for 10 min in 500 μl lysis buffer (50 mM hepes pH 8.0, 200 mM NaCl, 25% glycerol (Sigma), 0.5% Igepal CA-630 (Sigma), 1 mM β-mercaptoethanol (Bio-Rad), 1×PMSF (Sigma), 1× Protease Inhibitor cocktail tablet (Roche). Lysates were cleared by centrifugation at 17000 g for 5 min at 4° C., diluted in 2 ml NaCl (Sigma), and bound to pre-washed IgG Sepharose beads (Cytiva) for 2 h at 4° C. Beads were pre-washed 3× in binding buffer (10 mM Hepes pH 8.0, 0.15 M NaCl, 0.1% Igepal CA-630, 10% glycerol, 1×PMSF). After binding, beads were washed 3× in binding buffer and 1× in cleavage buffer (10 mM Hepes pH 8.0 (Sigma), 0.15M NaCl, 0.1% Igepal CA-630, 10% glycerol, 1×PMSF, 1 mM DTT). Beads were cleaved with AcTEV protease (Invitrogen) overnight at 4° C., and cleared by centrifugation at 1000 g for 1 min. Activity assays using immobilized RNA polymerase were performed using an RNA polymerase with an mOrange-tag on the PB2 subunit. The purified polymerase was immobilized using magnetic RFP-trap beads (Chromotek).

### Cell Fractionation

Fractionation of transfected cells into cytoplasmic, mitochondrial, and nuclear components was carried out using the Abcam Cell Fractionation Kit (Abcam) following the manufacturer's instructions, with volumes adjusted based on the number of cells. Samples of unfractionated whole cells in Buffer A were retained as input controls. Whole cells and sub-cellular fractions were dissolved in Trizol, for RNA extraction and analyzed as described above, or in 10%-SDS protein-loading buffer, for protein expression analysis by SDS-PAGE and western blot.

### Statistical Testing

Statistical testing was carried out using GraphPad Prism 9 software. Error bars represent standard deviations, and either individual data or group mean values are plotted. One-way analysis of variance (ANOVA) with Dunnett's test for multiple comparisons was used to compare multiple-group means to a normalized mean (e.g., IFN induction or RNA template replication). Two-way ANOVA with Sidak's test for multiple comparisons was used to compare multiple pairs of group means (e.g., between two cell types, HEK293 WT to HEK293 MAVS^{-/-}).

### Example 2: Fasta File 1. WSN mvRNA Sequences

1) -WSN_NP_61
SEQ ID NO: 31
AGCAAAAGCAGGGTAGATAATCACTCACAGAGTGACATAGAAAAATACCCT
TGTTTCTACT
2) -WSN_PB1_57
SEQ ID NO: 32
AGCGAAAGCAGGCAAACCATTTGAATGGATTTCATGAAAAAATGCCTTGT
TTCTACT
3) -WSN_PB1_57
SEQ ID NO: 33
AGCGAAAGCAGGCAAACCATTTGAATGTCCTTCATGAAAAAATGCCTTGT
TTCTACT
4) -WSN_PB1_66
SEQ ID NO: 34
AGCGAAAGCAGGCAAACCATTTGAATGTTTAGCTTGTCCTTCATGAAAAA
ATGCCTTGTTTCTACT
5) -WSN_NP_65
SEQ ID NO: 35
AGCAAAAGCAGGGTAGATAATCACTCACAGAGTGACATCTAAAGAAAAAT
ACCCTTGTTTCTACT
6) -WSN_HA_44
SEQ ID NO: 36
AGCAAAAGCAGGGGAATATAAGGAAAAACACCCTTGTTTCTACT
7) -WSN_NA_70
SEQ ID NO: 37
AGCAAAAGCAGGAGTTTAAATGAATCCAAACCTGACAAGTAGTTTGTTCA
AAAAACTCCTTGTTTCTACT
8) -WSN_PB1_62
SEQ ID NO: 38
AGCGAAAGCAGGCAAACCATTTGAATAGCTTGTCCTTCATGAAAAAATGC
CTTGTTTCTACT
9) -WSN_NP_70
SEQ ID NO: 39
AGCAAAAGCAGGGTAGATAATCACTCACAGAGTGACATCGAAATTAAAGA
AAAATACCCTTGTTTCTACT
10) -WSN_PB1_64
SEQ ID NO: 40
AGCGAAAGCAGGCAAACCATTTGAATGGATGTCAATCCGACTTAAAAAAT
GCCTTGTTTCTACT
11) -WSN_PB1_69
SEQ ID NO: 41
AGCGAAAGCAGGCAAACCATTIGAATGGATGTTAGCTTGTCCTTCATGAA
AAAATGCCTTGTTTCTACT
12) -WSN_NA_69
SEQ ID NO: 42
AGCAAAAGCAGGAGTTTAAATGAATCCAAACCAGAAAGTAGTTTGTTCAA
AAAACTCCTTGTTTCTACT
13) -WSN_PB1_62
SEQ ID NO: 43
AGCGAAAGCAGGCAAACCATTTGAATGGATGTCAATCCGACTTAAAATGC
CTTGTTTCTACT
14) -WSN_PB1_68
SEQ ID NO: 44
AGCGAAAGCAGGCAAACCATTTGAATGGATGTCAATCCGACTTCATGAAA
AAATGCCTTGTTTCTACT
15) -WSN_HA_60
SEQ ID NO: 45
AGCAAAAGCAGGGGAAATTAGGATTTCAGAAATATAAGGAAAAACACCCT
TGTTTCTACT
16) -WSN_PB1_68
SEQ ID NO: 46
AGCGAAAGCAGGCAAACCATTTGAATGGATGTCAATCCTCCTTCATGAAA
AAATGCCTTGTTTCTACT
17) -WSN_PB1_49
SEQ ID NO: 47
AGCGAAAGCAGGCAAACCATTTGAATTGAAAAAATGCCTTGTTTCTACT
18) -WSN_PB1_56
SEQ ID NO: 48
AGCGAAAGCAGGCAAACTTTAGCTTGTCCTTCATGAAAAAATGCCTTGTT
TCTACT
19) -WSN_PB2_60
SEQ ID NO: 49
AGCGAAAGCAGGTCAATTATATTCAATATCGAATAGTTTAAAAACGACCT
TGTTTCTACT
20) -WSN_PB1_60
SEQ ID NO: 50
AGCGAAAGCAGGCAAACCATTTGAATGGATGTCAATATGAAAAAATGCCT
TGTTTCTACT
21) -WSN_NP 74
SEQ ID NO: 51
AGCAAAAGCAGGGTAGATAATCACTCACAGAGTGACATCTACGACAATTA
AAGAAAAATACCCTTGTTTCTACT
22) -WSN_NA_73
SEQ ID NO: 52
AGCAAAAGCAGGAGTTTAAATGAATCCAAACCAGAAAACAAGTAGTTTGT
TCAAAAAACTCCTTGTTTCTACT
23) -WSN_NA_62
SEQ ID NO: 53
AGCAAAAGCAGGAGTTTAAATGAATCCAAACTAGTTTGTTCAAAAAACTC
CTTGTTTCTACT
24) -WSN_NA_63
SEQ ID NO: 54
AGCAAAAGCAGGAGTTTAACACCATTGACAAGTAGTTTGTTCAAAAAACT
CCTTGTTTCTACT
25) -WSN_PB2_55
SEQ ID NO: 55
AGCGAAAGCAGGTCAATTATATTCCGAATAGTTTAAAAACGACCTTGTTT
CTACT
26) -WSN_PB1_44
SEQ ID NO: 56
AGCGAAAGCAGGCAAACCATATGAAAAAATGCCTTGTTTCTACT
27) -WSN_PA_64
SEQ ID NO: 57
AGCGAAAGCAGGTACTGATTCAAATTGCTATCCATACTGTCCAAAAAAGT
ACCTTGTTTCTACT
28) -WSN_NP_70
SEQ ID NO: 58
AGCAAAAGCAGGGTAGATAATCACTCACAGAGTGACATCGAAATCATAGA
AAAATACCCTTGTTTCTACT
29) -WSN_HA_46
SEQ ID NO: 59
AGCAAAAGCAGGGGAAAATAAAAAGAAAAACACCCTTGTTTCTACT
30) -WSN_HA_61
SEQ ID NO: 60
AGCAAAAGCAGGGGAAAATAAAGATTTCAGAAATATAAGGAAAAACACCC
TTGTTTCTACT
31) -WSN_PB1_65
SEQ ID NO: 61
AGCGAAAGCAGGCAAACCATTTGAATGGATGTCAATCCGACTTGAAAAAA
TGCCTTGTTTCTACT
32) -WSN_PB1_61
SEQ ID NO: 62
AGCGAAAGCAGGCAAACCATTTGAATGGTTGTCCTTCATGAAAAAATGCC
TTGTTTCTACT
33) -WSN_PB1_67
SEQ ID NO: 63
AGCGAAAGCAGGCAAACCATTTGAATGATTTAGCTTGTCCTTCATGAAAA
AATGCCTTGTTTCTACT
34) -WSN_PB1_60
SEQ ID NO: 64
AGCGAAAGCAGGCAAACCATTTGTAGCTTGTCCTTCATGAAAAAATGCCT
TGTTTCTACT
35) -WSN_PB1_64
SEQ ID NO: 65
AGCGAAAGCAGGCAAACCATGTGAATTTAGCTTGTCCTTCATGAAAAAAT
GCCTTGTTTCTACT
36) -WSN_PA_66
SEQ ID NO: 66
AGCGAAAGCAGGTACTGATTTACTATTTGCTATCCATACTGTCCAAAAAA
GTACCTTGTTTCTACT
37) -WSN_NP 69
SEQ ID NO: 67
AGCAAAAGCAGGGTAGATAATCACTCACAGAGTGACATCGAAATCATGGC
AAATACCCTTGTTTCTACT
38) -WSN_NP_70
SEQ ID NO: 68
AGCAAAAGCAGGGTAGATAATCACTCACAGAGTGACATCGAAATCATGGC
AAAATACCCTTGTTTCTACT
39) -WSN_NP_66
SEQ ID NO: 69
AGCAAAAGCAGGGTAGATAATCACTCACAGAGTGACATCTTAAAGAAAAA
TACCCTTGTTTCTACT
40) -WSN_NP_64
SEQ ID NO: 70
AGCAAAAGCAGGGTAGATAATCACTCACAGAGTGACATTAAAGAAAAATA
CCCTTGTTTCTACT
41) -WSN_NP_57
SEQ ID NO: 71
AGCAAAAGCAGGGTAGATAATCACTCACAGAGTGACAAAAATACCCTTGT
TTCTACT
42) -WSN_NP 59
SEQ ID NO: 72
AGCAAAAGCAGGGTAGATAATCACTCACAGAGTTAAAGAAAAATACCCTT
GTTTCTACT
43) -WSN_NP_55
SEQ ID NO: 73
AGCAAAAGCAGGGTAGATAATCACTCACAGAGAGAAAAATACCCTTGTTT
CTACT
44) -WSN_NA_64
SEQ ID NO: 74
AGCAAAAGCAGGAGTTTAAATGAATCCAAACCATAGTTTGTTCAAAAAAC
TCCTTGTTTCTACT
45) -WSN_NA_71
SEQ ID NO: 75
AGCAAAAGCAGGAGTTTAAATGAATCCAACCATTGACAAGTAGTTTGTTC
AAAAAACTCCTTGTTTCTACT
46) -WSN_HA_64
SEQ ID NO: 76
AGCAAAAGCAGGGGAATGAGATTAGGATTTCAGAAATATAAGGAAAAACA
CCCTTGTTTCTACT
47) -WSN_PB2_62
SEQ ID NO: 77
AGCGAAAGCAGGTCAATTATATTCAATATGGAGAATAGTTTAAAAACGAC
CTTGTTTCTACT
48) -WSN_PB2_57
SEQ ID NO: 78
AGCGAAAGCAGGTCAATTATATTCAATATGTAGTTTAAAAACGACCTTGT
TTCTACT
49) -WSN_PB2_48
SEQ ID NO: 79
AGCGAAAGCAGGTCAATTATATTCATTAAAAACGACCTTGTTTCTACT
50) -WSN_PB2_54
SEQ ID NO: 80
AGCGAAAGCAGGTCAATTATAGTCGAATAGTTTAAAAACGACCTTGTTTC
TACT
51) -WSN_PB2_49
SEQ ID NO: 81
AGCGAAAGCAGGTCAATTAGAATAGTTTAAAAACGACCTTGTTTCTACT
52) -WSN_PB2_58
SEQ ID NO: 82
AGCGAAAGCAGGTCAATCAATTAGTGTCGAATAGTTTAAAAACGACCTTG
TTTCTACT
53) -WSN_PB1_68
SEQ ID NO: 83
AGCGAAAGCAGGCAAACCATTIGAATGGATGTCAATCCGACTTTACGAAA
AAATGCCTTGTTTCTACT
54) -WSN_PB1_73
SEQ ID NO: 84
AGCGAAAGCAGGCAAACCATTTGAATGGATGTCAATCCGACTTTCCTTCA
TGAAAAAATGCCTTGTTTCTACT
55) -WSN_PB1_61
SEQ ID NO: 85
AGCGAAAGCAGGCAAACCATTTGAATGGATGTCAATCCGACTTAAATGCC
TTGTTTCTACT
56) -WSN_PB1_63
SEQ ID NO: 86
AGCGAAAGCAGGCAAACCATTTGAATGGATGTCAATCCGACTTAAAAATG
CCTTGTTTCTACT
57) -WSN_PB1_80
SEQ ID NO: 87
AGCGAAAGCAGGCAAACCATTTGAATGGATGTCAATCCGAATTTAGCTTG
TCCTTCATGAAAAAATGCCTTGTTTCTACT
58) -WSN_PB1_47
SEQ ID NO: 88
AGCGAAAGCAGGCAAACCATTTGAATGAAAAATGCCTTGTTTCTACT
59) -WSN_PB1_48
SEQ ID NO: 89
AGCGAAAGCAGGCAAACCATTTGAATGAAAAAATGCCTTGTTTCTACT
60) -WSN_PB1_64
SEQ ID NO: 90
AGCGAAAGCAGGCAAACCATTTGAATGTAGCTTGTCCTTCATGAAAAAAT
GCCTTGTTTCTACT
61) -WSN_PB1_62
SEQ ID NO: 91
AGCGAAAGCAGGCAAACCATTTGATTAGCTTGTCCTTCATGAAAAAATGC
CTTGTTTCTACT
62) -WSN_PB1_43
SEQ ID NO: 92
AGCGAAAGCAGGCAAACCATTTAAAAAATGCCTTGTTTCTACT
63) -WSN_PB1_40
SEQ ID NO: 93
AGCGAAAGCAGGCAAACTGAAAAAATGCCTTGTTTCTACT
64) -WSN_PB1_50
SEQ ID NO: 94
AGCGAAAGCAGGCAAACTTGTCCTTCATGAAAAAATGCCTTGTTTCTACT
65) -WSN_NS 47
SEQ ID NO: 95
AGCAAAAGCAGGGTGACAAAGAAATAAAAAACACCCTTGTTTCTACT
66) -WSN_NP_72
SEQ ID NO: 96
AGCAAAAGCAGGGTAGATAATCACTCACAGAGTGACATCGAAATCATGGA
GAAAAATACCCTTGTTTCTACT
67) -WSN_NP_88
SEQ ID NO: 97
AGCAAAAGCAGGGTAGATAATCACTCACAGAGTGACATCGAAATCATGGG
GAGTACGACAATTAAAGAAAAATACCCTTGTTTCTACT
68) -WSN_NP_91
SEQ ID NO: 98
AGCAAAAGCAGGGTAGATAATCACTCACAGAGTGACATCGAAATAATGCA
GAGGAGTACGACAATTAAAGAAAAATACCCTTGTTTCTACT
69) -WSN_NP 81
SEQ ID NO: 99
AGCAAAAGCAGGGTAGATAATCACTCACAGAGTGACATCGAAAGAGTACG
ACAATTAAAGAAAAATACCCTTGTTTCTACT
70) -WSN_NP 62
SEQ ID NO: 100
AGCAAAAGCAGGGTAGATAATCACTCACAGAGTGACATCGGAAAAATACC
CTTGTTTCTACT
71) -WSN_NP_57
SEQ ID NO: 101
AGCAAAAGCAGGGTAGATAATCACTCACAGAGTGACATCAATACCCTTGT
TTCTACT
72) -WSN_NP_58
SEQ ID NO: 102
AGCAAAAGCAGGGTAGATAATCACTCACAGAGTGACATCAAATACCCTTG
TTTCTACT
73) -WSN_NP_81
SEQ ID NO: 103
AGCAAAAGCAGGGTAGATAATCACTCACAGAGTGAATGCAGAGGAGTACG
ACAATTAAAGAAAAATACCCTTGTTTCTACT
74) -WSN_NP_62
SEQ ID NO: 104
AGCAAAAGCAGGGTAGATAATCACTCATACGACAATTAAAGAAAAATACC
CTTGTTTCTACT
75) -WSN_NP_49
SEQ ID NO: 105
AGCAAAAGCAGGGTAGAGACAATTAAAGAAAAATACCCTTGTTTCTACT
76) -WSN_NA_72
SEQ ID NO: 106
AGCAAAAGCAGGAGTTTAAATGAATCCAAACCAGAAAAAAGTAGTTTGTT
CAAAAAACTCCTTGTTTCTACT
77) -WSN_NA__70
SEQ ID NO: 107
AGCAAAAGCAGGAGTTTAAATGAATCCAAACCAGACAAGTAGTTTGTTCA
AAAAACTCCTTGTTTCTACT
78) -WSN_NA_69
SEQ ID NO″ 108
AGCAAAAGCAGGAGTTTAAATGAATCCAAATTGACAAGTAGTTTGTTCAA
AAAACTCCTTGTTTCTACT
79) -WSN_NA_45
SEQ ID NO: 109
AGCAAAAGCAGGAGTTTAAATTTCAAAAAACTCCTTGTTTCTACT
80) -WSN_NA_52
SEQ ID NO: 110
AGCAAAAGCAGGAGTTTAAATTAGTTTGTTCAAAAAACTCCTTGTTTCTA
CT
81) -WSN_NA_62
SEQ ID NO: 111
AGCAAAAGCAGGAGTTTAAACCATTGACAAGTAGTTTGTTCAAAAAACTC
CTTGTTTCTACT
82) -WSN_M_70
SEQ ID NO: 112
AGCGAAAGCAGGTAGATATTGAAAGATGAGTCTTCTAACCGAGGTCGTAA
AAAACTACCTTGTTTCTACT
83) -WSN_M_47
SEQ ID NO: 113
AGCGAAAGCAGGTAGATATTGAAAGATAAAACTACCTTGTTTCTACT
84) -WSN_HA_52
SEQ ID NO: 114
AGCAAAAGCAGGGGAAAATAAAAACAACCAGAAAAACACCCTTGTTTCTA
CT
85) -WSN_HA_49
SEQ ID NO: 115
AGCAAAAGCAGGGGAAAATAAAAACAAGAAAAACACCCTTGTTTCTACT
86) -WSN_HA_48
SEQ ID NO: 116
AGCAAAAGCAGGGGAAAATAAAAACAGAAAAACACCCTTGTTTCTACT
87) -WSN_HA_63
SEQ ID NO: 117
AGCAAAAGCAGGGGAAAATAAAAACATTTCAGAAATATAAGGAAAAACAC
CCTTGTTTCTACT
88) -WSN_HA_67
SEQ ID NO: 118
AGCAAAAGCAGGGGAAAATAAAGATTAGGATTTCAGAAATATAAGGAAAA
ACACCCTTGTTTCTACT
89) -WSN_HA_61
SEQ ID NO: 119
AGCAAAAGCAGGGGAAAATTAGGATTTCAGAAATATAAGGAAAAACACCC
TTGTTTCTACT
90) -WSN_HA_58
SEQ ID NO: 120
AGCAAAAGCAGGGGAATAGGATTTCAGAAATATAAGGAAAAACACCCTTG
TTTCTACT
91) -WSN_PB2_57
SEQ ID NO: 121
AGCGAAAGCAGGTCAATTATATTCAATATGGAAAGAAAAAACGACCTTGT
TTCTACT
92) -WSN_PB2_69
SEQ ID NO: 122
AGCGAAAGCAGGTCAATTATATTCAATATGGAAAGAGTCGAATAGTTTAA
AAACGACCTTGTTTCTACT
93) -WSN_PB2_67
SEQ ID NO: 123
AGCGAAAGCAGGTCAATTATATTCAATATGTAGTGTCGAATAGTTTAAAA
ACGACCTTGTTTCTACT
94) -WSN_PB2_51
SEQ ID NO: 124
AGCGAAAGCAGGTCAATTATATTCAATATTAAAAACGACCTTGTTTCTAC
T
95) -WSN_PB2_65
SEQ ID NO: 125
AGCGAAAGCAGGTCAATTATATTCAATATAGTGTCGAATAGTTTAAAAAC
GACCTTGTTTCTACT
96) -WSN_PB2_59
SEQ ID NO: 126
AGCGAAAGCAGGTCAATTATATTCAATACGAATAGTTTAAAAACGACCTT
GTTTCTACT
97) -WSN_PB2_62
SEQ ID NO: 127
AGCGAAAGCAGGTCAATTATATTCAATGTGTCGAATAGTTTAAAAACGAC
CTTGTTTCTACT
98) -WSN_PB2_52
SEQ ID NO: 128
AGCGAAAGCAGGTCAATTATATTCATAGTTTAAAAACGACCTTGTTTCTA
CT
99) -WSN_PB2_63
SEQ ID NO: 129
AGCGAAAGCAGGTCAATTATATTCATTAGTGTCGAATAGTTTAAAAACGA
CCTTGTTTCTACT
100) -WSN_PB2_44
SEQ ID NO: 130
AGCGAAAGCAGGTCAATTATATTAAAAACGACCTTGTTTCTACT
101) -WSN_PB2_60
SEQ ID NO: 131
AGCGAAAGCAGGTCAATTATATTTAGTGTCGAATAGTTTAAAAACGACCT
TGTTTCTACT
102) -WSN_PB2_46
SEQ ID NO: 132
AGCGAAAGCAGGTCAATAATAGTTTAAAAACGACCTTGTTTCTACT
103) WSN_PB2_51
SEQ ID NO: 133-
AGCGAAAGCAGGTCAAGTGTCGAATAGTTTAAAAACGACCTTGTTTCTAC
T
104) -WSN_PB1_73
SEQ ID NO: 134
AGCGAAAGCAGGCAAACCATTTGAATGGATGTCAATCCGACTTTACTTTT
CTAAAAAATGCCTTGTTTCTACT
105) -WSN_PB1_69
SEQ ID NO: 135
AGCGAAAGCAGGCAAACCATTTGAATGGATGTCAATCCGACTTTACTTTT
AAAATGCCTTGTTTCTACT
106) -WSN_PB1_75
SEQ ID NO: 136
AGCGAAAGCAGGCAAACCATTTGAATGGATGTCAATCCGACTTTACTCTT
CATGAAAAAATGCCTTGTTTCTACT
107) -WSN_PB1_65
SEQ ID NO: 137
AGCGAAAGCAGGCAAACCATTTGAATGGATGTCAATCCGACTTTACAAAA
TGCCTTGTTTCTACT
108) -WSN_PB1_64
SEQ ID NO: 138
AGCGAAAGCAGGCAAACCATTTGAATGGATGTCAATCCGACTTTAAAAAT
GCCTTGTTTCTACT
109) -WSN_PB1_61
SEQ ID NO: 139
AGCGAAAGCAGGCAAACCATTTGAATGGATGTCAATCCGACTAAAATGCC
TTGTTTCTACT
110) -WSN_PB1_73
SEQ ID NO: 140
AGCGAAAGCAGGCAAACCATTTGAATGGATGTCAATCCGACTGTCCTTCA
TGAAAAAATGCCTTGTTTCTACT
111) -WSN_PB1_63
SEQ ID NO: 141
AGCGAAAGCAGGCAAACCATTTGAATGGATGTCAATCCCATGAAAAAATG
CCTTGTTTCTACT
112) -WSN_PB1_60
SEQ ID NO: 142
AGCGAAAGCAGGCAAACCATTTGAATGGATGTCAATCTGAAAAAATGCCT
TGTTTCTACT
113) -WSN_PB1_80
SEQ ID NO: 143
AGCGAAAGCAGGCAAACCATTTGAATGGATGTCAATCTGAATTTAGCTTG
TCCTTCATGAAAAAATGCCTTGTTTCTACT
114) -WSN_PB1_63
SEQ ID NO: 144
AGCGAAAGCAGGCAAACCATTTGAATGGATGTCACCTTCATGAAAAAATG
CCTTGTTTCTACT
115) -WSN_PB1_65
SEQ ID NO: 145
AGCGAAAGCAGGCAAACCATTTGAATGGATGTCAGTCCTTCATGAAAAAA
TGCCTTGTTTCTACT
116) -WSN_PB1_58
SEQ ID NO: 146
AGCGAAAGCAGGCAAACCATTTGAATGGATGTCCATGAAAAAATGCCTTG
TTTCTACT
117) -WSN_PB1_52
SEQ ID NO: 147
AGCGAAAGCAGGCAAACCATTTGAATGGATGTAAAAATGCCTTGTTTCTA
CT
118) -WSN_PB1_63
SEQ ID NO: 148
AGCGAAAGCAGGCAAACCATTTGAATGGACTTGTCCTTCATGAAAAAATG
CCTTGTTTCTACT
119) -WSN_PB1_59
SEQ ID NO: 149
AGCGAAAGCAGGCAAACCATTTGAATGGGTCCTTCATGAAAAAATGCCTT
GTTTCTACT
120) -WSN_PB1_52
SEQ ID NO: 150
AGCGAAAGCAGGCAAACCATTTGAATGCATGAAAAAATGCCTTGTTTCTA
CT
121) -WSN_PB1_48
SEQ ID NO: 151
AGCGAAAGCAGGCAAACCATTTTCATGAAAAAATGCCTTGTTTCTACT
122) -WSN_PB1_61
SEQ ID NO: 152
AGCGAAAGCAGGCAAACCATTTTTTAGCTTGTCCTTCATGAAAAAATGCC
TTGTTTCTACT
123) -WSN_PB1_57
SEQ ID NO: 153
AGCGAAAGCAGGCAAACCATTAGCTTGTCCTTCATGAAAAAATGCCTTGT
TTCTACT
124) -WSN_PB1_60
SEQ ID NO: 154
AGCGAAAGCAGGCAAACTGAATTTAGCTTGTCCTTCATGAAAAAATGCCT
TGTTTCTACT
125) -WSN_PB1_63
SEQ ID NO: 155
AGCGAAAGCAGGCAAACTAGTGAATTTAGCTTGTCCTTCATGAAAAAATG
CCTTGTTTCTACT
126) -WSN_PB1_56
SEQ ID NO: 156
AGCGAAAGCAGGCAAAATTTAGCTTGTCCTTCATGAAAAAATGCCTTGTT
TCTACT
127) -WSN_PA_63
SEQ ID NO: 157
AGCGAAAGCAGGTACTGATTCAAAATGGAAGATTTTGTTCCAAAAAAGTA
CCTTGTTTCTACT
128) -WSN_PA_58
SEQ ID NO: 158
AGCGAAAGCAGGTACTGATTCAAAATGGAAGATTTTAAAAAAGTACCTTG
TTTCTACT
129) -WSN_PA_53
SEQ ID NO: 159
AGCGAAAGCAGGTACTGATTCAAAATGGAAGATTAAAGTACCTTGTTTCT
ACT
130) -WSN_PA_63
SEQ ID NO: 160
AGCGAAAGCAGGTACTGATTTATTTGCTATCCATACTGTCCAAAAAAGTA
CCTTGTTTCTACT
131) -WSN_PA_44
SEQ ID NO: 161
AGCGAAAGCAGGTACTGAGTCCAAAAAAGTACCTTGTTTCTACT
132) -WSN_NS_59
SEQ ID NO: 162
AGCAAAAGCAGGGTGACAAAGACATAATGGATCCAAACACTAACACCCTT
GTTTCTACT
133) -WSN_NS_60
SEQ ID NO: 163
AGCAAAAGCAGGGTGACAAAGACATAATGGATCCAAATAAAAAACACCCT
TGTTTCTACT
134) -WSN_NS_52
SEQ ID NO: 164
AGCAAAAGCAGGGTGACAAAGACATAATGTAAAAAACACCCTTGTTTCTA
CT
135) -WSN_NS_48
SEQ ID NO: 165
AGCAAAAGCAGGGTGACAAAGACATTAAAAAACACCCTTGTTTCTACT
136) -WSN_NS_44
SEQ ID NO: 166
AGCAAAAGCAGGGTGACAAAGACAAAAACACCCTTGTTTCTACT
137) -WSN_NS_46
SEQ ID NO: 167
AGCAAAAGCAGGGTGACAAAGACAAAAAAACACCCTTGTTTCTACT
138) -WSN_NS_45
SEQ ID NO: 168
AGCAAAAGCAGGGTGACAAAGATAAAAAACACCCTTGTTTCTACT
139) -WSN_NS_67
SEQ ID NO: 169
AGCAAAAGCAGGGTGACAAAGTCTCGTTTCAGCTTATTTAATAATAAAAA
ACACCCTTGTTTCTACT
140) -WSN_NS_46
SEQ ID NO: 170
AGCAAAAGCAGGGTGACAAATAATAAAAAACACCCTTGTTTCTACT
141) -WSN_NS_63
SEQ ID NO: 171
AGCAAAAGCAGGGTGACACTCGTTTCAGCTTATTTAATAATAAAAAACAC
CCTTGTTTCTACT
142) -WSN_NP_94
SEQ ID NO: 172
AGCAAAAGCAGGGTAGATAATCACTCACAGAGTGACATCGAAATCATGGC
GACCAAAGGCACCAAACGATCTTACAAATACCCTTGTTTCTACT
143) -WSN_NP_71
SEQ ID NO: 173
AGCAAAAGCAGGGTAGATAATCACTCACAGAGTGACATCGAAATCATGGC
AAAAATACCCTTGTTTCTACT
144) -WSN_NP_74
SEQ ID NO: 174
AGCAAAAGCAGGGTAGATAATCACTCACAGAGTGACATCGAAATCATGGC
AAGAAAAATACCCTTGTTTCTACT
145) -WSN_NP_76
SEQ ID NO: 175
AGCAAAAGCAGGGTAGATAATCACTCACAGAGTGACATCGAAATCATGGC
TAAAGAAAAATACCCTTGTTTCTACT
146) -WSN_NP_91
SEQ ID NO: 176
AGCAAAAGCAGGGTAGATAATCACTCACAGAGTGACATCGAAATCATGGA
GAGGAGTACGACAATTAAAGAAAAATACCCTTGTTTCTACT
147) -WSN_NP_64
SEQ ID NO: 177
AGCAAAAGCAGGGTAGATAATCACTCACAGAGTGACATCGAAATAAAATA
CCCTTGTTTCTACT
148) -WSN_NP_65
SEQ ID NO: 178
AGCAAAAGCAGGGTAGATAATCACTCACAGAGTGACATCGAAAGAAAAAT
ACCCTTGTTTCTACT
149) -WSN_NP_63
SEQ ID NO: 179
AGCAAAAGCAGGGTAGATAATCACTCACAGAGTGACATCGAGAAAAATAC
CCTTGTTTCTACT
150) -WSN_NP_88
SEQ ID NO: 180
AGCAAAAGCAGGGTAGATAATCACTCACAGAGTGACATCGCAATGCAGAG
GAGTACGACAATTAAAGAAAAATACCCTTGTTTCTACT
151) -WSN_NP_60
SEQ ID NO: 181
AGCAAAAGCAGGGTAGATAATCACTCACAGAGTGACATCAAAAATACCCT
TGTTTCTACT
152) -WSN_NP_64
SEQ ID NO: 182
AGCAAAAGCAGGGTAGATAATCACTCACAGAGTGACATCAAAGAAAAATA
CCCTTGTTTCTACT
153) -WSN_NP_84
SEQ ID NO: 183
AGCAAAAGCAGGGTAGATAATCACTCACAGAGTGACATATGCAGAGGAGT
ACGACAATTAAAGAAAAATACCCTTGTTTCTACT
154) -WSN_NP_59
SEQ ID NO: 184
AGCAAAAGCAGGGTAGATAATCACTCACAGAGTGACAGAAAAATACCCTT
GTTTCTACT
155) -WSN_NP_71
SEQ ID NO: 185
AGCAAAAGCAGGGTAGATAATCACTCACAGAGTGACAACGACAATTAAAG
AAAAATACCCTTGTTTCTACT
156) -WSN_NP_64
SEQ ID NO: 186
AGCAAAAGCAGGGTAGATAATCACTCACAGAGTGAAATTAAAGAAAAATA
CCCTTGTTTCTACT
157) -WSN_NP_71
SEQ ID NO: 187
AGCAAAAGCAGGAGTTTAAATGAATCCAAACCAGAAAATAATAACAAAAC
TCCTTGTTTCTACT
168) -WSN_NA_66
SEQ ID NO: 198
AGCAAAAGCAGGAGTTTAAATGAATCCAAACCAGATAGTTTGTTCAAAAA
ACTCCTTGTTTCTACT
169) -WSN_NA_67
SEQ ID NO: 199
AGCAAAAGCAGGAGTTTAAATGAATCCAAACCAAAGTAGTTTGTTCAAAA
AACTCCTTGTTTCTACT
170) -WSN_NA_68
SEQ ID NO: 200
AGCAAAAGCAGGAGTTTAAATGAATCCAAACCACAAGTAGTTTGTTCAAA
AAACTCCTTGTTTCTACT
171) -WSN_NA_69
SEQ ID NO: 201
AGCAAAAGCAGGAGTTTAAATGAATCCAAACCAACAAGTAGTTTGTTCAA
AAAACTCCTTGTTTCTACT
172) -WSN_NA_72
SEQ ID NO: 202
AGCAAAAGCAGGAGTTTAAATGAATCCAAACCATTGACAAGTAGTTTGTT
CAAAAAACTCCTTGTTTCTACT
173) -WSN_NA_63
SEQ ID NO: 203
AGCAAAAGCAGGAGTTTAAATGAATCCAAACCTAGTTTGTTCAAAAAACT
CCTTGTTTCTACT
174) -WSN_NA_69
SEQ ID NO: 204
AGCAAAAGCAGGAGTTTAAATGAATCCAAACCGACAAGTAGTTTGTTCAA
AAAACTCCTTGTTTCTACT
175) -WSN_NA_65
SEQ ID NO: 205
AGCAAAAGCAGGAGTTTAAATGAATCCAAACAAGTAGTTTGTTCAAAAAA
CTCCTTGTTTCTACT
176)-WSN_NA_66
SEQ ID NO: 206
AGCAAAAGCAGGAGTTTAAATGAATCCAAAACAAGTAGTTTGTTCAAAAA
ACTCCTTGTTTCTACT
177) -WSN_NA_67
SEQ ID NO: 207
AGCAAAAGCAGGAGTTTAAATGAATCCAAAGACAAGTAGTTTGTTCAAAA
AACTCCTTGTTTCTACT
178) -WSN_NA_68
SEQ ID NO: 208
AGCAAAAGCAGGAGTTTAAATGAATCCAAATGACAAGTAGTTTGTTCAAA
AAACTCCTTGTTTCTACT
179) -WSN_NA_65
SEQ ID NO: 209
AGCAAAAGCAGGAGTTTAAATGAATCCAGACAAGTAGTTTGTTCAAAAAA
CTCCTTGTTTCTACT
180) -WSN_NA_68
SEQ ID NO: 210
AGCAAAAGCAGGAGTTTAAATGAATCCCATTGACAAGTAGTTTGTTCAAA
AAACTCCTTGTTTCTACT
181) -WSN_NA_55
SEQ ID NO: 211
AGCAAAAGCAGGAGTTTAAATGAGTAGTTTGTTCAAAAAACTCCTTGTTT
CTACT
182) -WSN_NA_63
SEQ ID NO: 212
AGCAAAAGCAGGAGTTTAAATGCATTGACAAGTAGTTTGTTCAAAAAACT
CCTTGTTTCTACT
183) -WSN_NA_49
SEQ ID NO: 213
AGCAAAAGCAGGAGTTTAAATTTTGTTCAAAAAACTCCTTGTTTCTACT
184) -WSN_NA_56
SEQ ID NO: 214
AGCAAAAGCAGGAGTTTAAATCAAGTAGTTTGTTCAAAAAACTCCTTGTT
TCTACT
185) -WSN_NA_43
SEQ ID NO: 215
AGCAAAAGCAGGAGTTTAATTCAAAAAACTCCTTGTTTCTACT
186) -WSN_NA_62
SEQ ID NO: 216
AGCAAAAGCAGGAGTTTACACCATTGACAAGTAGTTTGTTCAAAAAACTC
CTTGTTTCTACT
187) -WSN_M_78
SEQ ID NO: 217
AGCGAAAGCAGGTAGATATTGAAAGATGAGTCTTCTAACCGAGGTCGAAA
CGGAGTAAAAAACTACCTTGTTTCTACT
188) -WSN_M_61
SEQ ID NO: 218
AGCGAAAGCAGGTAGATATTGAAAGATTAGAGCTGGAGTAAAAAACTACC
TTGTTTCTACT
189) -WSN_M_50
SEQ ID NO: 219
AGCGAAAGCAGGTAGATATTGAAAGATTAAAAAACTACCTTGTTTCTACT
190) -WSN_M_43
SEQ ID NO: 220
AGCGAAAGCAGGTAGATATTTAAAAAACTACCTTGTTTCTACT
191) -WSN_M_46
SEQ ID NO: 221
AGCGAAAGCAGGTAGATATGGAGTAAAAAACTACCTTGTTTCTACT
192) -WSN_HA_77
SEQ ID NO: 222
AGCAAAAGCAGGGGAAAATAAAAACAACCAAAATGTAGGATTTCAGAAAT
ATAAGGAAAAACACCCTTGTTTCTACT
193) -WSN_HA_53
SEQ ID NO: 223
AGCAAAAGCAGGGGAAAATAAAAACAACCAAAATAAACACCCTTGTTTCT
ACT
194) -WSN_HA_61
SEQ ID NO: 224
AGCAAAAGCAGGGGAAAATAAAAACAACCAAAATATAAGGAAAAACACCC
TTGTTTCTACT
195) -WSN_HA_65
SEQ ID NO: 225
AGCAAAAGCAGGGGAAAATAAAAACAACCAAAATAAATATAAGGAAAAAC
ACCCTTGTTTCTACT
196) -WSN_HA_60
SEQ ID NO: 226
AGCAAAAGCAGGGGAAAATAAAAACAACCAAAAATAAGGAAAAACACCCT
TGTTTCTACT
197) -WSN_HA_87
SEQ ID NO: 227
AGCAAAAGCAGGGGAAAATAAAAACAACCAATATGCATCTGAGATTAGGA
TTTCAGAAATATAAGGAAAAACACCCTTGTTTCTACT
198) -WSN_HA_65
SEQ ID NO: 228
AGCAAAAGCAGGGGAAAATAAAAACAACCATCAGAAATATAAGGAAAAAC
ACCCTTGTTTCTACT
199) -WSN_HA_66
SEQ ID NO: 229
AGCAAAAGCAGGGGAAAATAAAAACAACCATTCAGAAATATAAGGAAAAA
CACCCTTGTTTCTACT
200) -WSN_HA_51
SEQ ID NO: 230
AGCAAAAGCAGGGGAAAATAAAAACAACCGAAAAACACCCTTGTTTCTAC
T
201) -WSN_HA_50
SEQ ID NO: 231
AGCAAAAGCAGGGGAAAATAAAAACAACGAAAAACACCCTTGTTTCTACT
202) -WSN_HA_46
SEQ ID NO: 232
AGCAAAAGCAGGGGAAAATAAAAACAAAAACACCCTTGTTTCTACT
203) -WSN_HA_65
SEQ ID NO: 233
AGCAAAAGCAGGGGAAAATAAAAACAGATTTCAGAAATATAAGGAAAAAC
ACCCTTGTTTCTACT
204) -WSN_HA_44
SEQ ID NO: 234
AGCAAAAGCAGGGGAAAATAAAAAAAAACACCCTTGTTTCTACT
205) -WSN_HA_56
SEQ ID NO: 235
AGCAAAAGCAGGGGAAAATAAAAAGAAATATAAGGAAAAACACCCTTGTT
TCTACT
206) -WSN_HA_60
SEQ ID NO: 236
AGCAAAAGCAGGGGAAAATAAAAATTCAGAAATATAAGGAAAAACACCCT
TGTTTCTACT
207) -WSN_HA_63
SEQ ID NO: 237
AGCAAAAGCAGGGGAAAATAAAAAGATTTCAGAAATATAAGGAAAAACAC
CCTTGTTTCTACT
208) -WSN_HA_44
SEQ ID NO: 238
AGCAAAAGCAGGGGAAAATAAGGAAAAACACCCTTGTTTCTACT
209) -WSN_HA_60
SEQ ID NO: 239
AGCAAAAGCAGGGGAAAATAGGATTTCAGAAATATAAGGAAAAACACCCT
TGTTTCTACT
210) -WSN_HA_53
SEQ ID NO: 240
AGCAAAAGCAGGGGAAAATCAGAAATATAAGGAAAAACACCCTTGTTTCT
ACT
211) -WSN_HA_58
SEQ ID NO: 241
AGCAAAAGCAGGGGAAAATGATTTCAGAAATATAAGGAAAAACACCCTTG
TTTCTACT
212) -WSN_HA_41
SEQ ID NO: 242
AGCAAAAGCAGGGGAAAAGGAAAAACACCCTTGTTTCTACT
213) -WSN_HA_43
SEQ ID NO: 243
AGCAAAAGCAGGGGAAAAAAGGAAAAACACCCTTGTTTCTACT
214) -WSN_HA_50
SEQ ID NO: 244
AGCAAAAGCAGGGGAAAAGAAATATAAGGAAAAACACCCTTGTTTCTACT
215) -WSN_HA_64
SEQ ID NO: 245
AGCAAAAGCAGGGGAAAAAGATTAGGATTTCAGAAATATAAGGAAAAACA
CCCTTGTTTCTACT

### Example 3: Fasta File 2. WSN mvRNA Sequences

1)
WSN_NP_61
SEQ ID NO: 246
AGCAAAAGCAGGGTAGATAATCACTCACAGAGTGACATAGAAAAATACCC
TTGTTTCTACT
2)
WSN_PB1_57
SEQ ID NO: 247
AGCGAAAGCAGGCAAACCATTTGAATGGATTTCATGAAAAAATGCCTTGT
TTCTACT
3)
WSN_PB1_57
SEQ ID NO: 248
AGCGAAAGCAGGCAAACCATTTGAATGTCCTTCATGAAAAAATGCCTTGT
TTCTACT
4)
WSN_PB1_62
SEQ ID NO: 249
AGCGAAAGCAGGCAAACCATTTGAATGGATGTCAATCCGACTTAAAATGC
CTTGTTTCTACT
5)
WSN_PB1_66
SEQ ID NO: 250
AGCGAAAGCAGGCAAACCATTTGAATGTTTAGCTTGTCCTTCATGAAAAA
ATGCCTTGTTTCTACT
6)
WSN_NP_58
SEQ ID NO: 251
AGCAAAAGCAGGGTAGATAATCACTCACAGAGTGACATCAAATACCCTTG
TTTCTACT
7)
WSN_HA_60
SEQ ID NO: 252
AGCAAAAGCAGGGGAAATTAGGATTTCAGAAATATAAGGAAAAACACCCT
TGTTTCTACT
8)
WSN_HA_44
SEQ ID NO: 253
AGCAAAAGCAGGGGAATATAAGGAAAAACACCCTTGTTTCTACT
9)
WSN_PB1_69
SEQ ID NO: 254
AGCGAAAGCAGGCAAACCATTTGAATGGATGTTAGCTTGTCCTTCATGAA
AAAATGCCTTGTTTCTACT
10)
WSN_PB1_61
SEQ ID NO: 255
AGCGAAAGCAGGCAAACCATTTGAATGGTTGTCCTTCATGAAAAAATGCC
TTGTTTCTACT
11)
WSN_NP_63
SEQ ID NO: 256
AGCAAAAGCAGGGTAGATAATCACTCACAGAGTGACTTAAAGAAAAATAC
CCTTGTTTCTACT
12)
WSN_NA_70
SEQ ID NO: 257
AGCAAAAGCAGGAGTTTAAATGAATCCAAACCTGACAAGTAGTTTGTTCA
AAAAACTCCTTGTTTCTACT
13)
WSN_NA_62
SEQ ID NO: 258
AGCAAAAGCAGGAGTTTACACCATTGACAAGTAGTTTGTTCAAAAAACTC
CTTGTTTCTACT
14)
WSN_PB1_65
SEQ ID NO: 259
AGCGAAAGCAGGCAAACCATTTGAATGGATGTCAATCCGACTTGAAAAAA
TGCCTTGTTTCTACT
15)
WSN_PB1_69
SEQ ID NO: 260
AGCGAAAGCAGGCAAACCATTTGAATGGATGTCAATCCGTCCTTCATGAA
AAAATGCCTTGTTTCTACT
16)
WSN_PB1_48
SEQ ID NO: 261
AGCGAAAGCAGGCAAACCATTTGAATGAAAAAATGCCTTGTTTCTACT
17)
WSN_PB1_62
SEQ ID NO: 262
AGCGAAAGCAGGCAAACCATTTGAATAGCTTGTCCTTCATGAAAAAATGC
CTTGTTTCTACT
18)
WSN_NP_71
SEQ ID NO: 263
AGCAAAAGCAGGGTAGATAATCACTCACAGAGTGACATCGAAATCATGGC
AAAAATACCCTTGTTTCTACT
19)
WSN_NP_76
SEQ ID NO: 264
AGCAAAAGCAGGGTAGATAATCACTCACAGAGTGACATCGAAATCATGGC
TAAAGAAAAATACCCTTGTTTCTACT
20)
WSN_NP_64
SEQ ID NO: 265
AGCAAAAGCAGGGTAGATAATCACTCACAGAGTGACATCGAAATAAAATA
CCCTTGTTTCTACT
21)
WSN_NP_55
SEQ ID NO: 266
AGCAAAAGCAGGGTAGATAATCACTCACAGAGAGAAAAATACCCTTGTTT
CTACT
22)
WSN_NP_44
SEQ ID NO: 267
AGCAAAAGCAGGGTAGATTAAAGAAAAATACCCTTGTTTCTACT
23)
WSN_HA 58
SEQ ID NO: 268
AGCAAAAGCAGGGGAATAGGATTTCAGAAATATAAGGAAAAACACCCTTG
TTTCTACT
24)
WSN_HA 64
SEQ ID NO: 269
AGCAAAAGCAGGGGAATGAGATTAGGATTTCAGAAATATAAGGAAAAACA
CCCTTGTTTCTACT
25)
WSN_PB2_80
SEQ ID NO: 270
AGCGAAAGCAGGTCAATTATATTCAATATGGAAGGCCATCAATTAGTGTC
GAATAGTTTAAAAACGACCTTGTTTCTACT
26)
WSN_PB1_77
SEQ ID NO: 271
AGCGAAAGCAGGCAAACCATTTGAATGGATGTCAATCCGACTTTACTTTT
CTCATGAAAAAATGCCTTGTTTCTACT
27)
WSN_PB1_65
SEQ ID NO: 272
AGCGAAAGCAGGCAAACCATTTGAATGGATGTCAATCCGACTTTACAAAA
TGCCTTGTTTCTACT
28)
WSN_PB1_64
SEQ ID NO: 273
AGCGAAAGCAGGCAAACCATTTGAATGGATGTCAATCCGACTTAAAAAAT
GCCTTGTTTCTACT
29)
WSN_PB1_68
SEQ ID NO: 274
AGCGAAAGCAGGCAAACCATTTGAATGGATGTCAATCCGACTTCATGAAA
AAATGCCTTGTTTCTACT
30)
WSN_PB1_64
SEQ ID NO: 275
AGCGAAAGCAGGCAAACCATTTGAATGGATGTCAATCCGACTGAAAAAAT
GCCTTGTTTCTACT
31)
WSN_PB1_58
SEQ ID NO: 276
AGCGAAAGCAGGCAAACCATTTGAATGGATGTCAATCCAAAAATGCCTTG
TTTCTACT
32)
WSN_PB1_64
SEQ ID NO: 277
AGCGAAAGCAGGCAAACCATTTGAATGGATGTCAATCCTCATGAAAAAAT
GCCTTGTTTCTACT
33)
WSN_PB1_68
SEQ ID NO: 278
AGCGAAAGCAGGCAAACCATTTGAATGGATGTCAATCCTCCTTCATGAAA
AAATGCCTTGTTTCTACT
34)
WSN_PB1_60
SEQ ID NO: 279
AGCGAAAGCAGGCAAACCATTTGAATGGATGTCAATCTGAAAAAATGCCT
TGTTTCTACT
35)
WSN_PB1_54
SEQ ID NO: 280
AGCGAAAGCAGGCAAACCATTTGAATGGATGTGAAAAAATGCCTTGTTTC
TACT
36)
WSN_PB1_50
SEQ ID NO: 281
AGCGAAAGCAGGCAAACCATTTGAATGGGAAAAAATGCCTTGTTTCTACT
37)
WSN_PB1_66
SEQ ID NO: 282
AGCGAAAGCAGGCAAACCATTTGAATGGTTAGCTTGTCCTTCATGAAAAA
ATGCCTTGTTTCTACT
38)
WSN_PB1_49
SEQ ID NO: 283
AGCGAAAGCAGGCAAACCATTTGAATTGAAAAAATGCCTTGTTTCTACT
39)
WSN_PB1_48
SEQ ID NO: 284
AGCGAAAGCAGGCAAACCATTTGCATGAAAAAATGCCTTGTTTCTACT
40)
WSN_PB1_43
SEQ ID NO: 285
AGCGAAAGCAGGCAAACCATTTAAAAAATGCCTTGTTTCTACT
41)
WSN_PB1_64
SEQ ID NO: 286
AGCGAAAGCAGGCAAACCATGTGAATTTAGCTTGTCCTTCATGAAAAAAT
GCCTTGTTTCTACT
42)
WSN_PB1_61
SEQ ID NO: 287
AGCGAAAGCAGGCAAACCTGAATTTAGCTTGTCCTTCATGAAAAAATGCC
TTGTTTCTACT
43)
WSN_PB1_40
SEQ ID NO: 288
AGCGAAAGCAGGCAAACTGAAAAAATGCCTTGTTTCTACT
44)
WSN_PB1_56
SEQ ID NO: 289
AGCGAAAGCAGGCAAACTTTAGCTTGTCCTTCATGAAAAAATGCCTTGTT
TCTACT
45)
WSN_PA_60
SEQ ID NO: 290
AGCGAAAGCAGGTACTGATTCAAAATGGCATACTGTCCAAAAAAGTACCT
TGTTTCTACT
46)
WSN_PA_66
SEQ ID NO: 291
AGCGAAAGCAGGTACTGATTCAAAATGTGCTATCCATACTGTCCAAAAAA
GTACCTTGTTTCTACT
47)
WSN_NS_80
SEQ ID NO: 292
AGCAAAAGCAGGGTGACAAAGACATAATGGATCCAAACACTGTGTCAAGC
TTTCAGATAAAAAACACCCTTGTTTCTACT
48)
WSN_NS 56
SEQ ID NO: 293
AGCAAAAGCAGGGTGACAAAGACATAATGGATCTAAAAAACACCCTTGTT
TCTACT
49)
WSN_NS_55
SEQ ID NO: 294
AGCAAAAGCAGGGTGACAAAGACATAATGTAATAAAAAACACCCTTGTTT
CTACT
50)
WSN_NP_66
SEQ ID NO: 295
AGCAAAAGCAGGGTAGATAATCACTCACAGAGTGACATCGTAAAGAAAAA
TACCCTTGTTTCTACT
51)
WSN_NP_60
SEQ ID NO: 296
AGCAAAAGCAGGGTAGATAATCACTCACAGAGTGACATCAAAAATACCCT
TGTTTCTACT
52)
WSN_NP_65
SEQ ID NO: 297
AGCAAAAGCAGGGTAGATAATCACTCACAGAGTGACATCTAAAGAAAAAT
ACCCTTGTTTCTACT
53)
WSN_NP_66
SEQ ID NO: 298
AGCAAAAGCAGGGTAGATAATCACTCACAGAGTGACATCTTAAAGAAAAA
TACCCTTGTTTCTACT
54)
WSN_NP_74
SEQ ID NO: 299
AGCAAAAGCAGGGTAGATAATCACTCACAGAGTGACATCTACGACAATTA
AAGAAAAATACCCTTGTTTCTACT
55)
WSN_NP_64
SEQ ID NO: 300
AGCAAAAGCAGGGTAGATAATCACTCACAGAGTGACATTAAAGAAAAATA
CCCTTGTTTCTACT
56)
WSN_NP_59
SEQ ID NO: 301
AGCAAAAGCAGGGTAGATAATCACTCACAGAGTGACAGAAAAATACCCTT
GTTTCTACT
57)
WSN_NP_72
SEQ ID NO: 302
AGCAAAAGCAGGGTAGATAATCACTCACAGAGTGACGTACGACAATTAAA
GAAAAATACCCTTGTTTCTACT
58)
WSN_NP_70
SEQ ID NO: 303
AGCAAAAGCAGGGTAGATAATCACTCACAGAGTGATACGACAATTAAAGA
AAAATACCCTTGTTTCTACT
59)
WSN_NP_57
SEQ ID NO: 304
AGCAAAAGCAGGGTAGATAATCACTCACAGAGTAAGAAAAATACCCTTGT
TTCTACT
60)
WSN_NP_44
SEQ ID NO: 305
AGCAAAAGCAGGGTAGATAATCACTAAATACCCTTGTTTCTACT
61)
WSN_NP_49
SEQ ID NO: 306
AGCAAAAGCAGGGTAGATAATCACTAAGAAAAATACCCTTGTTTCTACT
62)
WSN_NP_43
SEQ ID NO: 307
AGCAAAAGCAGGGTAGTTAAAGAAAAATACCCTTGTTTCTACT
63)
WSN_NA_75
SEQ ID NO: 308
AGCAAAAGCAGGAGTTTAAATGAATCCAAACCAGAAAATAAAAGTAGTTT
GTTCAAAAAACTCCTTGTTTCTACT
64)
WSN_NA_73
SEQ ID NO: 309
AGCAAAAGCAGGAGTTTAAATGAATCCAAACCAGAAAACAAGTAGTTTGT
TCAAAAAACTCCTTGTTTCTACT
65)
WSN_NA_79
SEQ ID NO: 310
AGCAAAAGCAGGAGTTTAAATGAATCCAAACCAGAAAACATTGACAAGTA
GTTTGTTCAAAAAACTCCTTGTTTCTACT
66)
WSN_NA_69
SEQ ID NO: 311
AGCAAAAGCAGGAGTTTAAATGAATCCAAACCAGAAAGTAGTTTGTTCAA
AAAACTCCTTGTTTCTACT
67)
WSN_NA_61
SEQ ID NO: 312
AGCAAAAGCAGGAGTTTAAATGAATCCAAACCAGATGTTCAAAAAACTCC
TTGTTTCTACT
68)
WSN_NA 60
SEQ ID NO: 313
AGCAAAAGCAGGAGTTTAAATGAATCCAAACCAGTGTTCAAAAAACTCCT
TGTTTCTACT
69)
WSN_NA_69
SEQ ID NO: 314
AGCAAAAGCAGGAGTTTAAATGAATCCAAACCAACAAGTAGTTTGTTCAA
AAAACTCCTTGTTTCTACT
70)
WSN_NA_66
SEQ ID NO: 315
AGCAAAAGCAGGAGTTTAAATGAATCCAAAACAAGTAGTTTGTTCAAAAA
ACTCCTTGTTTCTACT
71)
WSN_NA_60
SEQ ID NO: 316
AGCAAAAGCAGGAGTTTAAATGAATCCAATAGTTTGTTCAAAAAACTCCT
TGTTTCTACT
72)
WSN_NA_63
SEQ ID NO: 317
AGCAAAAGCAGGAGTTTAACACCATTGACAAGTAGTTTGTTCAAAAAACT
CCTTGTTTCTACT
73)
WSN_NA_62
SEQ ID NO: 318
AGCAAAAGCAGGAGTTTTCACCATTGACAAGTAGTTTGTTCAAAAAACTC
CTTGTTTCTACT
74)
WSN_NA_63
SEQ ID NO: 319
AGCAAAAGCAGGAGTCGTTCACCATTGACAAGTAGTTTGTTCAAAAAACT
CCTTGTTTCTACT
75)
WSN_M_71
SEQ ID NO: 320
AGCGAAAGCAGGTAGATATTGAAAGATGAGTCTTCCATAGAGCTGGAGTA
AAAAACTACCTTGTTTCTACT
76)
WSN_M_52
SEQ ID NO: 321
AGCGAAAGCAGGTAGATATTGAAAGATGAGAAAAAACTACCTTGTTTCTA
CT
77)
WSN_M_48
SEQ ID NO: 322
AGCGAAAGCAGGTAGATATTGAAAGATAAAAACTACCTTGTTTCTACT
78)
WSN_M 47
SEQ ID NO: 323
AGCGAAAGCAGGTAGATATTGAAATAAAAAACTACCTTGTTTCTACT
79)
WSN_HA_89
SEQ ID NO: 324
AGCAAAAGCAGGGGAAAATAAAAACAACCAAAATGAAGGCAGATTAGGAT
TTCAGAAATATAAGGAAAAACACCCTTGTTTCTACT
80)
WSN_HA_90
SEQ ID NO: 325
AGCAAAAGCAGGGGAAAATAAAAACAACCAAAATGAAGGCCTGAGATTAG
GATTTCAGAAATATAAGGAAAAACACCCTTGTTTCTACT
81)
WSN_HA_50
SEQ ID NO: 326
AGCAAAAGCAGGGGAAAATAAAAACAACGAAAAACACCCTTGTTTCTACT
82)
WSN_HA_63
SEQ ID NO: 327
AGCAAAAGCAGGGGAAAATAAAAACATTTCAGAAATATAAGGAAAAACAC
CCTTGTTTCTACT
83)
WSN_HA 44
SEQ ID NO: 328
AGCAAAAGCAGGGGAAAATAAAGAAAAACACCCTTGTTTCTACT
84)
WSN_HA_41
SEQ ID NO: 329
AGCAAAAGCAGGGGAAAATGAAAAACACCCTTGTTTCTACT
85)
WSN_HA_65
SEQ ID NO: 330
AGCAAAAGCAGGGGAAATGAGATTAGGATTTCAGAAATATAAGGAAAAAC
ACCCTTGTTTCTACT
86)
WSN_HA_66
SEQ ID NO: 331
AGCAAAAGCAGGGGAAACTGAGATTAGGATTTCAGAAATATAAGGAAAAA
CACCCTTGTTTCTACT
87)
WSN_HA_61
SEQ ID NO: 332
AGCAAAAGCAGGGGAAGATTAGGATTTCAGAAATATAAGGAAAAACACCC
TTGTTTCTACT

### Example 4: Fasta File 3. WSN mvRNA Sequences

1)
SEQ ID NO: 333
BM18_PB2, 1, 2325, 35: (18+17)
AGCRAAAGCAGGTCAATTACGACCTTGTTTCTACT
2)
SEQ ID NO: 334
BM18_M, 1, 1009, 36: (17+19)
AGCRAAAGCAGGTAGATAAACTACCTTGTTTCTACT
3)
SEQ ID NO: 335
BM18_M, 1, 1010, 35: (17+18)
AGCRAAAGCAGGTAGATAACTACCTTGTTTCTACT
4)
SEQ ID NO: 336
BM18_PB2, 1, 2324, 36: (18+18)
AGCRAAAGCAGGTCAATTAACGACCTTGTTTCTACT
5)
SEQ ID NO: 337
BM18_NS, 1, 872, 36: (17+19)
AGCRAAAGCAGGGTGACAAACACCCTTGTTTCTACT
6)
SEQ ID NO: 338
BM18_NP, 1, 1547, 37: (18+19)
AGCRAAAGCAGGGTAGATAAATACCCTTGTTTCTACT
7)
SEQ ID NO: 339
BM18_HA, 1, 1762, 36: (19+17)
AGCRAAAGCAGGGGAAAATACACCCTTGTTTCTACT
8)
SEQ ID NO: 340
BM18_NP, 1, 1541, 41: (16+25)
AGCRAAAGCAGGGTAGAAAGAAAAATACCCTTGTTTCTACT
9)
SEQ ID NO: 341
BM18_NP, 1, 1547, 35: (16+19)
AGCRAAAGCAGGGTAGAAATACCCTTGTTTCTACT
10)
SEQ ID NO: 342
BM18_PB2, 1, 2323, 37: (18+19)
AGCRAAAGCAGGTCAATTAAACGACCTTGTTTCTACT
11)
SEQ ID NO: 343
BM18_PB2, 1, 2319, 39: (16+23)
AGCRAAAGCAGGTCAATTAAAAACGACCTTGTTTCTACT
12)
SEQ ID NO: 344
BM18_HA, 1, 1761, 37: (19+18)
AGCRAAAGCAGGGGAAAATAACACCCTTGTTTCTACT
13)
SEQ ID NO: 345
BM18_NS, 1, 869, 39: (17+22)
AGCRAAAGCAGGGTGACAAAAAACACCCTTGTTTCTACT
14)
SEQ ID NO: 346
BM18_NP, 1, 1545, 39: (18+21)
AGCRAAAGCAGGGTAGATAAAAATACCCTTGTTTCTACT
15)
SEQ ID NO: 347
BM18_NP, 1, 1548, 34: (16+18)
AGCRAAAGCAGGGTAGAATACCCTTGTTTCTACT
16)
SEQ ID NO: 348
BM18_M, 1, 1008, 37: (17+20)
AGCRAAAGCAGGTAGATAAAACTACCTTGTTTCTACT
17)
SEQ ID NO: 349
BM18_NS, 1, 871, 37: (17+20)
AGCRAAAGCAGGGTGACAAAACACCCTTGTTTCTACT
18)
SEQ ID NO: 350
BM18_NS, 1, 873, 35: (17+18)
AGCRAAAGCAGGGTGACAACACCCTTGTTTCTACT
19)
SEQ ID NO: 351
BM18_HA, 1, 1760, 38: (19+19)
AGCRAAAGCAGGGGAAAATAAACACCCTTGTTTCTACT
20)
SEQ ID NO: 352
BM18_HA, 1, 1752, 44: (17+27)
AGCRAAAGCAGGGGAAAATAAGGAAAAACACCCTTGTTTCTACT
21)
SEQ ID NO: 353
BM18_NP, 1, 1546, 36: (16+20)
AGCRAAAGCAGGGTAGAAAATACCCTTGTTTCTACT
22)
SEQ ID NO: 354
BM18_M, 1, 1005, 39: (16+23)
AGCRAAAGCAGGTAGATAAAAAACTACCTTGTTTCTACT
23)
SEQ ID NO: 355
BM18_NP, 1, 1546, 38: (18+20)
AGCRAAAGCAGGGTAGATAAAATACCCTTGTTTCTACT
24)
SEQ ID NO: 356
BM18_PB1, 1, 2324, 36: (18+18)
AGCRAAAGCAGGCAAACCAAATGCCTTGTTTCTACT
25)
SEQ ID NO: 357
BM18_HA, 1, 1757, 41: (19+22)
AGCRAAAGCAGGGGAAAATGAAAAACACCCTTGTTTCTACT
26)
SEQ ID NO: 358
BM18_NP, 1, 1540, 43: (17+26)
AGCRAAAGCAGGGTAGATAAAGAAAAATACCCTTGTTTCTACT
27)
SEQ ID NO: 359
BM18_NP, 1, 1544, 38: (16+22)
AGCRAAAGCAGGGTAGGAAAAATACCCTTGTTTCTACT
28)
SEQ ID NO: 360
BM18_PB1, 1, 2323, 37: (18+19)
AGCRAAAGCAGGCAAACCAAAATGCCTTGTTTCTACT
29)
SEQ ID NO: 361
BM18_HA, 1, 1758, 37: (16+21)
AGCRAAAGCAGGGGAAAAAAACACCCTTGTTTCTACT
30)
SEQ ID NO: 362
BM18_NP, 1, 1549, 35: (18+17)
AGCRAAAGCAGGGTAGATATACCCTTGTTTCTACT
31)
SEQ ID NO: 363
BM18_PB2, 1, 2322, 38: (18+20)
AGCRAAAGCAGGTCAATTAAAACGACCTTGTTTCTACT
32)
SEQ ID NO: 364
BM18_M, 1, 1011, 34: (17+17)
AGCRAAAGCAGGTAGATACTACCTTGTTTCTACT
33)
SEQ ID NO: 365
BM18_HA, 1, 1759, 39: (19+20)
AGCRAAAGCAGGGGAAAATAAAACACCCTTGTTTCTACT
34)
SEQ ID NO: 366
BM18_PB2, 1, 2315, 44: (17+27)
AGCRAAAGCAGGTCAATTAGTTTAAAAACGACCTTGTTTCTACT
35)
SEQ ID NO: 367
BM18_NP, 1, 1543, 39: (16+23)
AGCRAAAGCAGGGTAGAGAAAAATACCCTTGTTTCTACT
36)
SEQ ID NO: 368
BM18_HA, 1, 1758, 38: (17+21)
AGCRAAAGCAGGGGAAAAAAAACACCCTTGTTTCTACT
37)
SEQ ID NO: 369
BM18_HA, 1, 1754, 42: (17+25)
AGCRAAAGCAGGGGAAAAAGGAAAAACACCCTTGTTTCTACT
38)
SEQ ID NO: 370
BM18_PB1, 1, 2325, 35: (18+17)
AGCRAAAGCAGGCAAACCAATGCCTTGTTTCTACT
39)
SEQ ID NO: 371
BM18_NS, 1, 867, 47: (23+24)
AGCRAAAGCAGGGTGACAAAGACATAAAAAACACCCTTGTTTCTACT
40)
SEQ ID NO: 372
BM18_NS, 1, 870, 38: (17+21)
AGCRAAAGCAGGGTGACAAAAACACCCTTGTTTCTACT
41)
SEQ ID NO: 373:
BM18_NS, 1, 874, 34: (17+17)
AGCRAAAGCAGGGTGACACACCCTTGTTTCTACT
42)
SEQ ID NO: 374
BM18_PB1, 1, 2321, 39: (18+21)
AGCRAAAGCAGGCAAACCAAAAAATGCCTTGTTTCTACT
43)
SEQ ID NO: 375
BM18_M, 1, 1007, 38: (17+21)
AGCRAAAGCAGGTAGATAAAAACTACCTTGTTTCTACT
44)
SEQ ID NO: 376
BM18_HA, 1, 1756, 42: (19+23)
AGCRAAAGCAGGGGAAAATGGAAAAACACCCTTGTTTCTACT
45)
SEQ ID NO: 377
BM18_NA, 1, 1439, 36: (17+19)
AGCRAAAGCAGGAGTTTAAAACTCCTTGTTTCTACT
46)
SEQ ID NO: 378
BM18_PB1, 1, 2322, 38: (18+20)
AGCRAAAGCAGGCAAACCAAAAATGCCTTGTTTCTACT
47)
SEQ ID NO: 379
BM18_M, 1, 1004, 41: (17+24)
AGCRAAAGCAGGTAGATGTAAAAAACTACCTTGTTTCTACT
48)
SEQ ID NO: 380
BM18_NP, 1, 1544, 40: (18+22)
AGCRAAAGCAGGGTAGATGAAAAATACCCTTGTTTCTACT
49)
SEQ ID NO: 381
BM18_NP, 1, 1542, 40: (16+24)
AGCRAAAGCAGGGTAGAAGAAAAATACCCTTGTTTCTACT
50)
SEQ ID NO: 382
BM18_HA, 1, 1754, 41: (16+25)
AGCRAAAGCAGGGGAAAAGGAAAAACACCCTTGTTTCTACT
51)
SEQ ID NO: 383
BM18_PB2, 1, 2313, 47: (18+29)
AGCRAAAGCAGGTCAATTAATAGTTTAAAAACGACCTTGTTTCTACT
52)
SEQ ID NO: 384
BM18_M, 1, 1005, 42: (19+23)
AGCRAAAGCAGGTAGATGTTAAAAAACTACCTTGTTTCTACT
53)
SEQ ID NO: 385
BM18_NP, 1, 1543, 41: (18+23)
AGCRAAAGCAGGGTAGATAGAAAAATACCCTTGTTTCTACT
54)
SEQ ID NO: 386
BM18_PB2, 1, 2313, 53: (24+29)
AGCRAAAGCAGGTCAATTATATTCAATAGTTTAAAAACGACCTTGTTT
CTACT
55)
SEQ ID NO: 387
BM18_PB2, 1, 2314, 46: (18+28)
AGCRAAAGCAGGTCAATTATAGTTTAAAAACGACCTTGTTTCTACT
56)
SEQ ID NO: 388
BM18_PB1, 1, 2322, 43: (23+20)
AGCRAAAGCAGGCAAACCATTTGAAAAATGCCTTGTTTCTACT
57)
SEQ ID NO: 389
BM18_PB1, 1, 2319, 44: (21+23)
AGCRAAAGCAGGCAAACCATTTGAAAAAATGCCTTGTTTCTACT
58)
SEQ ID NO: 390
BM18_M, 1, 1006, 43: (21+22)
AGCRAAAGCAGGTAGATGTTGAAAAAACTACCTTGTTTCTACT
59)
SEQ ID NO: 391
BM18_PA, 1, 2212, 48: (26+22)
AGCRAAAGCAGGTACTGATTCAAAATAAAAAAGTACCTTGTTTCTACT
60)
SEQ ID NO: 392
BM18_PA, 1, 2216, 39: (21+18)
AGCRAAAGCAGGTACTGATTCAAGTACCTTGTTTCTACT
61)
SEQ ID NO: 393
BM18_PA, 1, 2216, 34: (16+18)
AGCRAAAGCAGGTACTAAGTACCTTGTTTCTACT
62)
SEQ ID NO: 394
BM18_PA, 1, 2215, 35: (16+19)
AGCRAAAGCAGGTACTAAAGTACCTTGTTTCTACT
63)
SEQ ID NO: 395
BM18_PB2, 1, 2308, 60: (26+34)
AGCRAAAGCAGGTCAATTATATTCAATGTCGAATAGTTTAAAAACGA
CCTTGTTTCTACT
64)
SEQ ID NO: 396
BM18_HA, 1, 1746, 52: (19+33)
AGCRAAAGCAGGGGAAAATAGAAATATAAGGAAAAACACCCTTGTT
TCTACT
65)
SEQ ID NO: 397
BM18_HA, 1, 1741, 57: (19+38)
AGCRAAAGCAGGGGAAAATATTTCAGAAATATAAGGAAAAACACCC
TTGTTTCTACT
66)
SEQ ID NO: 398
BM18_HA, 1, 1758, 39: (18+21)
AGCRAAAGCAGGGGAAAAAAAAACACCCTTGTTTCTACT
67)
SEQ ID NO: 399
BM18_HA, 1, 1737, 60: (18+42)
AGCRAAAGCAGGGGAAAATAGGATTTCAGAAATATAAGGAAAAACA
CCCTTGTTTCTACT
68)
SEQ ID NO: 400
BM18_NS, 1, 869, 43: (21+22)
AGCRAAAGCAGGGTGACAAAGAAAAAACACCCTTGTTTCTACT
69)
SEQ ID NO: 401
BM18_M, 1, 1000, 46: (18+28)
AGCRAAAGCAGGTAGATGTGGAGTAAAAAACTACCTTGTTTCTACT
70)
SEQ ID NO: 402
BM18_NA, 1, 1409, 66: (17+49)
AGCRAAAGCAGGAGTTTCCATTCACCATTGACAAGTAGTTTGTTCA
AAAAACTCCTTGTTTCTACT
71)
SEQ ID NO: 403
BM18_NA, 1, 1423, 51: (16+35)
AGCRAAAGCAGGAGTTCAAGTAGTTTGTTCAAAAAACTCCTTGTTTC
TACT
72)
SEQ ID NO: 404
BM18_NP, 1, 1545, 38: (17+21)
AGCRAAAGCAGGGTAGAAAAAATACCCTTGTTTCTACT
73)
SEQ ID NO: 405
BM18_NP, 1, 1541, 42: (17+25)
AGCRAAAGCAGGGTAGAAAAGAAAAATACCCTTGTTTCTACT
74)
SEQ ID NO: 406
BM18_PA, 1, 2207, 52: (25+27)
AGCRAAAGCAGGTACTGATTCAAAATGTCCAAAAAAGTACCTTG
TTTCTACT
75)
SEQ ID NO: 407
BM18_PA, 1, 2211, 43: (20+23)
AGCRAAAGCAGGTACTGATTCAAAAAAGTACCTTGTTTCTACT
76)
SEQ ID NO: 408
BM18_PB1, 1, 2320, 45: (23+22)
AGCRAAAGCAGGCAAACCATTTGGAAAAAATGCCTTGTTTCTACT
77)
SEQ ID NO: 409
BM18_PB1, 1, 2302, 58: (18+40)
AGCRAAAGCAGGCAAACCATTTAGCTTGTCCTTCATGAAAAAATG
CCTTGTTTCTACT
78)
SEQ ID NO: 410
BM18_HA, 1, 1755, 43: (19+24)
AGCRAAAGCAGGGGAAAATAGGAAAAACACCCTTGTTTCTACT
79)
SEQ ID NO: 411
BM18_HA, 1, 1757, 38: (16+22)
AGCRAAAGCAGGGGAAGAAAAACACCCTTGTTTCTACT
80)
SEQ ID NO: 412
BM18_NS, 1, 864, 50: (23+27)
AGCRAAAGCAGGGTGACAAAGACATAATAAAAAACACCCTTGTTTC
TACT
81)
SEQ ID NO: 413
BM18_NS, 1, 869, 42: (20+22)
AGCRAAAGCAGGGTGACAAAAAAAAACACCCTTGTTTCTACT
82)
SEQ ID NO: 414
BM18_M, 1, 988, 73: (33+40)
AGCRAAAGCAGGTAGATGTTGAAAGATGAGTCTTCAACATAGAGCTGGA
GTAAAAAACTACCTTGTTTCTACT
83)
SEQ ID NO: 415
BM18_NA, 1, 1428, 62: (32+30)
AGCRAAAGCAGGAGTTTAAATGAATCCAAATCAGTTTGTTCAAAAAACT
CCTTGTTTCTACT
84)
SEQ ID NO: 416
BM18_NA, 1, 1437, 38: (17+21)
AGCRAAAGCAGGAGTTTAAAAAACTCCTTGTTTCTACT
85)
SEQ ID NO: 417
BM18_NP, 1, 1547, 58: (39+19)
AGCRAAAGCAGGGTAGATAATCACTCACTGAGTGACATCAAATACCCTTG
TTTCTACT
86)
SEQ ID NO: 418
BM18_NP, 1, 1546, 56: (36+20)
AGCRAAAGCAGGGTAGATAATCACTCACTGAGTGACAAAATACCCTTGTT
TCTACT
87)
SEQ ID NO: 419
BM18_NP, 1, 1535, 59: (28+31)
AGCRAAAGCAGGGTAGATAATCACTCACACAATTAAAGAAAAATACCCTT
GTTTCTACT
88)
SEQ ID NO: 420
BM18_NP, 1, 1540, 42: (16+26)
AGCRAAAGCAGGGTAGTAAAGAAAAATACCCTTGTTTCTACT
89)
SEQ ID NO: 421
BM18_PA, 1, 2213, 42: (21+21)
AGCRAAAGCAGGTACTGATTCAAAAAGTACCTTGTTTCTACT
90)
SEQ ID NO: 422
BM18_PA, 1, 2216, 35: (17+18)
AGCRAAAGCAGGTACTGAAGTACCTTGTTTCTACT
91)
SEQ ID NO: 423
BM18_PA, 1, 2217, 33: (16+17)
AGCRAAAGCAGGTACTAGTACCTTGTTTCTACT
92)
SEQ ID NO: 424
BM18_PB2, 1, 2312, 48: (18+30)
AGCRAAAGCAGGTCAATTGAATAGTTTAAAAACGACCTTGTTTCTACT
93)
SEQ ID NO: 425
BM18_PB2, 1, 2308, 51: (17+34)
AGCRAAAGCAGGTCAATTGTCGAATAGTTTAAAAACGACCTTGTTTCT
ACT
94)
SEQ ID NO: 426
BM18_PB1, 1, 2296, 71: (25+46)
AGCRAAAGCAGGCAAACCATTTGAATAGTGAATTTAGCTTGTCCTTCA
TGAAAAAATGCCTTGTTTCTACT
95)
SEQ ID NO: 427
BM18_PB1, 1, 2324, 40: (22+18)
AGCRAAAGCAGGCAAACCATTTAAATGCCTTGTTTCTACT
96)
SEQ ID NO: 428
BM18_PB1, 1, 2321, 43: (22+21)
AGCRAAAGCAGGCAAACCATTTAAAAAATGCCTTGTTTCTACT
97)
SEQ ID NO: 429
BM18_HA, 1, 1758, 46: (25+21)
AGCRAAAGCAGGGGAAAATAAAAACAAAAACACCCTTGTTTCTACT
98)
SEQ ID NO: 430
BM18_HA, 1, 1746, 55: (22+33)
AGCRAAAGCAGGGGAAAATAAAAGAAATATAAGGAAAAACACCCTTGT
TTCTACT
99)
SEQ ID NO: 431
BM18_HA, 1, 1731, 66: (18+48)
AGCRAAAGCAGGGGAAAATGAGATTAGGATTTCAGAAATATAAGGAAA
AACACCCTTGTTTCTACT
100)
SEQ ID NO: 432
BM18_HA, 1, 1757, 39: (17+22)
AGCRAAAGCAGGGGAAAGAAAAACACCCTTGTTTCTACT
101)
SEQ ID NO: 433
BM18_NS, 1, 873, 43: (25+18)
AGCRAAAGCAGGGTGACAAAGACATAACACCCTTGTTTCTACT
102)
SEQ ID NO: 434
BM18_NS, 1, 870, 46: (25+21)
AGCRAAAGCAGGGTGACAAAGACATAAAAACACCCTTGTTTCTACT
103)
SEQ ID NO: 435
BM18_NS, 1, 858, 57: (24+33)
AGCRAAAGCAGGGTGACAAAGACATATTTAATAATAAAAAACACCCT
TGTTTCTACT
104)
SEQ ID NO: 436
BM18_NS, 1, 872, 42: (23+19)
AGCRAAAGCAGGGTGACAAAGACAAACACCCTTGTTTCTACT
105)
SEQ ID NO: 437
BM18_NS, 1, 873, 39: (21+18)
AGCRAAAGCAGGGTGACAAAGAACACCCTTGTTTCTACT
106)
SEQ ID NO: 438
BM18_NS, 1, 863, 49: (21+28)
AGCRAAAGCAGGGTGACAAAGAATAATAAAAAACACCCTTGTTTC
TACT
107)
SEQ ID NO: 439
BM18_NS, 1, 866, 43: (18+25)
AGCRAAAGCAGGGTGACAAATAAAAAACACCCTTGTTTCTACT
108)
SEQ ID NO: 440
BM18_NS, 1, 866, 42: (17+25)
AGCRAAAGCAGGGTGACAATAAAAAACACCCTTGTTTCTACT
109)
SEQ ID NO: 441
BM18_M, 1, 1003, 56: (31+25)
AGCRAAAGCAGGTAGATGTTGAAAGATGAGTAGTAAAAAACTACCTT
GTTTCTACT
110)
SEQ ID NO: 442
BM18_M, 1, 1008, 48: (28+20)
AGCRAAAGCAGGTAGATGTTGAAAGATGAAAACTACCTTGTTTCT
ACT
111)
SEQ ID NO: 443
BM18_M, 1, 1006, 50: (28+22)
AGCRAAAGCAGGTAGATGTTGAAAGATGAAAAAACTACCTTGTTT
CTACT
112)
SEQ ID NO: 444
BM18_M, 1, 1005, 49: (26+23)
AGCRAAAGCAGGTAGATGTTGAAAGATAAAAAACTACCTTGTTTC
TACT
113)
SEQ ID NO: 445
BM18_M, 1, 1010, 43: (25+18)
AGCRAAAGCAGGTAGATGTTGAAAGAACTACCTTGTTTCTACT
114)
SEQ ID NO: 446
BM18_M, 1, 1006, 47: (25+22)
AGCRAAAGCAGGTAGATGTTGAAAGAAAAAACTACCTTGTTTC
TACT
115)
SEQ ID NO: 447
BM18_M, 1, 1007, 42: (21+21)
AGCRAAAGCAGGTAGATGTTGAAAAACTACCTTGTTTCTACT
116)
SEQ ID NO: 448
BM18_M, 1, 1002, 46: (20+26)
AGCRAAAGCAGGTAGATGTTGAGTAAAAAACTACCTTGTTTC
TACT
117)
SEQ ID NO: 449
BM18_M, 1, 1003, 44: (19+25)
AGCRAAAGCAGGTAGATGTAGTAAAAAACTACCTTGTTTCTACT
118)
SEQ ID NO: 450
BM18_M, 1, 1000, 45: (17+28)
AGCRAAAGCAGGTAGATTGGAGTAAAAAACTACCTTGTTTCTACT
119)
SEQ ID NO: 451
BM18_M, 1, 1005, 40: (17+23)
AGCRAAAGCAGGTAGATTAAAAAACTACCTTGTTTCTACT
120)
SEQ ID NO: 452
BM18_NA, 1, 1426, 64: (32+32)
AGCRAAAGCAGGAGTTTAAATGAATCCAAATCGTAGTTTGTTCAA
AAAACTCCTTGTTTCTACT
121)
SEQ ID NO: 453
BM18_NA, 1, 1418, 63: (23+40)
AGCRAAAGCAGGAGTTTAAATGAATTGACAAGTAGTTTGTTCAAA
AAACTCCTTGTTTCTACT
122)
SEQ ID NO: 454
BM18_NA, 1, 1420, 58: (20+38)
AGCRAAAGCAGGAGTTTAAATGACAAGTAGTTTGTTCAAAAAACTC
CTTGTTTCTACT
123)
SEQ ID NO: 455
BM18_NA, 1, 1421, 55: (18+37)
AGCRAAAGCAGGAGTTTAGACAAGTAGTTTGTTCAAAAAACTCCTT
GTTTCTACT
124)
SEQ ID NO: 456
BM18_NA, 1, 1438, 37: (17+20)
AGCRAAAGCAGGAGTTTAAAAACTCCTTGTTTCTACT
125)
SEQ ID NO: 457
BM18_NP, 1, 1548, 57: (39+18)
AGCRAAAGCAGGGTAGATAATCACTCACTGAGTGACATCAATAC
CCTTGTTTCTACT
126)
SEQ ID NO: 458
BM18_NP, 1, 1545, 60: (39+21)
AGCRAAAGCAGGGTAGATAATCACTCACTGAGTGACATCAAAAAT
ACCCTTGTTTCTACT
127)
SEQ ID NO: 459
BM18_NP, 1, 1547, 57: (38+19)
AGCRAAAGCAGGGTAGATAATCACTCACTGAGTGACATAAATACCC
TTGTTTCTACT
128)
SEQ ID NO: 460
BM18_NP, 1, 1535, 67: (36+31)
AGCRAAAGCAGGGTAGATAATCACTCACTGAGTGACACAATTAAAG
AAAAATACCCTTGTTTCTACT
129)
SEQ ID NO: 461
BM18_NP, 1, 1548, 39: (21+18)
AGCRAAAGCAGGGTAGATAATAATACCCTTGTTTCTACT
130)
SEQ ID NO: 462
BM18_NP, 1, 1542, 42: (18+24)
AGCRAAAGCAGGGTAGATAAGAAAAATACCCTTGTTTCTACT
131)
SEQ ID NO: 463
BM18_NP, 1, 1537, 47: (18+29)
AGCRAAAGCAGGGTAGATAATTAAAGAAAAATACCCTTGTTTCT
ACT
132)
SEQ ID NO: 464
BM18_NP, 1, 1535, 49: (18+31)
AGCRAAAGCAGGGTAGATACAATTAAAGAAAAATACCCTTGTTTC
TACT
133)
SEQ ID NO: 465
BM18_NP, 1, 1520, 62: (16+46)
AGCRAAAGCAGGGTAGATGCAGAGGAGTACGACAATTAAAGAAAAA
TACCCTTGTTTCTACT
134)
SEQ ID NO: 466
BM18_PA, 1, 2216, 56: (38+18)
AGCRAAAGCAGGTACTGATTCAAAATGGAAGACTTTGTAAGTACCTT
GTTTCTACT
135)
SEQ ID NO: 467
BM18_PA, 1, 2216, 54: (36+18)
AGCRAAAGCAGGTACTGATTCAAAATGGAAGACTTTAAGTACCTTGT
TTCTACT
136)
SEQ ID NO: 468
BM18_PA, 1, 2198, 61: (25+36)
AGCRAAAGCAGGTACTGATTCAAAATATCCATACTGTCCAAAAAAGT
ACCTTGTTTCTACT
137)
SEQ ID NO: 469
BM18_PA, 1, 2215, 40: (21+19)
AGCRAAAGCAGGTACTGATTCAAAGTACCTTGTTTCTACT
138)
SEQ ID NO: 470
BM18_PA, 1, 2209, 43: (18+25)
AGCRAAAGCAGGTACTGATCCAAAAAAGTACCTTGTTTCTACT
139)
SEQ ID NO: 471
BM18_PA, 1, 2212, 39: (17+22)
AGCRAAAGCAGGTACTGAAAAAAGTACCTTGTTTCTACT
140)
SEQ ID NO: 472
BM18_PB2, 1, 2312, 64: (34+30)
AGCRAAAGCAGGTCAATTATATTCAATATGGAAAGAATAGTTTAA
AAACGACCTTGTTTCTACT
141)
SEQ ID NO: 473
BM18_PB2, 1, 2313, 56: (27+29)
AGCRAAAGCAGGTCAATTATATTCAATAATAGTTTAAAAACGACCT
TGTTTCTACT
142)
SEQ ID NO: 474
BM18_PB2, 1, 2320, 48: (26+22)
AGCRAAAGCAGGTCAATTATATTCAATAAAAACGACCTTGTTTCTACT
143)
SEQ ID NO: 475
BM18_PB2, 1, 2305, 63: (26+37)
AGCRAAAGCAGGTCAATTATATTCAATAGTGTCGAATAGTTTAAAAA
CGACCTTGTTTCTACT
144)
SEQ ID NO: 476
BM18_PB2, 1, 2321, 45: (24+21)
AGCRAAAGCAGGTCAATTATATTCAAAAACGACCTTGTTTCTACT
145)
SEQ ID NO: 477
BM18_PB2, 1, 2316, 50: (24+26)
AGCRAAAGCAGGTCAATTATATTCAGTTTAAAAACGACCTTGTTT
CTACT
146)
SEQ ID NO: 478
BM18_PB2, 1, 2320, 41: (19+22)
AGCRAAAGCAGGTCAATTATAAAAACGACCTTGTTTCTACT
147)
SEQ ID NO: 479
BM18_PB2, 1, 2293, 67: (18+49)
AGCRAAAGCAGGTCAATTATGGCCATCAATTAGTGTCGAATAGTTTAAA
AACGACCTTGTTTCTACT
148)
SEQ ID NO: 480
BM18_PB2, 1, 2324, 35: (17+18)
AGCRAAAGCAGGTCAATAACGACCTTGTTTCTACT
149)
SEQ ID NO: 481
BM18_PB1, 1, 2319, 75: (52+23)
AGCRAAAGCAGGCAAACCATTTGAATGGATGTCAATCCGACTTTACTT
TTCTTGAAAAAATGCCTTGTTTCTACT
150)
SEQ ID NO: 482
BM18_PB1, 1, 2324, 62: (44+18)
AGCRAAAGCAGGCAAACCATTTGAATGGATGTCAATCCGACTTTAAAT
GCCTTGTTTCTACT
151)
SEQ ID NO: 483
BM18_PB1, 1, 2309, 75: (42+33)
AGCRAAAGCAGGCAAACCATTTGAATGGATGTCAATCCGACTTT
GTCCTTCATGAAAAAATGCCTTGTTTCTACT
152)
SEQ ID NO: 484
BM18_PB1, 1, 2324, 48: (30+18)
AGCRAAAGCAGGCAAACCATTTGAATGGATAAATGCCTTGTTT
CTACT
153)
SEQ ID NO: 485
BM18_PB1, 1, 2322, 50: (30+20)
AGCRAAAGCAGGCAAACCATTTGAATGGATAAAAATGCCTTGTTT
CTACT
154)
SEQ ID NO: 486
BM18_PB1, 1, 2318, 50: (26+24)
AGCRAAAGCAGGCAAACCATTTGAATATGAAAAAATGCCTTGTTT
CTACT
155)
SEQ ID NO: 487
BM18_PB1, 1, 2319, 45: (22+23)
AGCRAAAGCAGGCAAACCATTTTGAAAAAATGCCTTGTTTCTACT
156)
SEQ ID NO: 488
BM18_PB1, 1, 2299, 64: (21+43)
AGCRAAAGCAGGCAAACCATTTGAATTTAGCTTGTCCTTCATGAA
AAAATGCCTTGTTTCTACT
157)
SEQ ID NO: 489
BM18_PB1, 1, 2309, 53: (20+33)
AGCRAAAGCAGGCAAACCATTTGTCCTTCATGAAAAAATGCCTTG
TTTCTACT
158)
SEQ ID NO: 490
BM18_PB1, 1, 2320, 40: (18+22)
AGCRAAAGCAGGCAAACCGAAAAAATGCCTTGTTTCTACT
159)
SEQ ID NO: 491
BM18_PB1, 1, 2311, 49: (18+31)
AGCRAAAGCAGGCAAACCGTCCTTCATGAAAAAATGCCTTGTTT
CTACT
160)
SEQ ID NO: 492
BM18_PB1, 1, 2297, 63: (18+45)
AGCRAAAGCAGGCAAACCAGTGAATTTAGCTTGTCCTTCATGAA
AAAATGCCTTGTTTCTACT
161)
SEQ ID NO: 493
BM18_PB1, 1, 2317, 42: (17+25)
AGCRAAAGCAGGCAAACCATGAAAAAATGCCTTGTTTCTACT
162)
SEQ ID NO: 494
BM18_PB1, 1, 2290, 69: (17+52)
AGCRAAAGCAGGCAAACCAAAAGTAGTGAATTTAGCTTGTCCTTCA
TGAAAAAATGCCTTGTTTCTACT

### Example 5: Fasta File 4. WSN mvRNA Sequences

1)
BM18_PB2, 1, 2325, 35: (18+17)
SEQ ID NO: 495
AGCRAAAGCAGGTCAATTACGACCTTGTTTCTACT
2)
BM18_M, 1, 1010, 35: (17+18)
SEQ ID NO: 496
AGCRAAAGCAGGTAGATAACTACCTTGTTTCTACT
3)
BM18_M, 1, 1009, 36: (17+19)
SEQ ID NO: 497
AGCRAAAGCAGGTAGATAAACTACCTTGTTTCTACT
4)
BM18_PB2, 1, 2324, 36: (18+18)
SEQ ID NO: 498
AGCRAAAGCAGGTCAATTAACGACCTTGTTTCTACT
5)
BM18 NS, 1, 872, 36: (17+19)
SEQ ID NO: 499
AGCRAAAGCAGGGTGACAAACACCCTTGTTTCTACT
6)
BM18_NP, 1, 1548, 34: (16+18)
SEQ ID NO: 500
AGCRAAAGCAGGGTAGAATACCCTTGTTTCTACT
7)
BM18_NP, 1, 1547, 37: (18+19)
SEQ ID NO: 501
AGCRAAAGCAGGGTAGATAAATACCCTTGTTTCTACT
8)
BM18_NP, 1, 1547, 35: (16+19)
SEQ ID NO: 502
AGCRAAAGCAGGGTAGAAATACCCTTGTTTCTACT
9)
BM18_HA, 1, 1762, 36: (19+17)
SEQ ID NO: 503
AGCRAAAGCAGGGGAAAATACACCCTTGTTTCTACT
10)
BM18_NS, 1, 873, 35: (17+18)
SEQ ID NO: 504
AGCRAAAGCAGGGTGACAACACCCTTGTTTCTACT
11)
BM18_NP, 1, 1545, 39: (18+21)
SEQ ID NO: 505
AGCRAAAGCAGGGTAGATAAAAATACCCTTGTTTCTACT
12)
BM18_HA, 1, 1752, 44: (17+27)
SEQ ID NO: 506
AGCRAAAGCAGGGGAAAATAAGGAAAAACACCCTTGTTTCTACT
13)
BM18_PB2, 1, 2323, 37: (18+19)
SEQ ID NO: 507
AGCRAAAGCAGGTCAATTAAACGACCTTGTTTCTACT
14)
BM18_NS, 1, 871, 37: (17+20)
SEQ ID NO: 508
AGCRAAAGCAGGGTGACAAAACACCCTTGTTTCTACT
15)
BM18_HA, 1, 1761, 37: (19+18)
SEQ ID NO: 509
AGCRAAAGCAGGGGAAAATAACACCCTTGTTTCTACT
16)
BM18_NP, 1, 1546, 36: (16+20)
SEQ ID NO: 510
AGCRAAAGCAGGGTAGAAAATACCCTTGTTTCTACT
17)
BM18_HA, 1, 1757, 41: (19+22)
SEQ ID NO: 511
AGCRAAAGCAGGGGAAAATGAAAAACACCCTTGTTTCTACT
18)
BM18_NS, 1, 869, 39: (17+22)
SEQ ID NO: 512
AGCRAAAGCAGGGTGACAAAAAACACCCTTGTTTCTACT
19)
BM18_NP, 1, 1546, 38: (18+20)
SEQ ID NO: 513
AGCRAAAGCAGGGTAGATAAAATACCCTTGTTTCTACT
20)
BM18_PB2, 1, 2319, 39: (16+23)
SEQ ID NO: 514
AGCRAAAGCAGGTCAATTAAAAACGACCTTGTTTCTACT
21)
BM18_M, 1, 1008, 37: (17+20)
SEQ ID NO: 515
AGCRAAAGCAGGTAGATAAAACTACCTTGTTTCTACT
22)
BM18_HA, 1, 1760, 38: (19+19)
SEQ ID NO: 516
AGCRAAAGCAGGGGAAAATAAACACCCTTGTTTCTACT
23)
BM18_NP, 1, 1540, 43: (17+26)
SEQ ID NO: 517
AGCRAAAGCAGGGTAGATAAAGAAAAATACCCTTGTTTCTACT
24)
BM18_M, 1, 1005, 39: (16+23)
SEQ ID NO: 518
AGCRAAAGCAGGTAGATAAAAAACTACCTTGTTTCTACT
25)
BM18_PB1, 1, 2324, 36: (18+18)
SEQ ID NO: 519
AGCRAAAGCAGGCAAACCAAATGCCTTGTTTCTACT
26)
BM18_NP, 1, 1544, 40: (18+22)
SEQ ID NO: 520
AGCRAAAGCAGGGTAGATGAAAAATACCCTTGTTTCTACT
27)
BM18_NP, 1, 1549, 35: (18+17)
SEQ ID NO: 521
AGCRAAAGCAGGGTAGATATACCCTTGTTTCTACT
28)
BM18_M, 1, 1011, 34: (17+17)
SEQ ID NO: 522
AGCRAAAGCAGGTAGATACTACCTTGTTTCTACT
29)
BM18_PB1, 1, 2323, 37: (18+19)
SEQ ID NO: 523
AGCRAAAGCAGGCAAACCAAAATGCCTTGTTTCTACT
30)
BM18_HA, 1, 1758, 37: (16+21)
SEQ ID NO: 524
AGCRAAAGCAGGGGAAAAAAACACCCTTGTTTCTACT
31)
BM18_NP, 1, 1543, 41: (18+23)
SEQ ID NO: 525
AGCRAAAGCAGGGTAGATAGAAAAATACCCTTGTTTCTACT
32)
BM18_NS, 1, 874, 34: (17+17)
SEQ ID NO: 526
AGCRAAAGCAGGGTGACACACCCTTGTTTCTACT
33)
BM18_PA, 1, 2216, 34: (16+18)
SEQ ID NO: 527
AGCRAAAGCAGGTACTAAGTACCTTGTTTCTACT
34)
BM18_HA, 1, 1756, 42: (19+23)
SEQ ID NO: 528
AGCRAAAGCAGGGGAAAATGGAAAAACACCCTTGTTTCTACT
35)
BM18_PB2, 1, 2315, 44: (17+27)
SEQ ID NO: 529
AGCRAAAGCAGGTCAATTAGTTTAAAAACGACCTTGTTTCTACT
36)
BM18_NS, 1, 870, 38: (17+21)
SEQ ID NO: 530
AGCRAAAGCAGGGTGACAAAAACACCCTTGTTTCTACT
37)
BM18_PB2, 1, 2322, 38: (18+20)
SEQ ID NO: 531
AGCRAAAGCAGGTCAATTAAAACGACCTTGTTTCTACT
38)
BM18_PB1, 1, 2325, 35: (18+17)
SEQ ID NO: 532
AGCRAAAGCAGGCAAACCAATGCCTTGTTTCTACT
39)
BM18_HA, 1, 1758, 38: (17+21)
SEQ ID NO: 533
AGCRAAAGCAGGGGAAAAAAAACACCCTTGTTTCTACT
40)
BM18_HA, 1, 1754, 42: (17+25)
SEQ ID NO: 534
AGCRAAAGCAGGGGAAAAAGGAAAAACACCCTTGTTTCTACT
41)
BM18_HA, 1, 1759, 39: (19+20)
SEQ ID NO: 535
AGCRAAAGCAGGGGAAAATAAAACACCCTTGTTTCTACT
42)
BM18_NA, 1, 1439, 36: (17+19)
SEQ ID NO: 536
AGCRAAAGCAGGAGTTTAAAACTCCTTGTTTCTACT
43)
BM18_M, 1, 1007, 38: (17+21)
SEQ ID NO: 537
AGCRAAAGCAGGTAGATAAAAACTACCTTGTTTCTACT
44)
BM18_NA, 1, 1437, 38: (17+21)
AGCRAAAGCAGGAGTTTAAAAAACTCCTTGTTTCTACT
SEQ ID NO: 538
45)
BM18_PB1, 1, 2321, 39: (18+21)
SEQ ID NO: 539
AGCRAAAGCAGGCAAACCAAAAAATGCCTTGTTTCTACT
46)
BM18_NS, 1, 867, 47: (23+24)
SEQ ID NO: 540
AGCRAAAGCAGGGTGACAAAGACATAAAAAACACCCTTGTTTCTACT
47)
BM18_NP, 1, 1532, 62: (28+34)
SEQ ID NO: 541
AGCRAAAGCAGGGTAGATAATCACTCACACGACAATTAAAGAAAAATAC
CCTTGTTTCTACT
48)
BM18_NP, 1, 1541, 41: (16+25)
SEQ ID NO: 542
AGCRAAAGCAGGGTAGAAAGAAAAATACCCTTGTTTCTACT
49)
BM18_PB1, 1, 2322, 38: (18+20)
SEQ ID NO: 543
AGCRAAAGCAGGCAAACCAAAAATGCCTTGTTTCTACT
50)
BM18_NP, 1, 1531, 52: (17+35)
SEQ ID NO: 544
AGCRAAAGCAGGGTAGATACGACAATTAAAGAAAAATACCCTTGTTTCT
ACT
51)
BM18_PA, 1, 2212, 48: (26+22)
SEQ ID NO: 545
AGCRAAAGCAGGTACTGATTCAAAATAAAAAAGTACCTTGTTTCTACT
52)
BM18_HA, 1, 1752, 59: (32+27)
SEQ ID NO: 546
AGCRAAAGCAGGGGAAAATAAAAACAACCAAAATAAGGAAAAACACCCT
TGTTTCTACT
53)
BM18_HA, 1, 1754, 41: (16+25)
SEQ ID NO: 547
AGCRAAAGCAGGGGAAAAGGAAAAACACCCTTGTTTCTACT
54)
BM18_PA, 1, 2211, 43: (20+23)
SEQ ID NO: 548
AGCRAAAGCAGGTACTGATTCAAAAAAGTACCTTGTTTCTACT
55)
BM18_HA, 1, 1755, 43: (19+24)
SEQ ID NO: 549
AGCRAAAGCAGGGGAAAATAGGAAAAACACCCTTGTTTCTACT
56)
BM18_HA, 1, 1737, 60: (18+42)
SEQ ID NO: 550
AGCRAAAGCAGGGGAAAATAGGATTTCAGAAATATAAGGAAAAACACCC
TTGTTTCTACT
57)
BM18_PB2, 1, 2313, 47: (18+29)
SEQ ID NO: 551
AGCRAAAGCAGGTCAATTAATAGTTTAAAAACGACCTTGTTTCTACT
58)
BM18_NS, 1, 864, 50: (23+27)
SEQ ID NO: 552
AGCRAAAGCAGGGTGACAAAGACATAATAAAAAACACCCTTGTTTCTAC
T
59)
BM18_NP, 1, 1546, 56: (36+20)
SEQ ID NO: 553
AGCRAAAGCAGGGTAGATAATCACTCACTGAGTGACAAAATACCCTTGT
TTCTACT
60)
BM18_NP, 1, 1535, 59: (28+31)
SEQ ID NO: 554
AGCRAAAGCAGGGTAGATAATCACTCACACAATTAAAGAAAAATACCCT
TGTTTCTACT
61)
BM18_PA, 1, 2212, 44: (22+22)
SEQ ID NO: 555
AGCRAAAGCAGGTACTGATTCAAAAAAAGTACCTTGTTTCTACT
62)
BM18_PA, 1, 2215, 40: (21+19)
SEQ ID NO: 556
AGCRAAAGCAGGTACTGATTCAAAGTACCTTGTTTCTACT
63)
BM18_PA, 1, 2208, 47: (21+26)
SEQ ID NO: 557
AGCRAAAGCAGGTACTGATTCGTCCAAAAAAGTACCTTGTTTCTACT
64)
BM18_PA, 1, 2215, 35: (16+19)
SEQ ID NO: 558
AGCRAAAGCAGGTACTAAAGTACCTTGTTTCTACT
65)
BM18_PB2, 1, 2313, 53: (24+29)
SEQ ID NO: 559
AGCRAAAGCAGGTCAATTATATTCAATAGTTTAAAAACGACCTTGTTTC
TACT
66)
BM18_PB2, 1, 2314, 46: (18+28)
SEQ ID NO: 560
AGCRAAAGCAGGTCAATTATAGTTTAAAAACGACCTTGTTTCTACT
67)
BM18_PB2, 1, 2323, 36: (17+19)
SEQ ID NO: 561
AGCRAAAGCAGGTCAATAAACGACCTTGTTTCTACT
68)
BM18_HA, 1, 1748, 61: (30+31)
SEQ ID NO: 562
AGCRAAAGCAGGGGAAAATAAAAACAACCAAAATATAAGGAAAAACACC
CTTGTTTCTACT
69)
BM18_HA, 1, 1738, 66: (25+41)
SEQ ID NO: 563
AGCRAAAGCAGGGGAAAATAAAAACAGGATTTCAGAAATATAAGGAAAA
ACACCCTTGTTTCTACT
70)
BM18_HA, 1, 1758, 39: (18+21)
SEQ ID NO: 564
AGCRAAAGCAGGGGAAAAAAAAACACCCTTGTTTCTACT
71)
BM18_HA, 1, 1757, 39: (17+22)
SEQ ID NO: 565
AGCRAAAGCAGGGGAAAGAAAAACACCCTTGTTTCTACT
72)
BM18_HA, 1, 1738, 58: (17+41)
SEQ ID NO: 566
AGCRAAAGCAGGGGAAAAGGATTTCAGAAATATAAGGAAAAACACCCTT
GTTTCTACT
73)
BM18_NS, 1, 866, 55: (30+25)
SEQ ID NO: 567
AGCRAAAGCAGGGTGACAAAGACATAATGGAATAAAAAACACCCTTGTT
TCTACT
74)
BM18_M, 1, 1011, 35: (18+17)
SEQ ID NO: 568
AGCRAAAGCAGGTAGATGACTACCTTGTTTCTACT
75)
BM18_NA, 1, 1428, 57: (27+30)
SEQ ID NO: 569
AGCRAAAGCAGGAGTTTAAATGAATCCAGTTTGTTCAAAAAACTCCTTG
TTTCTACT
76)
BM18_NA, 1, 1427, 47: (16+31)
SEQ ID NO: 570
AGCRAAAGCAGGAGTTTAGTTTGTTCAAAAAACTCCTTGTTTCTACT
77)
BM18_NP, 1, 1548, 57: (39+18)
SEQ ID NO: 571
AGCRAAAGCAGGGTAGATAATCACTCACTGAGTGACATCAATACCCTTG
TTTCTACT
78)
BM18_NP, 1, 1535, 57: (26+31)
SEQ ID NO: 572
AGCRAAAGCAGGGTAGATAATCACTCACAATTAAAGAAAAATACCCTTG
TTTCTACT
79)
BM18_PA, 1, 2207, 52: (25+27)
SEQ ID NO: 573
AGCRAAAGCAGGTACTGATTCAAAATGTCCAAAAAAGTACCTTGTTTCT
ACT
80)
BM18_PA, 1, 2215, 36: (17+19)
SEQ ID NO: 574
AGCRAAAGCAGGTACTGAAAGTACCTTGTTTCTACT
81)
BM18_PA, 1, 2214, 36: (16+20)
SEQ ID NO: 575
AGCRAAAGCAGGTACTAAAAGTACCTTGTTTCTACT
82)
BM18_PB2, 1, 2320, 48: (26+22)
SEQ ID NO: 576
AGCRAAAGCAGGTCAATTATATTCAATAAAAACGACCTTGTTTCTACT
83)
BM18_PB1, 1, 2321, 43: (22+21)
SEQ ID NO: 577
AGCRAAAGCAGGCAAACCATTTAAAAAATGCCTTGTTTCTACT
84)
BM18_PB1, 1, 2319, 44: (21+23)
SEQ ID NO: 578
AGCRAAAGCAGGCAAACCATTTGAAAAAATGCCTTGTTTCTACT
85)
BM18_PB1, 1, 2320, 39: (17+22)
SEQ ID NO: 579
AGCRAAAGCAGGCAAACGAAAAAATGCCTTGTTTCTACT
86)
BM18_HA, 1, 1752, 45: (18+27)
SEQ ID NO: 580
AGCRAAAGCAGGGGAAAAATAAGGAAAAACACCCTTGTTTCTACT
87)
BM18_HA, 1, 1731, 66: (18+48)
SEQ ID NO: 581
AGCRAAAGCAGGGGAAAATGAGATTAGGATTTCAGAAATATAAGGAAAA
ACACCCTTGTTTCTACT
88)
BM18_NS, 1, 869, 52: (30+22)
SEQ ID NO: 582
AGCRAAAGCAGGGTGACAAAGACATAATGGAAAAAACACCCTTGTTTCT
ACT
89)
BM18_NS, 1, 872, 40: (21+19)
SEQ ID NO: 583
AGCRAAAGCAGGGTGACAAAGAAACACCCTTGTTTCTACT
90)
BM18_M, 1, 1004, 41: (17+24)
SEQ ID NO: 584
AGCRAAAGCAGGTAGATGTAAAAAACTACCTTGTTTCTACT
91)
BM18_NA, 1, 1433, 53: (28+25)
SEQ ID NO: 585
AGCRAAAGCAGGAGTTTAAATGAATCCAGTTCAAAAAACTCCTTGTTTC
TACT
92)
BM18_NP, 1, 1544, 61: (39+22)
SEQ ID NO: 586
AGCRAAAGCAGGGTAGATAATCACTCACTGAGTGACATCGAAAAATACC
CTTGTTTCTACT
93)
BM18_NP, 1, 1550, 37: (21+16)
SEQ ID NO: 587
AGCRAAAGCAGGGTAGATAATTACCCTTGTTTCTACT
94)
BM18_NP, 1, 1529, 55: (18+37)
SEQ ID NO: 588
AGCRAAAGCAGGGTAGATAGTACGACAATTAAAGAAAAATACCCTTGTT
TCTACT
95)
BM18_NP, 1, 1544, 38: (16+22)
SEQ ID NO: 589
AGCRAAAGCAGGGTAGGAAAAATACCCTTGTTTCTACT
96)
BM18_PA, 1, 2207, 64: (37+27)
SEQ ID NO: 590
AGCRAAAGCAGGTACTGATTCAAAATGGAAGACTTTGTGTCCAAAAAAG
TACCTTGTTTCTACT
97)
BM18_PA, 1, 2214, 41: (21+20)
SEQ ID NO: 591
AGCRAAAGCAGGTACTGATTCAAAAGTACCTTGTTTCTACT
98)
BM18_PA, 1, 2217, 33: (16+17)
SEQ ID NO: 592
AGCRAAAGCAGGTACTAGTACCTTGTTTCTACT
99)
BM18_PB2, 1, 2324, 35: (17+18)
SEQ ID NO: 593
AGCRAAAGCAGGTCAATAACGACCTTGTTTCTACT
100)
BM18_PB1, 1, 2294, 74: (26+48)
SEQ ID NO: 594
AGCRAAAGCAGGCAAACCATTTGAATAGTAGTGAATTTAGCTTGTCCTT
CATGAAAAAATGCCTTGTTTCTACT
101)
BM18_PB1, 1, 2326, 36: (20+16)
SEQ ID NO: 595
AGCRAAAGCAGGCAAACCATATGCCTTGTTTCTACT
102)
BM18_PB1, 1, 2320, 40: (18+22)
SEQ ID NO: 596
AGCRAAAGCAGGCAAACCGAAAAAATGCCTTGTTTCTACT
103)
BM18_PB1, 1, 2324, 35: (17+18)
SEQ ID NO: 597
AGCRAAAGCAGGCAAACAAATGCCTTGTTTCTACT
104)
BM18_PB1, 1, 2323, 36: (17+19)
SEQ ID NO: 598
AGCRAAAGCAGGCAAACAAAATGCCTTGTTTCTACT
105)
BM18_HA, 1, 1737, 75: (33+42)
SEQ ID NO: 599
AGCRAAAGCAGGGGAAAATAAAAACAACCAAAATAGGATTTCAGAAATA
TAAGGAAAAACACCCTTGTTTCTACT
106)
BM18_HA, 1, 1757, 43: (21+22)
SEQ ID NO: 600
AGCRAAAGCAGGGGAAAATAAGAAAAACACCCTTGTTTCTACT
107)
BM18_NS, 1, 868, 63: (40+23)
SEQ ID NO: 601
AGCRAAAGCAGGGTGACAAAGACATAATGGATTCTAACACTAAAAAACA
CCCTTGTTTCTACT
108)
BM18_NS, 1, 871, 45: (25+20)
SEQ ID NO: 602
AGCRAAAGCAGGGTGACAAAGACATAAAACACCCTTGTTTCTACT
109)
BM18_NS, 1, 862, 53: (24+29)
SEQ ID NO: 603
AGCRAAAGCAGGGTGACAAAGACATAATAATAAAAAACACCCTTGTTTC
TACT
110)
BM18_NS, 1, 873, 39: (21+18)
SEQ ID NO: 604
AGCRAAAGCAGGGTGACAAAGAACACCCTTGTTTCTACT
111)
BM18_NS, 1, 869, 43: (21+22)
SEQ ID NO: 605
AGCRAAAGCAGGGTGACAAAGAAAAAACACCCTTGTTTCTACT
112)
BM18_NS, 1, 837, 73: (19+54)
SEQ ID NO: 606
AGCRAAAGCAGGGTGACAAAAGAACTTTCTCGTTTCAGCTTATTTAATA
ATAAAAAACACCCTTGTTTCTACT
113)
BM18_NS, 1, 875, 33: (17+16)
SEQ ID NO: 607
AGCRAAAGCAGGGTGACCACCCTTGTTTCTACT
114)
BM18_M, 1, 1006, 69: (47+22)
SEQ ID NO: 608
AGCRAAAGCAGGTAGATGTTGAAAGATGAGTCTTCTAACCGAGGTCGAA
AAAACTACCTTGTTTCTACT
115)
BM18_M, 1, 1010, 45: (27+18)
SEQ ID NO: 609
AGCRAAAGCAGGTAGATGTTGAAAGATAACTACCTTGTTTCTACT
116)
BM18_M, 1, 1000, 54: (26+28)
SEQ ID NO: 610
AGCRAAAGCAGGTAGATGTTGAAAGATGGAGTAAAAAACTACCTTGTTT
CTACT
117)
BM18_M, 1, 1002, 43: (17+26)
SEQ ID NO: 611
AGCRAAAGCAGGTAGATGAGTAAAAAACTACCTTGTTTCTACT
118)
BM18_NA, 1, 1429, 56: (27+29)
SEQ ID NO: 612
AGCRAAAGCAGGAGTTTAAATGAATCCGTTTGTTCAAAAAACTCCTTGT
TTCTACT
119)
BM18_NA, 1, 1438, 36: (16+20)
SEQ ID NO: 613
AGCRAAAGCAGGAGTTAAAAACTCCTTGTTTCTACT
120)
BM18_NP, 1, 1548, 69: (51+18)
SEQ ID NO: 614
AGCRAAAGCAGGGTAGATAATCACTCACTGAGTGACATCAAAATCATGG
CGAATACCCTTGTTTCTACT
121)
BM18_NP, 1, 1541, 64: (39+25)
SEQ ID NO: 615
AGCRAAAGCAGGGTAGATAATCACTCACTGAGTGACATCAAAGAAAAAT
ACCCTTGTTTCTACT
122)
BM18_NP, 1, 1547, 55: (36+19)
SEQ ID NO: 616
AGCRAAAGCAGGGTAGATAATCACTCACTGAGTGACAAATACCCTTGTT
TCTACT
123)
BM18_NP, 1, 1545, 42: (21+21)
SEQ ID NO: 617
AGCRAAAGCAGGGTAGATAATAAAAATACCCTTGTTTCTACT
124)
BM18_NP, 1, 1550, 34: (18+16)
SEQ ID NO: 618
AGCRAAAGCAGGGTAGATTACCCTTGTTTCTACT
125)
BM18_NP, 1, 1542, 42: (18+24)
SEQ ID NO: 619
AGCRAAAGCAGGGTAGATAAGAAAAATACCCTTGTTTCTACT
126)
BM18_NP, 1, 1537, 47: (18+29)
SEQ ID NO: 620
AGCRAAAGCAGGGTAGATAATTAAAGAAAAATACCCTTGTTTCTACT
127)
BM18_NP, 1, 1535, 49: (18+31)
SEQ ID NO: 621
AGCRAAAGCAGGGTAGATACAATTAAAGAAAAATACCCTTGTTTCTACT
128)
BM18_NP, 1, 1539, 43: (16+27)
SEQ ID NO: 622
AGCRAAAGCAGGGTAGTTAAAGAAAAATACCCTTGTTTCTACT
129)
BM18_PA, 1, 2208, 57: (31+26)
SEQ ID NO: 623
AGCRAAAGCAGGTACTGATTCAAAATGGAAGGTCCAAAAAAGTACCTTG
TTTCTACT
130)
BM18_PA, 1, 2216, 39: (21+18)
SEQ ID NO: 624
AGCRAAAGCAGGTACTGATTCAAGTACCTTGTTTCTACT
131)
BM18_PA, 1, 2213, 41: (20+21)
SEQ ID NO: 625
AGCRAAAGCAGGTACTGATTAAAAAGTACCTTGTTTCTACT
132)
BM18_PA, 1, 2217, 34: (17+17)
SEQ ID NO: 626
AGCRAAAGCAGGTACTGAGTACCTTGTTTCTACT
133)
BM18_PA, 1, 2213, 37: (16+21)
SEQ ID NO: 627
AGCRAAAGCAGGTACTAAAAAGTACCTTGTTTCTACT
134)
BM18_PB2, 1, 2323, 48: (29+19)
SEQ ID NO: 628
AGCRAAAGCAGGTCAATTATATTCAATATAAACGACCTTGTTTCTACT
135)
BM18_PB2, 1, 2306, 61: (25+36)
AGCRAAAGCAGGTCAATTATATTCAAGTGTCGAATAGTTTAAAAACGAC
SEQ ID NO: 629
CTTGTTTCTACT
136)
BM18_PB2, 1, 2314, 48: (20+28)
SEQ ID NO: 630
AGCRAAAGCAGGTCAATTATATAGTTTAAAAACGACCTTGTTTCTACT
137)
BM18_PB2, 1, 2326, 34: (18+16)
SEQ ID NO: 631
AGCRAAAGCAGGTCAATTCGACCTTGTTTCTACT
138)
BM18_PB2, 1, 2293, 67: (18+49)
SEQ ID NO: 632
AGCRAAAGCAGGTCAATTATGGCCATCAATTAGTGTCGAATAGTTTAAA
AACGACCTTGTTTCTACT
139)
BM18_PB2, 1, 2325, 34: (17+17)
SEQ ID NO: 633
AGCRAAAGCAGGTCAATACGACCTTGTTTCTACT
140)
BM18_PB2, 1, 2308, 51: (17+34)
SEQ ID NO: 634
AGCRAAAGCAGGTCAATTGTCGAATAGTTTAAAAACGACCTTGTTTCTA
CT
141)
BM18_PB2, 1, 2320, 38: (16+22)
SEQ ID NO: 635
AGCRAAAGCAGGTCAATAAAAACGACCTTGTTTCTACT
142)
BM18_PB1, 1, 2314, 73: (45+28)
SEQ ID NO: 636
AGCRAAAGCAGGCAAACCATTTGAATGGATGTCAATCCGACTTTACTTC
ATGAAAAAATGCCTTGTTTCTACT
143)
BM18_PB1, 1, 2323, 49: (30+19)
SEQ ID NO: 637
AGCRAAAGCAGGCAAACCATTTGAATGGATAAAATGCCTTGTTTCTACT
144)
BM18_PB1, 1, 2321, 47: (26+21)
SEQ ID NO: 638
AGCRAAAGCAGGCAAACCATTTGAATAAAAAATGCCTTGTTTCTACT
145)
BM18_PB1, 1, 2320, 41: (19+22)
SEQ ID NO: 639
AGCRAAAGCAGGCAAACCAGAAAAAATGCCTTGTTTCTACT
146)
BM18_PB1, 1, 2325, 34: (17+17)
SEQ ID NO: 640
AGCRAAAGCAGGCAAACAATGCCTTGTTTCTACT
147)
BM18_PB1, 1, 2317, 42: (17+25)
SEQ ID NO: 641
AGCRAAAGCAGGCAAACCATGAAAAAATGCCTTGTTTCTACT

### Example 6: Python Script for Sliding Window Analysis

```
#Influenza virus RNA polymerase simulation script that searches for t-loops and checks up/down
stream for intermol bp
#Uses Python 3.8, biopython, openpyxl, and Vienna RNA 2.47; side packages were installed with
Anaconda3
#By AJ te Velthuis, Sept 2020
import sys
sys.path.append(“/users/USER/opt/anaconda3/lib/python3.8/site-packages”)
##input sequence. Paste sequence between quotation marks below.
RNA = “PASTE SEQUENCE HERE”
Name =“Template”
##output bubbles in txt file
f= open(“deltaGvalues.txt”,“w+”)
##specify polymerase properties
Footprint = 20
##specify NP footprint
NP = 24
TloopDuplex = 48
Duplex = int(TloopDuplex / 2)
##specify window and other comparisons
Uloop = “&” #Use & for co-fold to compute long-range interactions between upstream and
downstream sequences.
Swindow = 1 #size of sliding window
###########################################
##invert input sequence to start at 3′ end
NegRNA = RNA[::−1]
Length = len(NegRNA)
##########################################
##polymerase bubble properties
Bubble = Footprint + Duplex
##iteration start point of simulation; start at nt 2 otherwise downstream sequence is empty for co-fold
i = 1
##end bubble sequence and add 1, because sequence count starts at 0
End = int((Length − Footprint + 1) / Swindow)
##find polymerase bubble sequence; allow for small 3′ part to emerge, but
##then cap how long the emerging sequence can be by assuming that every 24 nt will be bound by
NP
##unclear if NP binds in chunks or progressively. Assume that at least 24 nt are needed based on data
from Ortin lab.
##stops 1 nt from end to avoid having no sequence in cofold
for i in range(1, End−1):
  if i <= NP:
    Upstream = i
    Downstream = 0
  else:
    Upstream = NP
    Downstream = i−NP
  #find upstream and downstream sequence of the bubble for intermolecular folding check
  Ahead = NegRNA[Footprint+i:Footprint+NP+i]
  Aheadinv = Ahead[::−1]
  #currently only takes 1 nt of downstream as 0 gives an error in cofold.
  Down = NegRNA[Downstream:i]
  Downinv = Down[::−1]
  ##find the two duplex ends and invert them so they are 5′ to 3′
  if i <= Duplex:
    Prime3 = NegRNA[0:Upstream]
  else:
    Prime3 = NegRNA[i−Duplex:i]
  Prime3inv = Prime3[::−1]
  if i <= End:
    Prime5 = NegRNA[Footprint+i:Bubble+i]
  else:
    Prime5 = NegRNA[Footprint+i:Length]
  Prime5inv = Prime5[::−1]
  #compute A/U content of nucleotides in active site. Assume window of 6 before and after active
site. Various print options are inactivated, but can be used for checking if script works.
  ActiveSite = i + 16
  #print(“location of bubble is ”, i+1)
  #print(“3 prime end 3′ to 5′ is ”, Prime3)
  #print(“5 prime end 5′ to 3′ is ”, Prime5inv)
  ActiveSiteSeqUp = NegRNA[ActiveSite−1:ActiveSite+4]
  ActiveSiteSeqDown = NegRNA[ActiveSite−5:ActiveSite]
  #seq_up_list = list(ActiveSiteSeqUp)
  seq_up_list = list(ActiveSiteSeqDown)
  at_count_up = seq_up_list.count(“a”) + seq_up_list.count (“t”) + seq_up_list.count(“A”) +
seq_up_list.count (“T”) + seq_up_list.count (“u”) + seq_up_list.count (“U”)
  at_frac_up = float(at_count_up)/5
  total_at_up = 100 * at_frac_up
##saving the A/U content is inactivated below
#print(total_at_up)
  #f.write(“%f\n” % (total_at_up))
  ##Vienna RNA package for RNA structure prediction
  import RNA
  Test = Prime3 + Uloop + Prime5
  NegTest = Test[::−1]
  #Output = (“>”+Name+“_polymerase_bubble_number_%d\n” % (i+1))
  ##To write bubble sequence to .txt file
  #f.write(Output)
  #f.write(NegTest)
  #f.write(“\r\n”)
  #use duplex fold to compute t-loop from Vienna package because it ignores intermol bp
  duplex = RNA.duplexfold(Prime5inv, Prime3inv)
  #print(“%s\n%s [%6.2f]” % (NegTest, duplex.structure, duplex.energy))
  ##print(“%6.2f” % (duplex.energy))
  #use cofold from Vienna package to check for bp in sequence upstream and downstream of t-loop.
Various options can be checked separately, including just upstream seq, just downstream seq, or both
seq
  Other = Aheadinv + Uloop + Downinv
  #Other = Aheadinv
  #Other = Downinv
  (ss, mfe_dimer) = RNA.cofold(Other)
  #print(“%s\n%s [%6.2f]” % (Other, ss, mfe_dimer))
  DDeltaG = duplex.energy − mfe_dimer ##compute DeltaDeltaG
  ##check if deltaG of t-loop is lower than deltaG of other structures
  if duplex.energy >= mfe_dimer:
    DeltaG = mfe_dimer * −1
  #elif duplex.energy >= 0:
    #DeltaG = 0
  else:
    #DeltaG = duplex.energy
    DeltaG = duplex.energy
  #DeltaG = mfe_dimer
  #DeltaG = duplex.energy
  #print(DeltaG)
  #print(DDeltaG)
  #print(duplex.energy)
  #print(“%s\n%s [ %6.2f ]” % (NegTest, ss, mfe))
  ##to write deltaG values to .txt file. Various options are available depending on what is being
analyzed.
  f.write(“%f\n” % (duplex.energy))
  #f.write(“%f\n” % (DeltaG))
  #f.write(“%f\n” % (mfe_dimer))
  #print(NegTest)
  #print(“−−−−−”)
  i =+ Swindow
####################################################
##close .txt file that deltaG values were written to
f.close( )
print (“Done %s” % (Name))
```

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLE 1
Sequences of mvRNA templates used.

| | Internal lab | | |
|---|---|---|---|
| Name | reference | Sequence 5′ and 3′ (vRNA) | SEQ ID NO. |
| NP71.1 | GC33 | AGUAGAAACAAGGGUAUUUUUCUUUAC | SEQ ID NO: 4 |
| | | UAGUUAGGUAGUAUACCUAGUAACUAG | |
| | | UCUACCCUGCUUUUGCU | |
| NP71.2 | GC50.1 | AGUAGAAACAAGGGUAUUUUUUUUAC | SEQ ID NO: 5 |
| | | UAGUCCGGUUGUUUUGGUUGCCACUAG | |
| | | UCUACCCUGCUUUUGCU | |
| NP71.3 | GC50.2 | AGUAGAAACAAGGGUAUUUUUCUUUAC | SEQ ID NO: 6 |
| | | UAGUCCGCUUGUAUAGCUUGCCACUAG | |
| | | UCUACCCUGCUUUUGCU | |
| NP71.4 | GC67 | AGUAGAAACAAGGGUAUUUUUCUUUAC | SEQ ID NO: 7 |
| | | UAGUCCGGCCGAUAUGGCCGCCACUAG | |
| | | UCUACCCUGCUUUUGCU | |
| NP71.5 | GC83 | AGUAGAAACAAGGGUAUUUUUCUUUAC | SEQ ID NO: 8 |
| | | UAGUCCGGCCGCCCCGGCCGCCACUAG | |
| | | UCUACCCUGCUUUUGCU | |
| NP71.6 | GC50.9 | AGUAGAAACAAGGGUAUUUUUCUUUAC | SEQ ID NO: 9 |
| | | UAGUCCGGCCGUUUUGGUUGCCACUAG | |
| | | UCUACCCUGCUUUUGCU | |
| NP71.7 | GC50.3 | AGUAGAAACAAGGGUAUUUUUCUUUAC | SEQ ID NO: 10 |
| | | UAGUCCGGUUCUUUUGGUUGCCACUAG | |
| | | UCUACCCUGCUUUUGCU | |
| NP71.8 | GC50.4 | AGUAGAAACAAGGGUAUUUUUCUUUAC | SEQ ID NO: 11 |
| | | UAGUCCGGUUGCUUUGGUUGCCACUAG | |
| | | UCUACCCUGCUUUUGCU | |
| NP47 | NP47 | AGUAGAAACAAGGGUAUUUUUCUUUAC | SEQ ID NO: 1 |
| | | UAGUCUACCCUGCUUUUGCU | |
| NP56 | NP56 | AGUAGAAACAAGGGUAUUUUUCUUUCU | SEQ ID NO: 2 |
| | | CGAGCGUACUAGUCUACCCUGCUUUUG | |
| | | CU | |
| NP76 | NP76 | AGUAGAAACAAGGGUAUUUUUCUUUAC | SEQ ID NO: 3 |
| | | UAGUGAUUUCGAUGUCACUCUGUGAGU | |
| | | GAUUAUCUACCCUGCUUUUGCU | |
| NP71.10 | GC50 13 | AGUAGAAACAAGGGUAUUUUUCUUUAC | SEQ ID NO: 12 |
| | | UAGUGGCAGCAAAAGCAGGGUAACUAG | |
| | | UCUACCCUGCUUUUGCU | |
| NP71.11 | GC50 15 | AGUAGAAACAAGGGUAUUUUUCUUUAC | SEQ ID NO: 13 |
| | | UAGUGGCAGCAAAAGCACCCAUACUAG | |
| | | UCUACCCUGCUUUUGCU | |
| NP71.12 | GC50 16 | AGUAGAAACAAGGGUAUUUUUCUUUAC | SEQ ID NO: 14 |
| | | UAGUGGCUCUAAAAGCACCCAUACUAG | |
| | | UCUACCCUGCUUUUGCU | |

TABLE 2
Cloned PB1 WSN mvRNAs.

| | Length | | SEQ ID |
|---|---|---|---|
| Name | (nt) | Sequence 5′ and 3′ (vRNA) | NO. |
| PB1 A | 57 | AGUAGAAACAAGGCAUUUUUUCAUGAA | SEQ ID |
| | | AUCCAUUCAAAUGGUUUGCCUGCUUUC | NO: 15 |
| | | GCU | |
| PB1 B | 57 | AGUAGAAACAAGGCAUUUUUUCAUGAA | SEQ ID |
| | | GGACAUUCAAAUGGUUUGCCUGCUUUC | NO: 16 |
| | | GCU | |
| PB1 C | 66 | AGUAGAAACAAGGCAUUUUUUCAUGAA | SEQ ID |
| | | GGACAAGCUAAACAUUCAAAUGGUUUG | NO: 17 |
| | | CCUGCUUUCGCU | |
| PB1 D | 67 | AGUAGAAACAAGGCAUUUUUUCAUGAA | SEQ ID |
| | | GGACAAGCUAAAUCAUUCAAAUGGUUU | NO: 18 |
| | | GCCUGCUUUCGCU | |
| PB1 E | 62 | AGUAGAAACAAGGCAUUUUAAGUCGGA | SEQ ID |
| | | UUGACAUCCAUUCAAAUGGUUUGCCUG | NO: 19 |
| | | CUUUCGCU | |
| PB1 F | 64 | AGUAGAAACAAGGCAUUUUUUCAGUCG | SEQ ID |
| | | GAUUGACAUCCAUUCAAAUGGUUUGCC | NO: 20 |
| | | UGCUUUCGCU | |
| PB1 G | 52 | AGUAGAAACAAGGCAUUUUUUCAUGCA | SEQ ID |
| | | UUCAAAUGGUUUGCCUGCUUUCGCU | NO: 21 |
| PB1 H | 60 | AGUAGAAACAAGGCAUUUUUUCAUGAA | SEQ ID |
| | | GGACAAGCUAAAUUCAGUUUGCCUGCU | NO: 22 |
| | | UUCGCU | |
| PB1 I | 40 | AGUAGAAACAAGGCAUUUUUUCAGUUU | SEQ ID |
| | | GCCUGCUUUCGCU | NO: 23 |
| PB1 J | 80 | AGUAGAAACAAGGCAUUUUUUCAUGAA | SEQ ID |
| | | GGACAAGCUAAAUUCGGAUUGACAUCC | NO: 24 |
| | | AUUCAAAUGGUUUGCCUGCUUUCGCU | |

TABLE 3
Cloned mvRNAs based on t-loop analysis.

| | Sequence 5′ and 3′ | SEQ ID |
|---|---|---|
| Name | (vRNA) | NO. |
| PA66 | AGUAGAAACAAGGUACUUUU | SEQ ID |
| | UUGGACAGUAUGGAUAGCAC | NO: 25 |
| | AUUUUGAAUCAGUACCUGCU | |
| | UUCGCU | |
| PA60 | AGUAGAAACAAGGUACUUUU | SEQ ID |
| | UUGGACAGUAUGCCAUUUUG | NO: 26 |
| | AAUCAGUACCUGCUUUCGCU | |
| HA61 | AGUAGAAACAAGGGUGUUUU | SEQ ID |
| | UCCUUAUAUUUCUGAAAUCC | NO: 27 |
| | UAAUCUUCCCCUGCUUUUGC | |
| | U | |
| HA58 | AGUAGAAACAAGGGUGUUUU | SEQ ID |
| | UCCUUAUAUUUCUGAAAUCC | NO: 28 |
| | UAUUCCCCUGCUUUUGCU | |
| HA63 | AGUAGAAACAAGGGUGUUUU | SEQ ID |
| | UCCUUAUAUUUCUGAAAUGU | NO: 29 |
| | UUUUAUUUUCCCCUGCUUUU | |
| | GCU | |
| HA64 | AGUAGAAACAAGGGUGUUUU | SEQ ID |
| | UCCUUAUAUUUCUGAAAUCC | NO: 30 |
| | UAAUCUCAUUCCCCUGCUUU | |
| | UGCU | |

1. An engineered ribonucleic acid (RNA) sequence comprising a stem-loop, wherein the stem-loop comprises a stem portion with at least 5 base pairs (bps) in length and a loop portion with about 20 nucleotides in length, wherein the loop portion matches a footprint of an RNA polymerase of a target virus, and wherein the stem loop is flanked by a 5′ promoter and a 3′ promoter RNA sequence of the target virus.

2. The engineered RNA sequence of claim 1, wherein the stem portion comprises at least 14 bps in length.

3. The engineered RNA sequence of claim 1, wherein the target virus comprises a negative-sense RNA virus.

4. The engineered RNA sequence of claim 3, wherein the negative-sense RNA virus comprises Influenza A virus (IAV), Ebola virus, Nipah virus, Hanta virus, Hendra virus, Lassa virus, or Rabies virus.

5. The engineered RNA sequence of claim 1, wherein the RNA sequence comprises between about 40 to about 80 nucleotides.

6. The engineered RNA sequence of claim 1, wherein the RNA sequence comprises at least 60% sequence identity to any one of SEQ ID NO: 4-30.

7-9. (canceled)

10. The engineered RNA sequence of claim 1, wherein the RNA sequence comprises any one of sequences selected from SEQ ID NO: 4-30.

11. The engineered RNA sequence of claim 1, wherein the footprint of the RNA polymerase comprises an area within the RNA polymerase capable of holding a designated number of nucleotides.

12. The engineered RNA sequence of claim 11, wherein the designated number of nucleotides comprises about 20 nucleotides.

13. The engineered RNA sequence of claim 1, wherein the 5′ promoter comprises about 12 nucleotides in length.

14. The engineered RNA sequence of claim 1, wherein the 3′ promoter comprises about 13 nucleotides in length.

15-38. (canceled)

39. A method of treating or preventing a viral infection, the method comprising:
engineering an RNA sequence comprising a stem-loop, wherein the stem-loop comprises a stem portion with at least 5 base pairs (bps) in length and a loop portion with about 20 nucleotides in length, wherein the loop portion matches a footprint of an RNA polymerase of a target virus, and wherein the stem loop is flanked by a 5′ promoter and a 3′ promoter RNA sequence of the target virus; and
administering the RNA sequence to a subject, wherein the RNA sequence contacts the RNA polymerase of the target virus, and wherein the RNA sequence reduces viral replication and/or activates the innate immune response in the subject relative to an untreated control subject.

40-48. (canceled)

49. The method of claim 39, wherein the RNA sequence forms a template loop (t-loop) around the RNA polymerase to reduce viral replication.

50. The method of claim 39, wherein the RNA sequence comprises an agonist to activate the innate immune response.

51. The method of claim 39, wherein the innate immune response comprises binding a host pathogen receptor to the RNA sequence.

52. The method of claim 51, wherein the host pathogen receptor comprises a retinoic acid-inducible gene I (RIG-I).

53-58. (canceled)

59. A computer-implemented method for generating at least one optimal ribonucleic acid (RNA) sequence for reducing viral replication and/or inducing activation of an innate response to a target virus, the computer-implemented method comprising:
retrieving, by one or more processors, an RNA polymerase of the target virus;
performing, by the one or more processors, a template loop (t-loop) analysis operation on the RNA polymerase;
determining, by the one or more processors, at least one optimal RNA sequence corresponding with the RNA polymerase of the target virus based on results of the t-loop analysis operation; and
outputting, by the one or more processors, a ΔG, a location of the ΔG, a t-loop structure, or combinations thereof.

60. The computer-implemented method of claim 59, further comprising:
receiving, by the one or more processors, one or more user-defined parameters associated with the RNA polymerase of the target virus; and
performing, by the one or more processors, the t-loop analysis operation based at least on the one or more user-defined parameters.

61. The computer-implemented method of claim 59, wherein the t-loop analysis operation is used interchangeably with a sliding window operation.

62. The computer-implemented method of claim 59, further comprising:
determining, by the one or more processors, at least one criterion of a composition for administration to a subject based on the at least one optimal RNA sequence.


SEQUENCE LISTING (680 sequences)

SEQ ID NO: 1 - RNA, 47 nt, synthetic construct
  misc_feature 1..47: note = Description of sequence: mvRNA template: NP47
  source 1..47: mol_type = other RNA
agtagaaacaagggtatttttctttactagtctaccctgcttttgct

SEQ ID NO: 2 - RNA, 56 nt, synthetic construct
  misc_feature 1..56: note = Description of sequence: mvRNA template: NP56
  source 1..56: mol_type = other RNA
agtagaaacaagggtatttttctttctcgagcgtactagtctaccctgcttttgct

SEQ ID NO: 3 - RNA, 76 nt, synthetic construct
  misc_feature 1..76: note = Description of sequence: mvRNA template: NP76
  source 1..76: mol_type = other RNA
agtagaaacaagggtatttttctttactagtgatttcgatgtcactctgtgagtgattatctaccctgcttttgct

SEQ ID NO: 4 - RNA, 71 nt, synthetic construct
  misc_feature 1..71: note = Description of sequence: mvRNA template: NP71.1
  source 1..71: mol_type = other RNA
agtagaaacaagggtatttttctttactagttaggtagtatacctagtaactagtctaccctgcttttgct

SEQ ID NO: 5 - RNA, 71 nt, synthetic construct
  misc_feature 1..71: note = Description of sequence: mvRNA template: NP71.2
  source 1..71: mol_type = other RNA
agtagaaacaagggtatttttctttactagtccggttgttttggttgccactagtctaccctgcttttgct

SEQ ID NO: 6 - RNA, 71 nt, synthetic construct
  misc_feature 1..71: note = Description of sequence: mvRNA template: NP71.3
  source 1..71: mol_type = other RNA
agtagaaacaagggtatttttctttactagtccgcttgtatagcttgccactagtctaccctgcttttgct

SEQ ID NO: 7 - RNA, 71 nt, synthetic construct
  misc_feature 1..71: note = Description of sequence: mvRNA template: NP71.4
  source 1..71: mol_type = other RNA
agtagaaacaagggtatttttctttactagtccggccgatatggccgccactagtctaccctgcttttgct

SEQ ID NO: 8 - RNA, 71 nt, synthetic construct
  misc_feature 1..71: note = Description of sequence: mvRNA template: NP71.5
  source 1..71: mol_type = other RNA
agtagaaacaagggtatttttctttactagtccggccgccccggccgccactagtctaccctgcttttgct

SEQ ID NO: 9 - RNA, 71 nt, synthetic construct
  misc_feature 1..71: note = Description of sequence: mvRNA template: NP71.6
  source 1..71: mol_type = other RNA
agtagaaacaagggtatttttctttactagtccggccgttttggttgccactagtctaccctgcttttgct

SEQ ID NO: 10 - RNA, 71 nt, synthetic construct
  misc_feature 1..71: note = Description of sequence: mvRNA template: NP71.7
  source 1..71: mol_type = other RNA
agtagaaacaagggtatttttctttactagtccggttcttttggttgccactagtctaccctgcttttgct

SEQ ID NO: 11 - RNA, 71 nt, synthetic construct
  misc_feature 1..71: note = Description of sequence: mvRNA template: NP71.8
  source 1..71: mol_type = other RNA
agtagaaacaagggtatttttctttactagtccggttgctttggttgccactagtctaccctgcttttgct

SEQ ID NO: 12 - RNA, 71 nt, synthetic construct
  misc_feature 1..71: note = Description of sequence: mvRNA template: NP71.10
  source 1..71: mol_type = other RNA
agtagaaacaagggtatttttctttactagtggcagcaaaagcagggtaactagtctaccctgcttttgct

SEQ ID NO: 13 - RNA, 71 nt, synthetic construct
  misc_feature 1..71: note = Description of sequence: mvRNA template: NP71.11
  source 1..71: mol_type = other RNA
agtagaaacaagggtatttttctttactagtggcagcaaaagcacccatactagtctaccctgcttttgct

SEQ ID NO: 14 - RNA, 71 nt, synthetic construct
  misc_feature 1..71: note = Description of sequence: mvRNA template: NP71.12
  source 1..71: mol_type = other RNA
agtagaaacaagggtatttttctttactagtggctctaaaagcacccatactagtctaccctgcttttgct

SEQ ID NO: 15 - RNA, 57 nt, synthetic construct
  misc_feature 1..57: note = Description of sequence: mvRNA template: PB1 A
  source 1..57: mol_type = other RNA
agtagaaacaaggcattttttcatgaaatccattcaaatggtttgcctgctttcgct

SEQ ID NO: 16 - RNA, 57 nt, synthetic construct
  misc_feature 1..57: note = Description of sequence: mvRNA template: PB1 B
  source 1..57: mol_type = other RNA
agtagaaacaaggcattttttcatgaaggacattcaaatggtttgcctgctttcgct

SEQ ID NO: 17 - RNA, 66 nt, synthetic construct
  misc_feature 1..66: note = Description of sequence: mvRNA template: PB1 C
  source 1..66: mol_type = other RNA
agtagaaacaaggcattttttcatgaaggacaagctaaacattcaaatggtttgcctgctttcgct

SEQ ID NO: 18 - RNA, 67 nt, synthetic construct
  misc_feature 1..67: note = Description of sequence: mvRNA template: PB1 D
  source 1..67: mol_type = other RNA
agtagaaacaaggcattttttcatgaaggacaagctaaatcattcaaatggtttgcctgctttcgct

SEQ ID NO: 19 - RNA, 62 nt, synthetic construct
  misc_feature 1..62: note = Description of sequence: mvRNA template: PB1 E
  source 1..62: mol_type = other RNA
agtagaaacaaggcattttaagtcggattgacatccattcaaatggtttgcctgctttcgct

SEQ ID NO: 20 - RNA, 64 nt, synthetic construct
  misc_feature 1..64: note = Description of sequence: mvRNA template: PB1 F
  source 1..64: mol_type = other RNA
agtagaaacaaggcattttttcagtcggattgacatccattcaaatggtttgcctgctttcgct

SEQ ID NO: 21 - RNA, 52 nt, synthetic construct
  misc_feature 1..52: note = Description of sequence: mvRNA template: PB1 G
  source 1..52: mol_type = other RNA
agtagaaacaaggcattttttcatgcattcaaatggtttgcctgctttcgct

SEQ ID NO: 22 - RNA, 60 nt, synthetic construct
  misc_feature 1..60: note = Description of sequence: mvRNA template: PB1 H
  source 1..60: mol_type = other RNA
agtagaaacaaggcattttttcatgaaggacaagctaaattcagtttgcctgctttcgct

SEQ ID NO: 23 - RNA, 40 nt, synthetic construct
  misc_feature 1..40: note = Description of sequence: mvRNA template: PB1 I
  source 1..40: mol_type = other RNA
agtagaaacaaggcattttttcagtttgcctgctttcgct

SEQ ID NO: 24 - RNA, 80 nt, synthetic construct
  misc_feature 1..80: note = Description of sequence: mvRNA template: PB1 J
  source 1..80: mol_type = other RNA
agtagaaacaaggcattttttcatgaaggacaagctaaattcggattgacatccattcaaatggtttgcctgctttcgct

SEQ ID NO: 25 - RNA, 66 nt, synthetic construct
  misc_feature 1..66: note = Description of sequence: mvRNA template: PA66
  source 1..66: mol_type = other RNA
agtagaaacaaggtacttttttggacagtatggatagcacattttgaatcagtacctgctttcgct

SEQ ID NO: 26 - RNA, 60 nt, synthetic construct
  misc_feature 1..60: note = Description of sequence: mvRNA template: PA60
  source 1..60: mol_type = other RNA
agtagaaacaaggtacttttttggacagtatgccattttgaatcagtacctgctttcgct

SEQ ID NO: 27 - RNA, 61 nt, synthetic construct
  misc_feature 1..61: note = Description of sequence: mvRNA template: HA61
  source 1..61: mol_type = other RNA
agtagaaacaagggtgtttttccttatatttctgaaatcctaatcttcccctgcttttgct

SEQ ID NO: 28 - RNA, 58 nt, synthetic construct
  misc_feature 1..58: note = Description of sequence: mvRNA template: HA58
  source 1..58: mol_type = other RNA
agtagaaacaagggtgtttttccttatatttctgaaatcctattcccctgcttttgct

SEQ ID NO: 29 - RNA, 63 nt, synthetic construct
  misc_feature 1..63: note = Description of sequence: mvRNA template: HA63
  source 1..63: mol_type = other RNA
agtagaaacaagggtgtttttccttatatttctgaaatgtttttattttcccctgcttttgct

SEQ ID NO: 30 - RNA, 64 nt, synthetic construct
  misc_feature 1..64: note = Description of sequence: mvRNA template: HA64
  source 1..64: mol_type = other RNA
agtagaaacaagggtgtttttccttatatttctgaaatcctaatctcattcccctgcttttgct

SEQ ID NO: 31 - RNA, 61 nt, Influenza A virus
  misc_feature 1..61: note = Description of sequence: mvRNA template: WSN_NP_61
  source 1..61: mol_type = other RNA
agcaaaagcagggtagataatcactcacagagtgacatagaaaaatacccttgtttctact

SEQ ID NO: 32 - RNA, 57 nt, Influenza A virus
  misc_feature 1..57: note = Description of sequence: mvRNA template: WSN_PB1_57
  source 1..57: mol_type = other RNA
agcgaaagcaggcaaaccatttgaatggatttcatgaaaaaatgccttgtttctact

SEQ ID NO: 33 - RNA, 57 nt, Influenza A virus
  misc_feature 1..57: note = Description of sequence: mvRNA template: WSN_PB1_57
  source 1..57: mol_type = other RNA
agcgaaagcaggcaaaccatttgaatgtccttcatgaaaaaatgccttgtttctact

SEQ ID NO: 34 - RNA, 66 nt, Influenza A virus
  misc_feature 1..66: note = Description of sequence: mvRNA template: WSN_PB1_66
  source 1..66: mol_type = other RNA
agcgaaagcaggcaaaccatttgaatgtttagcttgtccttcatgaaaaaatgccttgtttctact

SEQ ID NO: 35 - RNA, 65 nt, Influenza A virus
  misc_feature 1..65: note = Description of sequence: mvRNA template: WSN_NP_65
  source 1..65: mol_type = other RNA
agcaaaagcagggtagataatcactcacagagtgacatctaaagaaaaatacccttgtttctact

SEQ ID NO: 36 - RNA, 44 nt, Influenza A virus
  misc_feature 1..44: note = Description of sequence: mvRNA template: WSN_HA_44
  source 1..44: mol_type = other RNA
agcaaaagcaggggaatataaggaaaaacacccttgtttctact

SEQ ID NO: 37 - RNA, 70 nt, Influenza A virus
  misc_feature 1..70: note = Description of sequence: mvRNA template: WSN_NA_70
  source 1..70: mol_type = other RNA
agcaaaagcaggagtttaaatgaatccaaacctgacaagtagtttgttcaaaaaactccttgtttctact

SEQ ID NO: 38 - RNA, 62 nt, Influenza A virus
  misc_feature 1..62: note = Description of sequence: mvRNA template: WSN_PB1_62
  source 1..62: mol_type = other RNA
agcgaaagcaggcaaaccatttgaatagcttgtccttcatgaaaaaatgccttgtttctact

SEQ ID NO: 39 - RNA, 70 nt, Influenza A virus
  misc_feature 1..70: note = Description of sequence: mvRNA template: WSN_NP_70
  source 1..70: mol_type = other RNA
agcaaaagcagggtagataatcactcacagagtgacatcgaaattaaagaaaaatacccttgtttctact

SEQ ID NO: 40 - RNA, 64 nt, Influenza A virus
  misc_feature 1..64: note = Description of sequence: mvRNA template: WSN_PB1_64
  source 1..64: mol_type = other RNA
agcgaaagcaggcaaaccatttgaatggatgtcaatccgacttaaaaaatgccttgtttctact

SEQ ID NO: 41 - RNA, 69 nt, Influenza A virus
  misc_feature 1..69: note = Description of sequence: mvRNA template: WSN_PB1_69
  source 1..69: mol_type = other RNA
agcgaaagcaggcaaaccatttgaatggatgttagcttgtccttcatgaaaaaatgccttgtttctact

SEQ ID NO: 42 - RNA, 69 nt, Influenza A virus
  misc_feature 1..69: note = Description of sequence: mvRNA template: WSN_NA_69
  source 1..69: mol_type = other RNA
agcaaaagcaggagtttaaatgaatccaaaccagaaagtagtttgttcaaaaaactccttgtttctact

SEQ ID NO: 43 - RNA, 62 nt, Influenza A virus
  misc_feature 1..62: note = Description of sequence: mvRNA template: WSN_PB1_62
  source 1..62: mol_type = other RNA
agcgaaagcaggcaaaccatttgaatggatgtcaatccgacttaaaatgccttgtttctact

SEQ ID NO: 44 - RNA, 68 nt, Influenza A virus
  misc_feature 1..68: note = Description of sequence: mvRNA template: WSN_PB1_68
  source 1..68: mol_type = other RNA
agcgaaagcaggcaaaccatttgaatggatgtcaatccgacttcatgaaaaaatgccttgtttctact

SEQ ID NO: 45 - RNA, 60 nt, Influenza A virus
  misc_feature 1..60: note = Description of sequence: mvRNA template: WSN_HA_60
  source 1..60: mol_type = other RNA
agcaaaagcaggggaaattaggatttcagaaatataaggaaaaacacccttgtttctact

SEQ ID NO: 46 - RNA, 68 nt, Influenza A virus
  misc_feature 1..68: note = Description of sequence: mvRNA template: WSN_PB1_68
  source 1..68: mol_type = other RNA
agcgaaagcaggcaaaccatttgaatggatgtcaatcctccttcatgaaaaaatgccttgtttctact

SEQ ID NO: 47 - RNA, 49 nt, Influenza A virus
  misc_feature 1..49: note = Description of sequence: mvRNA template: WSN_PB1_49
  source 1..49: mol_type = other RNA
agcgaaagcaggcaaaccatttgaattgaaaaaatgccttgtttctact

SEQ ID NO: 48 - RNA, 56 nt, Influenza A virus
  misc_feature 1..56: note = Description of sequence: mvRNA template: WSN_PB1_56
  source 1..56: mol_type = other RNA
agcgaaagcaggcaaactttagcttgtccttcatgaaaaaatgccttgtttctact

SEQ ID NO: 49 - RNA, 60 nt, Influenza A virus
  misc_feature 1..60: note = Description of sequence: mvRNA template: WSN_PB2_60
  source 1..60: mol_type = other RNA
agcgaaagcaggtcaattatattcaatatcgaatagtttaaaaacgaccttgtttctact

SEQ ID NO: 50 - RNA, 60 nt, Influenza A virus
  misc_feature 1..60: note = Description of sequence: mvRNA template: WSN_PB1_60
  source 1..60: mol_type = other RNA
agcgaaagcaggcaaaccatttgaatggatgtcaatatgaaaaaatgccttgtttctact

SEQ ID NO: 51 - RNA, 74 nt, Influenza A virus
  misc_feature 1..74: note = Description of sequence: mvRNA template: WSN_NP_74
  source 1..74: mol_type = other RNA
agcaaaagcagggtagataatcactcacagagtgacatctacgacaattaaagaaaaatacccttgtttctact

SEQ ID NO: 52 - RNA, 73 nt, Influenza A virus
  misc_feature 1..73: note = Description of sequence: mvRNA template: WSN_NA_73
  source 1..73: mol_type = other RNA
agcaaaagcaggagtttaaatgaatccaaaccagaaaacaagtagtttgttcaaaaaactccttgtttctact

SEQ ID NO: 53 - RNA, 62 nt, Influenza A virus
  misc_feature 1..62: note = Description of sequence: mvRNA template: WSN_NA_62
  source 1..62: mol_type = other RNA
agcaaaagcaggagtttaaatgaatccaaactagtttgttcaaaaaactccttgtttctact

SEQ ID NO: 54 - RNA, 63 nt, Influenza A virus
  misc_feature 1..63: note = Description of sequence: mvRNA template: WSN_NA_63
  source 1..63: mol_type = other RNA
agcaaaagcaggagtttaacaccattgacaagtagtttgttcaaaaaactccttgtttctact

SEQ ID NO: 55 - RNA, 55 nt, Influenza A virus
  misc_feature 1..55: note = Description of sequence: mvRNA template: WSN_PB2_55
  source 1..55: mol_type = other RNA
agcgaaagcaggtcaattatattccgaatagtttaaaaacgaccttgtttctact

SEQ ID NO: 56 - RNA, 44 nt, Influenza A virus
  misc_feature 1..44: note = Description of sequence: mvRNA template: WSN_PB1_44
  source 1..44: mol_type = other RNA
agcgaaagcaggcaaaccatatgaaaaaatgccttgtttctact

SEQ ID NO: 57 - RNA, 64 nt, Influenza A virus
  misc_feature 1..64: note = Description of sequence: mvRNA template: WSN_PA_64
  source 1..64: mol_type = other RNA
agcgaaagcaggtactgattcaaattgctatccatactgtccaaaaaagtaccttgtttctact

SEQ ID NO: 58 - RNA, 70 nt, Influenza A virus
  misc_feature 1..70: note = Description of sequence: mvRNA template: WSN_NP_70
  source 1..70: mol_type = other RNA
agcaaaagcagggtagataatcactcacagagtgacatcgaaatcatagaaaaatacccttgtttctact

SEQ ID NO: 59 - RNA, 46 nt, Influenza A virus
  misc_feature 1..46: note = Description of sequence: mvRNA template: WSN_HA_46
  source 1..46: mol_type = other RNA
agcaaaagcaggggaaaataaaaagaaaaacacccttgtttctact

SEQ ID NO: 60 - RNA, 61 nt, Influenza A virus
  misc_feature 1..61: note = Description of sequence: mvRNA template: WSN_HA_61
  source 1..61: mol_type = other RNA
agcaaaagcaggggaaaataaagatttcagaaatataaggaaaaacacccttgtttctact

SEQ ID NO: 61 - RNA, 65 nt, Influenza A virus
  misc_feature 1..65: note = Description of sequence: mvRNA template: WSN_PB1_65
  source 1..65: mol_type = other RNA
agcgaaagcaggcaaaccatttgaatggatgtcaatccgacttgaaaaaatgccttgtttctact

SEQ ID NO: 62 - RNA, 61 nt, Influenza A virus
  misc_feature 1..61: note = Description of sequence: mvRNA template: WSN_PB1_61
  source 1..61: mol_type = other RNA
agcgaaagcaggcaaaccatttgaatggttgtccttcatgaaaaaatgccttgtttctact

SEQ ID NO: 63 - RNA, 67 nt, Influenza A virus
  misc_feature 1..67: note = Description of sequence: mvRNA template: WSN_PB1_67
  source 1..67: mol_type = other RNA
agcgaaagcaggcaaaccatttgaatgatttagcttgtccttcatgaaaaaatgccttgtttctact

SEQ ID NO: 64 - RNA, 60 nt, Influenza A virus
  misc_feature 1..60: note = Description of sequence: mvRNA template: WSN_PB1_60
  source 1..60: mol_type = other RNA
agcgaaagcaggcaaaccatttgtagcttgtccttcatgaaaaaatgccttgtttctact

SEQ ID NO: 65 - RNA, 64 nt, Influenza A virus
  misc_feature 1..64: note = Description of sequence: mvRNA template: WSN_PB1_64
  source 1..64: mol_type = other RNA
agcgaaagcaggcaaaccatgtgaatttagcttgtccttcatgaaaaaatgccttgtttctact

SEQ ID NO: 66 - RNA, 66 nt, Influenza A virus
  misc_feature 1..66: note = Description of sequence: mvRNA template: WSN_PA_66
  source 1..66: mol_type = other RNA
agcgaaagcaggtactgatttactatttgctatccatactgtccaaaaaagtaccttgtttctact

SEQ ID NO: 67 - RNA, 69 nt, Influenza A virus
  misc_feature 1..69: note = Description of sequence: mvRNA template: WSN_NP_69
  source 1..69: mol_type = other RNA
agcaaaagcagggtagataatcactcacagagtgacatcgaaatcatggcaaatacccttgtttctact

SEQ ID NO: 68 - RNA, 70 nt, Influenza A virus
  misc_feature 1..70: note = Description of sequence: mvRNA template: WSN_NP_70
  source 1..70: mol_type = other RNA
agcaaaagcagggtagataatcactcacagagtgacatcgaaatcatggcaaaatacccttgtttctact

SEQ ID NO: 69 - RNA, 66 nt, Influenza A virus
  misc_feature 1..66: note = Description of sequence: mvRNA template: WSN_NP_66
  source 1..66: mol_type = other RNA
agcaaaagcagggtagataatcactcacagagtgacatcttaaagaaaaatacccttgtttctact

SEQ ID NO: 70 - RNA, 64 nt, Influenza A virus
  misc_feature 1..64: note = Description of sequence: mvRNA template: WSN_NP_64
  source 1..64: mol_type = other RNA
agcaaaagcagggtagataatcactcacagagtgacattaaagaaaaatacccttgtttctact

SEQ ID NO: 71 - RNA, 57 nt, Influenza A virus
  misc_feature 1..57: note = Description of sequence: mvRNA template: WSN_NP_57
  source 1..57: mol_type = other RNA
agcaaaagcagggtagataatcactcacagagtgacaaaaatacccttgtttctact

SEQ ID NO: 72 - RNA, 59 nt, Influenza A virus
  misc_feature 1..59: note = Description of sequence: mvRNA template: WSN_NP_59
  source 1..59: mol_type = other RNA
agcaaaagcagggtagataatcactcacagagttaaagaaaaatacccttgtttctact

SEQ ID NO: 73 - RNA, 55 nt, Influenza A virus
  misc_feature 1..55: note = Description of sequence: mvRNA template: WSN_NP_55
  source 1..55: mol_type = other RNA
agcaaaagcagggtagataatcactcacagagagaaaaatacccttgtttctact

SEQ ID NO: 74 - RNA, 64 nt, Influenza A virus
  misc_feature 1..64: note = Description of sequence: mvRNA template: WSN_NA_64
  source 1..64: mol_type = other RNA
agcaaaagcaggagtttaaatgaatccaaaccatagtttgttcaaaaaactccttgtttctact

SEQ ID NO: 75 - RNA, 71 nt, Influenza A virus
  misc_feature 1..71: note = Description of sequence: mvRNA template: WSN_NA_71
  source 1..71: mol_type = other RNA
agcaaaagcaggagtttaaatgaatccaaccattgacaagtagtttgttcaaaaaactccttgtttctact

SEQ ID NO: 76 - RNA, 64 nt, Influenza A virus
  misc_feature 1..64: note = Description of sequence: mvRNA template: WSN_HA_64
  source 1..64: mol_type = other RNA
agcaaaagcaggggaatgagattaggatttcagaaatataaggaaaaacacccttgtttctact

SEQ ID NO: 77 - RNA, 62 nt, Influenza A virus
  misc_feature 1..62: note = Description of sequence: mvRNA template: WSN_PB2_62
  source 1..62: mol_type = other RNA
agcgaaagcaggtcaattatattcaatatggagaatagtttaaaaacgaccttgtttctact

SEQ ID NO: 78 - RNA, 57 nt, Influenza A virus
  misc_feature 1..57: note = Description of sequence: mvRNA template: WSN_PB2_57
  source 1..57: mol_type = other RNA
agcgaaagcaggtcaattatattcaatatgtagtttaaaaacgaccttgtttctact

SEQ ID NO: 79 - RNA, 48 nt, Influenza A virus
  misc_feature 1..48: note = Description of sequence: mvRNA template: WSN_PB2_48
  source 1..48: mol_type = other RNA
agcgaaagcaggtcaattatattcattaaaaacgaccttgtttctact

SEQ ID NO: 80 - RNA, 54 nt, Influenza A virus
  misc_feature 1..54: note = Description of sequence: mvRNA template: WSN_PB2_54
  source 1..54: mol_type = other RNA
agcgaaagcaggtcaattatagtcgaatagtttaaaaacgaccttgtttctact

SEQ ID NO: 81 - RNA, 49 nt, Influenza A virus
  misc_feature 1..49: note = Description of sequence: mvRNA template: WSN_PB2_49
  source 1..49: mol_type = other RNA
agcgaaagcaggtcaattagaatagtttaaaaacgaccttgtttctact

SEQ ID NO: 82 - RNA, 58 nt, Influenza A virus
  misc_feature 1..58: note = Description of sequence: mvRNA template: WSN_PB2_58
  source 1..58: mol_type = other RNA
agcgaaagcaggtcaatcaattagtgtcgaatagtttaaaaacgaccttgtttctact

SEQ ID NO: 83 - RNA, 68 nt, Influenza A virus
  misc_feature 1..68: note = Description of sequence: mvRNA template: WSN_PB1_68
  source 1..68: mol_type = other RNA
agcgaaagcaggcaaaccatttgaatggatgtcaatccgactttacgaaaaaatgccttgtttctact

SEQ ID NO: 84 - RNA, 73 nt, Influenza A virus
  misc_feature 1..73: note = Description of sequence: mvRNA template: WSN_PB1_73
  source 1..73: mol_type = other RNA
agcgaaagcaggcaaaccatttgaatggatgtcaatccgactttccttcatgaaaaaatgccttgtttctact

SEQ ID NO: 85 - RNA, 61 nt, Influenza A virus
  misc_feature 1..61: note = Description of sequence: mvRNA template: WSN_PB1_61
  source 1..61: mol_type = other RNA
agcgaaagcaggcaaaccatttgaatggatgtcaatccgacttaaatgccttgtttctact

SEQ ID NO: 86 - RNA, 63 nt, Influenza A virus
  misc_feature 1..63: note = Description of sequence: mvRNA template: WSN_PB1_63
  source 1..63: mol_type = other RNA
agcgaaagcaggcaaaccatttgaatggatgtcaatccgacttaaaaatgccttgtttctact

SEQ ID NO: 87 - RNA, 80 nt, Influenza A virus
  misc_feature 1..80: note = Description of sequence: mvRNA template: WSN_PB1_80
  source 1..80: mol_type = other RNA
agcgaaagcaggcaaaccatttgaatggatgtcaatccgaatttagcttgtccttcatgaaaaaatgccttgtttctact

SEQ ID NO: 88 - RNA, 47 nt, Influenza A virus
  misc_feature 1..47: note = Description of sequence: mvRNA template: WSN_PB1_47
  source 1..47: mol_type = other RNA
agcgaaagcaggcaaaccatttgaatgaaaaatgccttgtttctact

SEQ ID NO: 89 - RNA, 48 nt, Influenza A virus
  misc_feature 1..48: note = Description of sequence: mvRNA template: WSN_PB1_48
  source 1..48: mol_type = other RNA
agcgaaagcaggcaaaccatttgaatgaaaaaatgccttgtttctact

SEQ ID NO: 90 - RNA, 64 nt, Influenza A virus
  misc_feature 1..64: note = Description of sequence: mvRNA template: WSN_PB1_64
  source 1..64: mol_type = other RNA
agcgaaagcaggcaaaccatttgaatgtagcttgtccttcatgaaaaaatgccttgtttctact

SEQ ID NO: 91 - RNA, 62 nt, Influenza A virus
  misc_feature 1..62: note = Description of sequence: mvRNA template: WSN_PB1_62
  source 1..62: mol_type = other RNA
agcgaaagcaggcaaaccatttgattagcttgtccttcatgaaaaaatgccttgtttctact

SEQ ID NO: 92 - RNA, 43 nt, Influenza A virus
  misc_feature 1..43: note = Description of sequence: mvRNA template: WSN_PB1_43
  source 1..43: mol_type = other RNA
agcgaaagcaggcaaaccatttaaaaaatgccttgtttctact

SEQ ID NO: 93 - RNA, 40 nt, Influenza A virus
  misc_feature 1..40: note = Description of sequence: mvRNA template: WSN_PB1_40
  source 1..40: mol_type = other RNA
agcgaaagcaggcaaactgaaaaaatgccttgtttctact

SEQ ID NO: 94 - RNA, 50 nt, Influenza A virus
  misc_feature 1..50: note = Description of sequence: mvRNA template: WSN_PB1_50
  source 1..50: mol_type = other RNA
agcgaaagcaggcaaacttgtccttcatgaaaaaatgccttgtttctact

SEQ ID NO: 95 - RNA, 47 nt, Influenza A virus
  misc_feature 1..47: note = Description of sequence: mvRNA template: WSN_NS_47
  source 1..47: mol_type = other RNA
agcaaaagcagggtgacaaagaaataaaaaacacccttgtttctact

SEQ ID NO: 96 - RNA, 72 nt, Influenza A virus
  misc_feature 1..72: note = Description of sequence: mvRNA template: WSN_NP_72
  source 1..72: mol_type = other RNA
agcaaaagcagggtagataatcactcacagagtgacatcgaaatcatggagaaaaatacccttgtttctact

SEQ ID NO: 97 - RNA, 88 nt, Influenza A virus
  misc_feature 1..88: note = Description of sequence: mvRNA template: WSN_NP_88
  source 1..88: mol_type = other RNA
agcaaaagcagggtagataatcactcacagagtgacatcgaaatcatggggagtacgacaattaaagaaaaatacccttgtttctact

SEQ ID NO: 98 - RNA, 91 nt, Influenza A virus
  misc_feature 1..91: note = Description of sequence: mvRNA template: WSN_NP_91
  source 1..91: mol_type = other RNA
agcaaaagcagggtagataatcactcacagagtgacatcgaaataatgcagaggagtacgacaattaaagaaaaatacccttgtttctact

SEQ ID NO: 99 - RNA, 81 nt, Influenza A virus
  misc_feature 1..81: note = Description of sequence: mvRNA template: WSN_NP_81
  source 1..81: mol_type = other RNA
agcaaaagcagggtagataatcactcacagagtgacatcgaaagagtacgacaattaaagaaaaatacccttgtttctact

SEQ ID NO: 100 - RNA, 62 nt, Influenza A virus
  misc_feature 1..62: note = Description of sequence: mvRNA template: WSN_NP_62
  source 1..62: mol_type = other RNA
agcaaaagcagggtagataatcactcacagagtgacatcggaaaaatacccttgtttctact

SEQ ID NO: 101 - RNA, 57 nt, Influenza A virus
  misc_feature 1..57: note = Description of sequence: mvRNA template: WSN_NP_57
  source 1..57: mol_type = other RNA
agcaaaagcagggtagataatcactcacagagtgacatcaatacccttgtttctact

SEQ ID NO: 102 - RNA, 58 nt, Influenza A virus
  misc_feature 1..58: note = Description of sequence: mvRNA template: WSN_NP_58
  source 1..58: mol_type = other RNA
agcaaaagcagggtagataatcactcacagagtgacatcaaatacccttgtttctact

SEQ ID NO: 103 - RNA, 81 nt, Influenza A virus
  misc_feature 1..81: note = Description of sequence: mvRNA template: WSN_NP_81
  source 1..81: mol_type = other RNA
agcaaaagcagggtagataatcactcacagagtgaatgcagaggagtacgacaattaaagaaaaatacccttgtttctact

SEQ ID NO: 104 - RNA, 62 nt, Influenza A virus
  misc_feature 1..62: note = Description of sequence: mvRNA template: WSN_NP_62
  source 1..62: mol_type = other RNA
agcaaaagcagggtagataatcactcatacgacaattaaagaaaaatacccttgtttctact

SEQ ID NO: 105 - RNA, 49 nt, Influenza A virus
  misc_feature 1..49: note = Description of sequence: mvRNA template: WSN_NP_49
  source 1..49: mol_type = other RNA
agcaaaagcagggtagagacaattaaagaaaaatacccttgtttctact

SEQ ID NO: 106 - RNA, 72 nt, Influenza A virus
  misc_feature 1..72: note = Description of sequence: mvRNA template: WSN_NA_72
  source 1..72: mol_type = other RNA
agcaaaagcaggagtttaaatgaatccaaaccagaaaaaagtagtttgttcaaaaaactccttgtttctact

SEQ ID NO: 107 - RNA, 70 nt, Influenza A virus
  misc_feature 1..70: note = Description of sequence: mvRNA template: WSN_NA_70
  source 1..70: mol_type = other RNA
agcaaaagcaggagtttaaatgaatccaaaccagacaagtagtttgttcaaaaaactccttgtttctact

SEQ ID NO: 108 - RNA, 69 nt, Influenza A virus
  misc_feature 1..69: note = Description of sequence: mvRNA template: WSN_NA_69
  source 1..69: mol_type = other RNA
agcaaaagcaggagtttaaatgaatccaaattgacaagtagtttgttcaaaaaactccttgtttctact

SEQ ID NO: 109 - RNA, 45 nt, Influenza A virus
  misc_feature 1..45: note = Description of sequence: mvRNA template: WSN_NA_45
  source 1..45: mol_type = other RNA
agcaaaagcaggagtttaaatttcaaaaaactccttgtttctact

SEQ ID NO: 110 - RNA, 52 nt, Influenza A virus
  misc_feature 1..52: note = Description of sequence: mvRNA template: WSN_NA_52
  source 1..52: mol_type = other RNA
agcaaaagcaggagtttaaattagtttgttcaaaaaactccttgtttctact

SEQ ID NO: 111 - RNA, 62 nt, Influenza A virus
  misc_feature 1..62: note = Description of sequence: mvRNA template: WSN_NA_62
  source 1..62: mol_type = other RNA
agcaaaagcaggagtttaaaccattgacaagtagtttgttcaaaaaactccttgtttctact

SEQ ID NO: 112 - RNA, 70 nt, Influenza A virus
  misc_feature 1..70: note = Description of sequence: mvRNA template: WSN_M_70
  source 1..70: mol_type = other RNA
agcgaaagcaggtagatattgaaagatgagtcttctaaccgaggtcgtaaaaaactaccttgtttctact

SEQ ID NO: 113 - RNA, 47 nt, Influenza A virus
  misc_feature 1..47: note = Description of sequence: mvRNA template: WSN_M_47
  source 1..47: mol_type = other RNA
agcgaaagcaggtagatattgaaagataaaactaccttgtttctact

SEQ ID NO: 114 - RNA, 52 nt, Influenza A virus
  misc_feature 1..52: note = Description of sequence: mvRNA template: WSN_HA_52
  source 1..52: mol_type = other RNA
agcaaaagcaggggaaaataaaaacaaccagaaaaacacccttgtttctact

SEQ ID NO: 115 - RNA, 49 nt, Influenza A virus
  misc_feature 1..49: note = Description of sequence: mvRNA template: WSN_HA_49
  source 1..49: mol_type = other RNA
agcaaaagcaggggaaaataaaaacaagaaaaacacccttgtttctact

SEQ ID NO: 116 - RNA, 48 nt, Influenza A virus
  misc_feature 1..48: note = Description of sequence: mvRNA template: WSN_HA_48
  source 1..48: mol_type = other RNA
agcaaaagcaggggaaaataaaaacagaaaaacacccttgtttctact

SEQ ID NO: 117 - RNA, 63 nt, Influenza A virus
  misc_feature 1..63: note = Description of sequence: mvRNA template: WSN_HA_63
  source 1..63: mol_type = other RNA
agcaaaagcaggggaaaataaaaacatttcagaaatataaggaaaaacacccttgtttctact

SEQ ID NO: 118 - RNA, 67 nt, Influenza A virus
  misc_feature 1..67: note = Description of sequence: mvRNA template: WSN_HA_67
  source 1..67: mol_type = other RNA
agcaaaagcaggggaaaataaagattaggatttcagaaatataaggaaaaacacccttgtttctact

SEQ ID NO: 119 - RNA, 61 nt, Influenza A virus
  misc_feature 1..61: note = Description of sequence: mvRNA template: WSN_HA_61
  source 1..61: mol_type = other RNA
agcaaaagcaggggaaaattaggatttcagaaatataaggaaaaacacccttgtttctact

SEQ ID NO: 120 - RNA, 58 nt, Influenza A virus
  misc_feature 1..58: note = Description of sequence: mvRNA template: WSN_HA_58
  source 1..58: mol_type = other RNA
agcaaaagcaggggaataggatttcagaaatataaggaaaaacacccttgtttctact

SEQ ID NO: 121 - RNA, 57 nt, Influenza A virus
  misc_feature 1..57: note = Description of sequence: mvRNA template: WSN_PB2_57
  source 1..57: mol_type = other RNA
agcgaaagcaggtcaattatattcaatatggaaagaaaaaacgaccttgtttctact

SEQ ID NO: 122 - RNA, 69 nt, Influenza A virus
  misc_feature 1..69: note = Description of sequence: mvRNA template: WSN_PB2_69
  source 1..69: mol_type = other RNA
agcgaaagcaggtcaattatattcaatatggaaagagtcgaatagtttaaaaacgaccttgtttctact

SEQ ID NO: 123 - RNA, 67 nt, Influenza A virus
  misc_feature 1..67: note = Description of sequence: mvRNA template: WSN_PB2_67
  source 1..67: mol_type = other RNA
agcgaaagcaggtcaattatattcaatatgtagtgtcgaatagtttaaaaacgaccttgtttctact

SEQ ID NO: 124 - RNA, 51 nt, Influenza A virus
  misc_feature 1..51: note = Description of sequence: mvRNA template: WSN_PB2_51
  source 1..51: mol_type = other RNA
agcgaaagcaggtcaattatattcaatattaaaaacgaccttgtttctact

SEQ ID NO: 125 - RNA, 65 nt, Influenza A virus
  misc_feature 1..65: note = Description of sequence: mvRNA template: WSN_PB2_65
  source 1..65: mol_type = other RNA
agcgaaagcaggtcaattatattcaatatagtgtcgaatagtttaaaaacgaccttgtttctact

SEQ ID NO: 126 - RNA, 59 nt, Influenza A virus
  misc_feature 1..59: note = Description of sequence: mvRNA template: WSN_PB2_59
  source 1..59: mol_type = other RNA
agcgaaagcaggtcaattatattcaatacgaatagtttaaaaacgaccttgtttctact

SEQ ID NO: 127 - RNA, 62 nt, Influenza A virus
  misc_feature 1..62: note = Description of sequence: mvRNA template: WSN_PB2_62
  source 1..62: mol_type = other RNA
agcgaaagcaggtcaattatattcaatgtgtcgaatagtttaaaaacgaccttgtttctact

SEQ ID NO: 128 - RNA, 52 nt, Influenza A virus
  misc_feature 1..52: note = Description of sequence: mvRNA template: WSN_PB2_52
  source 1..52: mol_type = other RNA
agcgaaagcaggtcaattatattcatagtttaaaaacgaccttgtttctact

SEQ ID NO: 129 - RNA, 63 nt, Influenza A virus
  misc_feature 1..63: note = Description of sequence: mvRNA template: WSN_PB2_63
  source 1..63: mol_type = other RNA
agcgaaagcaggtcaattatattcattagtgtcgaatagtttaaaaacgaccttgtttctact

SEQ ID NO: 130 - RNA, 44 nt, Influenza A virus
  misc_feature 1..44: note = Description of sequence: mvRNA template: WSN_PB2_44
  source 1..44: mol_type = other RNA
agcgaaagcaggtcaattatattaaaaacgaccttgtttctact

SEQ ID NO: 131 - RNA, 60 nt, Influenza A virus
  misc_feature 1..60: note = Description of sequence: mvRNA template: WSN_PB2_60
  source 1..60: mol_type = other RNA
agcgaaagcaggtcaattatatttagtgtcgaatagtttaaaaacgaccttgtttctact

SEQ ID NO: 132 - RNA, 46 nt, Influenza A virus
  misc_feature 1..46: note = Description of sequence: mvRNA template: WSN_PB2_46
  source 1..46: mol_type = other RNA
agcgaaagcaggtcaataatagtttaaaaacgaccttgtttctact

SEQ ID NO: 133 - RNA, 51 nt, Influenza A virus
  misc_feature 1..51: note = Description of sequence: mvRNA template: WSN_PB2_51
  source 1..51: mol_type = other RNA
agcgaaagcaggtcaagtgtcgaatagtttaaaaacgaccttgtttctact

SEQ ID NO: 134 - RNA, 73 nt, Influenza A virus
  misc_feature 1..73: note = Description of sequence: mvRNA template: WSN_PB1_73
  source 1..73: mol_type = other RNA
agcgaaagcaggcaaaccatttgaatggatgtcaatccgactttacttttctaaaaaatgccttgtttctact

SEQ ID NO: 135 - RNA, 69 nt, Influenza A virus
  misc_feature 1..69: note = Description of sequence: mvRNA template: WSN_PB1_69
  source 1..69: mol_type = other RNA
agcgaaagcaggcaaaccatttgaatggatgtcaatccgactttacttttaaaatgccttgtttctact

SEQ ID NO: 136 - RNA, 75 nt, Influenza A virus
  misc_feature 1..75: note = Description of sequence: mvRNA template: WSN_PB1_75
  source 1..75: mol_type = other RNA
agcgaaagcaggcaaaccatttgaatggatgtcaatccgactttactcttcatgaaaaaatgccttgtttctact

SEQ ID NO: 137 - RNA, 65 nt, Influenza A virus
  misc_feature 1..65: note = Description of sequence: mvRNA template: WSN_PB1_65
  source 1..65: mol_type = other RNA
agcgaaagcaggcaaaccatttgaatggatgtcaatccgactttacaaaatgccttgtttctact

SEQ ID NO: 138 - RNA, 64 nt, Influenza A virus
  misc_feature 1..64: note = Description of sequence: mvRNA template: WSN_PB1_64
  source 1..64: mol_type = other RNA
agcgaaagcaggcaaaccatttgaatggatgtcaatccgactttaaaaatgccttgtttctact

SEQ ID NO: 139 - RNA, 61 nt, Influenza A virus
  misc_feature 1..61: note = Description of sequence: mvRNA template: WSN_PB1_61
  source 1..61: mol_type = other RNA
agcgaaagcaggcaaaccatttgaatggatgtcaatccgactaaaatgccttgtttctact

SEQ ID NO: 140 - RNA, 73 nt, Influenza A virus
  misc_feature 1..73: note = Description of sequence: mvRNA template: WSN_PB1_73
  source 1..73: mol_type = other RNA
agcgaaagcaggcaaaccatttgaatggatgtcaatccgactgtccttcatgaaaaaatgccttgtttctact

SEQ ID NO: 141 - RNA, 63 nt, Influenza A virus
  misc_feature 1..63: note = Description of sequence: mvRNA template: WSN_PB1_63
  source 1..63: mol_type = other RNA
agcgaaagcaggcaaaccatttgaatggatgtcaatcccatgaaaaaatgccttgtttctact

SEQ ID NO: 142 - RNA, 60 nt, Influenza A virus
  misc_feature 1..60: note = Description of sequence: mvRNA template: WSN_PB1_60
  source 1..60: mol_type = other RNA
agcgaaagcaggcaaaccatttgaatggatgtcaatctgaaaaaatgccttgtttctact

SEQ ID NO: 143 - RNA, 80 nt, Influenza A virus
  misc_feature 1..80: note = Description of sequence: mvRNA template: WSN_PB1_80
  source 1..80: mol_type = other RNA
agcgaaagcaggcaaaccatttgaatggatgtcaatctgaatttagcttgtccttcatgaaaaaatgccttgtttctact

SEQ ID NO: 144 - RNA, 63 nt, Influenza A virus
  misc_feature 1..63: note = Description of sequence: mvRNA template: WSN_PB1_63
  source 1..63: mol_type = other RNA
agcgaaagcaggcaaaccatttgaatggatgtcaccttcatgaaaaaatgccttgtttctact

SEQ ID NO: 145 - RNA, 65 nt, Influenza A virus
  misc_feature 1..65: note = Description of sequence: mvRNA template: WSN_PB1_65
  source 1..65: mol_type = other RNA
agcgaaagcaggcaaaccatttgaatggatgtcagtccttcatgaaaaaatgccttgtttctact

SEQ ID NO: 146 - RNA, 58 nt, Influenza A virus
  misc_feature 1..58: note = Description of sequence: mvRNA template: WSN_PB1_58
  source 1..58: mol_type = other RNA
agcgaaagcaggcaaaccatttgaatggatgtccatgaaaaaatgccttgtttctact

SEQ ID NO: 147 - RNA, 52 nt, Influenza A virus
  misc_feature 1..52: note = Description of sequence: mvRNA template: WSN_PB1_52
  source 1..52: mol_type = other RNA
agcgaaagcaggcaaaccatttgaatggatgtaaaaatgccttgtttctact

SEQ ID NO: 148 - RNA, 63 nt, Influenza A virus
  misc_feature 1..63: note = Description of sequence: mvRNA template: WSN_PB1_63
  source 1..63: mol_type = other RNA
agcgaaagcaggcaaaccatttgaatggacttgtccttcatgaaaaaatgccttgtttctact

SEQ ID NO: 149 - RNA, 59 nt, Influenza A virus
  misc_feature 1..59: note = Description of sequence: mvRNA template: WSN_PB1_59
  source 1..59: mol_type = other RNA
agcgaaagcaggcaaaccatttgaatgggtccttcatgaaaaaatgccttgtttctact

SEQ ID NO: 150 - RNA, 52 nt, Influenza A virus
  misc_feature 1..52: note = Description of sequence: mvRNA template: WSN_PB1_52
  source 1..52: mol_type = other RNA
agcgaaagcaggcaaaccatttgaatgcatgaaaaaatgccttgtttctact

SEQ ID NO: 151 - RNA, 48 nt, Influenza A virus
  misc_feature 1..48: note = Description of sequence: mvRNA template: WSN_PB1_48
  source 1..48: mol_type = other RNA
agcgaaagcaggcaaaccattttcatgaaaaaatgccttgtttctact

SEQ ID NO: 152 - RNA, 61 nt, Influenza A virus
  misc_feature 1..61: note = Description of sequence: mvRNA template: WSN_PB1_61
  source 1..61: mol_type = other RNA
agcgaaagcaggcaaaccattttttagcttgtccttcatgaaaaaatgccttgtttctact

SEQ ID NO: 153 - RNA, 57 nt, Influenza A virus
  misc_feature 1..57: note = Description of sequence: mvRNA template: WSN_PB1_57
  source 1..57: mol_type = other RNA
agcgaaagcaggcaaaccattagcttgtccttcatgaaaaaatgccttgtttctact

SEQ ID NO: 154 - RNA, 60 nt, Influenza A virus
  misc_feature 1..60: note = Description of sequence: mvRNA template: WSN_PB1_60
  source 1..60: mol_type = other RNA
agcgaaagcaggcaaactgaatttagcttgtccttcatgaaaaaatgccttgtttctact

SEQ ID NO: 155 - RNA, 63 nt, Influenza A virus
  misc_feature 1..63: note = Description of sequence: mvRNA template: WSN_PB1_63
  source 1..63: mol_type = other RNA
agcgaaagcaggcaaactagtgaatttagcttgtccttcatgaaaaaatgccttgtttctact

SEQ ID NO: 156 - RNA, 56 nt, Influenza A virus
  misc_feature 1..56: note = Description of sequence: mvRNA template: WSN_PB1_56
  source 1..56: mol_type = other RNA
agcgaaagcaggcaaaatttagcttgtccttcatgaaaaaatgccttgtttctact

SEQ ID NO: 157 - RNA, 63 nt, Influenza A virus
  misc_feature 1..63: note = Description of sequence: mvRNA template: WSN_PA_63
  source 1..63: mol_type = other RNA
agcgaaagcaggtactgattcaaaatggaagattttgttccaaaaaagtaccttgtttctact

SEQ ID NO: 158 - RNA, 58 nt, Influenza A virus
  misc_feature 1..58: note = Description of sequence: mvRNA template: WSN_PA_58
  source 1..58: mol_type = other RNA
agcgaaagcaggtactgattcaaaatggaagattttaaaaaagtaccttgtttctact

SEQ ID NO: 159 - RNA, 53 nt, Influenza A virus
  misc_feature 1..53: note = Description of sequence: mvRNA template: WSN_PA_53
  source 1..53: mol_type = other RNA
agcgaaagcaggtactgattcaaaatggaagattaaagtaccttgtttctact

SEQ ID NO: 160 - RNA, 63 nt, Influenza A virus
  misc_feature 1..63: note = Description of sequence: mvRNA template: WSN_PA_63
  source 1..63: mol_type = other RNA
agcgaaagcaggtactgatttatttgctatccatactgtccaaaaaagtaccttgtttctact

SEQ ID NO: 161 - RNA, 44 nt, Influenza A virus
  misc_feature 1..44: note = Description of sequence: mvRNA template: WSN_PA_44
  source 1..44: mol_type = other RNA
agcgaaagcaggtactgagtccaaaaaagtaccttgtttctact

SEQ ID NO: 162 - RNA, 59 nt, Influenza A virus
  misc_feature 1..59: note = Description of sequence: mvRNA template: WSN_NS_59
  source 1..59: mol_type = other RNA
agcaaaagcagggtgacaaagacataatggatccaaacactaacacccttgtttctact

SEQ ID NO: 163 - RNA, 60 nt, Influenza A virus
  misc_feature 1..60: note = Description of sequence: mvRNA template: WSN_NS_60
  source 1..60: mol_type = other RNA
agcaaaagcagggtgacaaagacataatggatccaaataaaaaacacccttgtttctact

SEQ ID NO: 164 - RNA, 52 nt, Influenza A virus
  misc_feature 1..52: note = Description of sequence: mvRNA template: WSN_NS_52
  source 1..52: mol_type = other RNA
agcaaaagcagggtgacaaagacataatgtaaaaaacacccttgtttctact

SEQ ID NO: 165 - RNA, 48 nt, Influenza A virus
  misc_feature 1..48: note = Description of sequence: mvRNA template: WSN_NS_48
  source 1..48: mol_type = other RNA
agcaaaagcagggtgacaaagacattaaaaaacacccttgtttctact

SEQ ID NO: 166 - RNA, 44 nt, Influenza A virus
  misc_feature 1..44: note = Description of sequence: mvRNA template: WSN_NS_44
  source 1..44: mol_type = other RNA
agcaaaagcagggtgacaaagacaaaaacacccttgtttctact

SEQ ID NO: 167 - RNA, 46 nt, Influenza A virus
  misc_feature 1..46: note = Description of sequence: mvRNA template: WSN_NS_46
  source 1..46: mol_type = other RNA
agcaaaagcagggtgacaaagacaaaaaaacacccttgtttctact

SEQ ID NO: 168 - RNA, 45 nt, Influenza A virus
  misc_feature 1..45: note = Description of sequence: mvRNA template: WSN_NS_45
  source 1..45: mol_type = other RNA
agcaaaagcagggtgacaaagataaaaaacacccttgtttctact

SEQ ID NO: 169 - RNA, 67 nt, Influenza A virus
  misc_feature 1..67: note = Description of sequence: mvRNA template: WSN_NS_67
  source 1..67: mol_type = other RNA
agcaaaagcagggtgacaaagtctcgtttcagcttatttaataataaaaaacacccttgtttctact

SEQ ID NO: 170 - RNA, 46 nt, Influenza A virus
  misc_feature 1..46: note = Description of sequence: mvRNA template: WSN_NS_46
  source 1..46: mol_type = other RNA
agcaaaagcagggtgacaaataataaaaaacacccttgtttctact

SEQ ID NO: 171 - RNA, 63 nt, Influenza A virus
  misc_feature 1..63: note = Description of sequence: mvRNA template: WSN_NS_63
  source 1..63: mol_type = other RNA
agcaaaagcagggtgacactcgtttcagcttatttaataataaaaaacacccttgtttctact

SEQ ID NO: 172 - RNA, 94 nt, Influenza A virus
  misc_feature 1..94: note = Description of sequence: mvRNA template: WSN_NP_94
  source 1..94: mol_type = other RNA
agcaaaagcagggtagataatcactcacagagtgacatcgaaatcatggcgaccaaaggcaccaaacgatcttacaaatacccttgtttctact

SEQ ID NO: 173 - RNA, 71 nt, Influenza A virus
  misc_feature 1..71: note = Description of sequence: mvRNA template: WSN_NP_71
  source 1..71: mol_type = other RNA
agcaaaagcagggtagataatcactcacagagtgacatcgaaatcatggcaaaaatacccttgtttctact

SEQ ID NO: 174 - RNA, 74 nt, Influenza A virus
  misc_feature 1..74: note = Description of sequence: mvRNA template: WSN_NP_74
  source 1..74: mol_type = other RNA
agcaaaagcagggtagataatcactcacagagtgacatcgaaatcatggcaagaaaaatacccttgtttctact

SEQ ID NO: 175 - RNA, 76 nt, Influenza A virus
  misc_feature 1..76: note = Description of sequence: mvRNA template: WSN_NP_76
  source 1..76: mol_type = other RNA
agcaaaagcagggtagataatcactcacagagtgacatcgaaatcatggctaaagaaaaatacccttgtttctact

SEQ ID NO: 176 - RNA, 91 nt, Influenza A virus
  misc_feature 1..91: note = Description of sequence: mvRNA template: WSN_NP_91
  source 1..91: mol_type = other RNA
agcaaaagcagggtagataatcactcacagagtgacatcgaaatcatggagaggagtacgacaattaaagaaaaatacccttgtttctact

SEQ ID NO: 177 - RNA, 64 nt, Influenza A virus
  misc_feature 1..64: note = Description of sequence: mvRNA template: WSN_NP_64
  source 1..64: mol_type = other RNA
agcaaaagcagggtagataatcactcacagagtgacatcgaaataaaatacccttgtttctact

SEQ ID NO: 178 - RNA, 65 nt, Influenza A virus
  misc_feature 1..65: note = Description of sequence: mvRNA template: WSN_NP_65
  source 1..65: mol_type = other RNA
agcaaaagcagggtagataatcactcacagagtgacatcgaaagaaaaatacccttgtttctact

SEQ ID NO: 179 - RNA, 63 nt, Influenza A virus
  misc_feature 1..63: note = Description of sequence: mvRNA template: WSN_NP_63
  source 1..63: mol_type = other RNA
agcaaaagcagggtagataatcactcacagagtgacatcgagaaaaatacccttgtttctact

SEQ ID NO: 180 - RNA, 88 nt, Influenza A virus
  misc_feature 1..88: note = Description of sequence: mvRNA template: WSN_NP_88
  source 1..88: mol_type = other RNA
agcaaaagcagggtagataatcactcacagagtgacatcgcaatgcagaggagtacgacaattaaagaaaaatacccttgtttctact

SEQ ID NO: 181 - RNA, 60 nt, Influenza A virus
  misc_feature 1..60: note = Description of sequence: mvRNA template: WSN_NP_60
  source 1..60: mol_type = other RNA
agcaaaagcagggtagataatcactcacagagtgacatcaaaaatacccttgtttctact

SEQ ID NO: 182 - RNA, 64 nt, Influenza A virus
  misc_feature 1..64: note = Description of sequence: mvRNA template: WSN_NP_64
  source 1..64: mol_type = other RNA
agcaaaagcagggtagataatcactcacagagtgacatcaaagaaaaatacccttgtttctact

SEQ ID NO: 183 - RNA, 84 nt, Influenza A virus
  misc_feature 1..84: note = Description of sequence: mvRNA template: WSN_NP_84
  source 1..84: mol_type = other RNA
agcaaaagcagggtagataatcactcacagagtgacatatgcagaggagtacgacaattaaagaaaaatacccttgtttctact

SEQ ID NO: 184 - RNA, 59 nt, Influenza A virus
  misc_feature 1..59: note = Description of sequence: mvRNA template: WSN_NP_59
  source 1..59: mol_type = other RNA
agcaaaagcagggtagataatcactcacagagtgacagaaaaatacccttgtttctact

SEQ ID NO: 185 - RNA, 71 nt, Influenza A virus
  misc_feature 1..71: note = Description of sequence: mvRNA template: WSN_NP_71
  source 1..71: mol_type = other RNA
agcaaaagcagggtagataatcactcacagagtgacaacgacaattaaagaaaaatacccttgtttctact

SEQ ID NO: 186 - RNA, 64 nt, Influenza A virus
  misc_feature 1..64: note = Description of sequence: mvRNA template: WSN_NP_64
  source 1..64: mol_type = other RNA
agcaaaagcagggtagataatcactcacagagtgaaattaaagaaaaatacccttgtttctact

SEQ ID NO: 187 - RNA, 71 nt, Influenza A virus
  misc_feature 1..71: note = Description of sequence: mvRNA template: WSN_NP_71
  source 1..71: mol_type = other RNA
agcaaaagcagggtagataatcactcacagagtgagtacgacaattaaagaaaaatacccttgtttctact

SEQ ID NO: 188 - RNA, 63 nt, Influenza A virus
  misc_feature 1..63: note = Description of sequence: mvRNA template: WSN_NP_63
  source 1..63: mol_type = other RNA
agcaaaagcagggtagataatcactcacagagtcaattaaagaaaaatacccttgtttctact

SEQ ID NO: 189 - RNA, 50 nt, Influenza A virus
  misc_feature 1..50: note = Description of sequence: mvRNA template: WSN_NP_50
  source 1..50: mol_type = other RNA
agcaaaagcagggtagataatcactaaagaaaaatacccttgtttctact

SEQ ID NO: 190 - RNA, 62 nt, Influenza A virus
  misc_feature 1..62: note = Description of sequence: mvRNA template: WSN_NP_62
  source 1..62: mol_type = other RNA
agcaaaagcagggtagataatcacgagtacgacaattaaagaaaaatacccttgtttctact

SEQ ID NO: 191 - RNA, 45 nt, Influenza A virus
  misc_feature 1..45: note = Description of sequence: mvRNA template: WSN_NP_45
  source 1..45: mol_type = other RNA
agcaaaagcagggtagataatcagaaaaatacccttgtttctact

SEQ ID NO: 192 - RNA, 50 nt, Influenza A virus
  misc_feature 1..50: note = Description of sequence: mvRNA template: WSN_NP_50
  source 1..50: mol_type = other RNA
agcaaaagcagggtagataatcattaaagaaaaatacccttgtttctact

SEQ ID NO: 193 - RNA, 42 nt, Influenza A virus
  misc_feature 1..42: note = Description of sequence: mvRNA template: WSN_NP_42
  source 1..42: mol_type = other RNA
agcaaaagcagggtagataagaaaaatacccttgtttctact

SEQ ID NO: 194 - RNA, 46 nt, Influenza A virus
  misc_feature 1..46: note = Description of sequence: mvRNA template: WSN_NP_46
  source 1..46: mol_type = other RNA
agcaaaagcagggtagatattaaagaaaaatacccttgtttctact

SEQ ID NO: 195 - RNA, 46 nt, Influenza A virus
  misc_feature 1..46: note = Description of sequence: mvRNA template: WSN_NP_46
  source 1..46: mol_type = other RNA
agcaaaagcagggtagaaattaaagaaaaatacccttgtttctact

SEQ ID NO: 196 - RNA, 86 nt, Influenza A virus
  misc_feature 1..86: note = Description of sequence: mvRNA template: WSN_NA_86
  source 1..86: mol_type = other RNA
agcaaaagcaggagtttaaatgaatccaaaccagaaaataataaccattggatcgatctgtatgcaaaaaactccttgtttctact

SEQ ID NO: 197 - RNA, 64 nt, Influenza A virus
  misc_feature 1..64: note = Description of sequence: mvRNA template: WSN_NA_64
  source 1..64: mol_type = other RNA
agcaaaagcaggagtttaaatgaatccaaaccagaaaataataacaaaactccttgtttctact

SEQ ID NO: 198 - RNA, 66 nt, Influenza A virus
  misc_feature 1..66: note = Description of sequence: mvRNA template: WSN_NA_66
  source 1..66: mol_type = other RNA
agcaaaagcaggagtttaaatgaatccaaaccagatagtttgttcaaaaaactccttgtttctact

SEQ ID NO: 199 - RNA, 67 nt, Influenza A virus
  misc_feature 1..67: note = Description of sequence: mvRNA template: WSN_NA_67
  source 1..67: mol_type = other RNA
agcaaaagcaggagtttaaatgaatccaaaccaaagtagtttgttcaaaaaactccttgtttctact

SEQ ID NO: 200 - RNA, 68 nt, Influenza A virus
  misc_feature 1..68: note = Description of sequence: mvRNA template: WSN_NA_68
  source 1..68: mol_type = other RNA
agcaaaagcaggagtttaaatgaatccaaaccacaagtagtttgttcaaaaaactccttgtttctact

SEQ ID NO: 201 - RNA, 69 nt, Influenza A virus
  misc_feature 1..69: note = Description of sequence: mvRNA template: WSN_NA_69
  source 1..69: mol_type = other RNA
agcaaaagcaggagtttaaatgaatccaaaccaacaagtagtttgttcaaaaaactccttgtttctact

SEQ ID NO: 202 - RNA, 72 nt, Influenza A virus
  misc_feature 1..72: note = Description of sequence: mvRNA template: WSN_NA_72
  source 1..72: mol_type = other RNA
agcaaaagcaggagtttaaatgaatccaaaccattgacaagtagtttgttcaaaaaactccttgtttctact

SEQ ID NO: 203 - RNA, 63 nt, Influenza A virus
  misc_feature 1..63: note = Description of sequence: mvRNA template: WSN_NA_63
  source 1..63: mol_type = other RNA
agcaaaagcaggagtttaaatgaatccaaacctagtttgttcaaaaaactccttgtttctact

SEQ ID NO: 204 - RNA, 69 nt, Influenza A virus
  misc_feature 1..69: note = Description of sequence: mvRNA template: WSN_NA_69
  source 1..69: mol_type = other RNA
agcaaaagcaggagtttaaatgaatccaaaccgacaagtagtttgttcaaaaaactccttgtttctact

SEQ ID NO: 205 - RNA, 65 nt, Influenza A virus
  misc_feature 1..65: note = Description of sequence: mvRNA template: WSN_NA_65
  source 1..65: mol_type = other RNA
agcaaaagcaggagtttaaatgaatccaaacaagtagtttgttcaaaaaactccttgtttctact

SEQ ID NO: 206 - RNA, 66 nt, Influenza A virus
  misc_feature 1..66: note = Description of sequence: mvRNA template: WSN_NA_66
  source 1..66: mol_type = other RNA
agcaaaagcaggagtttaaatgaatccaaaacaagtagtttgttcaaaaaactccttgtttctact

SEQ ID NO: 207 - RNA, 67 nt, Influenza A virus
  misc_feature 1..67: note = Description of sequence: mvRNA template: WSN_NA_67
  source 1..67: mol_type = other RNA
agcaaaagcaggagtttaaatgaatccaaagacaagtagtttgttcaaaaaactccttgtttctact

SEQ ID NO: 208 - RNA, 68 nt, Influenza A virus
  misc_feature 1..68: note = Description of sequence: mvRNA template: WSN_NA_68
  source 1..68: mol_type = other RNA
agcaaaagcaggagtttaaatgaatccaaatgacaagtagtttgttcaaaaaactccttgtttctact

SEQ ID NO: 209 - RNA, 65 nt, Influenza A virus
  misc_feature 1..65: note = Description of sequence: mvRNA template: WSN_NA_65
  source 1..65: mol_type = other RNA
agcaaaagcaggagtttaaatgaatccagacaagtagtttgttcaaaaaactccttgtttctact

SEQ ID NO: 210 - RNA, 68 nt, Influenza A virus
  misc_feature 1..68: note = Description of sequence: mvRNA template: WSN_NA_68
  source 1..68: mol_type = other RNA
agcaaaagcaggagtttaaatgaatcccattgacaagtagtttgttcaaaaaactccttgtttctact

SEQ ID NO: 211 - RNA, 55 nt, Influenza A virus
  misc_feature 1..55: note = Description of sequence: mvRNA template: WSN_NA_55
  source 1..55: mol_type = other RNA
agcaaaagcaggagtttaaatgagtagtttgttcaaaaaactccttgtttctact

SEQ ID NO: 212 - RNA, 63 nt, Influenza A virus
  misc_feature 1..63: note = Description of sequence: mvRNA template: WSN_NA_63
  source 1..63: mol_type = other RNA
agcaaaagcaggagtttaaatgcattgacaagtagtttgttcaaaaaactccttgtttctact

SEQ ID NO: 213 - RNA, 49 nt, Influenza A virus
  misc_feature 1..49: note = Description of sequence: mvRNA template: WSN_NA_49
  source 1..49: mol_type = other RNA
agcaaaagcaggagtttaaattttgttcaaaaaactccttgtttctact

SEQ ID NO: 214 - RNA, 56 nt, Influenza A virus
  misc_feature 1..56: note = Description of sequence: mvRNA template: WSN_NA_56
  source 1..56: mol_type = other RNA
agcaaaagcaggagtttaaatcaagtagtttgttcaaaaaactccttgtttctact

SEQ ID NO: 215 - RNA, 43 nt, Influenza A virus
  misc_feature 1..43: note = Description of sequence: mvRNA template: WSN_NA_43
  source 1..43: mol_type = other RNA
agcaaaagcaggagtttaattcaaaaaactccttgtttctact

SEQ ID NO: 216 - RNA, 62 nt, Influenza A virus
  misc_feature 1..62: note = Description of sequence: mvRNA template: WSN_NA_62
  source 1..62: mol_type = other RNA
agcaaaagcaggagtttacaccattgacaagtagtttgttcaaaaaactccttgtttctact

SEQ ID NO: 217 - RNA, 78 nt, Influenza A virus
  misc_feature 1..78: note = Description of sequence: mvRNA template: WSN_M_78
  source 1..78: mol_type = other RNA
agcgaaagcaggtagatattgaaagatgagtcttctaaccgaggtcgaaacggagtaaaaaactaccttgtttctact

SEQ ID NO: 218 - RNA, 61 nt, Influenza A virus
  misc_feature 1..61: note = Description of sequence: mvRNA template: WSN_M_61
  source 1..61: mol_type = other RNA
agcgaaagcaggtagatattgaaagattagagctggagtaaaaaactaccttgtttctact

SEQ ID NO: 219 - RNA, 50 nt, Influenza A virus
  misc_feature 1..50: note = Description of sequence: mvRNA template: WSN_M_50
  source 1..50: mol_type = other RNA
agcgaaagcaggtagatattgaaagattaaaaaactaccttgtttctact

SEQ ID NO: 220 - RNA, 43 nt, Influenza A virus
  misc_feature 1..43: note = Description of sequence: mvRNA template: WSN_M_43
  source 1..43: mol_type = other RNA
agcgaaagcaggtagatatttaaaaaactaccttgtttctact

SEQ ID NO: 221 - RNA, 46 nt, Influenza A virus
  misc_feature 1..46: note = Description of sequence: mvRNA template: WSN_M_46
  source 1..46: mol_type = other RNA
agcgaaagcaggtagatatggagtaaaaaactaccttgtttctact

SEQ ID NO: 222 - RNA, 77 nt, Influenza A virus
  misc_feature 1..77: note = Description of sequence: mvRNA template: WSN_HA_77
  source 1..77: mol_type = other RNA
agcaaaagcaggggaaaataaaaacaaccaaaatgtaggatttcagaaatataaggaaaaacacccttgtttctact

SEQ ID NO: 223 - RNA, 53 nt, Influenza A virus
  misc_feature 1..53: note = Description of sequence: mvRNA template: WSN_HA_53
  source 1..53: mol_type = other RNA
agcaaaagcaggggaaaataaaaacaaccaaaataaacacccttgtttctact

SEQ ID NO: 224 - RNA, 61 nt, Influenza A virus
  misc_feature 1..61: note = Description of sequence: mvRNA template: WSN_HA_61
  source 1..61: mol_type = other RNA
agcaaaagcaggggaaaataaaaacaaccaaaatataaggaaaaacacccttgtttctact

SEQ ID NO: 225 - RNA, 65 nt, Influenza A virus
  misc_feature 1..65: note = Description of sequence: mvRNA template: WSN_HA_65
  source 1..65: mol_type = other RNA
agcaaaagcaggggaaaataaaaacaaccaaaataaatataaggaaaaacacccttgtttctact

SEQ ID NO: 226 - RNA, 60 nt, Influenza A virus
  misc_feature 1..60: note = Description of sequence: mvRNA template: WSN_HA_60
  source 1..60: mol_type = other RNA
agcaaaagcaggggaaaataaaaacaaccaaaaataaggaaaaacacccttgtttctact

SEQ ID NO: 227 - RNA, 87 nt, Influenza A virus
  misc_feature 1..87: note = Description of sequence: mvRNA template: WSN_HA_87
  source 1..87: mol_type = other RNA
agcaaaagcaggggaaaataaaaacaaccaatatgcatctgagattaggatttcagaaatataaggaaaaacacccttgtttctact

SEQ ID NO: 228 - RNA, 65 nt, Influenza A virus
  misc_feature 1..65: note = Description of sequence: mvRNA template: WSN_HA_65
  source 1..65: mol_type = other RNA
agcaaaagcaggggaaaataaaaacaaccatcagaaatataaggaaaaacacccttgtttctact

SEQ ID NO: 229 - RNA, 66 nt, Influenza A virus
  misc_feature 1..66: note = Description of sequence: mvRNA template: WSN_HA_66
  source 1..66: mol_type = other RNA
agcaaaagcaggggaaaataaaaacaaccattcagaaatataaggaaaaacacccttgtttctact

SEQ ID NO: 230 - RNA, 51 nt, Influenza A virus
  misc_feature 1..51: note = Description of sequence: mvRNA template: WSN_HA_51
  source 1..51: mol_type = other RNA
agcaaaagcaggggaaaataaaaacaaccgaaaaacacccttgtttctact

SEQ ID NO: 231 - RNA, 50 nt, Influenza A virus
  misc_feature 1..50: note = Description of sequence: mvRNA template: WSN_HA_50
  source 1..50: mol_type = other RNA
agcaaaagcaggggaaaataaaaacaacgaaaaacacccttgtttctact

SEQ ID NO: 232 - RNA, 46 nt, Influenza A virus
  misc_feature 1..46: note = Description of sequence: mvRNA template: WSN_HA_46
  source 1..46: mol_type = other RNA
agcaaaagcaggggaaaataaaaacaaaaacacccttgtttctact

SEQ ID NO: 233 - RNA, 65 nt, Influenza A virus
  misc_feature 1..65: note = Description of sequence: mvRNA template: WSN_HA_65
  source 1..65: mol_type = other RNA
agcaaaagcaggggaaaataaaaacagatttcagaaatataaggaaaaacacccttgtttctact

SEQ ID NO: 234 - RNA, 44 nt, Influenza A virus
  misc_feature 1..44: note = Description of sequence: mvRNA template: WSN_HA_44
  source 1..44: mol_type = other RNA
agcaaaagcaggggaaaataaaaaaaaacacccttgtttctact

SEQ ID NO: 235 - RNA, 56 nt, Influenza A virus
  misc_feature 1..56: note = Description of sequence: mvRNA template: WSN_HA_56
  source 1..56: mol_type = other RNA
agcaaaagcaggggaaaataaaaagaaatataaggaaaaacacccttgtttctact

SEQ ID NO: 236 - RNA, 60 nt, Influenza A virus
  misc_feature 1..60: note = Description of sequence: mvRNA template: WSN_HA_60
  source 1..60: mol_type = other RNA
agcaaaagcaggggaaaataaaaattcagaaatataaggaaaaacacccttgtttctact

SEQ ID NO: 237 - RNA, 63 nt, Influenza A virus
  misc_feature 1..63: note = Description of sequence: mvRNA template: WSN_HA_63
  source 1..63: mol_type = other RNA
agcaaaagcaggggaaaataaaaagatttcagaaatataaggaaaaacacccttgtttctact

SEQ ID NO: 238 - RNA, 44 nt, Influenza A virus
  misc_feature 1..44: note = Description of sequence: mvRNA template: WSN_HA_44
  source 1..44: mol_type = other RNA
agcaaaagcaggggaaaataaggaaaaacacccttgtttctact

SEQ ID NO: 239 - RNA, 60 nt, Influenza A virus
  misc_feature 1..60: note = Description of sequence: mvRNA template: WSN_HA_60
  source 1..60: mol_type = other RNA
agcaaaagcaggggaaaataggatttcagaaatataaggaaaaacacccttgtttctact

SEQ ID NO: 240 - RNA, 53 nt, Influenza A virus
  misc_feature 1..53: note = Description of sequence: mvRNA template: WSN_HA_53
  source 1..53: mol_type = other RNA
agcaaaagcaggggaaaatcagaaatataaggaaaaacacccttgtttctact

SEQ ID NO: 241 - RNA, 58 nt, Influenza A virus
  misc_feature 1..58: note = Description of sequence: mvRNA template: WSN_HA_58
  source 1..58: mol_type = other RNA
agcaaaagcaggggaaaatgatttcagaaatataaggaaaaacacccttgtttctact

SEQ ID NO: 242 - RNA, 41 nt, Influenza A virus
  misc_feature 1..41: note = Description of sequence: mvRNA template: WSN_HA_41
  source 1..41: mol_type = other RNA
agcaaaagcaggggaaaaggaaaaacacccttgtttctact

SEQ ID NO: 243 - RNA, 43 nt, Influenza A virus
  misc_feature 1..43: note = Description of sequence: mvRNA template: WSN_HA_43
  source 1..43: mol_type = other RNA
agcaaaagcaggggaaaaaaggaaaaacacccttgtttctact

SEQ ID NO: 244 - RNA, 50 nt, Influenza A virus
  misc_feature 1..50: note = Description of sequence: mvRNA template: WSN_HA_50
  source 1..50: mol_type = other RNA
agcaaaagcaggggaaaagaaatataaggaaaaacacccttgtttctact

SEQ ID NO: 245 - RNA, 64 nt, Influenza A virus
  misc_feature 1..64: note = Description of sequence: mvRNA template: WSN_HA_64
  source 1..64: mol_type = other RNA
agcaaaagcaggggaaaaagattaggatttcagaaatataaggaaaaacacccttgtttctact

SEQ ID NO: 246 - RNA, 61 nt, Influenza A virus; identical to SEQ ID NO: 31
  misc_feature 1..61: note = Description of sequence: mvRNA template: WSN_NP_61
  source 1..61: mol_type = other RNA

SEQ ID NO: 247 - RNA, 57 nt, Influenza A virus; identical to SEQ ID NO: 32
  misc_feature 1..57: note = Description of sequence: mvRNA template: WSN_PB1_57
  source 1..57: mol_type = other RNA

SEQ ID NO: 248 - RNA, 57 nt, Influenza A virus; identical to SEQ ID NO: 33
  misc_feature 1..57: note = Description of sequence: mvRNA template: WSN_PB1_57
  source 1..57: mol_type = other RNA

SEQ ID NO: 249 - RNA, 62 nt, Influenza A virus; identical to SEQ ID NO: 43
  misc_feature 1..62: note = Description of sequence: mvRNA template: WSN_PB1_62
  source 1..62: mol_type = other RNA

SEQ ID NO: 250 - RNA, 66 nt, Influenza A virus; identical to SEQ ID NO: 34
  misc_feature 1..66: note = Description of sequence: mvRNA template: WSN_PB1_66
  source 1..66: mol_type = other RNA

SEQ ID NO: 251 - RNA, 58 nt, Influenza A virus; identical to SEQ ID NO: 102
  misc_feature 1..58: note = Description of sequence: mvRNA template: WSN_NP_58
  source 1..58: mol_type = other RNA

SEQ ID NO: 252 - RNA, 60 nt, Influenza A virus; identical to SEQ ID NO: 45
  misc_feature 1..60: note = Description of sequence: mvRNA template: WSN_HA_60
  source 1..60: mol_type = other RNA

SEQ ID NO: 253 - RNA, 44 nt, Influenza A virus; identical to SEQ ID NO: 36
  misc_feature 1..44: note = Description of sequence: mvRNA template: WSN_HA_44
  source 1..44: mol_type = other RNA

SEQ ID NO: 254 - RNA, 69 nt, Influenza A virus; identical to SEQ ID NO: 41
  misc_feature 1..69: note = Description of sequence: mvRNA template: WSN_PB1_69
  source 1..69: mol_type = other RNA

SEQ ID NO: 255 - RNA, 61 nt, Influenza A virus; identical to SEQ ID NO: 62
  misc_feature 1..61: note = Description of sequence: mvRNA template: WSN_PB1_61
  source 1..61: mol_type = other RNA

SEQ ID NO: 256 - RNA, 63 nt, Influenza A virus
  misc_feature 1..63: note = Description of sequence: mvRNA template: WSN_NP_63
  source 1..63: mol_type = other RNA
agcaaaagcagggtagataatcactcacagagtgacttaaagaaaaatacccttgtttctact

SEQ ID NO: 257 - RNA, 70 nt, Influenza A virus; identical to SEQ ID NO: 37
  misc_feature 1..70: note = Description of sequence: mvRNA template: WSN_NA_70
  source 1..70: mol_type = other RNA

SEQ ID NO: 258 - RNA, 62 nt, Influenza A virus; identical to SEQ ID NO: 216
  misc_feature 1..62: note = Description of sequence: mvRNA template: WSN_NA_62
  source 1..62: mol_type = other RNA

SEQ ID NO: 259 - RNA, 65 nt, Influenza A virus; identical to SEQ ID NO: 61
  misc_feature 1..65: note = Description of sequence: mvRNA template: WSN_PB1_65
  source 1..65: mol_type = other RNA

SEQ ID NO: 260 - RNA, 69 nt, Influenza A virus
  misc_feature 1..69: note = Description of sequence: mvRNA template: WSN_PB1_69
  source 1..69: mol_type = other RNA
agcgaaagcaggcaaaccatttgaatggatgtcaatccgtccttcatgaaaaaatgccttgtttctact

SEQ ID NO: 261 - RNA, 48 nt, Influenza A virus; identical to SEQ ID NO: 89
  misc_feature 1..48: note = Description of sequence: mvRNA template: WSN_PB1_48
  source 1..48: mol_type = other RNA

SEQ ID NO: 262 - RNA, 62 nt, Influenza A virus; identical to SEQ ID NO: 38
  misc_feature 1..62: note = Description of sequence: mvRNA template: WSN_PB1_62
  source 1..62: mol_type = other RNA

SEQ ID NO: 263 - RNA, 71 nt, Influenza A virus; identical to SEQ ID NO: 173
  misc_feature 1..71: note = Description of sequence: mvRNA template: WSN_NP_71
  source 1..71: mol_type = other RNA

SEQ ID NO: 264 - RNA, 76 nt, Influenza A virus; identical to SEQ ID NO: 175
  misc_feature 1..76: note = Description of sequence: mvRNA template: WSN_NP_76
  source 1..76: mol_type = other RNA

SEQ ID NO: 265 - RNA, 64 nt, Influenza A virus; identical to SEQ ID NO: 177
  misc_feature 1..64: note = Description of sequence: mvRNA template: WSN_NP_64
  source 1..64: mol_type = other RNA

SEQ ID NO: 266 - RNA, 55 nt, Influenza A virus; identical to SEQ ID NO: 73
  misc_feature 1..55: note = Description of sequence: mvRNA template: WSN_NP_55
  source 1..55: mol_type = other RNA

SEQ ID NO: 267 - RNA, 44 nt, Influenza A virus
  misc_feature 1..44: note = Description of sequence: mvRNA template: WSN_NP_44
  source 1..44: mol_type = other RNA
agcaaaagcagggtagattaaagaaaaatacccttgtttctact

SEQ ID NO: 268 - RNA, 58 nt, Influenza A virus; identical to SEQ ID NO: 120
  misc_feature 1..58: note = Description of sequence: mvRNA template: WSN_HA_58
  source 1..58: mol_type = other RNA

SEQ ID NO: 269 - RNA, 64 nt, Influenza A virus; identical to SEQ ID NO: 76
  misc_feature 1..64: note = Description of sequence: mvRNA template: WSN_HA_64
  source 1..64: mol_type = other RNA

SEQ ID NO: 270 - RNA, 80 nt, Influenza A virus
  misc_feature 1..80: note = Description of sequence: mvRNA template: WSN_PB2_80
  source 1..80: mol_type = other RNA
agcgaaagcaggtcaattatattcaatatggaaggccatcaattagtgtcgaatagtttaaaaacgaccttgtttctact

SEQ ID NO: 271 - RNA, 77 nt, Influenza A virus
  misc_feature 1..77: note = Description of sequence: mvRNA template: WSN_PB1_77
  source 1..77: mol_type = other RNA
agcgaaagcaggcaaaccatttgaatggatgtcaatccgactttacttttctcatgaaaaaatgccttgtttctact

SEQ ID NO: 272 - RNA, 65 nt, Influenza A virus; identical to SEQ ID NO: 137
  misc_feature 1..65: note = Description of sequence: mvRNA template: WSN_PB1_65
  source 1..65: mol_type = other RNA

SEQ ID NO: 273 - RNA, 64 nt, Influenza A virus; identical to SEQ ID NO: 40
  misc_feature 1..64: note = Description of sequence: mvRNA template: WSN_PB1_64
  source 1..64: mol_type = other RNA

SEQ ID NO: 274 - RNA, 68 nt, Influenza A virus; identical to SEQ ID NO: 44
  misc_feature 1..68: note = Description of sequence: mvRNA template: WSN_PB1_68
  source 1..68: mol_type = other RNA

SEQ ID NO: 275 - RNA, 64 nt, Influenza A virus
  misc_feature 1..64: note = Description of sequence: mvRNA template: WSN_PB1_64
  source 1..64: mol_type = other RNA
agcgaaagcaggcaaaccatttgaatggatgtcaatccgactgaaaaaatgccttgtttctact

SEQ ID NO: 276 - RNA, 58 nt, Influenza A virus
  misc_feature 1..58: note = Description of sequence: mvRNA template: WSN_PB1_58
  source 1..58: mol_type = other RNA
agcgaaagcaggcaaaccatttgaatggatgtcaatccaaaaatgccttgtttctact

SEQ ID NO: 277 - RNA, 64 nt, Influenza A virus
  misc_feature 1..64: note = Description of sequence: mvRNA template: WSN_PB1_64
  source 1..64: mol_type = other RNA
agcgaaagcaggcaaaccatttgaatggatgtcaatcctcatgaaaaaatgccttgtttctact

SEQ ID NO: 278 - RNA, 68 nt, Influenza A virus; identical to SEQ ID NO: 46
  misc_feature 1..68: note = Description of sequence: mvRNA template: WSN_PB1_68
  source 1..68: mol_type = other RNA

SEQ ID NO: 279 - RNA, 60 nt, Influenza A virus; identical to SEQ ID NO: 142
  misc_feature 1..60: note = Description of sequence: mvRNA template: WSN_PB1_60
  source 1..60: mol_type = other RNA

SEQ ID NO: 280 - RNA, 54 nt, Influenza A virus
  misc_feature 1..54: note = Description of sequence: mvRNA template: WSN_PB1_54
  source 1..54: mol_type = other RNA
agcgaaagcaggcaaaccatttgaatggatgtgaaaaaatgccttgtttctact

SEQ ID NO: 281 - RNA, 50 nt, Influenza A virus
  misc_feature 1..50: note = Description of sequence: mvRNA template: WSN_PB1_50
  source 1..50: mol_type = other RNA
agcgaaagcaggcaaaccatttgaatgggaaaaaatgccttgtttctact

SEQ ID NO: 282 - RNA, 66 nt, Influenza A virus
  misc_feature 1..66: note = Description of sequence: mvRNA template: WSN_PB1_66
  source 1..66: mol_type = other RNA
agcgaaagcaggcaaaccatttgaatggttagcttgtccttcatgaaaaaatgccttgtttctact

SEQ ID NO: 283 - RNA, 49 nt, Influenza A virus; identical to SEQ ID NO: 47
  misc_feature 1..49: note = Description of sequence: mvRNA template: WSN_PB1_49
  source 1..49: mol_type = other RNA

SEQ ID NO: 284 - RNA, 48 nt, Influenza A virus
  misc_feature 1..48: note = Description of sequence: mvRNA template: WSN_PB1_48
  source 1..48: mol_type = other RNA
agcgaaagcaggcaaaccatttgcatgaaaaaatgccttgtttctact

SEQ ID NO: 285 - RNA, 43 nt, Influenza A virus; identical to SEQ ID NO: 92
  misc_feature 1..43: note = Description of sequence: mvRNA template: WSN_PB1_43
  source 1..43: mol_type = other RNA

SEQ ID NO: 286 - RNA, 64 nt, Influenza A virus; identical to SEQ ID NO: 65
  misc_feature 1..64: note = Description of sequence: mvRNA template: WSN_PB1_64
  source 1..64: mol_type = other RNA

SEQ ID NO: 287 - RNA, 61 nt, Influenza A virus
  misc_feature 1..61: note = Description of sequence: mvRNA template: WSN_PB1_61
  source 1..61: mol_type = other RNA
agcgaaagcaggcaaacctgaatttagcttgtccttcatgaaaaaatgccttgtttctact

SEQ ID NO: 288 - RNA, 40 nt, Influenza A virus; identical to SEQ ID NO: 93
  misc_feature 1..40: note = Description of sequence: mvRNA template: WSN_PB1_40
  source 1..40: mol_type = other RNA

SEQ ID NO: 289 - RNA, 56 nt, Influenza A virus; identical to SEQ ID NO: 48
  misc_feature 1..56: note = Description of sequence: mvRNA template: WSN_PB1_56
  source 1..56: mol_type = other RNA

SEQ ID NO: 290 - RNA, 60 nt, Influenza A virus
  misc_feature 1..60: note = Description of sequence: mvRNA template: WSN_PA_60
  source 1..60: mol_type = other RNA
agcgaaagcaggtactgattcaaaatggcatactgtccaaaaaagtaccttgtttctact

SEQ ID NO: 291 - RNA, 66 nt, Influenza A virus
  misc_feature 1..66: note = Description of sequence: mvRNA template: WSN_PA_66
  source 1..66: mol_type = other RNA
agcgaaagcaggtactgattcaaaatgtgctatccatactgtccaaaaaagtaccttgtttctact

SEQ ID NO: 292 - RNA, 80 nt, Influenza A virus
  misc_feature 1..80: note = Description of sequence: mvRNA template: WSN_NS_80
  source 1..80: mol_type = other RNA
agcaaaagcagggtgacaaagacataatggatccaaacactgtgtcaagctttcagataaaaaacacccttgtttctact

SEQ ID NO: 293 - RNA, 56 nt, Influenza A virus
  misc_feature 1..56: note = Description of sequence: mvRNA template: WSN_NS_56
  source 1..56: mol_type = other RNA
agcaaaagcagggtgacaaagacataatggatctaaaaaacacccttgtttctact

SEQ ID NO: 294 - RNA, 55 nt, Influenza A virus
  misc_feature 1..55: note = Description of sequence: mvRNA template: WSN_NS_55
  source 1..55: mol_type = other RNA
agcaaaagcagggtgacaaagacataatgtaataaaaaacacccttgtttctact

SEQ ID NO: 295 - RNA, 66 nt, Influenza A virus
  misc_feature 1..66: note = Description of sequence: mvRNA template: WSN_NP_66
  source 1..66: mol_type = other RNA
agcaaaagcagggtagataatcactcacagagtgacatcgtaaagaaaaatacccttgtttctact

SEQ ID NO: 296 - RNA, 60 nt, Influenza A virus; identical to SEQ ID NO: 181
  misc_feature 1..60: note = Description of sequence: mvRNA template: WSN_NP_60
  source 1..60: mol_type = other RNA

SEQ ID NO: 297 - RNA, 65 nt, Influenza A virus; identical to SEQ ID NO: 35
  misc_feature 1..65: note = Description of sequence: mvRNA template: WSN_NP_65
  source 1..65: mol_type = other RNA

SEQ ID NO: 298 - RNA, 66 nt, Influenza A virus; identical to SEQ ID NO: 69
  misc_feature 1..66: note = Description of sequence: mvRNA template: WSN_NP_66
  source 1..66: mol_type = other RNA

SEQ ID NO: 299 - RNA, 74 nt, Influenza A virus; identical to SEQ ID NO: 51
  misc_feature 1..74: note = Description of sequence: mvRNA template: WSN_NP_74
  source 1..74: mol_type = other RNA

SEQ ID NO: 300 - RNA, 64 nt, Influenza A virus; identical to SEQ ID NO: 70
  misc_feature 1..64: note = Description of sequence: mvRNA template: WSN_NP_64
  source 1..64: mol_type = other RNA

SEQ ID NO: 301 - RNA, 59 nt, Influenza A virus; identical to SEQ ID NO: 184
  misc_feature 1..59: note = Description of sequence: mvRNA template: WSN_NP_59
  source 1..59: mol_type = other RNA

SEQ ID NO: 302 - RNA, 72 nt, Influenza A virus
  misc_feature 1..72: note = Description of sequence: mvRNA template: WSN_NP_72
  source 1..72: mol_type = other RNA
agcaaaagcagggtagataatcactcacagagtgacgtacgacaattaaagaaaaatacccttgtttctact

SEQ ID NO: 303 - RNA, 70 nt, Influenza A virus
  misc_feature 1..70: note = Description of sequence: mvRNA template: WSN_NP_70
  source 1..70: mol_type = other RNA
agcaaaagcagggtagataatcactcacagagtgatacgacaattaaagaaaaatacccttgtttctact

SEQ ID NO: 304 - RNA, 57 nt, Influenza A virus
  misc_feature 1..57: note = Description of sequence: mvRNA template: WSN_NP_57
  source 1..57: mol_type = other RNA
agcaaaagcagggtagataatcactcacagagtaagaaaaatacccttgtttctact

SEQ ID NO: 305 - RNA, 44 nt, Influenza A virus
  misc_feature 1..44: note = Description of sequence: mvRNA template: WSN_NP_44
  source 1..44: mol_type = other RNA
agcaaaagcagggtagataatcactaaatacccttgtttctact

SEQ ID NO: 306 - RNA, 49 nt, Influenza A virus
  misc_feature 1..49: note = Description of sequence: mvRNA template: WSN_NP_49
  source 1..49: mol_type = other RNA
agcaaaagcagggtagataatcactaagaaaaatacccttgtttctact

SEQ ID NO: 307 - RNA, 43 nt, Influenza A virus
  misc_feature 1..43: note = Description of sequence: mvRNA template: WSN_NP_43
  source 1..43: mol_type = other RNA
agcaaaagcagggtagttaaagaaaaatacccttgtttctact

SEQ ID NO: 308 - RNA, 75 nt, Influenza A virus
  misc_feature 1..75: note = Description of sequence: mvRNA template: WSN_NA_75
  source 1..75: mol_type = other RNA
agcaaaagcaggagtttaaatgaatccaaaccagaaaataaaagtagtttgttcaaaaaactccttgtttctact

SEQ ID NO: 309 - RNA, 73 nt, Influenza A virus; identical to SEQ ID NO: 52
  misc_feature 1..73: note = Description of sequence: mvRNA template: WSN_NA_73
  source 1..73: mol_type = other RNA

SEQ ID NO: 310 - RNA, 79 nt, Influenza A virus
  misc_feature 1..79: note = Description of sequence: mvRNA template: WSN_NA_79
  source 1..79: mol_type = other RNA
agcaaaagcaggagtttaaatgaatccaaaccagaaaacattgacaagtagtttgttcaaaaaactccttgtttctact

SEQ ID NO: 311 - RNA, 69 nt, Influenza A virus; identical to SEQ ID NO: 42
  misc_feature 1..69: note = Description of sequence: mvRNA template: WSN_NA_69
  source 1..69: mol_type = other RNA

SEQ ID NO: 312 - RNA, 61 nt, Influenza A virus
  misc_feature 1..61: note = Description of sequence: mvRNA template: WSN_NA_61
  source 1..61: mol_type = other RNA
agcaaaagcaggagtttaaatgaatccaaaccagatgttcaaaaaactccttgtttctact

SEQ ID NO: 313 - RNA, 60 nt, Influenza A virus
  misc_feature 1..60: note = Description of sequence: mvRNA template: WSN_NA_60
  source 1..60: mol_type = other RNA
agcaaaagcaggagtttaaatgaatccaaaccagtgttcaaaaaactccttgtttctact

SEQ ID NO: 314 - RNA, 69 nt, Influenza A virus; identical to SEQ ID NO: 201
  misc_feature 1..69: note = Description of sequence: mvRNA template: WSN_NA_69
  source 1..69: mol_type = other RNA

SEQ ID NO: 315 - RNA, 66 nt, Influenza A virus; identical to SEQ ID NO: 206
  misc_feature 1..66: note = Description of sequence: mvRNA template: WSN_NA_66
  source 1..66: mol_type = other RNA

SEQ ID NO: 316 - RNA, 60 nt, Influenza A virus
  misc_feature 1..60: note = Description of sequence: mvRNA template: WSN_NA_60
  source 1..60: mol_type = other RNA
agcaaaagcaggagtttaaatgaatccaatagtttgttcaaaaaactccttgtttctact

SEQ ID NO: 317 - RNA, 63 nt, Influenza A virus; identical to SEQ ID NO: 54
  misc_feature 1..63: note = Description of sequence: mvRNA template: WSN_NA_63
  source 1..63: mol_type = other RNA

SEQ ID NO: 318 - RNA, 62 nt, Influenza A virus
  misc_feature 1..62: note = Description of sequence: mvRNA template: WSN_NA_62
  source 1..62: mol_type = other RNA
agcaaaagcaggagttttcaccattgacaagtagtttgttcaaaaaactccttgtttctact

SEQ ID NO: 319 - RNA, 63 nt, Influenza A virus
  misc_feature 1..63: note = Description of sequence: mvRNA template: WSN_NA_63
  source 1..63: mol_type = other RNA
agcaaaagcaggagtcgttcaccattgacaagtagtttgttcaaaaaactccttgtttctact

SEQ ID NO: 320 - RNA, 71 nt, Influenza A virus
  misc_feature 1..71: note = Description of sequence: mvRNA template: WSN_M_71
  source 1..71: mol_type = other RNA
agcgaaagcaggtagatattgaaagatgagtcttccatagagctggagtaaaaaactaccttgtttctact

SEQ ID NO: 321 - RNA, 52 nt, Influenza A virus
  misc_feature 1..52: note = Description of sequence: mvRNA template: WSN_M_52
  source 1..52: mol_type = other RNA
agcgaaagcaggtagatattgaaagatgagaaaaaactaccttgtttctact

SEQ ID NO: 322 - RNA, 48 nt, Influenza A virus
  misc_feature 1..48: note = Description of sequence: mvRNA template: WSN_M_48
  source 1..48: mol_type = other RNA
agcgaaagcaggtagatattgaaagataaaaactaccttgtttctact

SEQ ID NO: 323 - RNA, 47 nt, Influenza A virus
  misc_feature 1..47: note = Description of sequence: mvRNA template: WSN_M_47
  source 1..47: mol_type = other RNA
agcgaaagcaggtagatattgaaataaaaaactaccttgtttctact

SEQ ID NO: 324 - RNA, 86 nt, Influenza A virus
  misc_feature 1..86: note = Description of sequence: mvRNA template: WSN_HA_89
  source 1..86: mol_type = other RNA
agcaaaagcaggggaaaataaaaacaaccaaaatgaaggcagattaggatttcagaaatataaggaaaaacacccttgtttctact

SEQ ID NO: 325 - RNA, 89 nt, Influenza A virus
  misc_feature 1..89: note = Description of sequence: mvRNA template: WSN_HA_90
  source 1..89: mol_type = other RNA
agcaaaagcaggggaaaataaaaacaaccaaaatgaaggcctgagattaggatttcagaaatataaggaaaaacacccttgtttctact

SEQ ID NO: 326 - RNA, 50 nt, Influenza A virus; identical to SEQ ID NO: 231
  misc_feature 1..50: note = Description of sequence: mvRNA template: WSN_HA_50
  source 1..50: mol_type = other RNA

SEQ ID NO: 327 - RNA, 63 nt, Influenza A virus; identical to SEQ ID NO: 117
  misc_feature 1..63: note = Description of sequence: mvRNA template: WSN_HA_63
  source 1..63: mol_type = other RNA

SEQ ID NO: 328 - RNA, 44 nt, Influenza A virus
  misc_feature 1..44: note = Description of sequence: mvRNA template: WSN_HA_44
  source 1..44: mol_type = other RNA
agcaaaagcaggggaaaataaagaaaaacacccttgtttctact

SEQ ID NO: 329 - RNA, 41 nt, Influenza A virus
  misc_feature 1..41: note = Description of sequence: mvRNA template: WSN_HA_41
  source 1..41: mol_type = other RNA
agcaaaagcaggggaaaatgaaaaacacccttgtttctact

SEQ ID NO: 330 - RNA, 65 nt, Influenza A virus
  misc_feature 1..65: note = Description of sequence: mvRNA template: WSN_HA_65
  source 1..65: mol_type = other RNA
agcaaaagcaggggaaatgagattaggatttcagaaatataaggaaaaacacccttgtttctact

SEQ ID NO: 331 - RNA, 66 nt, Influenza A virus
  misc_feature 1..66: note = Description of sequence: mvRNA template: WSN_HA_66
  source 1..66: mol_type = other RNA
agcaaaagcaggggaaactgagattaggatttcagaaatataaggaaaaacacccttgtttctact

SEQ ID NO: 332 - RNA, 61 nt, Influenza A virus
  misc_feature 1..61: note = Description of sequence: mvRNA template: WSN_HA_61
  source 1..61: mol_type = other RNA
agcaaaagcaggggaagattaggatttcagaaatataaggaaaaacacccttgtttctact

SEQ ID NO: 333 - RNA, 35 nt, Influenza A virus
  misc_feature 1..35: note = Description of sequence: mvRNA template: BM18_PB2,1,2325,35:(18+17)
  source 1..35: mol_type = other RNA
agcraaagcaggtcaattacgaccttgtttctact

SEQ ID NO: 334 - RNA, 36 nt, Influenza A virus
  misc_feature 1..36: note = Description of sequence: mvRNA template: BM18_M,1,1009,36:(17+19)
  source 1..36: mol_type = other RNA
agcraaagcaggtagataaactaccttgtttctact

SEQ ID NO: 335 - RNA, 35 nt, Influenza A virus
  misc_feature 1..35: note = Description of sequence: mvRNA template: BM18_M,1,1010,35:(17+18)
  source 1..35: mol_type = other RNA
agcraaagcaggtagataactaccttgtttctact

SEQ ID NO: 336 - RNA, 36 nt, Influenza A virus
  misc_feature 1..36: note = Description of sequence: mvRNA template: BM18_PB2,1,2324,36:(18+18)
  source 1..36: mol_type = other RNA
agcraaagcaggtcaattaacgaccttgtttctact

SEQ ID NO: 337 - RNA, 36 nt, Influenza A virus
  misc_feature 1..36: note = Description of sequence: mvRNA template: BM18_NS,1,872,36:(17+19)
  source 1..36: mol_type = other RNA
agcraaagcagggtgacaaacacccttgtttctact

SEQ ID NO: 338 - RNA, 37 nt, Influenza A virus
  misc_feature 1..37: note = Description of sequence: mvRNA template: BM18_NP,1,1547,37:(18+19)
  source 1..37: mol_type = other RNA
agcraaagcagggtagataaatacccttgtttctact

SEQ ID NO: 339 - RNA, 36 nt, Influenza A virus
  misc_feature 1..36: note = Description of sequence: mvRNA template: BM18_HA,1,1762,36:(19+17)
  source 1..36: mol_type = other RNA
agcraaagcaggggaaaatacacccttgtttctact

SEQ ID NO: 340 - RNA, 41 nt, Influenza A virus
  misc_feature 1..41: note = Description of sequence: mvRNA template: BM18_NP,1,1541,41:(16+25)
  source 1..41: mol_type = other RNA
agcraaagcagggtagaaagaaaaatacccttgtttctact

SEQ ID NO: 341 - RNA, 35 nt, Influenza A virus
  misc_feature 1..35: note = Description of sequence: mvRNA template: BM18_NP,1,1547,35:(16+19)
  source 1..35: mol_type = other RNA
agcraaagcagggtagaaatacccttgtttctact

SEQ ID NO: 342 - RNA, 37 nt, Influenza A virus
  misc_feature 1..37: note = Description of sequence: mvRNA template: BM18_PB2,1,2323,37:(18+19)
  source 1..37: mol_type = other RNA
agcraaagcaggtcaattaaacgaccttgtttctact

SEQ ID NO: 343 - RNA, 39 nt, Influenza A virus
  misc_feature 1..39: note = Description of sequence: mvRNA template: BM18_PB2,1,2319,39:(16+23)
  source 1..39: mol_type = other RNA
agcraaagcaggtcaattaaaaacgaccttgtttctact

SEQ ID NO: 344 - RNA, 37 nt, Influenza A virus
  misc_feature 1..37: note = Description of sequence: mvRNA template: BM18_HA,1,1761,37:(19+18)
  source 1..37: mol_type = other RNA
agcraaagcaggggaaaataacacccttgtttctact

SEQ ID NO: 345 - RNA, 39 nt, Influenza A virus
  misc_feature 1..39: note = Description of sequence: mvRNA template: BM18_NS,1,869,39:(17+22)
  source 1..39: mol_type = other RNA
agcraaagcagggtgacaaaaaacacccttgtttctact

SEQ ID NO: 346 - RNA, 39 nt, Influenza A virus
  misc_feature 1..39: note = Description of sequence: mvRNA template: BM18_NP,1,1545,39:(18+21)
  source 1..39: mol_type = other RNA
agcraaagcagggtagataaaaatacccttgtttctact

SEQ ID NO: 347 - RNA, 34 nt, Influenza A virus
  misc_feature 1..34: note = Description of sequence: mvRNA template: BM18_NP,1,1548,34:(16+18)
  source 1..34: mol_type = other RNA
agcraaagcagggtagaatacccttgtttctact

SEQ ID NO: 348 - RNA, 37 nt, Influenza A virus
  misc_feature 1..37: note = Description of sequence: mvRNA template: BM18_M,1,1008,37:(17+20)
  source 1..37: mol_type = other RNA
agcraaagcaggtagataaaactaccttgtttctact

SEQ ID NO: 349 - RNA, 37 nt, Influenza A virus
  misc_feature 1..37: note = Description of sequence: mvRNA template: BM18_NS,1,871,37:(17+20)
  source 1..37: mol_type = other RNA
agcraaagcagggtgacaaaacacccttgtttctact

SEQ ID NO: 350 - RNA, 35 nt, Influenza A virus
  misc_feature 1..35: note = Description of sequence: mvRNA template: BM18_NS,1,873,35:(17+18)
  source 1..35: mol_type = other RNA
agcraaagcagggtgacaacacccttgtttctact

SEQ ID NO: 351 - RNA, 38 nt, Influenza A virus
  misc_feature 1..38: note = Description of sequence: mvRNA template: BM18_HA,1,1760,38:(19+19)
  source 1..38: mol_type = other RNA
agcraaagcaggggaaaataaacacccttgtttctact

SEQ ID NO: 352 - RNA, 44 nt, Influenza A virus
  misc_feature 1..44: note = Description of sequence: mvRNA template: BM18_HA,1,1752,44:(17+27)
  source 1..44: mol_type = other RNA
agcraaagcaggggaaaataaggaaaaacacccttgtttctact

SEQ ID NO: 353 - RNA, 36 nt, Influenza A virus
  misc_feature 1..36: note = Description of sequence: mvRNA template: BM18_NP,1,1546,36:(16+20)
  source 1..36: mol_type = other RNA
agcraaagcagggtagaaaatacccttgtttctact

SEQ ID NO: 354 - RNA, 39 nt, Influenza A virus
  misc_feature 1..39: note = Description of sequence: mvRNA template: BM18_M,1,1005,39:(16+23)
  source 1..39: mol_type = other RNA
agcraaagcaggtagataaaaaactaccttgtttctact

SEQ ID NO: 355 - RNA, 38 nt, Influenza A virus
  misc_feature 1..38: note = Description of sequence: mvRNA template: BM18_NP,1,1546,38:(18+20)
  source 1..38: mol_type = other RNA
agcraaagcagggtagataaaatacccttgtttctact

SEQ ID NO: 356 - RNA, 36 nt, Influenza A virus
  misc_feature 1..36: note = Description of sequence: mvRNA template: BM18_PB1,1,2324,36:(18+18)
  source 1..36: mol_type = other RNA
agcraaagcaggcaaaccaaatgccttgtttctact

SEQ ID NO: 357 - RNA, 41 nt, Influenza A virus
  misc_feature 1..41: note = Description of sequence: mvRNA template: BM18_HA,1,1757,41:(19+22)
  source 1..41: mol_type = other RNA
agcraaagcaggggaaaatgaaaaacacccttgtttctact

SEQ ID NO: 358 - RNA, 43 nt, Influenza A virus
  misc_feature 1..43: note = Description of sequence: mvRNA template: BM18_NP,1,1540,43:(17+26)
  source 1..43: mol_type = other RNA
agcraaagcagggtagataaagaaaaatacccttgtttctact

SEQ ID NO: 359 - RNA, 38 nt, Influenza A virus
  misc_feature 1..38: note = Description of sequence: mvRNA template: BM18_NP,1,1544,38:(16+22)
  source 1..38: mol_type = other RNA
agcraaagcagggtaggaaaaatacccttgtttctact

SEQ ID NO: 360 - RNA, 37 nt, Influenza A virus
  misc_feature 1..37: note = Description of sequence: mvRNA template: BM18_PB1,1,2323,37:(18+19)
  source 1..37: mol_type = other RNA
agcraaagcaggcaaaccaaaatgccttgtttctact

SEQ ID NO: 361 - RNA, 37 nt, Influenza A virus
  misc_feature 1..37: note = Description of sequence: mvRNA template: BM18_HA,1,1758,37:(16+21)
  source 1..37: mol_type = other RNA
agcraaagcaggggaaaaaaacacccttgtttctact

SEQ ID NO: 362 - RNA, 35 nt, Influenza A virus
  misc_feature 1..35: note = Description of sequence: mvRNA template: BM18_NP,1,1549,35:(18+17)
  source 1..35: mol_type = other RNA
agcraaagcagggtagatatacccttgtttctact

SEQ ID NO: 363 - RNA, 38 nt, Influenza A virus
  misc_feature 1..38: note = Description of sequence: mvRNA template: BM18_PB2,1,2322,38:(18+20)
  source 1..38: mol_type = other RNA
agcraaagcaggtcaattaaaacgaccttgtttctact

SEQ ID NO: 364 - RNA, 34 nt, Influenza A virus
  misc_feature 1..34: note = Description of sequence: mvRNA template: BM18_M,1,1011,34:(17+17)
  source 1..34: mol_type = other RNA
agcraaagcaggtagatactaccttgtttctact

SEQ ID NO: 365 - RNA, 39 nt, Influenza A virus
  misc_feature 1..39: note = Description of sequence: mvRNA template: BM18_HA,1,1759,39:(19+20)
  source 1..39: mol_type = other RNA
agcraaagcaggggaaaataaaacacccttgtttctact

SEQ ID NO: 366 - RNA, 44 nt, Influenza A virus
  misc_feature 1..44: note = Description of sequence: mvRNA template: BM18_PB2,1,2315,44:(17+27)
  source 1..44: mol_type = other RNA
agcraaagcaggtcaattagtttaaaaacgaccttgtttctact

SEQ ID NO: 367 - RNA, 39 nt, Influenza A virus
  misc_feature 1..39: note = Description of sequence: mvRNA template: BM18_NP,1,1543,39:(16+23)
  source 1..39: mol_type = other RNA
agcraaagcagggtagagaaaaatacccttgtttctact

SEQ ID NO: 368 - RNA, 38 nt, Influenza A virus
  misc_feature 1..38: note = Description of sequence: mvRNA template: BM18_HA,1,1758,38:(17+21)
  source 1..38: mol_type = other RNA
agcraaagcaggggaaaaaaaacacccttgtttctact

SEQ ID NO: 369 - RNA, 42 nt, Influenza A virus
  misc_feature 1..42: note = Description of sequence: mvRNA template: BM18_HA,1,1754,42:(17+25)
  source 1..42: mol_type = other RNA
agcraaagcaggggaaaaaggaaaaacacccttgtttctact

SEQ ID NO: 370 - RNA, 35 nt, Influenza A virus
  misc_feature 1..35: note = Description of sequence: mvRNA template: BM18_PB1,1,2325,35:(18+17)
  source 1..35: mol_type = other RNA
agcraaagcaggcaaaccaatgccttgtttctact

SEQ ID NO: 371 - RNA, 47 nt, Influenza A virus
  misc_feature 1..47: note = Description of sequence: mvRNA template: BM18_NS,1,867,47:(23+24)
  source 1..47: mol_type = other RNA
agcraaagcagggtgacaaagacataaaaaacacccttgtttctact

SEQ ID NO: 372 - RNA, 38 nt, Influenza A virus
  misc_feature 1..38: note = Description of sequence: mvRNA template: BM18_NS,1,870,38:(17+21)
  source 1..38: mol_type = other RNA
agcraaagcagggtgacaaaaacacccttgtttctact

SEQ ID NO: 373 - RNA, 34 nt, Influenza A virus
  misc_feature 1..34: note = Description of sequence: mvRNA template: BM18_NS,1,874,34:(17+17)
  source 1..34: mol_type = other RNA
agcraaagcagggtgacacacccttgtttctact

SEQ ID NO: 374 - RNA, 39 nt, Influenza A virus
  misc_feature 1..39: note = Description of sequence: mvRNA template: BM18_PB1,1,2321,39:(18+21)
  source 1..39: mol_type = other RNA
agcraaagcaggcaaaccaaaaaatgccttgtttctact

SEQ ID NO: 375 - RNA, 38 nt, Influenza A virus
  misc_feature 1..38: note = Description of sequence: mvRNA template: BM18_M,1,1007,38:(17+21)
  source 1..38: mol_type = other RNA
agcraaagcaggtagataaaaactaccttgtttctact

SEQ ID NO: 376 - RNA, 42 nt, Influenza A virus
  misc_feature 1..42: note = Description of sequence: mvRNA template: BM18_HA,1,1756,42:(19+23)
  source 1..42: mol_type = other RNA
agcraaagcaggggaaaatggaaaaacacccttgtttctact

SEQ ID NO: 377 - RNA, 36 nt, Influenza A virus
  misc_feature 1..36: note = Description of sequence: mvRNA template: BM18_NA,1,1439,36:(17+19)
  source 1..36: mol_type = other RNA
agcraaagcaggagtttaaaactccttgtttctact

SEQ ID NO: 378 - RNA, 38 nt, Influenza A virus
  misc_feature 1..38: note = Description of sequence: mvRNA template: BM18_PB1,1,2322,38:(18+20)
  source 1..38: mol_type = other RNA
agcraaagcaggcaaaccaaaaatgccttgtttctact

SEQ ID NO: 379 - RNA, 41 nt, Influenza A virus
  misc_feature 1..41: note = Description of sequence: mvRNA template: BM18_M,1,1004,41:(17+24)
  source 1..41: mol_type = other RNA
agcraaagcaggtagatgtaaaaaactaccttgtttctact

SEQ ID NO: 380 - RNA, 40 nt, Influenza A virus
  misc_feature 1..40: note = Description of sequence: mvRNA template: BM18_NP,1,1544,40:(18+22)
  source 1..40: mol_type = other RNA
agcraaagcagggtagatgaaaaatacccttgtttctact

SEQ ID NO: 381 - RNA, 40 nt, Influenza A virus
  misc_feature 1..40: note = Description of sequence: mvRNA template: BM18_NP,1,1542,40:(16+24)
  source 1..40: mol_type = other RNA
agcraaagcagggtagaagaaaaatacccttgtttctact

SEQ ID NO: 382 - RNA, 41 nt, Influenza A virus
  misc_feature 1..41: note = Description of sequence: mvRNA template: BM18_HA,1,1754,41:(16+25)
  source 1..41: mol_type = other RNA
agcraaagcaggggaaaaggaaaaacacccttgtttctact

SEQ ID NO: 383 - RNA, 47 nt, Influenza A virus
  misc_feature 1..47: note = Description of sequence: mvRNA template: BM18_PB2,1,2313,47:(18+29)
  source 1..47: mol_type = other RNA
agcraaagcaggtcaattaatagtttaaaaacgaccttgtttctact

SEQ ID NO: 384 - RNA, 42 nt, Influenza A virus
  misc_feature 1..42: note = Description of sequence: mvRNA template: BM18_M,1,1005,42:(19+23)
  source 1..42: mol_type = other RNA
agcraaagcaggtagatgttaaaaaactaccttgtttctact

SEQ ID NO: 385 - RNA, 41 nt, Influenza A virus
  misc_feature 1..41: note = Description of sequence: mvRNA template: BM18_NP,1,1543,41:(18+23)
  source 1..41: mol_type = other RNA
agcraaagcagggtagatagaaaaatacccttgtttctact

SEQ ID NO: 386 - RNA, 53 nt, Influenza A virus
  misc_feature 1..53: note = Description of sequence: mvRNA template: BM18_PB2,1,2313,53:(24+29)
  source 1..53: mol_type = other RNA
agcraaagcaggtcaattatattcaatagtttaaaaacgaccttgtttctact

SEQ ID NO: 387 - RNA, 46 nt, Influenza A virus
  misc_feature 1..46: note = Description of sequence: mvRNA template: BM18_PB2,1,2314,46:(18+28)
  source 1..46: mol_type = other RNA
agcraaagcaggtcaattatagtttaaaaacgaccttgtttctact

SEQ ID NO: 388 - RNA, 43 nt, Influenza A virus
  misc_feature 1..43: note = Description of sequence: mvRNA template: BM18_PB1,1,2322,43:(23+20)
  source 1..43: mol_type = other RNA
agcraaagcaggcaaaccatttgaaaaatgccttgtttctact

SEQ ID NO: 389 - RNA, 44 nt, Influenza A virus
  misc_feature 1..44: note = Description of sequence: mvRNA template: BM18_PB1,1,2319,44:(21+23)
  source 1..44: mol_type = other RNA
agcraaagcaggcaaaccatttgaaaaaatgccttgtttctact

SEQ ID NO: 390 - RNA, 43 nt, Influenza A virus
  misc_feature 1..43: note = Description of sequence: mvRNA template: BM18_M,1,1006,43:(21+22)
  source 1..43: mol_type = other RNA
agcraaagcaggtagatgttgaaaaaactaccttgtttctact

SEQ ID NO: 391 - RNA, 48 nt, Influenza A virus
  misc_feature 1..48: note = Description of sequence: mvRNA template: BM18_PA,1,2212,48:(26+22)
  source 1..48: mol_type = other RNA
agcraaagcaggtactgattcaaaataaaaaagtaccttgtttctact

SEQ ID NO: 392 - RNA, 39 nt, Influenza A virus
  misc_feature 1..39: note = Description of sequence: mvRNA template: BM18_PA,1,2216,39:(21+18)
  source 1..39: mol_type = other RNA
agcraaagcaggtactgattcaagtaccttgtttctact

SEQ ID NO: 393 - RNA, 34 nt, Influenza A virus
  misc_feature 1..34: note = Description of sequence: mvRNA template: BM18_PA,1,2216,34:(16+18)
  source 1..34: mol_type = other RNA
agcraaagcaggtactaagtaccttgtttctact

SEQ ID NO: 394 - RNA, 35 nt, Influenza A virus
  misc_feature 1..35: note = Description of sequence: mvRNA template: BM18_PA,1,2215,35:(16+19)
  source 1..35: mol_type = other RNA
agcraaagcaggtactaaagtaccttgtttctact

SEQ ID NO: 395 - RNA, 60 nt, Influenza A virus
  misc_feature 1..60: note = Description of sequence: mvRNA template: BM18_PB2,1,2308,60:(26+34)
  source 1..60: mol_type = other RNA
agcraaagcaggtcaattatattcaatgtcgaatagtttaaaaacgaccttgtttctact

SEQ ID NO: 396 - RNA, 52 nt, Influenza A virus
  misc_feature 1..52: note = Description of sequence: mvRNA template: BM18_HA,1,1746,52:(19+33)
  source 1..52: mol_type = other RNA
agcraaagcaggggaaaatagaaatataaggaaaaacacccttgtttctact

SEQ ID NO: 397 - RNA, 57 nt, Influenza A virus
  misc_feature 1..57: note = Description of sequence: mvRNA template: BM18_HA,1,1741,57:(19+38)
  source 1..57: mol_type = other RNA
agcraaagcaggggaaaatatttcagaaatataaggaaaaacacccttgtttctact

SEQ ID NO: 398 - RNA, 39 nt, Influenza A virus
  misc_feature 1..39: note = Description of sequence: mvRNA template: BM18_HA,1,1758,39:(18+21)
  source 1..39: mol_type = other RNA
agcraaagcaggggaaaaaaaaacacccttgtttctact

SEQ ID NO: 399 - RNA, 60 nt, Influenza A virus
  misc_feature 1..60: note = Description of sequence: mvRNA template: BM18_HA,1,1737,60:(18+42)
  source 1..60: mol_type = other RNA
agcraaagcaggggaaaataggatttcagaaatataaggaaaaacacccttgtttctact

SEQ ID NO: 400 - RNA, 43 nt, Influenza A virus
  misc_feature 1..43: note = Description of sequence: mvRNA template: BM18_NS,1,869,43:(21+22)
  source 1..43: mol_type = other RNA
agcraaagcagggtgacaaagaaaaaacacccttgtttctact

SEQ ID NO: 401 - RNA, 46 nt, Influenza A virus
  misc_feature 1..46: note = Description of sequence: mvRNA template: BM18_M,1,1000,46:(18+28)
  source 1..46: mol_type = other RNA
agcraaagcaggtagatgtggagtaaaaaactaccttgtttctact

SEQ ID NO: 402 - RNA, 66 nt, Influenza A virus
  misc_feature 1..66: note = Description of sequence: mvRNA template: BM18_NA,1,1409,66:(17+49)
  source 1..66: mol_type = other RNA
agcraaagcaggagtttccattcaccattgacaagtagtttgttcaaaaaactccttgtttctact

SEQ ID NO: 403 - RNA, 51 nt, Influenza A virus
  misc_feature 1..51: note = Description of sequence: mvRNA template: BM18_NA,1,1423,51:(16+35)
  source 1..51: mol_type = other RNA
agcraaagcaggagttcaagtagtttgttcaaaaaactccttgtttctact

SEQ ID NO: 404 - RNA, 38 nt, Influenza A virus
  misc_feature 1..38: note = Description of sequence: mvRNA template: BM18_NP,1,1545,38:(17+21)
  source 1..38: mol_type = other RNA
agcraaagcagggtagaaaaaatacccttgtttctact

SEQ ID NO: 405 - RNA, 42 nt, Influenza A virus
  misc_feature 1..42: note = Description of sequence: mvRNA template: BM18_NP,1,1541,42:(17+25)
  source 1..42: mol_type = other RNA
agcraaagcagggtagaaaagaaaaatacccttgtttctact

SEQ ID NO: 406 - RNA, 52 nt, Influenza A virus
  misc_feature 1..52: note = Description of sequence: mvRNA template: BM18_PA,1,2207,52:(25+27)
  source 1..52: mol_type = other RNA
agcraaagcaggtactgattcaaaatgtccaaaaaagtaccttgtttctact

SEQ ID NO: 407 - RNA, 43 nt, Influenza A virus
  misc_feature 1..43: note = Description of sequence: mvRNA template: BM18_PA,1,2211,43:(20+23)
  source 1..43: mol_type = other RNA
agcraaagcaggtactgattcaaaaaagtaccttgtttctact

SEQ ID NO: 408 - RNA, 45 nt, Influenza A virus
  misc_feature 1..45: note = Description of sequence: mvRNA template: BM18_PB1,1,2320,45:(23+22)
  source 1..45: mol_type = other RNA
agcraaagcaggcaaaccatttggaaaaaatgccttgtttctact

SEQ ID NO: 409 - RNA, 58 nt, Influenza A virus
  misc_feature 1..58: note = Description of sequence: mvRNA template: BM18_PB1,1,2302,58:(18+40)
  source 1..58: mol_type = other RNA
agcraaagcaggcaaaccatttagcttgtccttcatgaaaaaatgccttgtttctact

SEQ ID NO: 410 - RNA, 43 nt, Influenza A virus
  misc_feature 1..43: note = Description of sequence: mvRNA template: BM18_HA,1,1755,43:(19+24)
  source 1..43: mol_type = other RNA
agcraaagcaggggaaaataggaaaaacacccttgtttctact

SEQ ID NO: 411 - RNA, 38 nt, Influenza A virus
  misc_feature 1..38: note = Description of sequence: mvRNA template: BM18_HA,1,1757,38:(16+22)
  source 1..38: mol_type = other RNA
agcraaagcaggggaagaaaaacacccttgtttctact

SEQ ID NO: 412 - RNA, 50 nt, Influenza A virus
  misc_feature 1..50: note = Description of sequence: mvRNA template: BM18_NS,1,864,50:(23+27)
  source 1..50: mol_type = other RNA
agcraaagcagggtgacaaagacataataaaaaacacccttgtttctact

SEQ ID NO: 413 - RNA, 42 nt, Influenza A virus
  misc_feature 1..42: note = Description of sequence: mvRNA template: BM18_NS,1,869,42:(20+22)
  source 1..42: mol_type = other RNA
agcraaagcagggtgacaaaaaaaaacacccttgtttctact

SEQ ID NO: 414 - RNA, 73 nt, Influenza A virus
  misc_feature 1..73: note = Description of sequence: mvRNA template: BM18_M,1,988,73:(33+40)
  source 1..73: mol_type = other RNA
agcraaagcaggtagatgttgaaagatgagtcttcaacatagagctggagtaaaaaactaccttgtttctact

SEQ ID NO: 415 - RNA, 62 nt, Influenza A virus
  misc_feature 1..62: note = Description of sequence: mvRNA template: BM18_NA,1,1428,62:(32+30)
  source 1..62: mol_type = other RNA
agcraaagcaggagtttaaatgaatccaaatcagtttgttcaaaaaactccttgtttctact

SEQ ID NO: 416 - RNA, 38 nt, Influenza A virus
  misc_feature 1..38: note = Description of sequence: mvRNA template: BM18_NA,1,1437,38:(17+21)
  source 1..38: mol_type = other RNA
agcraaagcaggagtttaaaaaactccttgtttctact

SEQ ID NO: 417 - RNA, 58 nt, Influenza A virus
  misc_feature 1..58: note = Description of sequence: mvRNA template: BM18_NP,1,1547,58:(39+19)
  source 1..58: mol_type = other RNA
agcraaagcagggtagataatcactcactgagtgacatcaaatacccttgtttctact

SEQ ID NO: 418 - RNA, 56 nt, Influenza A virus
  misc_feature 1..56: note = Description of sequence: mvRNA template: BM18_NP,1,1546,56:(36+20)
  source 1..56: mol_type = other RNA
agcraaagcagggtagataatcactcactgagtgacaaaatacccttgtttctact

SEQ ID NO: 419 - RNA, 59 nt, Influenza A virus
  misc_feature 1..59: note = Description of sequence: mvRNA template: BM18_NP,1,1535,59:(28+31)
  source 1..59: mol_type = other RNA
agcraaagcagggtagataatcactcacacaattaaagaaaaatacccttgtttctact

SEQ ID NO: 420 - RNA, 42 nt, Influenza A virus
  misc_feature 1..42: note = Description of sequence: mvRNA template: BM18_NP,1,1540,42:(16+26)
  source 1..42: mol_type = other RNA
agcraaagcagggtagtaaagaaaaatacccttgtttctact

SEQ ID NO: 421 - RNA, 42 nt, Influenza A virus
  misc_feature 1..42: note = Description of sequence: mvRNA template: BM18_PA,1,2213,42:(21+21)
  source 1..42: mol_type = other RNA
agcraaagcaggtactgattcaaaaagtaccttgtttctact

SEQ ID NO: 422 - RNA, 35 nt, Influenza A virus
  misc_feature 1..35: note = Description of sequence: mvRNA template: BM18_PA,1,2216,35:(17+18)
  source 1..35: mol_type = other RNA
agcraaagcaggtactgaagtaccttgtttctact

SEQ ID NO: 423 - RNA, 33 nt, Influenza A virus
  misc_feature 1..33: note = Description of sequence: mvRNA template: BM18_PA,1,2217,33:(16+17)
  source 1..33: mol_type = other RNA
agcraaagcaggtactagtaccttgtttctact

SEQ ID NO: 424 - RNA, 48 nt, Influenza A virus
  misc_feature 1..48: note = Description of sequence: mvRNA template: BM18_PB2,1,2312,48:(18+30)
  source 1..48: mol_type = other RNA
agcraaagcaggtcaattgaatagtttaaaaacgaccttgtttctact

SEQ ID NO: 425 - RNA, 51 nt, Influenza A virus
  misc_feature 1..51: note = Description of sequence: mvRNA template: BM18_PB2,1,2308,51:(17+34)
  source 1..51: mol_type = other RNA
agcraaagcaggtcaattgtcgaatagtttaaaaacgaccttgtttctact

SEQ ID NO: 426 - RNA, 71 nt, Influenza A virus
  misc_feature 1..71: note = Description of sequence: mvRNA template: BM18_PB1,1,2296,71:(25+46)
  source 1..71: mol_type = other RNA
agcraaagcaggcaaaccatttgaatagtgaatttagcttgtccttcatgaaaaaatgccttgtttctact

SEQ ID NO: 427 - RNA, 40 nt, Influenza A virus
  misc_feature 1..40: note = Description of sequence: mvRNA template: BM18_PB1,1,2324,40:(22+18)
  source 1..40: mol_type = other RNA
agcraaagcaggcaaaccatttaaatgccttgtttctact

SEQ ID NO: 428 - RNA, 43 nt, Influenza A virus
  misc_feature 1..43: note = Description of sequence: mvRNA template: BM18_PB1,1,2321,43:(22+21)
  source 1..43: mol_type = other RNA
agcraaagcaggcaaaccatttaaaaaatgccttgtttctact

SEQ ID NO: 429 - RNA, 46 nt, Influenza A virus
  misc_feature 1..46: note = Description of sequence: mvRNA template: BM18_HA,1,1758,46:(25+21)
  source 1..46: mol_type = other RNA
agcraaagcaggggaaaataaaaacaaaaacacccttgtttctact

SEQ ID NO: 430 - RNA, 55 nt, Influenza A virus
  misc_feature 1..55: note = Description of sequence: mvRNA template: BM18_HA,1,1746,55:(22+33)
  source 1..55: mol_type = other RNA
agcraaagcaggggaaaataaaagaaatataaggaaaaacacccttgtttctact

SEQ ID NO: 431 - RNA, 66 nt, Influenza A virus
  misc_feature 1..66: note = Description of sequence: mvRNA template: BM18_HA,1,1731,66:(18+48)
  source 1..66: mol_type = other RNA
agcraaagcaggggaaaatgagattaggatttcagaaatataaggaaaaacacccttgtttctact

SEQ ID NO: 432 - RNA, 39 nt, Influenza A virus
  misc_feature 1..39: note = Description of sequence: mvRNA template: BM18_HA,1,1757,39:(17+22)
  source 1..39: mol_type = other RNA
agcraaagcaggggaaagaaaaacacccttgtttctact

SEQ ID NO: 433 - RNA, 43 nt, Influenza A virus
  misc_feature 1..43: note = Description of sequence: mvRNA template: BM18_NS,1,873,43:(25+18)
  source 1..43: mol_type = other RNA
agcraaagcagggtgacaaagacataacacccttgtttctact

SEQ ID NO: 434 - RNA, 46 nt, Influenza A virus
  misc_feature 1..46: note = Description of sequence: mvRNA template: BM18_NS,1,870,46:(25+21)
  source 1..46: mol_type = other RNA
agcraaagcagggtgacaaagacataaaaacacccttgtttctact

SEQ ID NO: 435 - RNA, 57 nt, Influenza A virus
  misc_feature 1..57: note = Description of sequence: mvRNA template: BM18_NS,1,858,57:(24+33)
  source 1..57: mol_type = other RNA
agcraaagcagggtgacaaagacatatttaataataaaaaacacccttgtttctact

SEQ ID NO: 436 - RNA, 42 nt, Influenza A virus
  misc_feature 1..42: note = Description of sequence: mvRNA template: BM18_NS,1,872,42:(23+19)
  source 1..42: mol_type = other RNA
agcraaagcagggtgacaaagacaaacacccttgtttctact

SEQ ID NO: 437 - RNA, 39 nt, Influenza A virus
  misc_feature 1..39: note = Description of sequence: mvRNA template: BM18_NS,1,873,39:(21+18)
  source 1..39: mol_type = other RNA
agcraaagcagggtgacaaagaacacccttgtttctact

SEQ ID NO: 438 - RNA, 49 nt, Influenza A virus
  misc_feature 1..49: note = Description of sequence: mvRNA template: BM18_NS,1,863,49:(21+28)
  source 1..49: mol_type = other RNA
agcraaagcagggtgacaaagaataataaaaaacacccttgtttctact

SEQ ID NO: 439 - RNA, 43 nt, Influenza A virus
  misc_feature 1..43: note = Description of sequence: mvRNA template: BM18_NS,1,866,43:(18+25)
  source 1..43: mol_type = other RNA
agcraaagcagggtgacaaataaaaaacacccttgtttctact

SEQ ID NO: 440 - RNA, 42 nt, Influenza A virus
  misc_feature 1..42: note = Description of sequence: mvRNA template: BM18_NS,1,866,42:(17+25)
  source 1..42: mol_type = other RNA
agcraaagcagggtgacaataaaaaacacccttgtttctact

SEQ ID NO: 441 - RNA, 56 nt, Influenza A virus
  misc_feature 1..56: note = Description of sequence: mvRNA template: BM18_M,1,1003,56:(31+25)
  source 1..56: mol_type = other RNA
agcraaagcaggtagatgttgaaagatgagtagtaaaaaactaccttgtttctact

SEQ ID NO: 442 - RNA, 48 nt, Influenza A virus
  misc_feature 1..48: note = Description of sequence: mvRNA template: BM18_M,1,1008,48:(28+20)
  source 1..48: mol_type = other RNA
agcraaagcaggtagatgttgaaagatgaaaactaccttgtttctact

SEQ ID NO: 443 - RNA, 50 nt, Influenza A virus
  misc_feature 1..50: note = Description of sequence: mvRNA template: BM18_M,1,1006,50:(28+22)
  source 1..50: mol_type = other RNA
agcraaagcaggtagatgttgaaagatgaaaaaactaccttgtttctact

SEQ ID NO: 444 - RNA, 49 nt, Influenza A virus
  misc_feature 1..49: note = Description of sequence: mvRNA template: BM18_M,1,1005,49:(26+23)
  source 1..49: mol_type = other RNA
agcraaagcaggtagatgttgaaagataaaaaactaccttgtttctact

SEQ ID NO: 445 - RNA, 43 nt, Influenza A virus
  misc_feature 1..43: note = Description of sequence: mvRNA template: BM18_M,1,1010,43:(25+18)
  source 1..43: mol_type = other RNA
agcraaagcaggtagatgttgaaagaactaccttgtttctact

SEQ ID NO: 446 - RNA, 47 nt, Influenza A virus
  misc_feature 1..47: note = Description of sequence: mvRNA template: BM18_M,1,1006,47:(25+22)
  source 1..47: mol_type = other RNA
agcraaagcaggtagatgttgaaagaaaaaactaccttgtttctact

SEQ ID NO: 447 - RNA, 42 nt, Influenza A virus
  misc_feature 1..42: note = Description of sequence: mvRNA template: BM18_M,1,1007,42:(21+21)
  source 1..42: mol_type = other RNA
agcraaagcaggtagatgttgaaaaactaccttgtttctact

SEQ ID NO: 448 - RNA, 46 nt, Influenza A virus
  misc_feature 1..46: note = Description of sequence: mvRNA template: BM18_M,1,1002,46:(20+26)
  source 1..46: mol_type = other RNA
agcraaagcaggtagatgttgagtaaaaaactaccttgtttctact

SEQ ID NO: 449 - RNA, 44 nt, Influenza A virus
  misc_feature 1..44: note = Description of sequence: mvRNA template: BM18_M,1,1003,44:(19+25)
  source 1..44: mol_type = other RNA
agcraaagcaggtagatgtagtaaaaaactaccttgtttctact

SEQ ID NO: 450 - RNA, 45 nt, Influenza A virus
  misc_feature 1..45: note = Description of sequence: mvRNA template: BM18_M,1,1000,45:(17+28)
  source 1..45: mol_type = other RNA
agcraaagcaggtagattggagtaaaaaactaccttgtttctact

SEQ ID NO: 451 - RNA, 40 nt, Influenza A virus
  misc_feature 1..40: note = Description of sequence: mvRNA template: BM18_M,1,1005,40:(17+23)
  source 1..40: mol_type = other RNA
agcraaagcaggtagattaaaaaactaccttgtttctact

SEQ ID NO: 452 - RNA, 64 nt, Influenza A virus
  misc_feature 1..64: note = Description of sequence: mvRNA template: BM18_NA,1,1426,64:(32+32)
  source 1..64: mol_type = other RNA
agcraaagcaggagtttaaatgaatccaaatcgtagtttgttcaaaaaactccttgtttctact

SEQ ID NO: 453 - RNA, 63 nt, Influenza A virus
  misc_feature 1..63: note = Description of sequence: mvRNA template: BM18_NA,1,1418,63:(23+40)
  source 1..63: mol_type = other RNA
agcraaagcaggagtttaaatgaattgacaagtagtttgttcaaaaaactccttgtttctact

SEQ ID NO: 454 - RNA, 58 nt, Influenza A virus
  misc_feature 1..58: note = Description of sequence: mvRNA template: BM18_NA,1,1420,58:(20+38)
  source 1..58: mol_type = other RNA
agcraaagcaggagtttaaatgacaagtagtttgttcaaaaaactccttgtttctact

SEQ ID NO: 455 - RNA, 55 nt, Influenza A virus
  misc_feature 1..55: note = Description of sequence: mvRNA template: BM18_NA,1,1421,55:(18+37)
  source 1..55: mol_type = other RNA
agcraaagcaggagtttagacaagtagtttgttcaaaaaactccttgtttctact

SEQ ID NO: 456 - RNA, 37 nt, Influenza A virus
  misc_feature 1..37: note = Description of sequence: mvRNA template: BM18_NA,1,1438,37:(17+20)
  source 1..37: mol_type = other RNA
agcraaagcaggagtttaaaaactccttgtttctact

SEQ ID NO: 457 - RNA, 57 nt, Influenza A virus
  misc_feature 1..57: note = Description of sequence: mvRNA template: BM18_NP,1,1548,57:(39+18)
  source 1..57: mol_type = other RNA
agcraaagcagggtagataatcactcactgagtgacatcaatacccttgtttctact

SEQ ID NO: 458 - RNA, 60 nt, Influenza A virus
  misc_feature 1..60: note = Description of sequence: mvRNA template: BM18_NP,1,1545,60:(39+21)
  source 1..60: mol_type = other RNA
agcraaagcagggtagataatcactcactgagtgacatcaaaaatacccttgtttctact

SEQ ID NO: 459 - RNA, 57 nt, Influenza A virus
  misc_feature 1..57: note = Description of sequence: mvRNA template: BM18_NP,1,1547,57:(38+19)
  source 1..57: mol_type = other RNA
agcraaagcagggtagataatcactcactgagtgacataaatacccttgtttctact

SEQ ID NO: 460 - RNA, 67 nt, Influenza A virus
  misc_feature 1..67: note = Description of sequence: mvRNA template: BM18_NP,1,1535,67:(36+31)
  source 1..67: mol_type = other RNA
agcraaagcagggtagataatcactcactgagtgacacaattaaagaaaaatacccttgtttctact

SEQ ID NO: 461 - RNA, 39 nt, Influenza A virus
  misc_feature 1..39: note = Description of sequence: mvRNA template: BM18_NP,1,1548,39:(21+18)
  source 1..39: mol_type = other RNA
agcraaagcagggtagataataatacccttgtttctact

SEQ ID NO: 462 - RNA, 42 nt, Influenza A virus
  misc_feature 1..42: note = Description of sequence: mvRNA template: BM18_NP,1,1542,42:(18+24)
  source 1..42: mol_type = other RNA
agcraaagcagggtagataagaaaaatacccttgtttctact

SEQ ID NO: 463 - RNA, 47 nt, Influenza A virus
  misc_feature 1..47: note = Description of sequence: mvRNA template: BM18_NP,1,1537,47:(18+29)
  source 1..47: mol_type = other RNA
agcraaagcagggtagataattaaagaaaaatacccttgtttctact

SEQ ID NO: 464 - RNA, 49 nt, Influenza A virus
  misc_feature 1..49: note = Description of sequence: mvRNA template: BM18_NP,1,1535,49:(18+31)
  source 1..49: mol_type = other RNA
agcraaagcagggtagatacaattaaagaaaaatacccttgtttctact

SEQ ID NO: 465 - RNA, 62 nt, Influenza A virus
  misc_feature 1..62: note = Description of sequence: mvRNA template: BM18_NP,1,1520,62:(16+46)
  source 1..62: mol_type = other RNA
agcraaagcagggtagatgcagaggagtacgacaattaaagaaaaatacccttgtttctact

SEQ ID NO: 466 - RNA, 56 nt, Influenza A virus
  misc_feature 1..56: note = Description of sequence: mvRNA template: BM18_PA,1,2216,56:(38+18)
  source 1..56: mol_type = other RNA
agcraaagcaggtactgattcaaaatggaagactttgtaagtaccttgtttctact

SEQ ID NO: 467 - RNA, 54 nt, Influenza A virus
  misc_feature 1..54: note = Description of sequence: mvRNA template: BM18_PA,1,2216,54:(36+18)
  source 1..54: mol_type = other RNA
agcraaagcaggtactgattcaaaatggaagactttaagtaccttgtttctact

SEQ ID NO: 468 - RNA, 61 nt, Influenza A virus
  misc_feature 1..61: note = Description of sequence: mvRNA template: BM18_PA,1,2198,61:(25+36)
  source 1..61: mol_type = other RNA
agcraaagcaggtactgattcaaaatatccatactgtccaaaaaagtaccttgtttctact

SEQ ID NO: 469 - RNA, 40 nt, Influenza A virus
  misc_feature 1..40: note = Description of sequence: mvRNA template: BM18_PA,1,2215,40:(21+19)
  source 1..40: mol_type = other RNA
agcraaagcaggtactgattcaaagtaccttgtttctact

SEQ ID NO: 470 - RNA, 43 nt, Influenza A virus
  misc_feature 1..43: note = Description of sequence: mvRNA template: BM18_PA,1,2209,43:(18+25)
  source 1..43: mol_type = other RNA
agcraaagcaggtactgatccaaaaaagtaccttgtttctact

SEQ ID NO: 471 - RNA, 39 nt, Influenza A virus
  misc_feature 1..39: note = Description of sequence: mvRNA template: BM18_PA,1,2212,39:(17+22)
  source 1..39: mol_type = other RNA
agcraaagcaggtactgaaaaaagtaccttgtttctact

SEQ ID NO: 472 - RNA, 64 nt, Influenza A virus
  misc_feature 1..64: note = Description of sequence: mvRNA template: BM18_PB2,1,2312,64:(34+30)
  source 1..64: mol_type = other RNA
agcraaagcaggtcaattatattcaatatggaaagaatagtttaaaaacgaccttgtttctact

SEQ ID NO: 473 - RNA, 56 nt, Influenza A virus
  misc_feature 1..56: note = Description of sequence: mvRNA template: BM18_PB2,1,2313,56:(27+29)
  source 1..56: mol_type = other RNA
agcraaagcaggtcaattatattcaataatagtttaaaaacgaccttgtttctact

SEQ ID NO: 474 - RNA, 48 nt, Influenza A virus
  misc_feature 1..48: note = Description of sequence: mvRNA template: BM18_PB2,1,2320,48:(26+22)
  source 1..48: mol_type = other RNA
agcraaagcaggtcaattatattcaataaaaacgaccttgtttctact

SEQ ID NO: 475 - RNA, 63 nt, Influenza A virus
  misc_feature 1..63: note = Description of sequence: mvRNA template: BM18_PB2,1,2305,63:(26+37)
  source 1..63: mol_type = other RNA
agcraaagcaggtcaattatattcaatagtgtcgaatagtttaaaaacgaccttgtttctact

SEQ ID NO: 476 - RNA, 45 nt, Influenza A virus
  misc_feature 1..45: note = Description of sequence: mvRNA template: BM18_PB2,1,2321,45:(24+21)
  source 1..45: mol_type = other RNA
agcraaagcaggtcaattatattcaaaaacgaccttgtttctact

SEQ ID NO: 477 - RNA, 50 nt, Influenza A virus
  misc_feature 1..50: note = Description of sequence: mvRNA template: BM18_PB2,1,2316,50:(24+26)
  source 1..50: mol_type = other RNA
agcraaagcaggtcaattatattcagtttaaaaacgaccttgtttctact

SEQ ID NO: 478 - RNA, 41 nt, Influenza A virus
  misc_feature 1..41: note = Description of sequence: mvRNA template: BM18_PB2,1,2320,41:(19+22)
  source 1..41: mol_type = other RNA
agcraaagcaggtcaattataaaaacgaccttgtttctact

SEQ ID NO: 479 - RNA, 67 nt, Influenza A virus
  misc_feature 1..67: note = Description of sequence: mvRNA template: BM18_PB2,1,2293,67:(18+49)
  source 1..67: mol_type = other RNA
agcraaagcaggtcaattatggccatcaattagtgtcgaatagtttaaaaacgaccttgtttctact

SEQ ID NO: 480 - RNA, 35 nt, Influenza A virus
  misc_feature 1..35: note = Description of sequence: mvRNA template: BM18_PB2,1,2324,35:(17+18)
  source 1..35: mol_type = other RNA
agcraaagcaggtcaataacgaccttgtttctact

SEQ ID NO: 481 - RNA, 75 nt, Influenza A virus
  misc_feature 1..75: note = Description of sequence: mvRNA template: BM18_PB1,1,2319,75:(52+23)
  source 1..75: mol_type = other RNA
agcraaagcaggcaaaccatttgaatggatgtcaatccgactttacttttcttgaaaaaatgccttgtttctact

SEQ ID NO: 482 - RNA, 62 nt, Influenza A virus
  misc_feature 1..62: note = Description of sequence: mvRNA template: BM18_PB1,1,2324,62:(44+18)
  source 1..62: mol_type = other RNA
agcraaagcaggcaaaccatttgaatggatgtcaatccgactttaaatgccttgtttctact

SEQ ID NO: 483 - RNA, 75 nt, Influenza A virus
  misc_feature 1..75: note = Description of sequence: mvRNA template: BM18_PB1,1,2309,75:(42+33)
  source 1..75: mol_type = other RNA
agcraaagcaggcaaaccatttgaatggatgtcaatccgactttgtccttcatgaaaaaatgccttgtttctact

SEQ ID NO: 484 - RNA, 48 nt, Influenza A virus
  misc_feature 1..48: note = Description of sequence: mvRNA template: BM18_PB1,1,2324,48:(30+18)
  source 1..48: mol_type = other RNA
agcraaagcaggcaaaccatttgaatggataaatgccttgtttctact

SEQ ID NO: 485 - RNA, 50 nt, Influenza A virus
  misc_feature 1..50: note = Description of sequence: mvRNA template: BM18_PB1,1,2322,50:(30+20)
  source 1..50: mol_type = other RNA
agcraaagcaggcaaaccatttgaatggataaaaatgccttgtttctact

SEQ ID NO: 486 - RNA, 50 nt, Influenza A virus
  misc_feature 1..50: note = Description of sequence: mvRNA template: BM18_PB1,1,2318,50:(26+24)
  source 1..50: mol_type = other RNA
agcraaagcaggcaaaccatttgaatatgaaaaaatgccttgtttctact

SEQ ID NO: 487 - RNA, 45 nt, Influenza A virus
  misc_feature 1..45: note = Description of sequence: mvRNA template: BM18_PB1,1,2319,45:(22+23)
  source 1..45: mol_type = other RNA
agcraaagcaggcaaaccattttgaaaaaatgccttgtttctact

SEQ ID NO: 488 - RNA, 64 nt, Influenza A virus
  misc_feature 1..64: note = Description of sequence: mvRNA template: BM18_PB1,1,2299,64:(21+43)
  source 1..64: mol_type = other RNA
agcraaagcaggcaaaccatttgaatttagcttgtccttcatgaaaaaatgccttgtttctact

SEQ ID NO: 489 - RNA, 53 nt, Influenza A virus
  misc_feature 1..53: note = Description of sequence: mvRNA template: BM18_PB1,1,2309,53:(20+33)
  source 1..53: mol_type = other RNA
agcraaagcaggcaaaccatttgtccttcatgaaaaaatgccttgtttctact

SEQ ID NO: 490 - RNA, 40 nt, Influenza A virus
  misc_feature 1..40: note = Description of sequence: mvRNA template: BM18_PB1,1,2320,40:(18+22)
  source 1..40: mol_type = other RNA
agcraaagcaggcaaaccgaaaaaatgccttgtttctact

SEQ ID NO: 491 - RNA, 49 nt, Influenza A virus
  misc_feature 1..49: note = Description of sequence: mvRNA template: BM18_PB1,1,2311,49:(18+31)
  source 1..49: mol_type = other RNA
agcraaagcaggcaaaccgtccttcatgaaaaaatgccttgtttctact

SEQ ID NO: 492 - RNA, 63 nt, Influenza A virus
  misc_feature 1..63: note = Description of sequence: mvRNA template: BM18_PB1,1,2297,63:(18+45)
  source 1..63: mol_type = other RNA
agcraaagcaggcaaaccagtgaatttagcttgtccttcatgaaaaaatgccttgtttctact

SEQ ID NO: 493 - RNA, 42 nt, Influenza A virus
  misc_feature 1..42: note = Description of sequence: mvRNA template: BM18_PB1,1,2317,42:(17+25)
  source 1..42: mol_type = other RNA
agcraaagcaggcaaaccatgaaaaaatgccttgtttctact

SEQ ID NO: 494 - RNA, 69 nt, Influenza A virus
  misc_feature 1..69: note = Description of sequence: mvRNA template: BM18_PB1,1,2290,69:(17+52)
  source 1..69: mol_type = other RNA
agcraaagcaggcaaaccaaaagtagtgaatttagcttgtccttcatgaaaaaatgccttgtttctact

SEQ ID NO: 495 - RNA, 35 nt, Influenza A virus; identical to SEQ ID NO: 333
  misc_feature 1..35: note = Description of sequence: mvRNA template: BM18_PB2,1,2325,35:(18+17)
  source 1..35: mol_type = other RNA

SEQ ID NO: 496 - RNA, 35 nt, Influenza A virus; identical to SEQ ID NO: 335
  misc_feature 1..35: note = Description of sequence: mvRNA template: BM18_M,1,1010,35:(17+18)
  source 1..35: mol_type = other RNA

SEQ ID NO: 497 - RNA, 36 nt, Influenza A virus; identical to SEQ ID NO: 334
  misc_feature 1..36: note = Description of sequence: mvRNA template: BM18_M,1,1009,36:(17+19)
  source 1..36: mol_type = other RNA

SEQ ID NO: 498 - RNA, 36 nt, Influenza A virus; identical to SEQ ID NO: 336
  misc_feature 1..36: note = Description of sequence: mvRNA template: BM18_PB2,1,2324,36:(18+18)
  source 1..36: mol_type = other RNA

SEQ ID NO: 499 - RNA, 36 nt, Influenza A virus; identical to SEQ ID NO: 337
  misc_feature 1..36: note = Description of sequence: mvRNA template: BM18_NS,1,872,36:(17+19)
  source 1..36: mol_type = other RNA

SEQ ID NO: 500 - RNA, 34 nt, Influenza A virus; identical to SEQ ID NO: 347
  misc_feature 1..34: note = Description of sequence: mvRNA template: BM18_NP,1,1548,34:(16+18)
  source 1..34: mol_type = other RNA

SEQ ID NO: 501 - RNA, 37 nt, Influenza A virus; identical to SEQ ID NO: 338
  misc_feature 1..37: note = Description of sequence: mvRNA template: BM18_NP,1,1547,37:(18+19)
  source 1..37: mol_type = other RNA

SEQ ID NO: 502 - RNA, 35 nt, Influenza A virus; identical to SEQ ID NO: 341
  misc_feature 1..35: note = Description of sequence: mvRNA template: BM18_NP,1,1547,35:(16+19)
  source 1..35: mol_type = other RNA

SEQ ID NO: 503 - RNA, 36 nt, Influenza A virus; identical to SEQ ID NO: 339
  misc_feature 1..36: note = Description of sequence: mvRNA template: BM18_HA,1,1762,36:(19+17)
  source 1..36: mol_type = other RNA

SEQ ID NO: 504 - RNA, 35 nt, Influenza A virus; identical to SEQ ID NO: 350
  misc_feature 1..35: note = Description of sequence: mvRNA template: BM18_NS,1,873,35:(17+18)
  source 1..35: mol_type = other RNA

SEQ ID NO: 505 - RNA, 39 nt, Influenza A virus; identical to SEQ ID NO: 346
  misc_feature 1..39: note = Description of sequence: mvRNA template: BM18_NP,1,1545,39:(18+21)
  source 1..39: mol_type = other RNA

SEQ ID NO: 506 - RNA, 44 nt, Influenza A virus; identical to SEQ ID NO: 352
  misc_feature 1..44: note = Description of sequence: mvRNA template: BM18_HA,1,1752,44:(17+27)
  source 1..44: mol_type = other RNA

SEQ ID NO: 507 - RNA, 37 nt, Influenza A virus; identical to SEQ ID NO: 342
  misc_feature 1..37: note = Description of sequence: mvRNA template: BM18_PB2,1,2323,37:(18+19)
  source 1..37: mol_type = other RNA

SEQ ID NO: 508 - RNA, 37 nt, Influenza A virus; identical to SEQ ID NO: 349
  misc_feature 1..37: note = Description of sequence: mvRNA template: BM18_NS,1,871,37:(17+20)
  source 1..37: mol_type = other RNA

SEQ ID NO: 509 - RNA, 37 nt, Influenza A virus; identical to SEQ ID NO: 344
  misc_feature 1..37: note = Description of sequence: mvRNA template: BM18_HA,1,1761,37:(19+18)
  source 1..37: mol_type = other RNA

SEQ ID NO: 510 - RNA, 36 nt, Influenza A virus; identical to SEQ ID NO: 353
  misc_feature 1..36: note = Description of sequence: mvRNA template: BM18_NP,1,1546,36:(16+20)
  source 1..36: mol_type = other RNA

SEQ ID NO: 511 - RNA, 41 nt, Influenza A virus; identical to SEQ ID NO: 357
  misc_feature 1..41: note = Description of sequence: mvRNA template: BM18_HA,1,1757,41:(19+22)
  source 1..41: mol_type = other RNA

SEQ ID NO: 512 - RNA, 39 nt, Influenza A virus; identical to SEQ ID NO: 345
  misc_feature 1..39: note = Description of sequence: mvRNA template: BM18_NS,1,869,39:(17+22)
  source 1..39: mol_type = other RNA

SEQ ID NO: 513 - RNA, 38 nt, Influenza A virus; identical to SEQ ID NO: 355
  misc_feature 1..38: note = Description of sequence: mvRNA template: BM18_NP,1,1546,38:(18+20)
  source 1..38: mol_type = other RNA

SEQ ID NO: 514 - RNA, 39 nt, Influenza A virus; identical to SEQ ID NO: 343
  misc_feature 1..39: note = Description of sequence: mvRNA template: BM18_PB2,1,2319,39:(16+23)
  source 1..39: mol_type = other RNA

SEQ ID NO: 515 - RNA, 37 nt, Influenza A virus; identical to SEQ ID NO: 348
  misc_feature 1..37: note = Description of sequence: mvRNA template: BM18_M,1,1008,37:(17+20)
  source 1..37: mol_type = other RNA

SEQ ID NO: 516 - RNA, 38 nt, Influenza A virus; identical to SEQ ID NO: 351
  misc_feature 1..38: note = Description of sequence: mvRNA template: BM18_HA,1,1760,38:(19+19)
  source 1..38: mol_type = other RNA

SEQ ID NO: 517 - RNA, 43 nt, Influenza A virus; identical to SEQ ID NO: 358
  misc_feature 1..43: note = Description of sequence: mvRNA template: BM18_NP,1,1540,43:(17+26)
  source 1..43: mol_type = other RNA

SEQ ID NO: 518 - RNA, 39 nt, Influenza A virus; identical to SEQ ID NO: 354
  misc_feature 1..39: note = Description of sequence: mvRNA template: BM18_M,1,1005,39:(16+23)
  source 1..39: mol_type = other RNA

SEQ ID NO: 519 - RNA, 36 nt, Influenza A virus; identical to SEQ ID NO: 356
  misc_feature 1..36: note = Description of sequence: mvRNA template: BM18_PB1,1,2324,36:(18+18)
  source 1..36: mol_type = other RNA

SEQ ID NO: 520 - RNA, 40 nt, Influenza A virus; identical to SEQ ID NO: 380
  misc_feature 1..40: note = Description of sequence: mvRNA template: BM18_NP,1,1544,40:(18+22)
  source 1..40: mol_type = other RNA

SEQ ID NO: 521 - RNA, 35 nt, Influenza A virus; identical to SEQ ID NO: 362
  misc_feature 1..35: note = Description of sequence: mvRNA template: BM18_NP,1,1549,35:(18+17)
  source 1..35: mol_type = other RNA

SEQ ID NO: 522 - RNA, 34 nt, Influenza A virus; identical to SEQ ID NO: 364
  misc_feature 1..34: note = Description of sequence: mvRNA template: BM18_M,1,1011,34:(17+17)
  source 1..34: mol_type = other RNA

SEQ ID NO: 523 - RNA, 37 nt, Influenza A virus; identical to SEQ ID NO: 360
  misc_feature 1..37: note = Description of sequence: mvRNA template: BM18_PB1,1,2323,37:(18+19)
  source 1..37: mol_type = other RNA

SEQ ID NO: 524 - RNA, 37 nt, Influenza A virus; identical to SEQ ID NO: 361
  misc_feature 1..37: note = Description of sequence: mvRNA template: BM18_HA,1,1758,37:(16+21)
  source 1..37: mol_type = other RNA

SEQ ID NO: 525 - RNA, 41 nt, Influenza A virus; identical to SEQ ID NO: 385
  misc_feature 1..41: note = Description of sequence: mvRNA template: BM18_NP,1,1543,41:(18+23)
  source 1..41: mol_type = other RNA

SEQ ID NO: 526 - RNA, 34 nt, Influenza A virus; identical to SEQ ID NO: 373
  misc_feature 1..34: note = Description of sequence: mvRNA template: BM18_NS,1,874,34:(17+17)
  source 1..34: mol_type = other RNA

SEQ ID NO: 527 - RNA, 34 nt, Influenza A virus; identical to SEQ ID NO: 393
  misc_feature 1..34: note = Description of sequence: mvRNA template: BM18_PA,1,2216,34:(16+18)
  source 1..34: mol_type = other RNA

SEQ ID NO: 528 - RNA, 42 nt, Influenza A virus; identical to SEQ ID NO: 376
  misc_feature 1..42: note = Description of sequence: mvRNA template: BM18_HA,1,1756,42:(19+23)
  source 1..42: mol_type = other RNA

SEQ ID NO: 529 - RNA, 44 nt, Influenza A virus; identical to SEQ ID NO: 366
  misc_feature 1..44: note = Description of sequence: mvRNA template: BM18_PB2,1,2315,44:(17+27)
  source 1..44: mol_type = other RNA

SEQ ID NO: 530 - RNA, 38 nt, Influenza A virus; identical to SEQ ID NO: 372
  misc_feature 1..38: note = Description of sequence: mvRNA template: BM18_NS,1,870,38:(17+21)
  source 1..38: mol_type = other RNA

SEQ ID NO: 531 - RNA, 38 nt, Influenza A virus; identical to SEQ ID NO: 363
  misc_feature 1..38: note = Description of sequence: mvRNA template: BM18_PB2,1,2322,38:(18+20)
  source 1..38: mol_type = other RNA

SEQ ID NO: 532 - RNA, 35 nt, Influenza A virus; identical to SEQ ID NO: 370
  misc_feature 1..35: note = Description of sequence: mvRNA template: BM18_PB1,1,2325,35:(18+17)
  source 1..35: mol_type = other RNA

SEQ ID NO: 533 - RNA, 38 nt, Influenza A virus; identical to SEQ ID NO: 368
  misc_feature 1..38: note = Description of sequence: mvRNA template: BM18_HA,1,1758,38:(17+21)
  source 1..38: mol_type = other RNA

SEQ ID NO: 534 - RNA, 42 nt, Influenza A virus; identical to SEQ ID NO: 369
  misc_feature 1..42: note = Description of sequence: mvRNA template: BM18_HA,1,1754,42:(17+25)
  source 1..42: mol_type = other RNA

SEQ ID NO: 535 - RNA, 39 nt, Influenza A virus; identical to SEQ ID NO: 365
  misc_feature 1..39: note = Description of sequence: mvRNA template: BM18_HA,1,1759,39:(19+20)
  source 1..39: mol_type = other RNA

SEQ ID NO: 536 - RNA, 36 nt, Influenza A virus; identical to SEQ ID NO: 377
  misc_feature 1..36: note = Description of sequence: mvRNA template: BM18_NA,1,1439,36:(17+19)
  source 1..36: mol_type = other RNA

SEQ ID NO: 537 - RNA, 38 nt, Influenza A virus; identical to SEQ ID NO: 375
  misc_feature 1..38: note = Description of sequence: mvRNA template: BM18_M,1,1007,38:(17+21)
  source 1..38: mol_type = other RNA

SEQ ID NO: 538 - RNA, 38 nt, Influenza A virus; identical to SEQ ID NO: 416
  misc_feature 1..38: note = Description of sequence: mvRNA template: BM18_NA,1,1437,38:(17+21)
  source 1..38: mol_type = other RNA

SEQ ID NO: 539 - RNA, 39 nt, Influenza A virus; identical to SEQ ID NO: 374
  misc_feature 1..39: note = Description of sequence: mvRNA template: BM18_PB1,1,2321,39:(18+21)
  source 1..39: mol_type = other RNA

SEQ ID NO: 540 - RNA, 47 nt, Influenza A virus; identical to SEQ ID NO: 371
  misc_feature 1..47: note = Description of sequence: mvRNA template: BM18_NS,1,867,47:(23+24)
  source 1..47: mol_type = other RNA

SEQ ID NO: 541 - RNA, 62 nt, Influenza A virus
  misc_feature 1..62: note = Description of sequence: mvRNA template: BM18_NP,1,1532,62:(28+34)
  source 1..62: mol_type = other RNA
agcraaagcagggtagataatcactcacacgacaattaaagaaaaatacccttgtttctact

SEQ ID NO: 542 - RNA, 41 nt, Influenza A virus; identical to SEQ ID NO: 340
  misc_feature 1..41: note = Description of sequence: mvRNA template: BM18_NP,1,1541,41:(16+25)
  source 1..41: mol_type = other RNA

SEQ ID NO: 543 - RNA, 38 nt, Influenza A virus; identical to SEQ ID NO: 378
  misc_feature 1..38: note = Description of sequence: mvRNA template: BM18_PB1,1,2322,38:(18+20)
  source 1..38: mol_type = other RNA

SEQ ID NO: 544 - RNA, 52 nt, Influenza A virus
  misc_feature 1..52: note = Description of sequence: mvRNA template: BM18_NP,1,1531,52:(17+35)
  source 1..52: mol_type = other RNA
agcraaagcagggtagatacgacaattaaagaaaaatacccttgtttctact

SEQ ID NO: 545 - RNA, 48 nt, Influenza A virus; identical to SEQ ID NO: 391
  misc_feature 1..48: note = Description of sequence: mvRNA template: BM18_PA,1,2212,48:(26+22)
  source 1..48: mol_type = other RNA

SEQ ID NO: 546 - RNA, 59 nt, Influenza A virus
  misc_feature 1..59: note = Description of sequence: mvRNA template: BM18_HA,1,1752,59:(32+27)
  source 1..59: mol_type = other RNA
agcraaagcaggggaaaataaaaacaaccaaaataaggaaaaacacccttgtttctact

SEQ ID NO: 547 - RNA, 41 nt, Influenza A virus; identical to SEQ ID NO: 382
  misc_feature 1..41: note = Description of sequence: mvRNA template: BM18_HA,1,1754,41:(16+25)
  source 1..41: mol_type = other RNA

SEQ ID NO: 548 - RNA, 43 nt, Influenza A virus; identical to SEQ ID NO: 407
  misc_feature 1..43: note = Description of sequence: mvRNA template: BM18_PA,1,2211,43:(20+23)
  source 1..43: mol_type = other RNA

SEQ ID NO: 549 - RNA, 43 nt, Influenza A virus; identical to SEQ ID NO: 410
  misc_feature 1..43: note = Description of sequence: mvRNA template: BM18_HA,1,1755,43:(19+24)
  source 1..43: mol_type = other RNA

SEQ ID NO: 550 - RNA, 60 nt, Influenza A virus; identical to SEQ ID NO: 399
  misc_feature 1..60: note = Description of sequence: mvRNA template: BM18_HA,1,1737,60:(18+42)
  source 1..60: mol_type = other RNA

SEQ ID NO: 551 - RNA, 47 nt, Influenza A virus; identical to SEQ ID NO: 383
  misc_feature 1..47: note = Description of sequence: mvRNA template: BM18_PB2,1,2313,47:(18+29)
  source 1..47: mol_type = other RNA

SEQ ID NO: 552 - RNA, 50 nt, Influenza A virus; identical to SEQ ID NO: 412
  misc_feature 1..50: note = Description of sequence: mvRNA template: BM18_NS,1,864,50:(23+27)
  source 1..50: mol_type = other RNA

SEQ ID NO: 553 - RNA, 56 nt, Influenza A virus; identical to SEQ ID NO: 418
  misc_feature 1..56: note = Description of sequence: mvRNA template: BM18_NP,1,1546,56:(36+20)
  source 1..56: mol_type = other RNA

SEQ ID NO: 554 - RNA, 59 nt, Influenza A virus; identical to SEQ ID NO: 419
  misc_feature 1..59: note = Description of sequence: mvRNA template: BM18_NP,1,1535,59:(28+31)
  source 1..59: mol_type = other RNA

SEQ ID NO: 555 - RNA, 44 nt, Influenza A virus
  misc_feature 1..44: note = Description of sequence: mvRNA template: BM18_PA,1,2212,44:(22+22)
  source 1..44: mol_type = other RNA
agcraaagcaggtactgattcaaaaaaagtaccttgtttctact

SEQ ID NO: 556 - RNA, 40 nt, Influenza A virus; identical to SEQ ID NO: 469
  misc_feature 1..40: note = Description of sequence: mvRNA template: BM18_PA,1,2215,40:(21+19)
  source 1..40: mol_type = other RNA

SEQ ID NO: 557 - RNA, 47 nt, Influenza A virus
  misc_feature 1..47: note = Description of sequence: mvRNA template: BM18_PA,1,2208,47:(21+26)
  source 1..47: mol_type = other RNA
agcraaagcaggtactgattcgtccaaaaaagtaccttgtttctact

SEQ ID NO: 558 - RNA, 35 nt, Influenza A virus; identical to SEQ ID NO: 394
  misc_feature 1..35: note = Description of sequence: mvRNA template: BM18_PA,1,2215,35:(16+19)
  source 1..35: mol_type = other RNA

SEQ ID NO: 559 - RNA, 53 nt, Influenza A virus; identical to SEQ ID NO: 386
  misc_feature 1..53: note = Description of sequence: mvRNA template: BM18_PB2,1,2313,53:(24+29)
  source 1..53: mol_type = other RNA

SEQ ID NO: 560 - RNA, 46 nt, Influenza A virus; identical to SEQ ID NO: 387
  misc_feature 1..46: note = Description of sequence: mvRNA template: BM18_PB2,1,2314,46:(18+28)
  source 1..46: mol_type = other RNA

SEQ ID NO: 561 - RNA, 36 nt, Influenza A virus
  misc_feature 1..36: note = Description of sequence: mvRNA template: BM18_PB2,1,2323,36:(17+19)
  source 1..36: mol_type = other RNA
agcraaagcaggtcaataaacgaccttgtttctact

SEQ ID NO: 562 - RNA, 61 nt, Influenza A virus
  misc_feature 1..61: note = Description of sequence: mvRNA template: BM18_HA,1,1748,61:(30+31)
  source 1..61: mol_type = other RNA
agcraaagcaggggaaaataaaaacaaccaaaatataaggaaaaacacccttgtttctact

SEQ ID NO: 563 - RNA, 66 nt, Influenza A virus
  misc_feature 1..66: note = Description of sequence: mvRNA template: BM18_HA,1,1738,66:(25+41)
  source 1..66: mol_type = other RNA
agcraaagcaggggaaaataaaaacaggatttcagaaatataaggaaaaacacccttgtttctact

SEQ ID NO: 564 - RNA, 39 nt, Influenza A virus; identical to SEQ ID NO: 398
  misc_feature 1..39: note = Description of sequence: mvRNA template: BM18_HA,1,1758,39:(18+21)
  source 1..39: mol_type = other RNA

SEQ ID NO: 565 - RNA, 39 nt, Influenza A virus; identical to SEQ ID NO: 432
  misc_feature 1..39: note = Description of sequence: mvRNA template: BM18_HA,1,1757,39:(17+22)
  source 1..39: mol_type = other RNA

SEQ ID NO: 566 - RNA, 58 nt, Influenza A virus
  misc_feature 1..58: note = Description of sequence: mvRNA template: BM18_HA,1,1738,58:(17+41)
  source 1..58: mol_type = other RNA
agcraaagcaggggaaaaggatttcagaaatataaggaaaaacacccttgtttctact

SEQ ID NO: 567 - RNA, 55 nt, Influenza A virus
  misc_feature 1..55: note = Description of sequence: mvRNA template: BM18_NS,1,866,55:(30+25)
  source 1..55: mol_type = other RNA
agcraaagcagggtgacaaagacataatggaataaaaaacacccttgtttctact

SEQ ID NO: 568 - RNA, 35 nt, Influenza A virus
  misc_feature 1..35: note = Description of sequence: mvRNA template: BM18_M,1,1011,35:(18+17)
  source 1..35: mol_type = other RNA
agcraaagcaggtagatgactaccttgtttctact

SEQ ID NO: 569 - RNA, 57 nt, Influenza A virus
  misc_feature 1..57: note = Description of sequence: mvRNA template: BM18_NA,1,1428,57:(27+30)
  source 1..57: mol_type = other RNA
agcraaagcaggagtttaaatgaatccagtttgttcaaaaaactccttgtttctact

SEQ ID NO: 570 - RNA, 47 nt, Influenza A virus
  misc_feature 1..47: note = Description of sequence: mvRNA template: BM18_NA,1,1427,47:(16+31)
  source 1..47: mol_type = other RNA
agcraaagcaggagtttagtttgttcaaaaaactccttgtttctact

SEQ ID NO: 571 - RNA, 57 nt, Influenza A virus; identical to SEQ ID NO: 457
  misc_feature 1..57: note = Description of sequence: mvRNA template: BM18_NP,1,1548,57:(39+18)
  source 1..57: mol_type = other RNA

SEQ ID NO: 572 - RNA, 57 nt, Influenza A virus
  misc_feature 1..57: note = Description of sequence: mvRNA template: BM18_NP,1,1535,57:(26+31)
  source 1..57: mol_type = other RNA
agcraaagcagggtagataatcactcacaattaaagaaaaatacccttgtttctact

SEQ ID NO: 573 - RNA, 52 nt, Influenza A virus; identical to SEQ ID NO: 406
  misc_feature 1..52: note = Description of sequence: mvRNA template: BM18_PA,1,2207,52:(25+27)
  source 1..52: mol_type = other RNA

SEQ ID NO: 574 - RNA, 36 nt, Influenza A virus
  misc_feature 1..36: note = Description of sequence: mvRNA template: BM18_PA,1,2215,36:(17+19)
  source 1..36: mol_type = other RNA
agcraaagcaggtactgaaagtaccttgtttctact

SEQ ID NO: 575 - RNA, 36 nt, Influenza A virus
  misc_feature 1..36: note = Description of sequence: mvRNA template: BM18_PA,1,2214,36:(16+20)
  source 1..36: mol_type = other RNA
agcraaagcaggtactaaaagtaccttgtttctact

SEQ ID NO: 576 - RNA, 48 nt, Influenza A virus; identical to SEQ ID NO: 474
  misc_feature 1..48: note = Description of sequence: mvRNA template: BM18_PB2,1,2320,48:(26+22)
  source 1..48: mol_type = other RNA

SEQ ID NO: 577 - RNA, 43 nt, Influenza A virus; identical to SEQ ID NO: 428
  misc_feature 1..43: note = Description of sequence: mvRNA template: BM18_PB1,1,2321,43:(22+21)
  source 1..43: mol_type = other RNA

SEQ ID NO: 578 - RNA, 44 nt, Influenza A virus; identical to SEQ ID NO: 389
  misc_feature 1..44: note = Description of sequence: mvRNA template: BM18_PB1,1,2319,44:(21+23)
  source 1..44: mol_type = other RNA

SEQ ID NO: 579 - RNA, 39 nt, Influenza A virus
  misc_feature 1..39: note = Description of sequence: mvRNA template: BM18_PB1,1,2320,39:(17+22)
  source 1..39: mol_type = other RNA
agcraaagcaggcaaacgaaaaaatgccttgtttctact

SEQ ID NO: 580 - RNA, 45 nt, Influenza A virus
  misc_feature 1..45: note = Description of sequence: mvRNA template: BM18_HA,1,1752,45:(18+27)
  source 1..45: mol_type = other RNA
agcraaagcaggggaaaaataaggaaaaacacccttgtttctact

SEQ ID NO: 581 - RNA, 66 nt, Influenza A virus; identical to SEQ ID NO: 431
  misc_feature 1..66: note = Description of sequence: mvRNA template: BM18_HA,1,1731,66:(18+48)
  source 1..66: mol_type = other RNA

SEQ ID NO: 582 - RNA, 52 nt, Influenza A virus
  misc_feature 1..52: note = Description of sequence: mvRNA template: BM18_NS,1,869,52:(30+22)
  source 1..52: mol_type = other RNA
agcraaagcagggtgacaaagacataatggaaaaaacacccttgtttctact

SEQ ID NO: 583 - RNA, 40 nt, Influenza A virus
  misc_feature 1..40: note = Description of sequence: mvRNA template: BM18_NS,1,872,40:(21+19)
  source 1..40: mol_type = other RNA
agcraaagcagggtgacaaagaaacacccttgtttctact

SEQ ID NO: 584 - RNA, 41 nt, Influenza A virus; identical to SEQ ID NO: 379
  misc_feature 1..41: note = Description of sequence: mvRNA template: BM18_M,1,1004,41:(17+24)
  source 1..41: mol_type = other RNA

SEQ ID NO: 585 - RNA, 53 nt, Influenza A virus
  misc_feature 1..53: note = Description of sequence: mvRNA template: BM18_NA,1,1433,53:(28+25)
  source 1..53: mol_type = other RNA
agcraaagcaggagtttaaatgaatccagttcaaaaaactccttgtttctact

SEQ ID NO: 586 - RNA, 61 nt, Influenza A virus
  misc_feature 1..61: note = Description of sequence: mvRNA template: BM18_NP,1,1544,61:(39+22)
  source 1..61: mol_type = other RNA
agcraaagcagggtagataatcactcactgagtgacatcgaaaaatacccttgtttctact

SEQ ID NO: 587 - RNA, 37 nt, Influenza A virus
  misc_feature 1..37: note = Description of sequence: mvRNA template: BM18_NP,1,1550,37:(21+16)
  source 1..37: mol_type = other RNA
agcraaagcagggtagataattacccttgtttctact

SEQ ID NO: 588 - RNA, 55 nt, Influenza A virus
  misc_feature 1..55: note = Description of sequence: mvRNA template: BM18_NP,1,1529,55:(18+37)
  source 1..55: mol_type = other RNA
agcraaagcagggtagatagtacgacaattaaagaaaaatacccttgtttctact

SEQ ID NO: 589 - RNA, 38 nt, Influenza A virus; identical to SEQ ID NO: 359
  misc_feature 1..38: note = Description of sequence: mvRNA template: BM18_NP,1,1544,38:(16+22)
  source 1..38: mol_type = other RNA

SEQ ID NO: 590 - RNA, 64 nt, Influenza A virus
  misc_feature 1..64: note = Description of sequence: mvRNA template: BM18_PA,1,2207,64:(37+27)
  source 1..64: mol_type = other RNA
agcraaagcaggtactgattcaaaatggaagactttgtgtccaaaaaagtaccttgtttctact

SEQ ID NO: 591 - RNA, 41 nt, Influenza A virus
  misc_feature 1..41: note = Description of sequence: mvRNA template: BM18_PA,1,2214,41:(21+20)
  source 1..41: mol_type = other RNA
agcraaagcaggtactgattcaaaagtaccttgtttctact

SEQ ID NO: 592 - RNA, 33 nt, Influenza A virus; identical to SEQ ID NO: 423
  misc_feature 1..33: note = Description of sequence: mvRNA template: BM18_PA,1,2217,33:(16+17)
  source 1..33: mol_type = other RNA

SEQ ID NO: 593 - RNA, 35 nt, Influenza A virus; identical to SEQ ID NO: 480
  misc_feature 1..35: note = Description of sequence: mvRNA template: BM18_PB2,1,2324,35:(17+18)
  source 1..35: mol_type = other RNA

SEQ ID NO: 594 - RNA, 74 nt, Influenza A virus
  misc_feature 1..74: note = Description of sequence: mvRNA template: BM18_PB1,1,2294,74:(26+48)
  source 1..74: mol_type = other RNA
agcraaagcaggcaaaccatttgaatagtagtgaatttagcttgtccttcatgaaaaaatgccttgtttctact

SEQ ID NO: 595 - RNA, 36 nt, Influenza A virus
  misc_feature 1..36: note = Description of sequence: mvRNA template: BM18_PB1,1,2326,36:(20+16)
  source 1..36: mol_type = other RNA
agcraaagcaggcaaaccatatgccttgtttctact

SEQ ID NO: 596 - RNA, 40 nt, Influenza A virus; identical to SEQ ID NO: 490
  misc_feature 1..40: note = Description of sequence: mvRNA template: BM18_PB1,1,2320,40:(18+22)
  source 1..40: mol_type = other RNA

SEQ ID NO: 597 - RNA, 35 nt, Influenza A virus
  misc_feature 1..35: note = Description of sequence: mvRNA template: BM18_PB1,1,2324,35:(17+18)
  source 1..35: mol_type = other RNA
agcraaagcaggcaaacaaatgccttgtttctact

SEQ ID NO: 598 - RNA, 36 nt, Influenza A virus
  misc_feature 1..36: note = Description of sequence: mvRNA template: BM18_PB1,1,2323,36:(17+19)
  source 1..36: mol_type = other RNA
agcraaagcaggcaaacaaaatgccttgtttctact

SEQ ID NO: 599 - RNA, 75 nt, Influenza A virus
  misc_feature 1..75: note = Description of sequence: mvRNA template: BM18_HA,1,1737,75:(33+42)
  source 1..75: mol_type = other RNA
agcraaagcaggggaaaataaaaacaaccaaaataggatttcagaaatataaggaaaaacacccttgtttctact

SEQ ID NO: 600 - RNA, 43 nt, Influenza A virus
  misc_feature 1..43: note = Description of sequence: mvRNA template: BM18_HA,1,1757,43:(21+22)
  source 1..43: mol_type = other RNA
agcraaagcaggggaaaataagaaaaacacccttgtttctact

SEQ ID NO: 601 - RNA, 63 nt, Influenza A virus
  misc_feature 1..63: note = Description of sequence: mvRNA template: BM18_NS,1,868,63:(40+23)
  source 1..63: mol_type = other RNA
agcraaagcagggtgacaaagacataatggattctaacactaaaaaacacccttgtttctact

SEQ ID NO: 602 - RNA, 45 nt, Influenza A virus
  misc_feature 1..45: note = Description of sequence: mvRNA template: BM18_NS,1,871,45:(25+20)
  source 1..45: mol_type = other RNA
agcraaagcagggtgacaaagacataaaacacccttgtttctact

SEQ ID NO: 603 - RNA, 53 nt, Influenza A virus
  misc_feature 1..53: note = Description of sequence: mvRNA template: BM18_NS,1,862,53:(24+29)
  source 1..53: mol_type = other RNA
agcraaagcagggtgacaaagacataataataaaaaacacccttgtttctact

SEQ ID NO: 604 - RNA, 39 nt, Influenza A virus; identical to SEQ ID NO: 437
  misc_feature 1..39: note = Description of sequence: mvRNA template: BM18_NS,1,873,39:(21+18)
  source 1..39: mol_type = other RNA

SEQ ID NO: 605 - RNA, 43 nt, Influenza A virus; identical to SEQ ID NO: 400
  misc_feature 1..43: note = Description of sequence: mvRNA template: BM18_NS,1,869,43:(21+22)
  source 1..43: mol_type = other RNA

SEQ ID NO: 606 - RNA, 73 nt, Influenza A virus
  misc_feature 1..73: note = Description of sequence: mvRNA template: BM18_NS,1,837,73:(19+54)
  source 1..73: mol_type = other RNA
agcraaagcagggtgacaaaagaactttctcgtttcagcttatttaataataaaaaacacccttgtttctact

SEQ ID NO: 607 - RNA, 33 nt, Influenza A virus
  misc_feature 1..33: note = Description of sequence: mvRNA template: BM18_NS,1,875,33:(17+16)
  source 1..33: mol_type = other RNA
agcraaagcagggtgaccacccttgtttctact

SEQ ID NO: 608 - RNA, 69 nt, Influenza A virus
  misc_feature 1..69: note = Description of sequence: mvRNA template: BM18_M,1,1006,69:(47+22)
  source 1..69: mol_type = other RNA
agcraaagcaggtagatgttgaaagatgagtcttctaaccgaggtcgaaaaaactaccttgtttctact

SEQ ID NO: 609 - RNA, 45 nt, Influenza A virus
  misc_feature 1..45: note = Description of sequence: mvRNA template: BM18_M,1,1010,45:(27+18)
  source 1..45: mol_type = other RNA
agcraaagcaggtagatgttgaaagataactaccttgtttctact

SEQ ID NO: 610 - RNA, 54 nt, Influenza A virus
  misc_feature 1..54: note = Description of sequence: mvRNA template: BM18_M,1,1000,54:(26+28)
  source 1..54: mol_type = other RNA
agcraaagcaggtagatgttgaaagatggagtaaaaaactaccttgtttctact

SEQ ID NO: 611 - RNA, 43 nt, Influenza A virus
  misc_feature 1..43: note = Description of sequence: mvRNA template: BM18_M,1,1002,43:(17+26)
  source 1..43: mol_type = other RNA
agcraaagcaggtagatgagtaaaaaactaccttgtttctact

SEQ ID NO: 612 - RNA, 56 nt, Influenza A virus
  misc_feature 1..56: note = Description of sequence: mvRNA template: BM18_NA,1,1429,56:(27+29)
  source 1..56: mol_type = other RNA
agcraaagcaggagtttaaatgaatccgtttgttcaaaaaactccttgtttctact

SEQ ID NO: 613 - RNA, 36 nt, Influenza A virus
  misc_feature 1..36: note = Description of sequence: mvRNA template: BM18_NA,1,1438,36:(16+20)
  source 1..36: mol_type = other RNA
agcraaagcaggagttaaaaactccttgtttctact

SEQ ID NO: 614 - RNA, 69 nt, Influenza A virus
  misc_feature 1..69: note = Description of sequence: mvRNA template: BM18_NP,1,1548,69:(51+18)
  source 1..69: mol_type = other RNA
agcraaagcagggtagataatcactcactgagtgacatcaaaatcatggcgaatacccttgtttctact

SEQ ID NO: 615 - RNA, 64 nt, Influenza A virus
  misc_feature 1..64: note = Description of sequence: mvRNA template: BM18_NP,1,1541,64:(39+25)
  source 1..64: mol_type = other RNA
agcraaagcagggtagataatcactcactgagtgacatcaaagaaaaatacccttgtttctact

SEQ ID NO: 616 - RNA, 55 nt, Influenza A virus
  misc_feature 1..55: note = Description of sequence: mvRNA template: BM18_NP,1,1547,55:(36+19)
  source 1..55: mol_type = other RNA
agcraaagcagggtagataatcactcactgagtgacaaatacccttgtttctact

SEQ ID NO: 617 - RNA, 42 nt, Influenza A virus
  misc_feature 1..42: note = Description of sequence: mvRNA template: BM18_NP,1,1545,42:(21+21)
  source 1..42: mol_type = other RNA
agcraaagcagggtagataataaaaatacccttgtttctact

SEQ ID NO: 618 - RNA, 34 nt, Influenza A virus
  misc_feature 1..34: note = Description of sequence: mvRNA template: BM18_NP,1,1550,34:(18+16)
  source 1..34: mol_type = other RNA
agcraaagcagggtagattacccttgtttctact

SEQ ID NO: 619 - RNA, 42 nt, Influenza A virus; identical to SEQ ID NO: 462
  misc_feature 1..42: note = Description of sequence: mvRNA template: BM18_NP,1,1542,42:(18+24)
  source 1..42: mol_type = other RNA

SEQ ID NO: 620 - RNA, 47 nt, Influenza A virus; identical to SEQ ID NO: 463
  misc_feature 1..47: note = Description of sequence: mvRNA template: BM18_NP,1,1537,47:(18+29)
  source 1..47: mol_type = other RNA

SEQ ID NO: 621 - RNA, 49 nt, Influenza A virus; identical to SEQ ID NO: 464
  misc_feature 1..49: note = Description of sequence: mvRNA template: BM18_NP,1,1535,49:(18+31)
  source 1..49: mol_type = other RNA

SEQ ID NO: 622 - RNA, 43 nt, Influenza A virus
  misc_feature 1..43: note = Description of sequence: mvRNA template: BM18_NP,1,1539,43:(16+27)
  source 1..43: mol_type = other RNA
agcraaagcagggtagttaaagaaaaatacccttgtttctact

SEQ ID NO: 623 - RNA, 57 nt, Influenza A virus
  misc_feature 1..57: note = Description of sequence: mvRNA template: BM18_PA,1,2208,57:(31+26)
  source 1..57: mol_type = other RNA
agcraaagcaggtactgattcaaaatggaaggtccaaaaaagtaccttgtttctact

SEQ ID NO: 624 - RNA, 39 nt, Influenza A virus; identical to SEQ ID NO: 392
  misc_feature 1..39: note = Description of sequence: mvRNA template: BM18_PA,1,2216,39:(21+18)
  source 1..39: mol_type = other RNA

SEQ ID NO: 625 - RNA, 41 nt, Influenza A virus
  misc_feature 1..41: note = Description of sequence: mvRNA template: BM18_PA,1,2213,41:(20+21)
  source 1..41: mol_type = other RNA
agcraaagcaggtactgattaaaaagtaccttgtttctact

SEQ ID NO: 626 - RNA, 34 nt, Influenza A virus
  misc_feature 1..34: note = Description of sequence: mvRNA template: BM18_PA,1,2217,34:(17+17)
  source 1..34: mol_type = other RNA
agcraaagcaggtactgagtaccttgtttctact

SEQ ID NO: 627 - RNA, 37 nt, Influenza A virus
  misc_feature 1..37: note = Description of sequence: mvRNA template: BM18_PA,1,2213,37:(16+21)
  source 1..37: mol_type = other RNA
agcraaagcaggtactaaaaagtaccttgtttctact

SEQ ID NO: 628 - RNA, 48 nt, Influenza A virus
  misc_feature 1..48: note = Description of sequence: mvRNA template: BM18_PB2,1,2323,48:(29+19)
  source 1..48: mol_type = other RNA
agcraaagcaggtcaattatattcaatataaacgaccttgtttctact

SEQ ID NO: 629 - RNA, 61 nt, Influenza A virus
  misc_feature 1..61: note = Description of sequence: mvRNA template: BM18_PB2,1,2306,61:(25+36)
  source 1..61: mol_type = other RNA
agcraaagcaggtcaattatattcaagtgtcgaatagtttaaaaacgaccttgtttctact

SEQ ID NO: 630 - RNA, 48 nt, Influenza A virus
  misc_feature 1..48: note = Description of sequence: mvRNA template: BM18_PB2,1,2314,48:(20+28)
  source 1..48: mol_type = other RNA
agcraaagcaggtcaattatatagtttaaaaacgaccttgtttctact

SEQ ID NO: 631 - RNA, 34 nt, Influenza A virus
  misc_feature 1..34: note = Description of sequence: mvRNA template: BM18_PB2,1,2326,34:(18+16)
  source 1..34: mol_type = other RNA
agcraaagcaggtcaattcgaccttgtttctact

SEQ ID NO: 632 - RNA, 67 nt, Influenza A virus; identical to SEQ ID NO: 479
  misc_feature 1..67: note = Description of sequence: mvRNA template: BM18_PB2,1,2293,67:(18+49)
  source 1..67: mol_type = other RNA

SEQ ID NO: 633 - RNA, 34 nt, Influenza A virus
  misc_feature 1..34: note = Description of sequence: mvRNA template: BM18_PB2,1,2325,34:(17+17)
  source 1..34: mol_type = other RNA
agcraaagcaggtcaatacgaccttgtttctact

SEQ ID NO: 634 - RNA, 51 nt, Influenza A virus; identical to SEQ ID NO: 425
  misc_feature 1..51: note = Description of sequence: mvRNA template: BM18_PB2,1,2308,51:(17+34)
  source 1..51: mol_type = other RNA

SEQ ID NO: 635 - RNA, 38 nt, Influenza A virus
  misc_feature 1..38: note = Description of sequence: mvRNA template: BM18_PB2,1,2320,38:(16+22)
  source 1..38: mol_type = other RNA
agcraaagcaggtcaataaaaacgaccttgtttctact

SEQ ID NO: 636 - RNA, 73 nt, Influenza A virus
  misc_feature 1..73: note = Description of sequence: mvRNA template: BM18_PB1,1,2314,73:(45+28)
  source 1..73: mol_type = other RNA
agcraaagcaggcaaaccatttgaatggatgtcaatccgactttacttcatgaaaaaatgccttgtttctact

SEQ ID NO: 637 - RNA, 49 nt, Influenza A virus
  misc_feature 1..49: note = Description of sequence: mvRNA template: BM18_PB1,1,2323,49:(30+19)
  source 1..49: mol_type = other RNA
agcraaagcaggcaaaccatttgaatggataaaatgccttgtttctact

SEQ ID NO: 638 - RNA, 47 nt, Influenza A virus
  misc_feature 1..47: note = Description of sequence: mvRNA template: BM18_PB1,1,2321,47:(26+21)
  source 1..47: mol_type = other RNA
agcraaagcaggcaaaccatttgaataaaaaatgccttgtttctact

SEQ ID NO: 639 - RNA, 41 nt, Influenza A virus
  misc_feature 1..41: note = Description of sequence: mvRNA template: BM18_PB1,1,2320,41:(19+22)
  source 1..41: mol_type = other RNA
agcraaagcaggcaaaccagaaaaaatgccttgtttctact

SEQ ID NO: 640 - RNA, 34 nt, Influenza A virus
  misc_feature 1..34: note = Description of sequence: mvRNA template: BM18_PB1,1,2325,34:(17+17)
  source 1..34: mol_type = other RNA
agcraaagcaggcaaacaatgccttgtttctact

SEQ ID NO: 641 - RNA, 42 nt, Influenza A virus; identical to SEQ ID NO: 493
  misc_feature 1..42: note = Description of sequence: mvRNA template: BM18_PB1,1,2317,42:(17+25)
  source 1..42: mol_type = other RNA

SEQ ID NO: 642 - protein, 40 aa, Influenza A virus
  REGION 1..40: note = Description of sequence: IAV PB1 C-terminus
YDAVATTHSWIPKRNRSILNTSQRGILEDEQMYQKCCNLF

SEQ ID NO: 643 - protein, 40 aa, Influenza A virus
  REGION 1..40: note = Description of sequence: IBV PB1 C-terminus
YDAVSGTHSWRTKRNRSILNTDQRNMILEEQCYAKCCNLF

SEQ ID NO: 644 - protein, 40 aa, Influenza A virus
  REGION 1..40: note = Description of sequence: ICV PB1 C-terminus
NEAVVSTHSFRTRANRTLLNTDMRAMMAEEKRYQMVCDIF

SEQ ID NO: 645 - RNA, 47 nt, synthetic construct
  misc_feature 1..47: note = Description of sequence: NP71.2 c3
  source 1..47: mol_type = other RNA
agtagaaacaagggtattttttgccactagtctaccctgcttttgct

SEQ ID NO: 646 - RNA, 45 nt, synthetic construct
  misc_feature 1..45: note = Description of sequence: NP71.2 c4
  source 1..45: mol_type = other RNA
agtagaaacaagggtattttgccactagtctaccctgcttttgct

SEQ ID NO: 647 - RNA, 45 nt, synthetic construct
  misc_feature 1..45: note = Description of sequence: NP71.2 c9
  source 1..45: mol_type = other RNA
agtagaaacaagggtatttttccactagtctaccctgcttttgct

SEQ ID NO: 648 - RNA, 47 nt, synthetic construct
  misc_feature 1..47: note = Description of sequence: NP71.2 c11
  source 1..47: mol_type = other RNA
agtagaaacaagggtatttttcttcactagtctaccctgcttttgct

SEQ ID NO: 649 - RNA, 45 nt, synthetic construct
  misc_feature 1..45: note = Description of sequence: NP71.2 c12
  source 1..45: mol_type = other RNA
agtagaaacaagggtatttttctactagtctaccctgcttttgct

SEQ ID NO: 650 - RNA, 25 nt, synthetic construct
  misc_feature 1..25: note = Description of sequence: Sliding window sequence
  source 1..25: mol_type = other RNA
aaagaaaaatacccttgtttctact

SEQ ID NO: 651 - RNA, 26 nt, synthetic construct
  misc_feature 1..26: note = Description of sequence: Sliding window sequence
  source 1..26: mol_type = other RNA
taaagaaaaatacccttgtttctact

SEQ ID NO: 652 - RNA, 27 nt, synthetic construct
  misc_feature 1..27: note = Description of sequence: Sliding window sequence
  source 1..27: mol_type = other RNA
gtaaagaaaaatacccttgtttctact

SEQ ID NO: 653 - RNA, 36 nt, synthetic construct
  misc_feature 1..36: note = Description of sequence: Sliding window sequence
  source 1..36: mol_type = other RNA
aaacaaccggactagtaaagaaaaatacccttgttt

SEQ ID NO: 654 - RNA, 18 nt, synthetic construct
  misc_feature 1..18: note = Description of sequence: NP71 t-loop alignment sequence
  source 1..18: mol_type = other RNA
tcatctttgttcccataa

SEQ ID NO: 655 - RNA, 18 nt, synthetic construct
  misc_feature 1..18: note = Description of sequence: NP71.2 t-loop alignment sequence
  source 1..18: mol_type = other RNA
aaccaaaacaaccggact

SEQ ID NO: 656 - RNA, 18 nt, synthetic construct
  misc_feature 1..18: note = Description of sequence: NP71.3 t-loop alignment sequence
  source 1..18: mol_type = other RNA
aagctatacaagcggact

SEQ ID NO: 657 - RNA, 18 nt, synthetic construct
  misc_feature 1..18: note = Description of sequence: NP71.7 t-loop alignment sequence
  source 1..18: mol_type = other RNA
aaccaaagcaaccggact

SEQ ID NO: 658 - RNA, 18 nt, synthetic construct
  misc_feature 1..18: note = Description of sequence: NP71.8 t-loop alignment sequence
  source 1..18: mol_type = other RNA
aaccaaaataaccaaact

SEQ ID NO: 659 - RNA, 18 nt, synthetic construct
  misc_feature 1..18: note = Description of sequence: NP71.6 t-loop alignment sequence
  source 1..18: mol_type = other RNA
aaccaaaacggccggact

SEQ ID NO: 660 - RNA, 18 nt, synthetic construct
  misc_feature 1..18: note = Description of sequence: NP71.1 t-loop alignment sequence
  source 1..18: mol_type = other RNA
taggtatactacctaact

SEQ ID NO: 661 - RNA, 18 nt, synthetic construct
  misc_feature 1..18: note = Description of sequence: NP71.4 t-loop alignment sequence
  source 1..18: mol_type = other RNA
ggccatatcggccggact

SEQ ID NO: 662 - RNA, 18 nt, synthetic construct
  misc_feature 1..18: note = Description of sequence: NP71.5 t-loop alignment sequence
  source 1..18: mol_type = other RNA
ggccggggcggccggact

SEQ ID NO: 663-664 - skipped (2 entries)

SEQ ID NO: 665 - RNA, 81 nt, synthetic construct
  misc_feature 1..81: note = Description of sequence: Python script output sequence
  source 1..81: mol_type = other RNA
  misc_structure 9..80: note = loop structure
  misc_difference 8..78: note = n is a t g or c
  misc_difference 29..47: note = Position may be absent
ctagtaannnnnnnnnnnnnnnnnnnnnnnnnnnnnnnnnnnnnnnnnnnnnnnnnnnnnnnnnnnnnnnnnnnnnnnact

SEQ ID NO: 666 - RNA, 82 nt, synthetic construct
  misc_feature 1..82: note = Description of sequence: Python script output sequence
  source 1..82: mol_type = other RNA
  misc_structure 9..80: note = loop structure
  misc_difference 29..47: note = Position may be absent
  misc_difference 8..78: note = n is a t g or c
actagtannnnnnnnnnnnnnnnnnnnnnnnnnnnnnnnnnnnnnnnnnnnnnnnnnnnnnnnnnnnnnnnnnnnnnntact

SEQ ID NO: 667 - RNA, 83 nt, synthetic construct
  misc_feature 1..83: note = Description of sequence: Python script output sequence
  source 1..83: mol_type = other RNA
  misc_structure 9..80: note = loop structure
  misc_difference 29..47: note = Position may be absent
  misc_difference 8..78: note = n is a t g or c
gactagtnnnnnnnnnnnnnnnnnnnnnnnnnnnnnnnnnnnnnnnnnnnnnnnnnnnnnnnnnnnnnnnnnnnnnnnctact

SEQ ID NO: 668 - RNA, 84 nt, synthetic construct
  misc_feature 1..84: note = Description of sequence: Python script output sequence
  source 1..84: mol_type = other RNA
  misc_structure 9..80: note = loop structure
  misc_difference 29..47: note = Position may be absent
  misc_difference 8..78: note = n is a t g or c
ggactagnnnnnnnnnnnnnnnnnnnnnnnnnnnnnnnnnnnnnnnnnnnnnnnnnnnnnnnnnnnnnnnnnnnnnnntctact

SEQ ID NO: 669 - RNA, 85 nt, synthetic construct
  misc_feature 1..85: note = Description of sequence: Python script output sequence
  source 1..85: mol_type = other RNA
  misc_structure 9..80: note = loop structure
  misc_difference 8..78: note = n is a t g or c
  misc_difference 29..47: note = Position may be absent
cggactannnnnnnnnnnnnnnnnnnnnnnnnnnnnnnnnnnnnnnnnnnnnnnnnnnnnnnnnnnnnnnnnnnnnnnttctact

SEQ ID NO: 670 - RNA, 85 nt, synthetic construct
  misc_feature 1..85: note = Description of sequence: Python script output sequence
  source 1..85: mol_type = other RNA
  misc_structure 9..80: note = loop structure
  misc_difference 29..47: note = Position may be absent
  misc_difference 8..78: note = n is a t g or c
gcggactnnnnnnnnnnnnnnnnnnnnnnnnnnnnnnnnnnnnnnnnnnnnnnnnnnnnnnnnnnnnnnnnnnnnnnntttctac

SEQ ID NO: 671 - RNA, 85 nt, synthetic construct
  misc_feature 1..85: note = Description of sequence: Python script output sequence
  source 1..85: mol_type = other RNA
  misc_structure 9..80: note = loop structure
  misc_difference 29..47: note = Position may be absent
  misc_difference 8..78: note = n is a t g or c
agcggacnnnnnnnnnnnnnnnnnnnnnnnnnnnnnnnnnnnnnnnnnnnnnnnnnnnnnnnnnnnnnnnnnnnnnnngtttcta

SEQ ID NO: 672 - RNA, 85 nt, synthetic construct
  misc_feature 1..85: note = Description of sequence: Python script output sequence
  source 1..85: mol_type = other RNA
  misc_structure 9..80: note = loop structure
  misc_difference 29..47: note = Position may be absent
  misc_difference 8..78: note = n is a t g or c
aagcggannnnnnnnnnnnnnnnnnnnnnnnnnnnnnnnnnnnnnnnnnnnnnnnnnnnnnnnnnnnnnnnnnnnnnntgtttct

SEQ ID NO: 673 - RNA, 85 nt, synthetic construct
  misc_feature 1..85: note = Description of sequence: Python script output sequence
  source 1..85: mol_type = other RNA
  misc_structure 9..80: note = loop structure
  misc_difference 29..47: note = Position may be absent
  misc_difference 8..78: note = n is a t g or c
caagcggnnnnnnnnnnnnnnnnnnnnnnnnnnnnnnnnnnnnnnnnnnnnnnnnnnnnnnnnnnnnnnnnnnnnnnnttgtttc

SEQ ID NO: 674 - RNA, 85 nt, synthetic construct
  misc_feature 1..85: note = Description of sequence: Python script output sequence
  source 1..85: mol_type = other RNA
  misc_structure 9..80: note = loop structure
  misc_difference 29..47: note = Position may be absent
  misc_difference 8..78: note = n is a t g or c
acaagcgnnnnnnnnnnnnnnnnnnnnnnnnnnnnnnnnnnnnnnnnnnnnnnnnnnnnnnnnnnnnnnnnnnnnnnncttgttt

SEQ ID NO: 675 - RNA, 85 nt, synthetic construct
  misc_feature 1..85: note = Description of sequence: Python script output sequence
  source 1..85: mol_type = other RNA
  misc_structure 9..80: note = loop structure
  misc_difference 29..47: note = Position may be absent
  misc_difference 8..78: note = n is a t g or c
tacaagcnnnnnnnnnnnnnnnnnnnnnnnnnnnnnnnnnnnnnnnnnnnnnnnnnnnnnnnnnnnnnnnnnnnnnnnccttgtt

SEQ ID NO: 676 - RNA, 85 nt, synthetic construct
  misc_feature 1..85: note = Description of sequence: Python script output sequence
  source 1..85: mol_type = other RNA
  misc_structure 9..80: note = loop structure
  misc_difference 29..47: note = Position may be absent
  misc_difference 8..78: note = n is a t g or c
atacaagnnnnnnnnnnnnnnnnnnnnnnnnnnnnnnnnnnnnnnnnnnnnnnnnnnnnnnnnnnnnnnnnnnnnnnncccttgt

SEQ ID NO: 677 - RNA, 85 nt, synthetic construct
  misc_feature 1..85: note = Description of sequence: Python script output sequence
  source 1..85: mol_type = other RNA
  misc_structure 9..80: note = loop structure
  misc_difference 29..47: note = Position may be absent
  misc_difference 8..78: note = n is a t g or c
tatacaannnnnnnnnnnnnnnnnnnnnnnnnnnnnnnnnnnnnnnnnnnnnnnnnnnnnnnnnnnnnnnnnnnnnnnacccttg

SEQ ID NO: 678 - RNA, 85 nt, synthetic construct
  misc_feature 1..85: note = Description of sequence: Python script output sequence
  source 1..85: mol_type = other RNA
  misc_structure 9..80: note = loop structure
  misc_difference 29..47: note = Position may be absent
  misc_difference 8..78: note = n is a t g or c
ctatacannnnnnnnnnnnnnnnnnnnnnnnnnnnnnnnnnnnnnnnnnnnnnnnnnnnnnnnnnnnnnnnnnnnnnntaccctt

SEQ ID NO: 679 - RNA, 85 nt, synthetic construct
  misc_feature 1..85: note = Description of sequence: Python script output sequence
  source 1..85: mol_type = other RNA
  misc_structure 9..80: note = loop structure
  misc_difference 29..47: note = Position may be absent
  misc_difference 8..78: note = n is a t g or c
gctatacnnnnnnnnnnnnnnnnnnnnnnnnnnnnnnnnnnnnnnnnnnnnnnnnnnnnnnnnnnnnnnnnnnnnnnnataccct

SEQ ID NO: 680 - RNA, 85 nt, synthetic construct
  misc_feature 1..85: note = Description of sequence: Python script output sequence
  source 1..85: mol_type = other RNA
  misc_structure 9..80: note = loop structure
  misc_difference 29..47: note = Position may be absent
  misc_difference 8..78: note = n is a t g or c
agctatannnnnnnnnnnnnnnnnnnnnnnnnnnnnnnnnnnnnnnnnnnnnnnnnnnnnnnnnnnnnnnnnnnnnnnaataccc